(19) United States
(12) Patent Application Publication
(10) Pub. No.: US 2022/0259228 A1
(43) Pub. Date: Aug. 18, 2022

(54) SUBSTITUTED DIAZAHETERO-BICYCLIC COMPOUNDS AND THEIR USE

(71) Applicants: Bayer Aktiengesellschaft, Leverkusen (DE); Bayer Pharma Aktiengesellschaft, Berlin (DE)

(72) Inventors: Martina DELBECK, Heiligenhaus (DE); Michael Hahn, Langenfeld (DE); Thomas Müller, Langenfeld (DE); Klemens Lustig, Wuppertal (DE); Johanna Anlahr, Dortmund (DE); Udo Albus, Florstadt (DE); Doris Gehring, Kelkheim (DE); Björn Rosenstein, Bad Soden-Salmünster (DE); Karl Collins, Düsseldorf (DE); Niels Lindner, Wuppertal (DE); Janine Nicolai, Essen (DE); Moritz Beck-Broichsitter, Darmstadt (DE)

(73) Assignees: Bayer Aktiengesellschaft, Leverkusen (DE); Bayer Pharma Aktiengesellschaft, Berlin (DE)

(21) Appl. No.: 17/447,795

(22) Filed: Sep. 15, 2021

Related U.S. Application Data
(63) Continuation of application No. 16/319,106, filed on Jan. 18, 2019, now abandoned.

(30) Foreign Application Priority Data
Jul. 20, 2016 (EP) 16180315.0
Dec. 14, 2016 (EP) 16203964.8

(51) Int. Cl. C07D 519/00; A61P 11/00; G16C 20/64; A61K 9/00; A61K 9/08; A61K 31/439; A61K 31/444; A61K 31/5377; A61K 31/5386; A61K 31/55; A61K 45/06

(52) U.S. Cl. CPC C07D 519/00; A61P 11/00; G16C 20/64; A61K 9/0043; A61K 9/0053; A61K 9/08; C07B 2200/07; A61K 31/444; A61K 31/5377; A61K 31/5386; A61K 31/55; A61K 45/06; A61K 31/439

(57) ABSTRACT

The present application relates to novel (imidazo[1,2-a]pyridin-3-yl)methyl-substituted diazaheterobicyclic compounds, to processes for preparation thereof, to the use thereof alone or in combinations for treatment and/or prevention of diseases, and to the use thereof for production of medicaments for treatment and/or prevention of diseases, especially for treatment and/or prevention of respiratory disorders including sleep-related respiratory disorders such as obstructive sleep apnoea and central sleep apnoea and snoring. The present application further relates to a method of discovering a compound having TASK-1- and/or TASK-3-blocking properties.

The present application relates to novel (imidazo[1,2-a]pyridin-3-yl)methyl-substituted diazaheterobicyclic compounds, to processes for preparation thereof, to the use thereof alone or in combinations for treatment and/or prevention of diseases, and to the use thereof for production of medicaments for treatment and/or prevention of diseases, especially for treatment and/or prevention of respiratory disorders including sleep-related respiratory disorders such as obstructive sleep apnoea and central sleep apnoea and snoring. The present application further relates to a method of discovering a compound having TASK-1- and/or TASK-3-blocking properties.

Potassium channels are virtually ubiquitous membrane proteins which are involved in a large number of different physiological processes. This also includes the regulation of the membrane potential and the electric excitability of neurons and muscle cells. Potassium channels are divided into three major groups which differ in the number of transmembrane domains (2, 4 or 6). The group of potassium channels where two pore-forming domains are flanked by four transmembrane domains is referred to as K2P channels. Functionally, the K2P channels mediate, substantially time- and voltage-independently, K⁺ background currents, and their contribution to the maintenance of the resting membrane potential is crucial. The family of the K2P channels includes 15 members which are divided into six subfamilies, based on similarities in sequence, structure and function: TWIK, TREK, TASK, TALK, THIK and TRESK.

Of particular interest are TASK-1 (KCNK3 or K2P3.1) and TASK-3 (KCNK9 or K2P9.1) of the TASK (TWIK-related acid-sensitive K⁺ channel) subfamily. Functionally, these channels are characterized in that, during maintenance of voltage-independent kinetics, they have “leak” or “background” streams flowing through them, and they respond to numerous physiological and pathological influences by increasing or decreasing their activity. A characteristic feature of TASK channels is the sensitive reaction to a change of the extracellular pH: at acidic pH the channels are inhibited, and at alkaline pH they are activated.

TASK-1 is expressed mainly in the central nervous system and in the cardiovascular system. Relevant expression of TASK-1 was demonstrated in the brain, in spinal ganglia, in motoneurons of the Nervus hypoglossus and Nervus trigeminus, in the heart, Glomus caroticum, the pulmonary artery, aorta, lung, pancreas, placenta, uterus, kidney, adrenal gland, small intestine and stomach, and also on T lymphocytes. TASK-3 is expressed mainly in the central nervous system. Relevant expression of TASK-3 was demonstrated in the brain, in motoneurons of the Nervus hypoglossus and Nervus trigeminus and in neuroepithelial cells of the Glomus caroticum and the lung, and also on T lymphocytes. A lower expression is found in the heart, stomach, testicular tissue and adrenal gland.

TASK-1 and TASK-3 channels play a role in respiratory regulation. Both channels are expressed in the respiratory neurons of the respiratory centre in the brain stem, inter alia in neurons which generate the respiratory rhythm (ventral respiratory group with pre-Bötzinger complex), and in the noradrenergic Locus caeruleus, and also in serotonergic neurons of the raphe nuclei. Owing to the pH dependency, here the TASK channels have the function of a sensor which translates changes in extracellular pH into corresponding cellular signals [Bayliss et al., Pflugers Arch. 467, 917-929 (2015)]. TASK-1 and TASK-3 are also expressed in the Glomus caroticum, a peripheral chemoreceptor which measures pH, O₂ and CO₂ content of the blood and transmits signals to the respiratory centre in the brain stem to regulate respiration. It was shown that TASK-1 knock-out mice have a reduced ventilatory response (increase of respiratory rate and tidal volume) to hypoxia and normoxic hypercapnia [Trapp et al., J. Neurosci. 28, 8844-8850 (2008)]. Furthermore, TASK-1 and TASK-3 channels were demonstrated in motoneurons of the Nervus hypoglossus, the XIIth cranial nerve, which has an important role in keeping the upper airways open [Berg et al., J. Neurosci. 24, 6693-6702 (2004)].

In a sleep apnoea model in the anaesthetized pig, intranasal administration of a potassium channel blocker which blocks the TASK-1 channel in the nanomolar range led to inhibition of collapsibility of the pharyngeal respiratory musculature and sensitization of the negative pressure reflex of the upper airways. It is assumed that intranasal administration of the potassium channel blocker depolarizes mechanoreceptors in the upper airways and, via activation of the negative pressure reflex, leads to increased activity of the musculature of the upper airways, thus stabilizing the upper airways and preventing collapse. By virtue of this stabilization of the upper airways, the TASK channel blockade may be of great importance for obstructive sleep apnoea and also for snoring [Wirth et al., Sleep f, 699-708 (2013); Kiper et al., Pflugers Arch. 467, 1081-1090 (2015)].

Obstructive sleep apnoea (OSA) is a sleep-related respiratory disorder which is characterized by repeat episodes of obstruction of the upper airways. When breathing in, the patency of the upper airways is ensured by the interaction of two opposite forces. The dilative effects of the musculature of the upper airways counteract the negative intraluminal pressure, which constricts the lumen. The active contraction of the diaphragm and the other auxiliary respiratory muscles generates a negative pressure in the airways, thus constituting the driving force for breathing. The stability of the upper respiratory tract is substantially determined by the coordination and contraction property of the dilating muscles of the upper airways.

The Musculus genioglossus plays a decisive role in the pathogenesis of obstructive sleep apnoea. The activity of the Musculus genioglossus increases with decreasing pressure in the pharynx in the sense of a dilative compensation mechanism. Innervated by the Nervus hypoglossus, it drives the tongue forward and downward, thus widening the pharyngeal airway [Verse et al., Somnologie 3, 14-20 (1999)]. Tensioning of the dilating muscles of the upper airways is modulated inter alia via mechanoreceptors/stretch receptors in the nasal cavity/pharynx [Bouillette et al., J. Appl. Physiol. Respir. Environ. Exerc. Physiol. 46, 772-779 (1979)]. In sleeping patients suffering from serious sleep apnoea, under local anaesthesia of the upper airway an additional reduction of the activity of the Musculus genioglossus can be observed [Berry et al., Am. J. Respir. Crit. Care Med. 156, 127-132 (1997)]. Patients suffering from obstructive sleep apnoea have high mortality and morbidity as a result of cardiovascular disorders such as hypertension, myocardial infarction and stroke [Vrints et al., Acta Clin. Belg. 68, 169-178 (2013)].

In the case of central sleep apnoea, owing to impaired brain function and impaired respiratory regulation there are episodic inhibitions of the respiratory drive. Central respiratory disorders result in mechanical respiratory arrests, i.e. during these episodes there is no breathing activity; temporarily, all respiratory muscles including the diaphragm are at rest. In the case of central sleep apnoea, there is no obstruction of the upper airways.

In the case of primary snoring, there is likewise no obstruction of the upper airways. However, owing to the constriction of the upper airways, the flow rate of the air that is inhaled and exhaled increases. This, combined with the relaxed musculature, causes the soft tissues of the oral cavity and the pharynx to flutter in the stream of air. This gentle vibration then generates the typical snoring noises.

Obstructive snoring (upper airway resistance syndrome, heavy snoring, hypopnoea syndrome) is caused by repeat partial obstruction of the upper airways during sleep. This results in an increased respiratory resistance and thus in an increase in work of breathing with considerable fluctuations in intrathoracic pressure. During inspiration, the negative intrathoracic pressure may reach values similar to those that are encountered as a result of complete airway obstruction during obstructive sleep apnoea. The pathophysiological consequences for heart, circulation and sleep quality correspond to those of obstructive sleep apnoea. As in obstructive sleep apnoea, the pathogenesis is assumed to be an impaired reflex mechanism of the pharynx-dilating muscles during inspiration when sleeping. Frequently, obstructive snoring is the preliminary stage of obstructive sleep apnoea [Hollandt et al., HNO 48, 628-634 (2000)].

In addition, TASK channels also appear to play a role in the apoptosis of neurons. In the animal model of myelin oligodendrocyte glycoprotein (MOG)-induced autoimmune encephalomyelitis, an animal model of multiple sclerosis, TASK-1 knock-out mice showed reduced neuronal degeneration. By preventing neuronal apoptosis, inhibition of TASK channels appears to act neuroprotectively, and may thus be of interest for the treatment of neurodegenerative disorders [Bittner et al., Brain 132, 2501-2516 (2009)].

Furthermore, it has been described that T lymphocytes express TASK-1 and TASK-3 channels and that inhibition of these channels leads to reduced cytokine production and proliferation after stimulation of T lymphocytes. The selective inhibition of TASK channels on T lymphocytes improved the course of the disease in an animal model of multiple sclerosis. The blockade of TASK channels may therefore also be of importance for treatment of autoimmune disorders [Meuth et al., J. Biol. Chem. 283, 14559-14579 (2008)].

TASK-1 and TASK-3 are also expressed in the heart [Rinné et al., J. Mol. Cell. Cardiol. 81, 71-80 (2015)]. Since TASK-1 is expressed particularly strongly in the nervous stimuli conduction system and in the atrium, this channel may have a role in disrupting stimuli conduction or triggering supraventricular arrhythmias. In the heart, TASK-1 appears to contribute to a background current which for its part contributes to maintenance of the resting potential, to action potential duration and to repolarization [Kim et al., Am. J. Physiol. 277, H1669-1678 (1999)]. Using human heart muscle cells, it was shown that blockade of the TASK-1 ion current results in a longer action potential [Limberg et al., Cell. Physiol. Biochem. 28, 613-624 (2011)]. Furthermore, for TASK-1 knock-out mice a prolonged QT time was demonstrated [Decher et al., Cell. Physiol. Biochem. 28, 77-86 (2011)]. Inhibition of TASK channels may therefore be of importance for the treatment of cardiac arrhythmias, in particular atrial fibrillation.

In certain vessels, TASK channels also appear to play a role in the regulation of the vascular tone. A relevant expression of TASK-1 was noticed in smooth muscles of pulmonary and mesenteric arteries. In studies on smooth muscle cells of human pulmonary arteries, it was shown that TASK-1 plays a role in the regulation of the pulmonary vascular tone. TASK-1 may be involved in hypoxic and acidosis-induced pulmonary vasoconstriction [Tang et al., Am. J. Respir. Cell. Mol. Biol. 41, 476-483 (2009)].

In glomerulosa cells of the adrenal cortex, TASK-1 plays a role in potassium conductivity [Czirjak et al., Mol. Endocrinol. 14, 863-874 (2000)].

Possibly, TASK channels also play an important role in apoptosis and tumorigenesis. In breast cancer, colon cancer and lung cancer biopsies and also in metastasizing prostate cancer and in melanoma cells, TASK-3 has been found to be strongly overexpressed [Mu et al., Cancer Cell 3, 297-302 (2003); Kim et al., APMIS 112, 588-594 (2004); Pocsai et al., Cell. Mol. Life Sci. 63, 2364-2376 (2006)]. A point mutation at the TASK-3 channel, which switches off the channel function, simultaneously cancels the tumour-forming action (proliferation, tumour growth, apoptosis resistance) [Mu et al., Cancer Cell 3, 297-302 (2003)]. Overexpression of TASK-3 and TASK-1 in a murine fibroblast cell line (C8 cells) inhibits intracellular apoptosis routes [Liu et al., Brain Res. 1031, 164-173 (2005)]. Accordingly, the blockade of TASK channels may also be relevant for the treatment of various neoplastic disorders.

Therefore, it is an object of the present invention to provide novel substances which act as potent and selective blockers of TASK-1 and TASK-3 channels and, as such, are suitable in particular for the treatment and/or prevention of respiratory disorders including sleep-related respiratory disorders such as obstructive and central sleep apnoea and snoring, and also other disorders.

US 2002/0022624-A1 describes various azaindole derivatives including imidazo[1,2-a]pyridines as substance P antagonists for the treatment of CNS disorders. WO 02/066478-A1 discloses substituted imidazo[1,2-a]pyridines as GnRH antagonists for treatment of sex hormone-dependent disorders. WO 2004/035578-A1 discloses 3-(aminomethyl)imidazo[1,2-a]pyridine derivatives as inhibitors of NO synthase which can be employed for the treatment of various disorders. WO 02/02557-A2 and WO 2009/143156-A2 claim 2-phenylimidazo[1,2-a]pyridine derivatives which, as modulators of GABA_{A} receptors, are suitable for treating CNS disorders. WO 2011/113606-A1 and WO 2012/143796-A2 disclose bicyclic imidazole derivatives suitable for the treatment of bacterial infections and inflammatory disorders. EP 2 671 582-A1 discloses bicyclic imidazole derivatives and options for their therapeutic use as inhibitors of T type calcium channels. WO 2012/130322-A1 describes 2,6-diaryl-3-(piperazinomethyl)imidazo[1,2-a]pyridine derivatives which, by virtue of their HIF-1 inhibiting activity, are suitable in particular for the treatment of inflammatory and hyperproliferative disorders. WO 2014/187922-A1 discloses various 2-phenyl-3-(piperazinomethyl)imidazo[1,2-a]pyridine derivatives as inhibitors of glucose transporters (GLUT) which can be employed for treating inflammatory, proliferative, metabolic, neurological and/or autoimmune disorders. WO 2015/144605-A1 describes acylated bicyclic amine compounds suitable as inhibitors of autotaxin and of lysophosphatidic acid production for the treatment of various disorders. WO 2016/084866-A1 and WO 2016/088813-A1 disclose acylated diazabicyclic compounds which, owing to their antagonistic effect on orexin receptors, can be used for treatment of neurodegenerative disorders, mental disorders and eating and sleep disorders, especially insomnia.

The present invention provides compounds of the general formula (I)

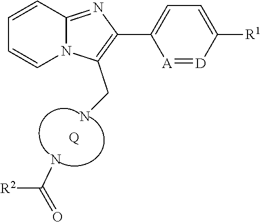

in which
the ring Q is a diazaheterobicyclic system of the formula

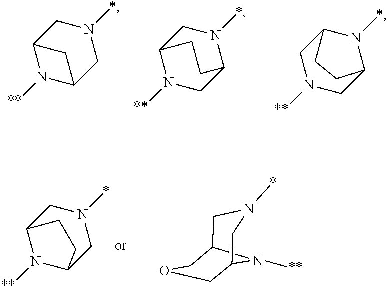

in which * denotes the bond to the adjoining methylene group and ** the bond to the carbonyl group,
A and D are each CH or one of these ring members is CH and the other is N,
R¹ is halogen, cyano, (C₁-C₄)-alkyl, cyclopropyl or cyclobutyl
   where (C₁-C₄)-alkyl may be up to trisubstituted by fluorine and cyclopropyl and cyclobutyl may be up to disubstituted by fluorine,
and
R² is (C₄-C₆)-cycloalkyl in which a ring CH₂ group may be replaced by —O—,
or
R² is a phenyl group of the formula (a), a pyridyl group of the formula (b) or (c) or an azole group of the formula (d)

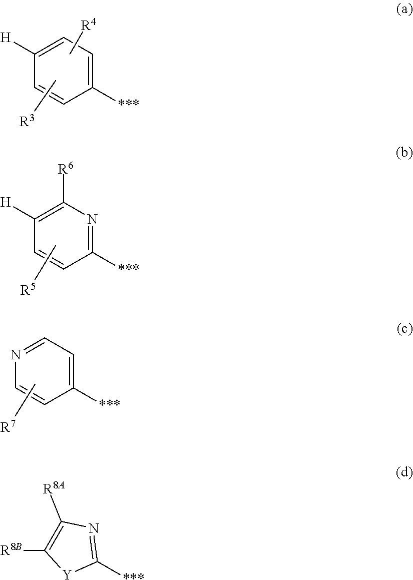

in which *** marks the bond to the adjacent carbonyl group and
R³ is hydrogen, fluorine, chlorine, bromine or methyl,
R⁴ is hydrogen, fluorine, chlorine, bromine, cyano, (C₁-C₃)-alkyl or (C₁-C₃)-alkoxy,
   where (C₁-C₃)-alkyl and (C₁-C₃)-alkoxy may be up to trisubstituted by fluorine,
R⁵ is hydrogen, fluorine, chlorine, bromine or methyl,
R⁶ is hydrogen, (C₁-C₃)-alkoxy, cyclobutyloxy, oxetan-3-yloxy, tetrahydrofuran-3-yloxy or tetrahydro-2H-pyran-4-yloxy,
   where (C₁-C₃)-alkoxy may be substituted up to three times by fluorine,
R⁷ is hydrogen, fluorine, chlorine, bromine, (C₁-C₃)-alkyl or (C₁-C₃)-alkoxy,
R^{8A} and R^{8B} are the same or different and are independently hydrogen or (C₁-C₃)-alkyl
and
Y is N(R⁹), O or S, in which
   R⁹ is hydrogen or (C₁-C₃)-alkyl,
or
R² is an —OR¹⁰ or —NR¹¹R¹² group in which
   R¹⁰ is (C₁-C₄)-alkyl or (C₄-C₆)-cycloalkyl,
   R¹¹ is hydrogen or (C₁-C₃)-alkyl
   and
   R¹² is (C₁-C₆)-alkyl, (C₃-C₆)-cycloalkyl, phenyl or benzyl,
      where (C₁-C₆)-alkyl may be up to trisubstituted by fluorine,
      and
      where phenyl and the phenyl group in benzyl may be up to disubstituted, identically or differently, by a radical selected from the group of fluorine, chlorine, methyl, ethyl and trifluoromethyl,
   or
   R¹¹ and R¹² are joined to one another and, together with the nitrogen atom to which they are bonded, form a pyrrolidine, piperidine, morpholine or thiomorpholine ring,
and the salts, solvates and solvates of the salts thereof.

Inventive compounds are the compounds of the formula (I) and the salts, solvates and solvates of the salts thereof, the compounds of the formulae (I-A), (I-B) and (I-C)) below that are encompassed by formula (I) and the salts, solvates and solvates of the salts thereof, and the compounds cited hereinafter as working examples that are encompassed by formula (I) and the salts, solvates and solvates of the salts thereof, if the compounds cited hereinafter that are encompassed by formula (I) are not already salts, solvates and solvates of the salts.

Preferred salts in the context of the present invention are physiologically acceptable salts of the compounds of the invention. Also encompassed are salts which are not themselves suitable for pharmaceutical applications but can be used, for example, for the isolation, purification or storage of the compounds of the invention.

Physiologically acceptable salts of the compounds of the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, benzenesulphonic acid, toluenesulphonic acid, naphthalenedisulphonic acid, formic acid, acetic acid, trifluoroacetic acid, propionic acid, succinic acid, fumaric acid, maleic acid, lactic acid, tartaric acid, malic acid, citric acid, gluconic acid, benzoic acid and embonic acid.

Solvates in the context of the invention are described as those forms of the compounds of the invention which form a complex in the solid or liquid state by coordination with solvent molecules. Hydrates are a specific form of the solvates in which the coordination is with water. Solvates preferred in the context of the present invention are hydrates.

The compounds of the invention may, depending on their structure, exist in different stereoisomeric forms, i.e. in the form of configurational isomers or else, if appropriate, as conformational isomers (enantiomers and/or diastereomers, including those in the case of atropisomers). The present invention therefore encompasses the enantiomers and diastereomers, and the respective mixtures thereof. The stereoisomerically homogeneous constituents can be isolated from such mixtures of enantiomers and/or diastereomers in a known manner; chromatography processes are preferably employed for the purpose, especially HPLC chromatography on chiral or achiral separation phases. In the case of chiral amines as intermediates or end products, separation is alternatively also possible via diastereomeric salts using enantiomerically pure carboxylic acids.

If the compounds of the invention can occur in tautomeric forms, the present invention encompasses all the tautomeric forms.

The present invention also encompasses all suitable isotopic variants of the compounds of the invention. An isotopic variant of a compound of the invention is understood here to mean a compound in which at least one atom within the compound of the invention has been exchanged for another atom of the same atomic number, but with a different atomic mass from the atomic mass which usually or predominantly occurs in nature. Examples of isotopes which can be incorporated into a compound of the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²³I, ¹²⁹I and ¹³¹I. Particular isotopic variants of a compound according to the invention, especially those in which one or more radioactive isotopes have been incorporated, may be beneficial, for example, for the examination of the mechanism of action or of the active ingredient distribution in the body; due to the comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds of the invention may therefore possibly also constitute a preferred embodiment of the present invention. Isotopic variants of the compounds of the invention can be prepared by commonly used processes known to those skilled in the art, for example by the methods described further down and the procedures described in the working examples, by using corresponding isotopic modifications of the respective reagents and/or starting compounds.

The present invention additionally also encompasses prodrugs of the compounds of the invention. The term “prodrugs” refers here to compounds which may themselves be biologically active or inactive, but are converted while present in the body, for example by a metabolic or hydrolytic route, to compounds of the invention.

In the context of the present invention, unless specified otherwise, the substituents and radicals are defined as follows:

In the context of the invention, (C₁-C₆)-alkyl is a straight-chain or branched alkyl radical having 1 to 6 carbon atoms. Examples include: methyl, ethyl, n-propyl, isopropyl, n-butyl, isobutyl, sec-butyl, tert-butyl, n-pentyl, 2-pentyl, 3-pentyl, neopentyl, n-hexyl, 2-hexyl and 3-hexyl.

In the context of the invention, (C₁-C₄)-alkyl is a straight-chain or branched alkyl radical having 1 to 4 carbon atoms. Examples include: methyl, ethyl, n-propyl, isopropyl, n-butyl, isobutyl, sec-butyl and tert-butyl.

In the context of the invention, (C₁-C₃)-alkyl is a straight-chain or branched alkyl radical having 1 to 3 carbon atoms. Examples include: methyl, ethyl, n-propyl and isopropyl.

(C₁-C₃)-Alkoxy in the context of the invention is a straight-chain or branched alkoxy radical having 1 to 3 carbon atoms. Examples include: methoxy, ethoxy, n-propoxy and isopropoxy.

(C₃-C₆)-Cycloalkyl in the context of the invention is a monocyclic saturated cycloalkyl group having 3 to 6 ring carbon atoms. Examples include: cyclopropyl, cyclobutyl, cyclopentyl and cyclohexyl.

(C₄-C₆)-Cycloalkyl in the context of the invention is a monocyclic saturated cycloalkyl group having 4 to 6 carbon atoms. Examples include: cyclobutyl, cyclopentyl and cyclohexyl.

Halogen in the context of the invention includes fluorine, chlorine, bromine and iodine. Preference is given to fluorine, chlorine or bromine.

In the context of the present invention, all radicals which occur more than once are defined independently of one another. When radicals in the compounds of the invention are substituted, the radicals may be mono- or polysubstituted, unless specified otherwise.

Substitution by one substituent or by two identical or different substituents is preferred.

Particular preference is given to substitution by one substituent.

Preference is given in the context of the present invention to compounds of the formula (I) in which
the ring Q is a diazaheterobicyclic system of the formula

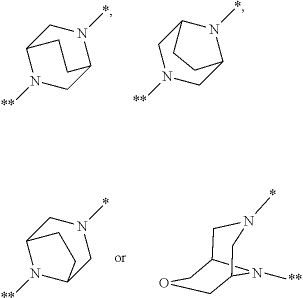

in which * denotes the bond to the adjoining methylene group and ** the bond to the carbonyl group,
A and D are each CH or one of these ring members is CH and the other is N,
R¹ is fluorine, chlorine, bromine, methyl, isopropyl, tert-butyl, cyclopropyl or cyclobutyl
and
R² is cyclobutyl, cyclopentyl or cyclohexyl
or
R² is a phenyl group of the formula (a), a pyridyl group of the formula (b) or an azole group of the formula (d)

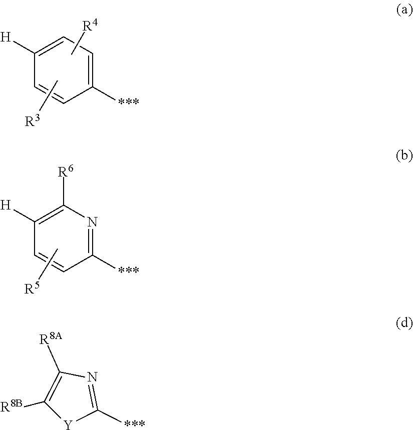

in which *** marks the bond to the adjacent carbonyl group and
R³ is hydrogen, fluorine or chlorine,
R⁴ is fluorine, chlorine, cyano, (C₁-C₃)-alkyl, (C₁-C₃)-alkoxy or trifluoromethoxy,
R⁵ is hydrogen, fluorine, chlorine, bromine or methyl,
R⁶ is (C₁-C₃)-alkoxy which may be up to trisubstituted by fluorine, or cyclobutyloxy,
R^{8A} and R^{8B} are independently hydrogen or methyl
and
Y is N(CH₃), O or S,
or
R² is a —NR¹¹R¹² group in which
   R¹¹ is hydrogen or (C₁-C₃)-alkyl
   and
   R¹² is (C₁-C₆)-alkyl or phenyl,
      where phenyl may be up to disubstituted, identically or differently, by a radical selected from the group of fluorine, chlorine, methyl, ethyl and trifluoromethyl,
   or
   R¹¹ and R¹² are joined to one another and, together with the nitrogen atom to which they are bonded, form a pyrrolidine, piperidine or thiomorpholine ring,
and the salts, solvates and solvates of the salts thereof.

A further preferred embodiment of the present invention comprises compounds of the formula (I) in which
the ring Q is a diazaheterobicyclic system of the formula

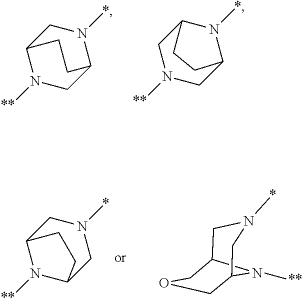

in which * denotes the bond to the adjoining methylene group and ** the bond to the carbonyl group,
A is CH or N,
D is CH,
R¹ is fluorine, chlorine, bromine, methyl, isopropyl, tert-butyl, cyclopropyl or cyclobutyl
and
R² is cyclobutyl, cyclopentyl or cyclohexyl
or
R² is a phenyl group of the formula (a), a pyridyl group of the formula (b) or an azole group of the formula (d)

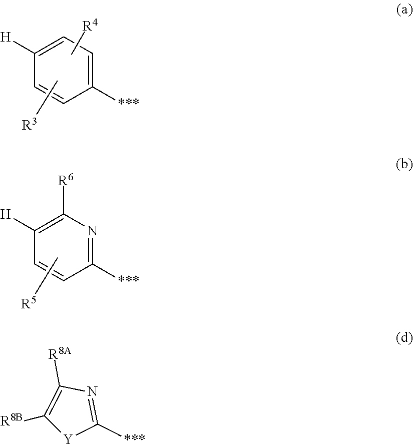

in which *** marks the bond to the adjacent carbonyl group and
R³ is hydrogen, fluorine or chlorine,
R⁴ is fluorine, chlorine, cyano, (C₁-C₃)-alkyl, (C₁-C₃)-alkoxy or trifluoromethoxy,
R⁵ is hydrogen, fluorine, chlorine, bromine or methyl,
R⁶ is (C₁-C₃)-alkoxy which may be up to trisubstituted by fluorine, or cyclobutyloxy,
R^{8A} and R^{8B} are independently hydrogen or methyl
and
Y is N(CH₃), O or S,
or
R² is a —NR¹¹R¹² group in which
   R¹¹ is hydrogen or (C₁-C₃)-alkyl
   and
   R¹² is (C₁-C₆)-alkyl or phenyl,
      where phenyl may be up to disubstituted, identically or differently, by a radical selected from the group of fluorine, chlorine, methyl, ethyl and trifluoromethyl,
   or
   R¹¹ and R¹² are joined to one another and, together with the nitrogen atom to which they are bonded, form a pyrrolidine, piperidine or thiomorpholine ring,
and the salts, solvates and solvates of the salts thereof.

A particular embodiment of the present invention relates to compounds of the formula (I) in which
the ring Q is a diazaheterobicyclic system of the formula

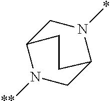

in which * denotes the bond to the adjoining methylene group and ** the bond to the carbonyl group,
and the salts, solvates and solvates of the salts thereof.

A further particular embodiment of the present invention relates to compounds of the formula (I) in which
the ring Q is a diazaheterobicyclic system of the formula

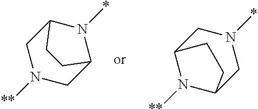

in which * denotes the bond to the adjoining methylene group and ** the bond to the carbonyl group,
and the salts, solvates and solvates of the salts thereof.

A further particular embodiment of the present invention relates to compounds of the formula (I) in which
the ring Q is a diazaheterobicyclic system of the formula

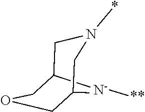

in which * denotes the bond to the adjoining methylene group and ** the bond to the carbonyl group,
and the salts, solvates and solvates of the salts thereof.

A further particular embodiment of the present invention relates to compounds of the formula (I) in which
A and D are each CH,
and the salts, solvates and solvates of the salts thereof.

A further particular embodiment of the present invention relates to compounds of the formula (I) in which
A is N and D is CH,
and the salts, solvates and solvates of the salts thereof.

A further particular embodiment of the present invention relates to compounds of the formula (I) in which
R¹ is chlorine, bromine or isopropyl,
and the salts, solvates and solvates of the salts thereof.

A further particular embodiment of the present invention relates to compounds of the formula (I) in which
R² is cyclobutyl, cyclopentyl or cyclohexyl,
and the salts, solvates and solvates of the salts thereof.

A further particular embodiment of the present invention relates to compounds of the formula (I) in which
R² is a phenyl group of the formula (a)

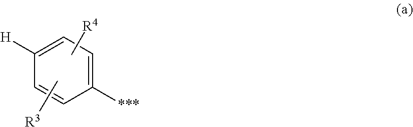

in which *** marks the bond to the adjacent carbonyl group,
R³ is hydrogen, fluorine or chlorine
and
R⁴ is fluorine, chlorine, (C₁-C₃)-alkyl or (C₁-C₃)-alkoxy,
and the salts, solvates and solvates of the salts thereof.

A further particular embodiment of the present invention relates to compounds of the formula (I) in which
R² is a pyridyl group of the formula (b)

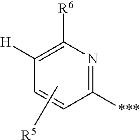

in which *** marks the bond to the adjacent carbonyl group,
R⁵ is hydrogen, fluorine, chlorine, bromine or methyl
and
R⁶ is (C₁-C₃)-alkoxy which may be up to trisubstituted by fluorine, or cyclobutyloxy,
and the salts, solvates and solvates of the salts thereof.

In the context of the present invention, particular preference is given to compounds of the formula (I) in which
the ring Q is a diazaheterobicyclic system of the formula

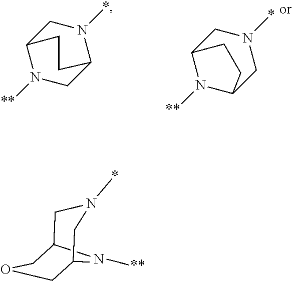

in which * denotes the bond to the adjoining methylene group and ** the bond to the carbonyl group,
A and D are each CH or one of these ring members is CH and the other is N,
R¹ is chlorine, bromine, isopropyl or cyclopropyl
and
R² is cyclobutyl, cyclopentyl or cyclohexyl
or
R² is a phenyl group of the formula (a), a pyridyl group of the formula (b) or an azole group of the formula (d)

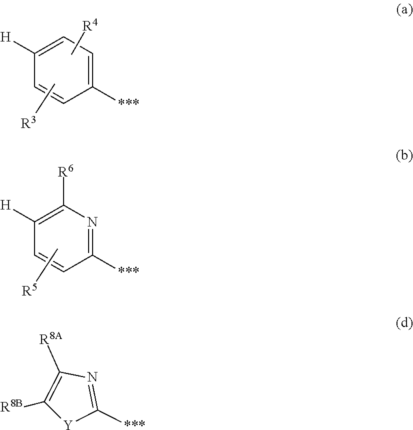

in which *** marks the bond to the adjacent carbonyl group and
R³ is hydrogen, fluorine or chlorine,
R⁴ is fluorine, chlorine, methyl, isopropyl, methoxy or ethoxy,
R⁵ is hydrogen, fluorine, chlorine, bromine or methyl,
R⁶ is methoxy, difluoromethoxy, trifluoromethoxy, isopropoxy or cyclobutyloxy,
R^{8A} and R^{8B} are each hydrogen
and
Y is N(CH₃),
and the salts, solvates and solvates of the salts thereof.

A further particularly preferred embodiment of the present invention comprises compounds of the formula (I) in which
the ring Q is a diazaheterobicyclic system of the formula

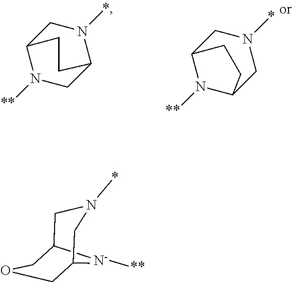

in which * denotes the bond to the adjoining methylene group and ** the bond to the carbonyl group,
A is CH or N,
D is CH,
R¹ is chlorine, bromine, isopropyl or cyclopropyl
and
R² is cyclobutyl, cyclopentyl or cyclohexyl
or
R² is a phenyl group of the formula (a), a pyridyl group of the formula (b) or an azole group of the formula (d)

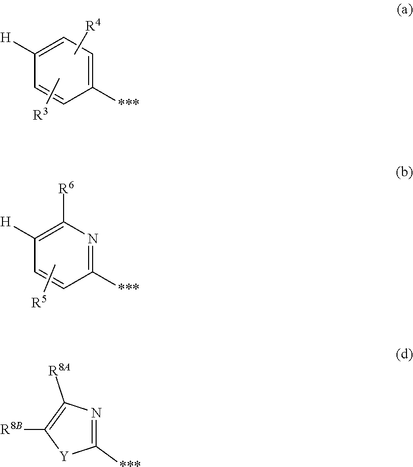

in which *** marks the bond to the adjacent carbonyl group and
R³ is hydrogen, fluorine or chlorine,
R⁴ is fluorine, chlorine, methyl, isopropyl, methoxy or ethoxy,
R⁵ is hydrogen, fluorine, chlorine, bromine or methyl,
R⁶ is methoxy, difluoromethoxy, trifluoromethoxy, isopropoxy or cyclobutyloxy,
R^{8A} and R^{8B} are each hydrogen
and
Y is N(CH₃),
and the salts, solvates and solvates of the salts thereof.

The individual radical definitions specified in the respective combinations or preferred combinations of radicals are, independently of the respective combinations of the radicals specified, also replaced as desired by radical definitions of other combinations. Particular preference is given to combinations of two or more of the abovementioned preferred ranges.

The invention furthermore provides a process for preparing the compounds of the formula (I) according to the invention, characterized in that a compound of the formula (II)

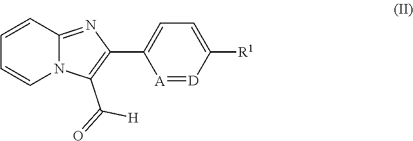

in which A, D and R¹ have the definitions given above
is reacted in the presence of a suitable reducing agent either
[A] with a compound of the formula (III)

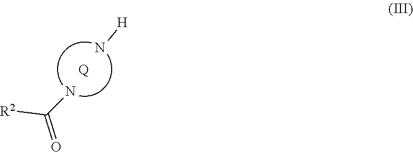

in which R² and the ring Q have the definitions given above
to give a compound of the formula (I)
or
[B] with a protected diazaheterobicyclic system of the formula (IV)

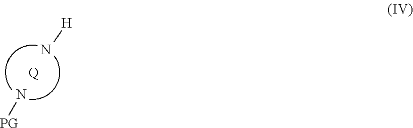

in which the ring Q has the definition given above
and
PG is a suitable amino protecting group, for example tert-butoxycarbonyl, benzyloxycarbonyl or (9H-fluoren-9-ylmethoxy)carbonyl
to give initially a compound of the formula (V)

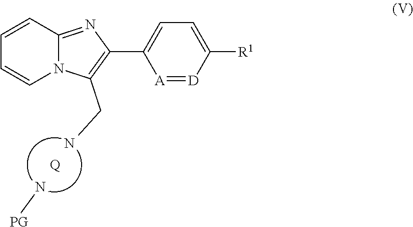

in which A, D, PG, R¹ and the ring Q have the definitions given above,
then the protecting group PG is removed and the resulting compound of the formula (VI)

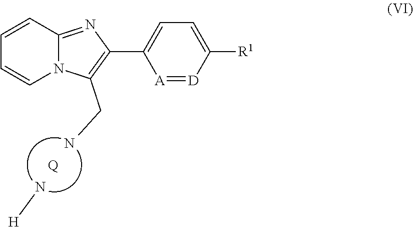

in which A, D, R¹ and the ring Q have the definitions given above
is then reacted, depending on the specific definition of the R² radical,
[B-1] with a carboxylic acid of the formula (VII)

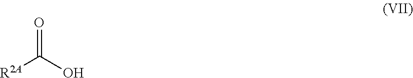

where
R^{2A} is (C₄-C₆)-cycloalkyl in which a ring CH₂ group may be replaced by —O—, or is a phenyl group of the formula (a), a pyridyl group of the formula (b) or (c) or an azole group of the formula (d) as described above, with activation of the carboxylic acid function in (VII) or is reacted with the corresponding acid chloride of the formula (VIII)

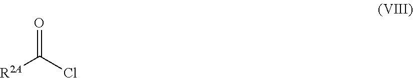

in which R^{A} has the definition given above
to give a compound of the formula (I-A)

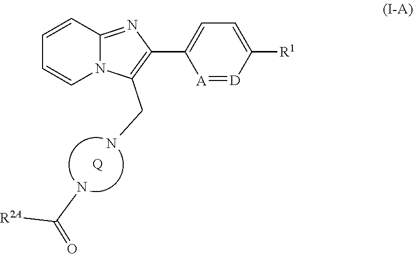

in which A, D, R¹, R^{2A} and the ring Q have the definitions given above
or
[B-2] with a chloroformate or carbamoyl chloride of the formula (IX)

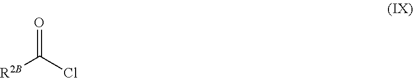

where
R^{2B} is the —OR¹⁰ or —NR^{11A}R¹² group in which
   R¹⁰ and R¹² have the definitions given above
   and
   R^{11A} has the definition of R¹¹ given above, but is not hydrogen,
to give a compound of the formula (I-B)

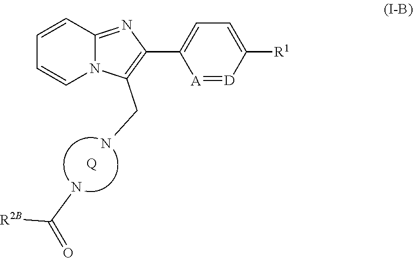

in which A, D, R¹, R^{2B} and the ring Q have the definitions given above
or
[B-3] with an isocyanate of the formula (X)

R¹²—N═C═O (X)
in which R¹² has the definition given above
to give a compound of the formula (I-C)

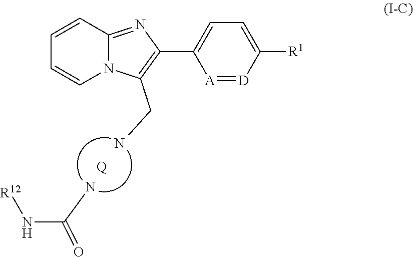

in which A, D, R¹, R¹² and the ring Q have the definitions given above
   and the compounds of the formulae (I), (I-A), (I-B) or (I-C) thus obtained are optionally separated into their enantiomers and/or diastereomers and/or optionally converted with the appropriate (i) solvents and/or (ii) acids to the solvates, salts and/or solvates of the salts thereof.

Suitable reducing agents for the process steps [A] (II)+(III)→(I) and [B] (II)+(IV)→(V) [reductive aminations] for such purposes are customary alkali metal borohydrides such as sodium borohydride, sodium cyanoborohydride or sodium triacetoxyborohydride; preference is given to using sodium triacetoxyborohydride. The addition of an acid, such as acetic acid in particular, and/or of a dehydrating agent, for example molecular sieve or trimethyl orthoformate or triethyl orthoformate, may be advantageous in these reactions.

Suitable solvents for these reactions are especially alcohols such as methanol, ethanol, n-propanol or isopropanol, ethers such as diisopropyl ether, methyl tert-butyl ether, tetrahydrofuran, 1,4-dioxane or 1,2-dimethoxyethane, polar aprotic solvents such as acetonitrile or N,N-dimethylformamide (DMF) or mixtures of such solvents; preference is given to using tetrahydrofuran. The reactions are generally effected within a temperature range of 0° C. to +50° C.

The protecting group PG used in compound (IV) may be a standard amino protecting group, for example tert-butoxycarbonyl (Boc), benzyloxycarbonyl (Z) or (9H-fluoren-9-ylmethoxy)carbonyl (Fmoc); preference is given to using tert-butoxycarbonyl (Boc). The detachment of the protecting group in process step [B] (V)→(VI) is effected by known methods. Thus, the tert-butoxycarbonyl group is typically detached by treatment with a strong acid such as hydrogen chloride, hydrogen bromide or trifluoroacetic acid, in an inert solvent such as diethyl ether, 1,4-dioxane, dichloromethane or acetic acid. In the case of benzyloxycarbonyl as protecting group, this is preferably removed by hydrogenolysis in the presence of a suitable palladium catalyst such as palladium on activated carbon. The (9H-fluoren-9-ylmethoxy)carbonyl group is generally detached with the aid of a secondary amine base such as diethylamine or piperidine [see, for example, T. W. Greene and P. G. M. Wuts, Protective Groups in Organic Synthesis, Wiley, New York, 1999; P. J. Kocienski, Protecting Groups, 3^{rd} edition, Thieme, 2005].

Particular compounds of the formula (V), especially those in which PG is tert-butoxycarbonyl, likewise have significant inhibitory activity with respect to TASK-1 and/or TASK-3, and in this respect are also encompassed by the scope of definition of the present invention, i.e. the compounds of the formula (I).

The process step [B-1] (VI)+(VII)→(I-A) [amide formation] is conducted by known methods with the aid of a condensing or activating agent. Suitable agents of this kind are, for example, carbodiimides such as N,N′-diethyl-, N,N′-dipropyl-, N,N′-diisopropyl-, N,N′-dicyclohexylcarbodiimide (DCC) or N-(3-dimethylaminopropyl)-N′-ethylcarbodiimide hydrochloride (EDC), phosgene derivatives such as N,N′-carbonyldiimidazole (CDI) or isobutyl chloroformate, 1,2-oxazolium compounds such as 2-ethyl-5-phenyl-1,2-oxazolium 3-sulphate or 2-tert-butyl-5-methylisoxazolium perchlorate, acylamino compounds such as 2-ethoxy-1-ethoxycarbonyl-1,2-dihydroquinoline, α-chlorenamines such as 1-chloro-N,N,2-trimethylprop-1-en-1-amine, 1,3,5-triazine derivatives such as 4-(4,6-dimethoxy-1,3,5-triazin-2-yl)-4-methylmorpholinium chloride, phosphorus compounds such as n-propanephosphonic anhydride (PPA), diethyl cyanophosphonate, diphenylphosphoryl azide (DPPA), bis(2-oxo-3-oxazolidinyl)phosphoryl chloride, benzotriazol-1-yloxytris(dimethylamino)phosphonium hexafluorophosphate or benzotriazol-1-yloxytris(pyrrolidino)phosphonium hexafluorophosphate (PyBOP), or uronium compounds such as O-(benzotriazol-1-yl)-N,N,N′,N′-tetramethyluronium tetrafluoroborate (TBTU), O-(1H-6-chlorobenzotriazol-1-yl)-1,1,3,3-tetramethyluronium tetrafluoroborate (TCTU), O-(benzotriazol-1-yl)-N,N,N,N′-tetramethyluronium hexafluorophosphate (HBTU), O-(7-azabenzotriazol-1-yl)-N,N,N,N′-tetramethyluronium hexafluorophosphate (HATU) or 2-(2-oxo-1-(2H)-pyridyl)-1,1,3,3-tetramethyluronium tetrafluoroborate (TPTU), optionally in combination with further auxiliaries such as 1-hydroxybenzotriazole (HOBt) or N-hydroxysuccinimide (HOSu), and also as base an alkali metal carbonate, for example sodium carbonate or potassium carbonate, or a tertiary amine base such as triethylamine, N,N-diisopropylethylamine, N-methylmorpholine (NMM), N-methylpiperidine (NMP), pyridine or 4-N,N-dimethylaminopyridine (DMAP). The condensing agent or activating agent used with preference is O-(7-azabenzotriazol-1-yl)-N,N,N,N′-tetramethyluronium hexafluorophosphate (HATU) in combination with N,N-diisopropylethylamine as base.

The alternative process via the carbonyl chloride (VIII) [(VI)+(VIII)→(I-A)] is generally effected in the presence of a base such as sodium carbonate, potassium carbonate, triethylamine, N,N-diisopropylethylamine, N-methylmorpholine (NMM), N-methylpiperidine (NMP), pyridine, 2,6-dimethylpyridine, 4-N,N-dimethylaminopyridine (DMAP), 1,5-diazabicyclo[4.3.0]non-5-ene (DBN) or 1,8-diazabicyclo[5.4.0]undec-7-ene (DBU); preference is given to using triethylamine or N,N-diisopropylethylamine.

Suitable inert solvents for these amide-forming reactions are, for example, ethers such as diethyl ether, diisopropyl ether, methyl tert-butyl ether, tetrahydrofuran, 1,4-dioxane, 1,2-dimethoxyethane or bis(2-methoxyethyl) ether, hydrocarbons such as benzene, toluene, xylene, pentane, hexane or cyclohexane, halohydrocarbons such as dichloromethane, trichloromethane, carbon tetrachloride, 1,2-dichloroethane, trichloroethylene or chlorobenzene, or polar aprotic solvents such as acetone, methyl ethyl ketone, ethyl acetate, acetonitrile, butyronitrile, pyridine, dimethyl sulphoxide (DMSO), N,N-dimethylformamide (DMF), N,N′-dimethylpropyleneurea (DMPU) or N-methylpyrrolidinone (NMP); it is also possible to use mixtures of such solvents. Preference is given to using dichloromethane, 1,2-dichloroethane, tetrahydrofuran, N,N-dimethylformamide or mixtures of these solvents. The reactions are generally conducted within a temperature range of from −20° C. to +60° C., preferably at from 0° C. to +40° C.

The process [B-2] (VI)+(IX)→(I-B) [formation of urethanes or substituted ureas] is conducted under similar reaction conditions with regard to solvent, addition of base and temperature as described above for the amide formation [B-1] (VI)+(VIII)→(I-A).

The reaction [B-3] (VI)+(X)→(I-C) is likewise effected in one of the above-listed inert solvents or solvent mixtures at a temperature in the range from 0° C. to +60° C.; the addition of a base in this reaction can optionally be dispensed with.

The amine compound (VI) can also be used in the process steps [B-1] (VI)+(VII) or (VIII)→(I-A), [B-2] (VI)+(IX)→(I-B) and [B-3] (VI)+(X)→(I-C) in the form of a salt, for example as hydrochloride or trifluoroacetate. In such a case, the conversion is effected in the presence of an appropriately increased amount of the respective auxiliary base used.

The processes described above can be conducted at standard, elevated or reduced pressure (for example in the range from 0.5 to 5 bar); in general, the reactions are each conducted at standard pressure.

Separation of the compounds of the invention into the corresponding enantiomers and/or diastereomers can, as appropriate, also be effected at the early stage of the compounds (III), (IV), (V) or (VI), which are then converted further in separated form in accordance with the process steps described above. Such a separation of stereoisomers can be conducted by customary methods known to the person skilled in the art. In the context of the present invention, preference is given to using chromatographic methods on chiral or achiral separation phases; in the case of chiral amines as intermediates or end products, separation can alternatively be effected via diastereomeric salts with the aid of enantiomerically pure carboxylic acids.

For their part, the compounds of the formula (II) can be prepared by processes known from the literature by condensing 2-aminopyridine (XI)

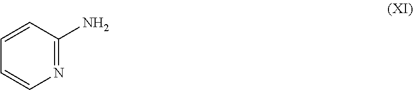

under the influence of a base with a compound of the formula (XII)

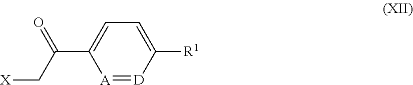

in which A, D and R¹ have the definitions given above
and
X is a suitable leaving group, for example chlorine, bromine or iodine,
to give an imidazo[1,2-a]pyridine derivative of the formula (XIII)

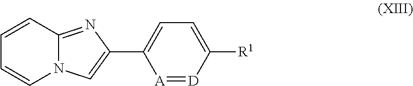

in which A, D and R¹ have the definitions given above
and then formylating this with a mixture of N,N-dimethylformamide and phosphorus oxychloride to give (II).

The condensation reaction (XI)+(XII)→(XIII) is typically conducted in an alcoholic solvent such as methanol, ethanol, n-propanol, isopropanol or n-butanol, in an ether such as diethyl ether, diisopropyl ether, methyl tert-butyl ether, tetrahydrofuran, 1,4-dioxane, 1,2-dimethoxyethane or bis(2-methoxyethyl) ether, in a dipolar aprotic solvent such as N,N-dimethylformamide (DMF), N,N′-dimethylpropyleneurea (DMPU) or N-methylpyrrolidinone (NMP), or else in water, at a temperature in the range from +50° C. to +150° C.; preference is given to using ethanol or water as solvent.

Bases suitable for this reaction are especially alkali metal hydrogencarbonates or carbonates such as sodium hydrogencarbonate or potassium hydrogencarbonate or lithium carbonate, sodium carbonate, potassium carbonate or caesium carbonate, alkali metal hydroxides such as sodium hydroxide or potassium hydroxide, or else alumina; preference is given to using sodium hydrogencarbonate or sodium hydroxide. Optionally—if the reaction temperature is increased correspondingly—the reaction can also be effected without addition of a base.

The regioselective formylation (XIII)→(II) is effected under the standard conditions of a Vilsmaier-Haack reaction by treatment of (XIII) with a preformed mixture of N,N-dimethylformamide and phosphorus oxychloride which is used in a large excess and simultaneously also serves as solvent. The reaction is generally conducted within a temperature range of from 0° C. to +100° C.

The compounds of the formulae (III), (IV), (VII), (VIII), (IX), (X), (XI) and (XII) are either commercially available or described as such in the literature, or they can be prepared in a simple manner from other commercially available compounds by methods familiar to the person skilled in the art and known from the literature. Numerous detailed procedures and further literature references can also be found in the experimental section, in the section on the preparation of the starting compounds and intermediates.

The preparation of the compounds of the invention can be illustrated by way of example by the following reaction schemes:

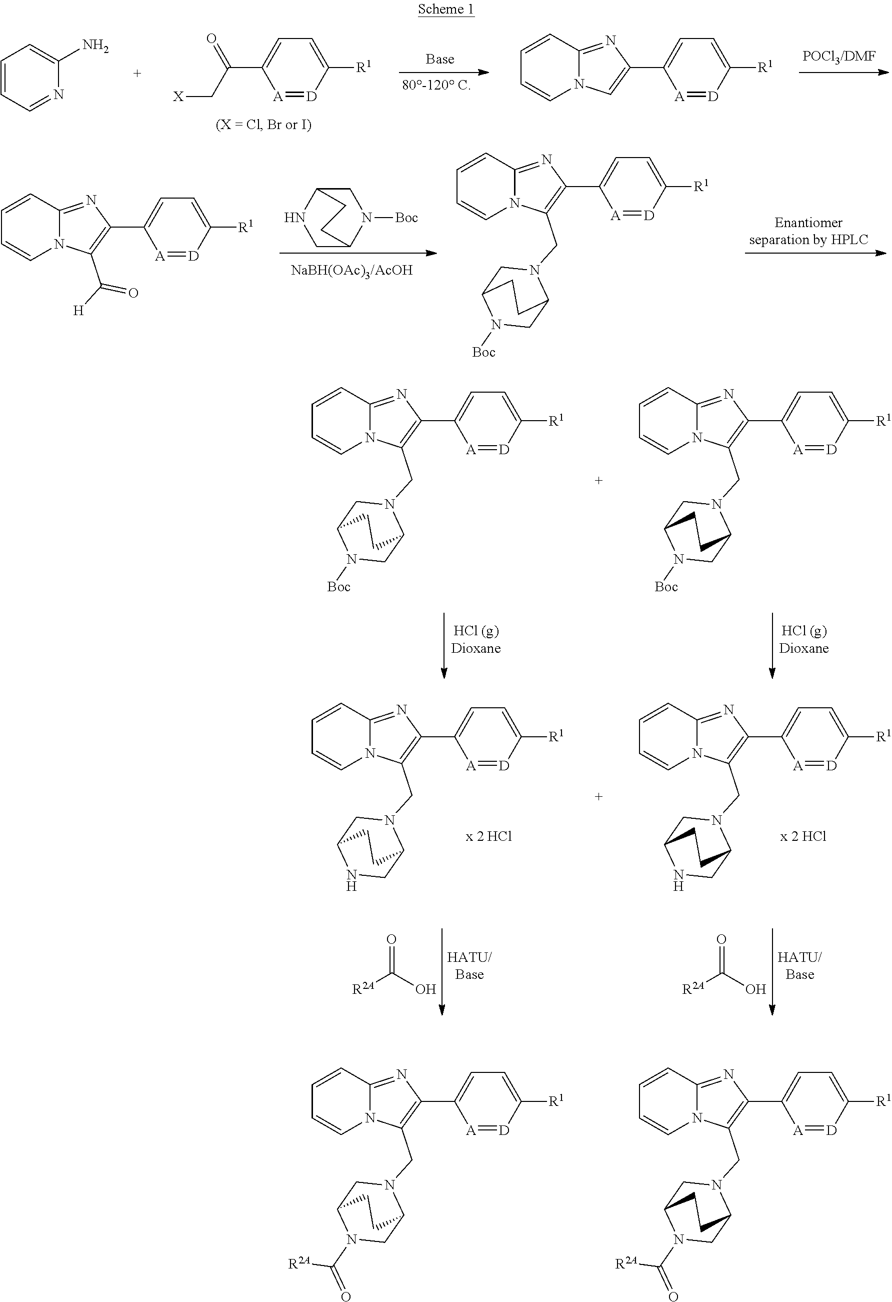

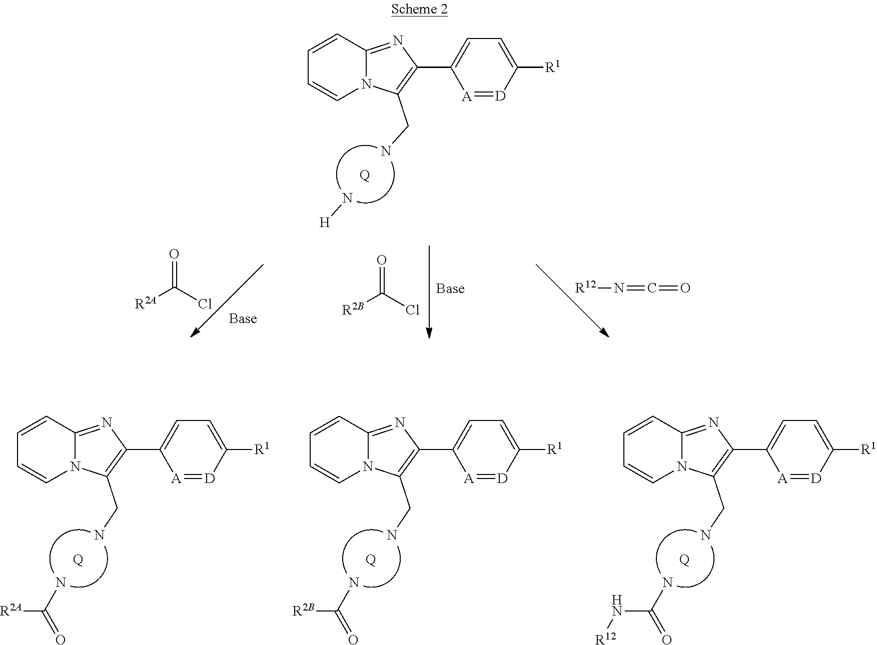

The compounds of the invention have valuable pharmacological properties and can be used for prevention and treatment of disorder in humans and animals.

The compounds of the invention are potent and selective blockers of TASK-1 and TASK-3 channels and are therefore suitable for the treatment and/or prevention of disorders and pathological processes, in particular those caused by activation of TASK-1 and/or TASK-3 or by activated TASK-1 and/or TASK-3, and of disorders secondary to damage caused by TASK-1 and/or TASK-3.

For the purposes of the present invention, this includes in particular disorders from the group of the respiratory disorders and sleep-related respiratory disorders, such as obstructive sleep apnoea (in adults and children), primary snoring, obstructive snoring (upper airway resistance syndrome, heavy snoring, hypopnoea syndrome), central sleep apnoea, mixed sleep apnoea, Cheyne-Stokes respiration, primary sleep apnoea of infancy, apparent life-threatening event, central sleep apnoea as a result of the use of medicaments or the use of other substances, obesity hypoventilation syndrome, disrupted central respiratory drive, sudden infant death, primary alveolar hypoventilation syndrome, postoperative hypoxia and apnoea, muscular respiratory disorders, respiratory disorders following long-term ventilation, respiratory disorders during adaptation in high mountains, acute and chronic pulmonary diseases with hypoxia and hypercapnia, sleep-related non-obstructive alveolar hypoventilation and the congenital central alveolar hypoventilation syndrome.

The compounds of the invention can additionally be used for treatment and/or prevention of neurodegenerative disorders such as dementia, dementia with Lewy bodies, Alzheimer's disease, Parkinson's disease, Huntington's disease, Pick's disease, Wilson's disease, progressive supranuclear paresis, corticobasal degeneration, tauopathy, frontotemporal dementia and parkinsonism linked to chromosome 17, multisystem atrophy, spinocerebellar ataxias, spinobulbar muscular atrophy of the Kennedy type, Friedreich's ataxia, dentatorubral-pallidoluysian atrophy, amyotrophic lateral sclerosis, primary lateral sclerosis, spinal muscular atrophy, Creutzfeldt-Jakob disease and variants of Creutzfeldt-Jakob disease, infantile neuroaxonal dystrophy, neurodegeneration with brain iron accumulation, frontotemporal lobar degeneration with ubiquitin proteasome system and familial encephalopathy with neuroserpin inclusions.

In addition, the compounds of the invention can be used for treatment and/or prevention of neuroinflammatory and neuroimmunological disorders of the central nervous system (CNS), for example multiple sclerosis (Encephalomyelitis disseminata), transverse myelitis, Neuromyelitis optica, acute disseminated encephalomyelitis, optic neuritis, meningitis, encephalitis, demyelinating diseases and also inflammatory vascular changes in the central nervous system.

Moreover, the compounds of the invention are suitable for the treatment and/or prevention of neoplastic disorders such as, for example, skin cancer, breast cancer, lung cancer, colon cancer and prostate cancer.

The compounds of the invention are also suitable for treatment and/or prevention of cardiac arhythmias, for example atrial and ventricular arrhythmias, conduction defects such as first- to third-degree atrio-ventricular blocks, supraventricular tachyarrhythmia, atrial fibrillation, atrial flutter, ventricular fibrillation, ventricular flutter, ventricular tachyarhythmia, Torsade de pointes tachycardia, atrial and ventricular extrasystoles, AV-junctional extrasystoles, sick sinus syndrome, syncopes and AV nodal re-entrant tachycardia.

Further cardiovascular disorders where the compounds of the invention can be employed for treatment and/or prevention are, for example, heart failure, coronary heart disease, stable and unstable angina pectoris, high blood pressure (hypertension), pulmonary-arterial hypertension (PAH) and other forms of pulmonary hypertension (PH), renal hypertension, peripheral and cardial vascular disorders, Wolff-Parkinson-White syndrome, acute coronary syndrome (ACS), autoimmune cardiac disorders (pericarditis, endocarditis, valvolitis, aortitis, cardiomyopathies), boxer cardiomyopathy, aneurysms, shock such as cardiogenic shock, septic shock and anaphylactic shock, furthermore thromboembolic disorders and ischaemias such as myocardial ischaemia, myocardial infarction, stroke, cardiac hypertrophy, transient and ischaemic attacks, preeclampsia, inflammatory cardiovascular disorders, spasms of the coronary arteries and peripheral arteries, oedema formation such as, for example, pulmonary oedema, cerebral oedema, renal oedema or oedema caused by heart failure, peripheral circulatory disturbances, reperfusion damage, arterial and venous thromboses, microalbuminuria, myocardial insufficiency, endothelial dysfunction, micro- and macrovascular damage (vasculitis), and also to prevent restenoses, for example after thrombolysis therapies, percutaneous transluminal angioplasties (PTA), percutaneous transluminal coronary angioplasties (PTCA), heart transplants and bypass operations.

In the context of the present invention, the term “heart failure” encompasses both acute and chronic forms of heart failure, and also specific or related disease types thereof, such as acute decompensated heart failure, right heart failure, left heart failure, global failure, ischaemic cardiomyopathy, dilatative cardiomyopathy, hypertrophic cardiomyopathy, idiopathic cardiomyopathy, congenital heart defects, heart valve defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders and diastolic and systolic heart failure.

The compounds of the invention can additionally be used for treatment and/or prevention of asthmatic disorders of varying severity with intermittent or persistent characteristics (refractive asthma, bronchial asthma, allergic asthma, intrinsic asthma, extrinsic asthma, medicament- or dust-induced asthma), of various forms of bronchitis (chronic bronchitis, infectious bronchitis, eosinophilic bronchitis), of bronchiectasis, pneumonia, farmer's lung and related disorders, coughs and colds (chronic inflammatory cough, iatrogenic cough), inflammation of the nasal mucosa (including medicament-related rhinitis, vasomotoric rhinitis and seasonal allergic rhinitis, for example hay fever) and of polyps.

The compounds of the invention are also suitable for treatment and/or prevention of renal disorders, in particular renal insufficiency and kidney failure. In the context of the present invention, the terms “renal insufficiency” and “kidney failure” encompass both acute and chronic manifestations thereof and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and immunocomplex-induced kidney disorders, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphataemia and/or need for dialysis. The present invention also encompasses the use of the compounds of the invention for treatment and/or prevention of sequelae of renal insufficiency, for example hypertension, pulmonary oedema, heart failure, uraemia, anaemia, electrolyte disturbances (for example hyperkalaemia, hyponatraemia) and disturbances in bone and carbohydrate metabolism.

In addition, the compounds of the invention are suitable for treatment and/or prevention of disorders of the urogenital system, for example benign prostate syndrome (BPS), benign prostate hyperplasia (BPH), benign prostate enlargement (BPE), bladder outlet obstruction (BOO), lower urinary tract syndromes (LUTS), neurogenic overactive bladder (OAB), incontinence, for example mixed urinary incontinence, urge urinary incontinence, stress urinary incontinence or overflow urinary incontinence (MUI, UUI, SUI, OUI), pelvic pain, and also erectile dysfunction and female sexual dysfunction.

The compounds of the invention are further suitable for treatment and/or prevention of inflammatory disorders and autoimmune disorders such as, for example, rheumatoid disorders, inflammatory eye disorders, chronic obstructive pulmonary disease (COPD), acute respiratory distress syndrome (ARDS), acute lung injury (ALI), alpha-1-antitrypsin deficiency (AATD), pulmonary emphysema (e.g. pulmonary emphysema induced by cigarette smoke), cystic fibrosis (CF), sepsis (SIRS), multiple organ failure (MODS, MOF), inflammatory disorders of the kidney, chronic intestinal inflammations (IBD, Crohn's disease, ulcerative colitis), pancreatitis, peritonitis, cystitis, urethritis, prostatitis, epidimytitis, oophoritis, salpingitis and vulvovaginitis, and also for the treatment and/or prevention of fibrotic disorders of internal organs such as, for example, the lung, the heart, the kidney, the bone marrow and especially the liver, of dermatological fibroses and of fibrotic disorders of the eye. In the context of the present invention, the term “fibrotic disorders” includes in particular disorders such as hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis, peritoneal fibrosis and similar fibrotic disorders, scleroderma, morphoea, keloids, hypertrophic scarring, naevi, diabetic retinopathy, proliferative vitroretinopathy and disorders of the connective tissue (for example sarcoidosis). The compounds of the invention can likewise be used for promotion of wound healing, for controlling postoperative scarring, for example following glaucoma operations and cosmetically for ageing or keratinized skin.

In addition, the compounds of the invention can be used for treatment and/or prevention of arteriosclerosis, impaired lipid metabolism and dyslipidaemias (hypolipoproteinaemia, hypertriglyceridaemia, hyperlipidaemia, combined hyperlipidaemias, hypercholesterolaemia, abetalipoproteinaemia, sitosterolaemia), xanthomatosis, Tangier disease, adiposity, obesity, metabolic disorders (metabolic syndrome, hyperglycaemia, insulin-dependent diabetes, non-insulin-dependent diabetes, gestation diabetes, hyperinsulinaemia, insulin resistance, glucose intolerance and diabetic sequelae, such as retinopathy, nephropathy and neuropathy), of anaemias such as haemolytic anaemias, in particular haemoglobinopathies such as sickle cell anaemia and thalassaemias, megaloblastic anaemias, iron deficiency anaemias, anaemias owing to acute blood loss, displacement anaemias and aplastic anaemias, of disorders of the gastrointestinal tract and the abdomen (glossitis, gingivitis, periodontitis, oesophagitis, eosinophilic gastroenteritis, mastocytosis, Crohn's disease, colitis, proctitis, anus pruritis, diarrhoea, coeliac disease, hepatitis, hepatic fibrosis, cirrhosis of the liver, pancreatitis and cholecystitis), of disorders of the central nervous system (stroke, epilepsy, depression), immune disorders, thyroid disorders (hyperthyreosis), skin disorders (psoriasis, acne, eczema, neurodermatitis, various forms of dermatitis, keratitis, bullosis, vasculitis, cellulitis, panniculitis, lupus erythematosus, erythema, lymphomas, skin cancer, Sweet syndrome, Weber-Christian syndrome, scar formation, wart formation, chilblains), of inflammatory eye diseases (saccoidosis, blepharitis, conjunctivitis, iritis, uveitis, chorioiditis, ophthalmitis), of viral diseases (caused by influenza, adeno and corona viruses, for example HPV, HCMV, HIV, SARS), of disorders of the skeletal bone and the joints and also the skeletal muscle, of inflammatory arterial lesions (various forms of arteritis, for example endarteritis, mesarteritis, periarteritis, panarteritis, arteritis rheumatica, arteritis deformans, arteritis temporalis, arteritis cranialis, arteritis gigantocellularis and arteritis granulomatosa, and also Horton syndrome, Churg-Strauss syndrome and Takayasu arteritis), of Muckle-Well syndrome, of Kikuchi disease, of polychondritis, dermatosclerosis and also other disorders having an inflammatory or immunological component, for example cataract, cachexia, osteoporosis, gout, incontinence, leprosy, Sezary syndrome and paraneoplastic syndrome, in the event of rejection reactions after organ transplants and for wound healing and angiogenesis particularly in the case of chronic wounds.

By virtue of their property profile, the compounds of the invention are preferably suitable for treatment and/or prevention of respiratory disorders, in particular of sleep-related respiratory disorders such as obstructive and central sleep apnoea and also primary and obstructive snoring, for treatment and/or prevention of cardiac arrhythmias and also for treatment and/or prevention of neurodegenerative, neuroinflammatory and neuroimmunological disorders.

The aforementioned well-characterized diseases in humans can also occur with comparable etiology in other mammals and can likewise be treated therein with the compounds of the present invention.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

The present invention thus further provides for the use of the compounds of the invention for treatment and/or prevention of disorders, especially of the aforementioned disorders.

The present invention further provides for the use of the compounds of the invention for production of a medicament for treatment and/or prevention of disorders, especially of the aforementioned disorders.

The present invention further provides a medicament comprising at least one of the compounds of the invention for treatment and/or prevention of disorders, especially of the aforementioned disorders.

The present invention further provides for the use of the compounds of the invention in a method for treatment and/or prevention of disorders, especially of the aforementioned disorders.

The present invention further provides a process for treatment and/or prevention of disorders, especially of the aforementioned disorders, using an effective amount of at least one of the compounds of the invention.

The compounds of the invention can be used alone or, if required, in combination with one or more other pharmacologically active substances, provided that this combination does not lead to undesirable and unacceptable side effects. The present invention therefore further provides medicaments comprising at least one of the compounds of the invention and one or more further drugs, especially for treatment and/or prevention of the aforementioned disorders. Preferred examples of combination active ingredients suitable for this purpose include:
respiratory stimulants, by way of example and with preference theophylline, doxapram, nikethamide or caffeine;
psychostimulants, by way of example and with preference modafinil or armodafinil;
amphetamines and amphetamine derivatives, by way of example and with preference amphetamine, metamphetamine or methylphenidate;
serotonin reuptake inhibitors, by way of example and with preference fluoxetine, paroxetine, citalopram, escitalopram, sertraline, fluvoxamine or trazodone;
serotonin precursors, by way of example and with preference L-tryptophan;
selective serotonin noradrenaline reuptake inhibitors, by way of example and with preference venlafaxine or duloxetine;
noradrenergic and specific serotonergic antidepressants, by way of example and with preference mirtazapine;
selective noradrenaline reuptake inhibitors, by way of example and with preference reboxetine;
tricyclic antidepressants, by way of example and with preference amitriptyline, protriptyline, doxepine, trimipramine, imipramine, clomipramine or desipramine;
alpha2-adrenergic agonists, by way of example and with preference clonidine;
GABA agonists, by way of example and with preference baclofen;
alpha sympathomimetics, by way of example and with preference xylometazoline, oxymetazoline, phenylephrine, naphazoline, tetryzoline or tramazoline;
glucocorticoids, by way of example and with preference fluticasone, budesonide, beclometasone, mometasone, tixocortol or triamcinolone;
cannabinoid receptor agonists;
carboanhydrase inhibitors, by way of example and with preference acetazolamide, methazolamide or diclofenamide;
opioid and benzodiazepine receptor antagonists, by way of example and with preference flumazenil, naloxone or naltrexone;
cholinesterase inhibitors, by way of example and with preference neostigmine, pyridostigmine, physostigmine, donepezil, galantamine or rivastigmine;
N-methyl-D-aspartate and glutamate antagonists, by way of example and with preference amantadine, memantine or sabeluzole;
nicotine receptor agonists;
leukotriene receptor antagonists, by way of example and with preference montelukast or tripelukast;
dopamine receptor antagonists, by way of example and with preference dromperidone, metoclopramide or benzamide, butyrophenone or phenothiazine derivatives;
appetite suppressants, by way of example and with preference sibutramine, topiramate, phentermine, lipase inhibitors or cannabinoid receptor antagonists;
proton pump inhibitors, by way of example and with preference pantoprazole, omeprazole, esomeprazole, lansoprazole or rabeprazole;
organic nitrates and NO donors, for example sodium nitroprusside, nitroglycerin, isosorbide mononitrate, isosorbide dinitrate, molsidomine or SIN-1, and inhaled NO;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 5 inhibitors such as sildenafil, vardenafil, tadalafil, udenafil, dasantafil, avanafil, mirodenafil or lodenafil;
NO- and haem-independent activators of soluble guanylate cyclase (sGC), such as in particular the compounds described in WO 01/19355, WO 01/19776, WO 01/19778, WO 01/19780, WO 02/070462 and WO 02/070510;
NO-independent but haem-dependent stimulators of soluble guanylate cyclase (sGC), such as in particular riociguat, vericiguat and the compounds described in WO 00/06568, WO 00/06569, WO 02/42301, WO 03/095451, WO 2011/147809, WO 2012/004258, WO 2012/028647 and WO 2012/059549;
prostacyclin analogues and IP receptor agonists, by way of example and with preference iloprost, beraprost, treprostinil, epoprostenol or selexipag;
endothelin receptor antagonists, by way of example and with preference bosentan, darusentan, ambrisentan or sitaxsentan;
compounds which inhibit human neutrophile elastase (HNE), by way of example and with preference sivelestat or DX-890 (reltran);
compounds which inhibit the degradation and alteration of the extracellular matrix, by way of example and with preference inhibitors of the matrix metalloproteases (MMPs), especially inhibitors of stromelysin, collagenases, gelatinases and aggrecanases (in this context particularly of MMP-1, MMP-3, MMP-8, MMP-9, MMP-10, MMP-11 and MMP-13) and of metalloelastase (MMP-12);
compounds which block the binding of serotonin to its receptors, by way of example and with preference antagonists of the 5-HT_{2B} receptor such as PRX-08066;
antagonists of growth factors, cytokines and chemokines, by way of example and with preference antagonists of TGF-β, CTGF, IL-1, IL-4, IL-5, IL-6, IL-8, IL-13 and integrins;
Rho kinase-inhibiting compounds, by way of example and with preference fasudil, Y-27632, SLx-2119, BF-66851, BF-66852, BF-66853, KI-23095 or BA-1049;
compounds which influence the energy metabolism of the heart, by way of example and with preference etomoxir, dichloroacetate, ranolazine or trimetazidine;
compounds which inhibit the signal transduction cascade, by way of example and with preference from the group of the kinase inhibitors, in particular from the group of the tyrosine kinase and/or serine/threonine kinase inhibitors, by way of example and with preference nintedanib, dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib or tandutinib;
anti-obstructive agents as used, for example, for treatment of chronic obstructive pulmonary disease (COPD) or bronchial asthma, by way of example and with preference from the group of the inhalatively or systemically administered agonists of the beta-adrenergic receptor (beta-mimetics) and the inhalatively administered anti-muscarinergic substances;
antiinflammatory, immunomodulating, immunosuppressive and/or cytotoxic agents, by way of example and with preference from the group of the systemically or inhalatively administered corticosteroids and also dimethyl fumarate, fingolimod, glatiramer acetate, β-interferons, natalizumab, teriflunomide, mitoxantrone, immunoglobulins, acetylcysteine, montelukast, tripelukast, azathioprine, cyclophosphamide, hydroxycarbamide, azithromycin, interferon-y, pirfenidone or etanercept;
antifibrotic agents, by way of example and with preference lysophosphatidic acid receptor 1 (LPA-1) antagonists, CTGF inhibitors, IL-4 antagonists, IL-13 antagonists, TGF-β antagonists or pirfenidone;
antithrombotic agents, by way of example and with preference from the group of platelet aggregation inhibitors, the anticoagulants and the profibrinolytic substances;
hypotensive active ingredients, by way of example and with preference from the group of the calcium antagonists, angiotensin AII antagonists, ACE inhibitors, vasopeptidase inhibitors, endothelin antagonists, renin inhibitors, alpha receptor blockers, beta receptor blockers, mineralocorticoid receptor antagonists and also the diuretics; and/or
active ingredients that alter lipid metabolism, by way of example and with preference from the group of the thyroid receptor agonists, cholesterol synthesis inhibitors, by way of example and preferably HMG-CoA reductase inhibitors or squalene synthesis inhibitors, the ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

In a preferred embodiment of the invention, the compounds of the invention are administered in combination with a beta-adrenergic receptor agonist, by way of example and with preference albuterol, isoproterenol, metaproterenol, terbutalin, fenoterol, formoterol, reproterol, salbutamol or salmeterol.

In a preferred embodiment of the invention, the compounds of the invention are administered in combination with an antimuscarinergic substance, by way of example and with preference ipratropium bromide, tiotropium bromide or oxitropium bromide.

In a preferred embodiment of the invention, the compounds of the invention are administered in combination with a corticosteroid, by way of example and with preference prednisone, prednisolone, methylprednisolone, triamcinolone, dexamethasone, betamethasone, beclomethasone, flunisolide, budesonide or fluticasone.

Antithrombotic agents are preferably understood to mean compounds from the group of the platelet aggregation inhibitors, the anticoagulants and the profibrinolytic substances.

In a preferred embodiment of the invention, the compounds of the invention are administered in combination with a platelet aggregation inhibitor, by way of example and with preference aspirin, clopidogrel, ticlopidine or dipyridamole.

In a preferred embodiment of the invention, the compounds of the invention are administered in combination with a thrombin inhibitor, by way of example and with preference ximelagatran, melagatran, dabigatran, bivalirudin or clexane.

In a preferred embodiment of the invention, the compounds of the invention are administered in combination with a GPIIb/IIIa antagonist, by way of example and with preference tirofiban or abciximab.

In a preferred embodiment of the invention, the compounds of the invention are administered in combination with a factor Xa inhibitor, by way of example and with preference rivaroxaban, apixaban, fidexaban, razaxaban, fondaparinux, idraparinux, DU-176b, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or with a low molecular weight (LMW) heparin derivative.

In a preferred embodiment of the invention, the compounds of the invention are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha receptor blockers, beta receptor blockers, mineralocorticoid receptor antagonists, and the diuretics.

In a preferred embodiment of the invention, the compounds of the invention are administered in combination with a calcium antagonist, by way of example and with preference nifedipine, amlodipine, verapamil or diltiazem.

In a preferred embodiment of the invention, the compounds of the invention are administered in combination with an alpha-1 receptor blocker, by way of example and with preference prazosin.

In a preferred embodiment of the invention, the compounds of the invention are administered in combination with a beta receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the inventive compounds are administered in combination with an angiotensin AII antagonist, preferred examples being losartan, candesartan, valsartan, telmisartan or embusartan.

In a preferred embodiment of the invention, the compounds of the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds of the invention are administered in combination with an endothelin antagonist, by way of example and with preference bosentan, darusentan, ambrisentan or sitaxsentan.

In a preferred embodiment of the invention, the compounds of the invention are administered in combination with a renin inhibitor, by way of example and with preference aliskiren, SPP-600 or SPP-800.

In a preferred embodiment of the invention, the compounds of the invention are administered in combination with a mineralocorticoid receptor antagonist, by way of example and with preference spironolactone, eplerenone or finerenone.

In a preferred embodiment of the invention, the compounds of the invention are administered in combination with a diuretic, by way of example and with preference furosemide, bumetanide, torsemide, bendroflumethiazide, chlorothiazide, hydrochlorothiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbers, bile acid reabsorption inhibitors, lipase inhibitors and the lipoprotein(a) antagonists.

In a preferred embodiment of the invention, the compounds of the invention are administered in combination with a CETP inhibitor, by way of example and with preference torcetrapib (CP-529 414), JJT-705 or CETP vaccine (Avant).

In a preferred embodiment of the invention, the compounds of the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds of the invention are administered in combination with a squalene synthesis inhibitor, by way of example and with preference BMS-188494 or TAK-475.

In a preferred embodiment of the invention, the compounds of the invention are administered in combination with an ACAT inhibitor, by way of example and with preference avasimibe, melinamide, pactimibe, eflucimibe or SMP-797.

In a preferred embodiment of the invention, the compounds of the invention are administered in combination with an MTP inhibitor, by way of example and with preference implitapide, BMS-201038, R-103757 or JTT-130.

In a preferred embodiment of the invention, the compounds of the invention are administered in combination with a PPAR-gamma agonist, by way of example and with preference pioglitazone or rosiglitazone.

In a preferred embodiment of the invention, the compounds of the invention are administered in combination with a PPAR-delta agonist, by way of example and with preference GW 501516 or BAY 68-5042.

In a preferred embodiment of the invention, the compounds of the invention are administered in combination with a cholesterol absorption inhibitor, by way of example and with preference ezetimibe, tiqueside or pamaqueside.

In a preferred embodiment of the invention, the compounds of the invention are administered in combination with a lipase inhibitor, by way of example and with preference orlistat.

In a preferred embodiment of the invention, the compounds of the invention are administered in combination with a polymeric bile acid adsorber, by way of example and with preference cholestyramine, colestipol, colesolvam, CholestaGel or colestimide.

In a preferred embodiment of the invention, the compounds of the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds of the invention are administered in combination with a lipoprotein(a) antagonist, by way of example and with preference gemcabene calcium (CI-1027) or nicotinic acid.

Particular preference is given to combinations of the compounds of the invention with one or more further active ingredients selected from the group consisting of respiratory stimulants, psychostimulants, serotonin reuptake inhibitors, noradrenergic, serotonergic and tricyclic antidepressants, sGC stimulators, mineralocorticoid receptor antagonists, antiinflammatory drugs, immunomodulators, immunosuppressives and cytotoxic drugs.

If required, the substances of the invention can also be employed in conjunction with the use of one or more medical technical devices or auxiliaries, provided that this does not lead to unwanted and unacceptable side-effects. Medical devices and auxiliaries suitable for such a combined application are, by way of example and with preference:
devices for positive airway pressure ventilation, by way of example and with preference CPAP (continuous positive airway pressure) devices, BiPAP (bilevel positive airway pressure) devices and IPPV (intermittent positive pressure ventilation) devices;
neurostimulators of the Nervus hypoglossus;
intraoral auxiliaries, by way of example and with preference protrusion braces;
nasal disposable valves;
nasal stents.

The present invention further provides medicaments which comprise at least one compound of the invention, typically together with one or more inert, non-toxic, pharmaceutically suitable excipients, and for the use thereof for the aforementioned purposes.

The compounds of the invention can act systemically and/or locally. For this purpose, they can be administered in a suitable manner, for example by the oral, parenteral, pulmonal, intrapulmonal (inhalative), nasal, intranasal, pharyngeal, lingual, sublingual, buccal, rectal, dermal, transdermal, conjunctival or otic route, or as an implant or stent.

The compounds of the invention can be administered in administration forms suitable for these administration routes.

Suitable administration forms for oral administration are those which work according to the prior art and release the compounds of the invention rapidly and/or in a modified manner and which contain the compounds of the invention in crystalline and/or amorphized and/or dissolved form, for example tablets (uncoated or coated tablets, for example with gastric juice-resistant or retarded-dissolution or insoluble coatings which control the release of the compound of the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can bypass an absorption step (e.g. take place intravenously, intraarterially, intracardially, intraspinally or intralumbally) or include an absorption (e.g. take place inhalatively, intramuscularly, subcutaneously, intracutaneously, percutaneously or intraperitoneally). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers, metered aerosols), nasal drops, solutions or sprays, throat sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral, intravenous, intranasal and pharyngeal administration.

In one embodiment, administration is by the intranasal route. In one embodiment, intranasal administration is effected with the aid of nose drops or a nasal spray. In one embodiment, intranasal administration is effected with the aid of a nasal spray.

The compounds of the invention can be converted to the administration forms mentioned. This can be accomplished in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctors.

In general, it has been found to be advantageous in the case of parenteral administration to administer amounts of about 0.001 to 1 mg/kg, preferably about 0.01 to 0.5 mg/kg, of body weight to achieve effective results. In the case of oral administration the dosage is about 0.01 to 100 mg/kg, preferably about 0.01 to 20 mg/kg and most preferably 0.1 to 10 mg/kg of body weight.

In one embodiment, the dosage in the case of intranasal administration is about 0.1 μg to 500 μg per day. In a further embodiment, the dosage in the case of intranasal administration is about 1 μg to 250 μg per day. In a further embodiment, the dosage in the case of intranasal administration is about 1 μg to 120 μg per day. In a further embodiment, the dose of about 0.1 μg to 500 μg per day, or of about 1 μg to 250 μg per day, or of about 1 μg to 120 μg per day, is administered once daily by the intranasal route before sleeping. In one embodiment, the dose of about 0.1 μg to 500 μg per day, or of about 1 μg to 250 μg per day, or of about 1 μg to 120 μg per day, is administered once daily with half to each nostril. In one embodiment, the dose of about 0.1 μg to 500 μg per day, or of about 1 μg to 250 μg per day, or of about 1 μg to 120 μg per day, is administered once daily with half to each nostril before sleeping.

It may nevertheless be necessary in some cases to deviate from the stated amounts, specifically as a function of body weight, route of administration, individual response to the active ingredient, nature of the preparation and time or interval over which administration takes place. Thus in some cases it may be sufficient to manage with less than the abovementioned minimum amount, while in other cases the upper limit mentioned must be exceeded. In the case of administration of greater amounts, it may be advisable to divide them into several individual doses over the day.

The invention further relates to a method of discovering a compound having TASK-1- and/or TASK-3-blocking properties, wherein the method comprises subjecting at least one compound to at least one assay selected from the group consisting of:
determining the inhibitory concentration (IC₅₀) in relation to the K⁺ conductivity of a TASK-1 or TASK-3 channel,
determining the washout rate
and
determining the maximum possible bioavailability.

The washout rate is defined as the washout of a compound according to the invention from the TASK-1 channel in % h⁻¹, measured by means of electrophysiological analysis on TASK-1-expressing Xenopus laevis oocytes via the two-electrode voltage clamp technique according to the description in section B-4.

The maximum possible bioavailability of a compound is determined on the basis of its hepatic extraction rate, which is determined by the degradation of the starting compound in an in vitro clearance assay with hepatocytes. The calculation is effected via what is called the “well-stirred model”. It is assumed here that all three aqueous systems in the liver (blood, interstitial fluid and intercellular fluid) are well-stirred and can be described as one compartment. In this model, distribution is effected by passive diffusion only. In the simplified screening model, the protein binding of the substance is neglected. The concentration of the compound decreases through elimination, in this case through degradation of the compound. The maximum possible bioavailability thus determined is frequently also referred to as “Fₘₐₓ well-stirred”. Protocols for determination of maximum possible bioavailability are disclosed, for example, in Rowland & Tozer, Clinical Pharmacokinetics and Pharmacodynamics, 4^{th} edition, Appendix E, page 705 ff.

In the context of the present invention, it has been found that, surprisingly, the specific combination of assays claimed can be used to find compounds having suitability for the prevention and/or treatment of obstructive sleep apnoea from a pool of compounds conforming to the following profile:
sufficient efficacy,
sufficiently long duration of action,
suitability for nasal administration and
high clearance rate of compounds not bound to the target.

Obstructive sleep apnoea (OSA) is caused by a reduction in the muscle activity of the upper respiratory tract. The Musculus genioglossus (a muscle at the base of the tongue) is the most important of the dilating muscles of the upper respiratory tract and is activated in the manner of a reflex by negative pressure in the upper respiratory tract, in order thus to counteract a collapse of the upper respiratory tract. Pressure-sensitive nerve endings/mechanoreceptors in the pharynx and in the upper respiratory tract recognize the onset of reduced pressure in the upper respiratory tract during the respiratory cycle. This feedback from the mechanoreceptors is responsible for the predominant portion of the dilating muscle reactions in the upper respiratory tract. TASK-1, also called K2P3.1, and TASK-3, also called K2P9.1, are members of the superfamily of the potassium channel proteins that have two pore-forming P domains. TASK-1 and TASK-3 mediate background potassium currents that stabilize the resting potential and accelerate the repolarization of the action potential. The blockage of TASK-1 and/or TASK-3 by means of a suitable compound can lead to sensitization of the mechanoreceptors of the upper respiratory tract, which in turn activates the Musculus genioglossus and prevents collapse of the upper respiratory tract.

The nasal administration of suitable compounds permits the quickest access to this mechanism of action. Nasal administration is therefore, in accordance with the invention, a preferred mode of administration of a compound having TASK-1- and/or TASK-3-blocking properties.

Obstructive sleep apnoea, moreover, is a state that can occur over the entire duration of sleep. The inventors have found that it can be desirable, for increasing patient compliance, to find a compound that has a long duration of action in order thus to protect the patient from OSA even over prolonged sleep phases. Such a long duration of action can be achieved, for example, by virtue of a low dissociation rate (K_{off}) of the compound in question from the TASK-1 and/or TASK-3 channel. As a correlate for the K_{off} value, the washout rate was determined in the present invention.

In addition, the inventors recognized that, while nasal administration is fundamentally suitable for introducing sufficient concentrations of the compound into the target tissue, this mode of administration also prevents the molecules not bound to the target channel(s) from becoming systemically available to a relevant degree, which makes systemic side effects less likely. For this reason, the inventors have found that a high clearance rate of molecules of the compound not bound to the target is advantageous.

The inventive combination of assays is suitable for finding compounds that fulfil the above specific profile of requirements. This assay combination is not suggested by the prior art. The search for a compound having a long duration of action and simultaneously a high clearance rate is, moreover, a difficult undertaking since the two properties are contradictory to one another. One ought to assume that a medicament can have either a long duration of action or a high clearance rate. However, the inventors have succeeded for the first time, with the aid of the combination of assays according to the invention, in combining a long local duration of action with a high systemic clearance rate. Unbound molecules of the compound that are still in the bloodstream are excreted or, for example, metabolized in the liver.

In one embodiment, the compound is subjected to at least one further assay selected from the group consisting of:
determining the brain/plasma concentration ratio C_{br}/Cₚ,
determining c Log D [pH 7.5] and/or c Log P and/or tPSA.

The unbound concentrations in the brain should be at a minimum in order that central side effects are unlikely. For determination thereof, the total concentrations in the brain and plasma are first determined and the unbound concentrations are ascertained with the aid of the free fractions in the brain and plasma (dialysis), and C_{br}/Cₚ is calculated in this way. Ultimately, central side effects are detected in safety pharmacology.

The partition coefficient log P and the distribution coefficient log D describe the concentration ratio of a compound in a mixture of two immiscible phases at equilibrium. This ratio is thus a measure of the difference in the solubility of the compound in these two phases. Water is frequently one of the phases, while the second phase is a hydrophobic solvent such as 1-octanol. Log P relates to a given compound in uncharged form, whereas log D takes account of all uncharged and charged forms of the compound, optionally at a defined pH. Since the charged form barely enters the hydrophobic phase, there is a change in distribution with pH if it affects the charge of the compound. In the pH range in which the compound is uncharged, log D=log P. In the pH range in which a significant proportion of the compound is in charged form, log D becomes a function of log P, pH and pKa. Log D can be expressed as follows:

log D=log P−log(1+10^{(pH-pKa)})

Both values thus give a measure of the hydrophobicity of the compound being sought, which in turn affects the retention time of the compound in cell membranes, for example.

c Log D and c Log P are respectively log D and log P values precalculated on the basis of the incremental contributions of the respective molecular fragments.

The expression “tPSA” refers to the topological polar surface area and is a measure of the total surface area of all polar atoms in a molecule. tPSA is a frequently used parameter for the determination of the ability of a compound to pass through cell membranes. It is generally reported in ångström². Compounds having a high tPSA have a tendency to poor permeation through cell membranes.

In a further embodiment, the compound is subjected to at least one further assay selected from the group consisting of:
determining the selectivity for TASK-1 and/or TASK-3 with respect to other K⁺ channels,
determining passive apparent permeability (cPAPP, passive),
determining blood clearance (CL_{blood}).

Passive apparent permeability is a measure of the in vivo absorption of a compound.

Also envisaged is a method of producing a compound having TASK-1- and/or TASK-3-blocking properties and suitability for nasal administration, wherein the method comprises:
producing and/or providing a library of compounds,
testing at least one compound from this library in one or more of the above-described assays,
isolating at least one compound after this step, and optionally
converting the at least one compound to a pharmaceutical formulation suitable for nasal administration.

In one embodiment of this method, the compound has to fulfil at least one of the conditions fixed in the following group:
a) the inhibitory concentration (IC₅₀) in relation to the K⁺ conductivity of the TASK-1 or TASK-3 channel is ≤200 nM, measured by the two-electrode voltage clamp technique (TEVC) in Xenopus laevis oocytes that have been injected with TASK-1 cRNA or TASK-3 cRNA;
b) the washout rate is ≤50% h⁻¹, measured by the two-electrode voltage clamp technique (TEVC) in Xenopus laevis oocytes that have been injected with TASK-1 cRNA or TASK-3 cRNA;
c) the maximum possible bioavailability (“Fₘₐₓ well-stirred”) is ≤40%, measured by means of the hepatocyte in vitro clearance test described herein;
d) the brain/plasma concentration ratio C_{br}/Cₚ is ≤1, measured after nasal and/or intravenous administration of the compound to rats and subsequent LC-MS/MS analysis of processed plasma and brain tissue samples;
e) c Log D [pH 7.5] is between ≥2.5 and ≤5;
f) c Log P is between ≥1 and ≤5;
g) tPSA is between ≥25 and ≤100 Å²;
h) the inhibitory concentration (IC₅₀) relating to the K⁺ conductivity of the TASK-1 or TASK-3 channel is at least 10³ times less than that relating to the cardiac hERG K⁺ channel, measured by the two-electrode voltage clamp technique (TEVC) in Xenopus laevis oocytes (which leads to a high selectivity of the compound in favour of the TASK-1 and/or TASK-3 channel);
i) cPAPP, passive is ≥100, measured in Caco-2 cells based on the determination of apparent permeability (PAPP);
j) blood clearance (C_{blood}) is ≥60% of the species-specific liver perfusion;
k) oral bioavailability is ≤40%, expressed as the quotient of AUC_{standard} (peroral administration)/AUC_{standard} (intravenous administration).

Methods relating to two-electrode voltage clamp technique (TEVC) measurements in Xenopus laevis oocytes are described in Experiment B-4. This employs the methods of Decher et al., FEBS Lett. 42, 84-89 (2001) and Stühmer, Methods Enzymol. 207, 319-339 (1992).

The determination of c Log P and c Log D is effected in accordance with the invention by a standard method as described, for example, in Comer and Tam, “Lipophilicity Profiles: Theory and Measurement”, in: Testa, van de Waterbed, Folkers & Guy, Pharmacokinetic Optimization in Drug Research: Biological, Physicochemical and Computational Strategies, Weinheim, Wiley-VCH, pp. 275-304. The method used in accordance with the invention for calculation of the tPSA value is described in detail in Ertl et al., J. Med. Chem. 4, 3714-3717 (2000). The method is based on the summation of the tabulated literature values for the surface contributions of the polar components of the molecule.

The apparent permeability (PAPP) is determined, for example, according to Artursson and Karlsson, Biochem. Biophys. Res. Commun. 175 (3), 880-885 (1991). In order to exclude the influence of transporters from the calculation in the method, the apparent permeabilities both from the apical to the basolateral side and from the basolateral to the apical side are determined. The values are added and divided by two.

The parameters AUC_{standard} (peroral administration) and AUC_{standard} (intravenous administration) used for the calculation of oral bioavailability are determined by means of standard methods. The determination of blood clearance (CL_{blood}) in % in species-specific liver perfusion is conducted in accordance with the invention by generally customary in vivo tests with intravenous substance administration, for example by the standard PK methods described in Rowland & Tozer, Clinical Pharmacokinetics and Pharmacodynamics, 4^{th} edition.

In a further embodiment, it is envisaged that the compound will be suitable for the prevention or treatment of obstructive sleep apnoea (OSA) or one or more symptoms associated therewith.

In a further embodiment, it is envisaged that the compound will be suitable for nasal administration.

In a further embodiment, it is envisaged that the compound will bring about inhibition of upper airway collapsibility in a pig model of OSA.

In a further embodiment, it is envisaged that the duration of inhibition of the collapsibility of the upper respiratory tract in the OSA pig model after intranasal administration of between 0.3 μg and 300 μg of the compound will be more than 240 min, measured at a reduced pressure of 100 cm water column.

The invention also provides a compound having TASK-1- and/or TASK-3-blocking properties obtainable by the screening method described above.

In one embodiment, it is envisaged that the compound will have at least one functional feature selected from the following group:
a) the inhibitory concentration (IC₅₀) relating to the K⁺ conductivity of the TASK-1 or TASK-3 channel is ≤200 nM;
b) the washout rate is ≤50% h⁻¹;
c) the maximum possible bioavailability (“Fₘₐₓ well-stirred”) is ≤40%.

In a further embodiment, it is envisaged that the compound will have at least one of the further features mentioned above, especially selected from features d)-k).

In a further embodiment, it is envisaged that the compound will be an (imidazo[1,2-a]pyridin-3-yl)methyl-substituted diazaheterobicyclic compound.

In one embodiment, the compounds disclosed in EP patent application 15199270.8 and in EP patent application 15199268.2 are not included.

In one embodiment of the method or of the compound, the washout rate of the compound is preferably ≤40% h⁻¹, more preferably ≤30% h⁻¹ and most preferably ≤20% h⁻¹.

The invention also provides a compound that competes with a compound according to the above description for interaction with TASK-1 and/or TASK-3. The term “interaction” relates to at least one feature from the group consisting of:
reduction in the K⁺ conductivity of the TASK-1 or TASK-3 channel,
binding to one or more epitopes and/or domains of TASK-1 and/or TASK-3.

The working examples which follow illustrate the invention. The invention is not restricted to the examples.

### A. EXAMPLES

### Abbreviations and Acronyms

abs. absolute
Ac acetyl
aq. aqueous, aqueous solution
Boc tert-butoxycarbonyl
br. broad (in NMR signal)
Ex. Example
Bu butyl
c concentration
cat. catalytic
CI chemical ionization (in MS)
d doublet (in NMR)
d day(s)
DCI direct chemical ionization (in MS)
dd doublet of doublets (in NMR)
DMF N,N-dimethylformamide
DMSO dimethyl sulphoxide
dq doublet of quartets (in NMR)
dt doublet of triplets (in NMR)
EI electron impact ionization (in MS)
eq. equivalent(s)
ESI electrospray ionization (in MS)
Et ethyl
h hour(s)
HATU O-(7-azabenzotriazol-1-yl)-N,N,N′,N′-tetramethyluronium hexafluorophosphate
HOBt 1-hydroxy-1H-benzotriazole hydrate
HPLC high-pressure, high-performance liquid chromatography
iPr isopropyl
conc. concentrated (in the case of a solution)
LC liquid chromatography
LC-MS liquid chromatography-coupled mass spectrometry
Lit. literature (reference)
m multiplet (in NMR)
Me methyl
min minute(s)
MS mass spectrometry
NMR nuclear magnetic resonance spectrometry
Ph phenyl
Pr propyl
q quartet (in NMR)
quant. quantitative (in chemical yield)
RP reverse phase (in HPLC)
RT room temperature
Rₜ retention time (in HPLC, LC-MS)
s singlet (in NMR)
t triplet (in NMR)
tBu tert-butyl
TFA trifluoroacetic acid
THF tetrahydrofuran
UV ultraviolet spectrometry
v/v volume to volume ratio (of a solution)

LC-MS and HPLC Methods:

Method 1 (LC-MS):

Instrument: Waters Acquity SQD UPLC System; column: Waters Acquity UPLC HSS T3 1.8 μm, 50 mm×1 mm; eluent A: 1 l water+0.25 ml 99% formic acid, eluent B: 1 l acetonitrile+0.25 ml 99% formic acid; gradient: 0.0 min 90% A→1.2 min 5% A→2.0 min 5% A; temperature: 50° C.; flow rate: 0.40 ml/min; UV detection: 208-400 nm.

Method 2 (LC-MS):

MS instrument: Thermo Scientific FT-MS; UHPLC instrument: Thermo Scientific UltiMate 3000; column: Waters HSS T3 C18 1.8 μm, 75 mm×2.1 mm; eluent A: 1 l water+0.01% formic acid, eluent B: 1 l acetonitrile+0.01% formic acid; gradient: 0.0 min 10% B→2.5 min 95% B→3.5 min 95% B; temperature: 50° C.; flow rate: 0.90 ml/min; UV detection: 210 nm/optimum integration path 210-300 nm.

Method 3 (LC-MS):

MS instrument: Waters Micromass QM; HPLC instrument: Agilent 1100 Series; column: Agilent ZORBAX Extend-C18 3.5 μm, 50 mm×3.0 mm; eluent A: 1 l water+0.01 mol ammonium carbonate, eluent B: 1 l acetonitrile; gradient: 0.0 min 98% A→0.2 min 98% A→3.0 min 5% A→4.5 min 5% A; temperature: 40° C.; flow rate: 1.75 ml/min; UV detection: 210 nm.

Method 4 (LC-MS):

MS instrument: Waters Micromass Quattro Micro; HPLC instrument: Waters UPLC Acquity; column: Waters BEH C18 1.7 μm, 50 mm×2.1 mm; eluent A: 1 l water+0.01 mol ammonium formate, eluent B: 1 l acetonitrile; gradient: 0.0 min 95% A→0.1 min 95% A→2.0 min 15% A→2.5 min 15% A→2.51 min 10% A→3.0 min 10% A; temperature: 40° C.; flow rate: 0.5 ml/min; UV detection: 210 nm.

Method 5 (LC-MS):

Instrument: Agilent MS Quad 6150 with HPLC Agilent 1290; column: Waters Acquity UPLC HSS T3 1.8 μm, 50 mm×2.1 mm; eluent A: 1 l water+0.25 ml 99% formic acid, eluent B: 1 l acetonitrile+0.25 ml 99% formic acid; gradient: 0.0 min 90% A→0.3 min 90% A→1.7 min 5% A→3.0 min 5% A; flow rate: 1.20 ml/min; temperature: 50° C.; UV detection: 205-305 nm.

Method 6 (preparative HPLC):

Instrument: Abimed Gilson 305; column: Reprosil C18 10 μm, 250 mm×30 mm; eluent A: water, eluent B: acetonitrile; gradient: 0-3 min 10% B, 3-27 min 10% B→95% B, 27-34.5 min 95% B, 34.5-35.5 min 95% B→10% B, 35.5-36.5 min 10% B; flow rate: 50 ml/min; room temperature; UV detection: 210 nm.

Method 7 (LC-MS):

MS instrument: Waters SQD; HPLC instrument: Waters UPLC; column: Zorbax SB-Aq (Agilent), 50 mm×2.1 mm, 1.8 μm; eluent A: water+0.025% formic acid, eluent B: acetonitrile+0.025% formic acid; gradient: 0.0 min 98% A→0.9 min 25% A→1.0 min 5% A→1.4 min 5% A→1.41 min 98% A→1.5 min 98% A; oven: 40° C.; flow rate: 0.60 ml/min; UV detection: DAD, 210 nm.

Further Details:

The descriptions of the coupling patterns of ¹H NMR signals which follow are guided by the visual appearance of the signals in question and do not necessarily correspond to a strict, physically correct interpretation. In general, the stated chemical shift refers to the centre of the signal in question; in the case of broad multiplets, an interval is generally given.

Melting points and melting point ranges, if stated, are uncorrected.

In cases where the reaction products were obtained by trituration, stirring or recrystallization, it was frequently possible to isolate further amounts of product from the respective mother liquor by chromatography. However, a description of this chromatography is dispensed with hereinbelow unless a large part of the total yield could only be isolated in this step.

All reactants or reagents whose preparation is not described explicitly hereinafter were purchased commercially from generally accessible sources. For all other reactants or reagents whose preparation is likewise not described hereinafter and which were not commercially obtainable or were obtained from sources which are not generally accessible, a reference is given to the published literature in which their preparation is described.

### Starting Compounds and Intermediates

### Example 1A

### 2-(4-Chlorophenyl)imidazo[1,2-a]pyridine

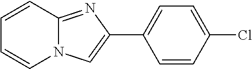 [SMILES: Clc1ccc(-c2cn3ccccc3n2)cc1]

To a solution of 20 g (85.65 mmol) of 2-bromo-1-(4-chlorophenyl)ethanone and 8.87 g (94.22 mmol) of pyridin-2-amine in 200 ml of ethanol were added 10.95 g (130 mmol) of sodium hydrogencarbonate, and the mixture was stirred at 80° C. for 5 hours. The mixture was then cooled, first to room temperature and then to 0° C. (ice bath). The resulting precipitate was filtered off and washed repeatedly with an ethanol/water mixture (2:1). The solid was then dried under reduced pressure at 40° C. overnight. 19.8 g of the target product were obtained, which was used in subsequent reactions without further purification.

¹H-NMR (400 MHz, DMSO-d₆, δ/ppm): 6.87-6.94 (m, 1H), 7.23-7.29 (m, 1H), 7.50 (d, 2H), 7.58 (d, 1H), 7.99 (d, 2H), 8.43 (s, 1H), 8.53 (d, 1H).

LC-MS (Method 1): Rₜ=0.58 min; m/z=229/231 (M+H)⁺.

### Example 2A

### 2-(5-Chloropyridin-2-yl)imidazo[1,2-a]pyridine

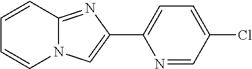 [SMILES: Clc1ccc(-c2cn3ccccc3n2)nc1]

5 g (32.14 mmol) of 1-(5-chloropyridin-2-yl)ethanone, 6.96 g (73.92 mmol) of pyridin-2-amine and 9.79 g (38.56 mmol) of iodine were stirred at 120° C. for 2 h. After cooling to room temperature, 15 ml of water and 1.93 g (48 mmol) of sodium hydroxide were added and then the reaction mixture was stirred at 100° C. for another 1 h. Thereafter, the mixture was cooled to room temperature and the precipitate obtained was filtered off and washed repeatedly with water. The solids were dissolved in cyclohexane/ethyl acetate (1:1), silica gel was added, the mixture was concentrated to dryness again and the residue was purified by column chromatography on silica gel (eluent: cyclohexane/ethyl acetate 1:1). 4.32 g (18.81 mmol, 59% of theory) of the target compound were obtained.

¹H-NMR (400 MHz, DMSO-d₆, δ/ppm): 6.95 (t, 1H), 7.30 (t, 1H), 7.61 (d, 1H), 8.00 (dd, 1H), 8.12 (d, 1H), 8.50 (s, 1H), 8.59 (d, 1H), 8.65 (d, 1H).

LC-MS (Method 1): Rₜ=0.50 min; m/z=230/232 (M+H)⁺.

### Example 3A

### 2-(6-Isopropylpyridin-3-yl)imidazo[1,2-a]pyridine

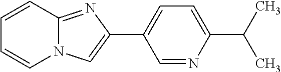 [SMILES: CC(C)c1ccc(-c2cn3ccccc3n2)cn1]

5 g (30.63 mmol) of 1-(6-isopropylpyridin-3-yl)ethanone [CAS Registry Number 80394-97-4], 6.63 g (70.46 mmol) of pyridin-2-amine and 9.33 g (36.76 mmol) of iodine were stirred at 120° C. for 2 h. After cooling to room temperature, 50 ml of water and 46 ml (46 mmol) of 1 M sodium hydroxide solution were added and then the reaction mixture was stirred at 100° C. for another 1 h. Thereafter, the mixture was cooled to room temperature, in the course of which an oily liquid separated out. The reaction mixture was partitioned between water and ethyl acetate, and the organic phase was removed. The latter was twice washed with water, dried over magnesium sulphate and then concentrated. The oil obtained was subjected to chromatographic purification using neutral alumina (eluent: cyclohexane/ethyl acetate 1:1). The material thus obtained was further purified by two column chromatography runs on silica gel (Biotage SNAP cartridge KP-NH column; eluent: cyclohexane/ethyl acetate 1:2). 1.62 g (6.83 mmol, 22% of theory) of the target compound were obtained.

¹H-NMR (400 MHz, DMSO-d₆, δ/ppm): 1.27 (d, 6H), 2.98-3.12 (m, 1H), 6.91 (t, 1H), 7.27 (t, 1H), 7.35 (d, 1H), 7.60 (d, 1H), 8.22 (dd, 1H), 8.45 (s, 1H), 8.54 (dd, 1H), 9.06 (d, 1H).

LC-MS (Method 2): Rₜ=0.86 min; m/z=238 (M+H)⁺.

Analogously to Example 1A, the following compounds were prepared from the reactants specified in each case:

| Example | Name/Structure/Starting materials | Analytical data |
|---|---|---|
| 4A | 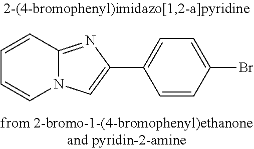 [SMILES: Brc1ccc(-c2cn3ccccc3n2)cc1] | ¹H-NMR (400 MHz, DMSO-d₆, δ/ppm): 6.88-6.94 (m, 1H), 7.23-7.29 (m, 1H), 7.58 (d, 1H), 7.63 (d, 2H), 7.92 (d, 2H), 8.44 (s, 1H), 8.53 (d, 1H). LC-MS (Method 1): Rₜ = 0.63 min; m/z = 273/275 (M + H)⁺. |
| 5A | 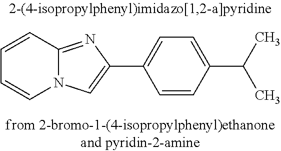 [SMILES: CC(C)c1ccc(-c2cn3ccccc3n2)cc1] | ¹H-NMR (400 MHz, DMSO-d₆, δ/ppm): 1.23 (d, 6H), 2.85-2.96 (m, 1H), 6.88 (t, 1H), 7.19-7.26 (m, 1H), 7.31 (d, 2H), 7.56 (d, 1H), 7.88 (d, 2H), 8.34 (s, 1H), 8.51 (d, 1H). LC-MS (Method 1): Rₜ = 0.68 min; m/z = 237 (M + H)⁺. |

### Example 6A

### 2-(4-Chlorophenyl)imidazo[1,2-a]pyridine-3-carbaldehyde

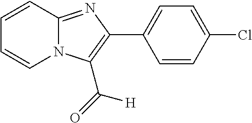 [SMILES: O=Cc1c(-c2ccc(Cl)cc2)nc2ccccn12]

300 ml of DMF were cooled to 0° C. 44 ml (470.08 mmol) of phosphorus oxychloride were then slowly added dropwise. The reaction solution was then slowly warmed to room temperature and stirred at this temperature for another hour. 43 g (188.03 mmol) of 2-(4-chlorophenyl)imidazo[1,2-a]pyridine were then added in portions. During the addition, the reaction solution warmed to 35° C. After the addition had ended, the reaction mixture was heated to 80° C. and stirred at this temperature for 2 hours. After cooling to room temperature, the solution was slowly added to 3 litres of ice-water. The resulting solid was filtered off with suction, washed repeatedly with water and dried in a high-vacuum drying cabinet at 40° C. overnight. 39.6 g (154.27 mmol, 82% of theory) of the target product were obtained.

¹H-NMR (400 MHz, DMSO-d₆, δ/ppm): 7.37 (t, 1H), 7.63 (d, 2H), 7.78 (t, 1H), 7.90-7.99 (m, 3H), 9.58 (d, 1H), 10.02 (s, 1H).

LC-MS (Method 1): Rₜ=0.97 min; m/z=257/259 (M+H)⁺.

### Example 7A

### 2-(5-Chloropyridin-2-yl)imidazo[1,2-a]pyridine-3-carbaldehyde

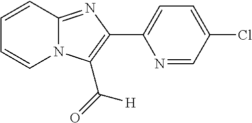 [SMILES: O=Cc1c(-c2ccc(Cl)cn2)nc2ccccn12]

80 ml of DMF were cooled to 0° C. 4.4 ml (47.02 mmol) of phosphorus oxychloride were then slowly added dropwise. The reaction solution was then slowly warmed to room temperature and stirred at this temperature for another hour. 4.32 g (18.81 mmol) of 2-(5-chloropyridin-2-yl)imidazo[1,2-a]pyridine were then added in portions. When the addition had ended, the reaction mixture was heated to 80° C. and stirred at this temperature for 1 h. After cooling to room temperature, the solution was gradually added to ice-water. Ethyl acetate was added and, after thorough shaking, the organic phase was removed. The latter was washed with saturated sodium chloride solution, dried over magnesium sulphate and concentrated to dryness. The resulting residue was purified by column chromatography on silica gel (eluent: cyclohexane/ethyl acetate 2:1). 4.46 g (17.31 mmol, 92% of theory) of the target compound were obtained.

¹H-NMR (400 MHz, DMSO-d₆, δ/ppm): 7.36 (td, 1H), 7.76 (ddd, 1H), 7.94 (d, 1H), 8.15 (dd, 1H), 8.35 (d, 1H), 8.81 (d, 1H), 9.60 (d, 1H), 10.87 (s, 1H).

LC-MS (Method 1): Rₜ=0.92 min; m/z=258/260 (M+H)⁺.

### Example 8A

### 2-(6-Isopropylpyridin-3-yl)imidazo[1,2-a]pyridine-3-carbaldehyde

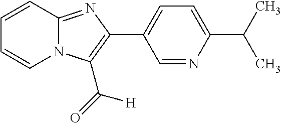 [SMILES: CC(C)c1ccc(-c2nc3ccccn3c2C=O)cn1]

20 ml of DMF were cooled to 0° C. 1.6 ml (17.07 mmol) of phosphorus oxychloride were then slowly added dropwise. The reaction solution was then slowly warmed to room temperature and stirred at this temperature for another hour. 1.62 g (6.83 mmol) of 2-(6-isopropylpyridin-3-yl)imidazo[1,2-a]pyridine were then added. When the addition had ended, the reaction mixture was heated to 80° C. and stirred at this temperature for 1 h. After cooling to room temperature, the solution was gradually added to ice-water. The pH of the solution was gradually adjusted from pH 1 to pH 4 by addition of 1 M sodium hydroxide solution while stirring. The solution was then extracted repeatedly with ethyl acetate, and the combined organic phases were dried over magnesium sulphate and concentrated to dryness. The residue obtained was purified by column chromatography on silica gel (Biotage SNAP cartridge KP-NH column; eluent: cyclohexane/ethyl acetate 1:1). In this way, two fractions of the target compound were isolated: fraction 1: 850 mg (pure), fraction 2: 640 mg (still contaminated). The latter fraction was purified once again under the same chromatography conditions, which gave a further 350 mg of the pure target compound. A total of 1.20 g (4.52 mmol, 66% of theory) of the target compound were obtained.

LC-MS (Method 2): Rₜ=1.37 min; m/z=266 (M+H)⁺.

Analogously to Example 6A, the following compounds were prepared from the reactant specified in each case:

| Example | Name/Structure/Starting material | Analytical data |
|---|---|---|
| 9A | 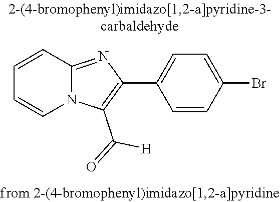 [SMILES: O=Cc1c(-c2ccc(Br)cc2)nc2ccccn12] | ¹H-NMR (400 MHz, DMSO-d₆, δ/ppm): 7.35 (t, 1H), 7.72-7.80 (m, 3H), 7.85-7.95 (m, 3H), 9.58 (d, 1H), 10.02 (s, 1H). LC-MS (Method 2): Rₜ = 1.76 min; m/z = 301/303 (M + H)⁺. |
| 10A | 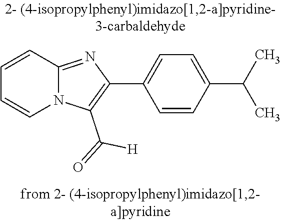 [SMILES: CC(C)c1ccc(-c2nc3ccccn3c2C=O)cc1] | ¹H-NMR (400 MHz, DMSO-d₆, δ/ppm): 1.27 (d, 6H), 2.93-3.05 (m, 1H), 7.33 (t, 1H), 7.44 (d, 2H), 7.74 (t, 1H), 7.85 (d, 2H), 7.91 (d, 1H), 9.58 (d, 1H), 10.03 (s, 1H). LC-MS (Method 1): Rₜ = 1.03 min; m/z = 265 (M + H)⁺. |

### Example 11A

### 2-{[2-(4-Chlorophenyl)imidazo[1,2-a]pyridin-3-yl]methyl}-2,5-diazabicyclo[2.2.2]octane dihydrochloride (Enantiomer 1))

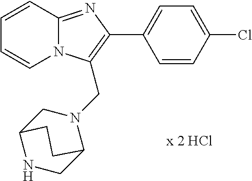 [SMILES: Clc1ccc(-c2nc3ccccn3c2CN2CC3CCC2CN3)cc1]

To 2.64 g (5.83 mmol) of tert-butyl 5-{[2-(4-chlorophenyl)imidazo[1,2-a]pyridin-3-yl]methyl}-2,5-diazabicyclo[2.2.2]octane-2-carboxylate (enantiomer 1) were added, while stirring, 14.6 ml of a 4 M solution of hydrogen chloride in dioxane. The mixture was stirred at room temperature overnight. The solids obtained were then filtered off with suction, washed repeatedly with diethyl ether and dried under high vacuum at 40° C. 3.55 g of a solid material were obtained, which was used in subsequent reactions without further purification.

LC-MS (Method 5): Rₜ=0.44 min; m/z=353/355 (M+H)⁺.

### Example 12A

### 2-{[2-(5-Chloropyridin-2-yl)imidazo[1,2-a]pyridin-3-yl]methyl}-2,5-diazabicyclo[2.2.2]octane dihydrochloride (Enantiomer 1)

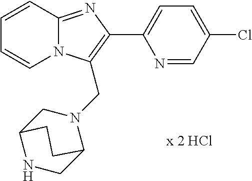 [SMILES: Clc1ccc(-c2nc3ccccn3c2CN2CC3CCC2CN3)nc1]

To 450 mg (0.99 mmol) of tert-butyl 5-{[2-(5-chloropyridin-2-yl)imidazo[1,2-a]pyridin-3-yl]methyl}-2,5-diazabicyclo[2.2.2]octane-2-carboxylate (enantiomer 1) were added, while stirring, 1.49 ml of a 4 M solution of hydrogen chloride in dioxane and a further 5 ml of dioxane. The mixture was stirred at room temperature overnight. The solids obtained were then filtered off with suction, washed repeatedly with diethyl ether and dried under high vacuum at 40° C. 464 mg of a solid material were obtained, which was used in subsequent reactions without further purification.

LC-MS (Method 2): Rₜ=0.70 min; m/z=354 (M+H)⁺.

### Example 13A

### 2-{[2-(6-Isopropylpyridin-3-yl)imidazo[1,2-a]pyridin-3-yl]methyl}-2,5-diazabicyclo[2.2.2]octane dihydrochloride (Racemate)

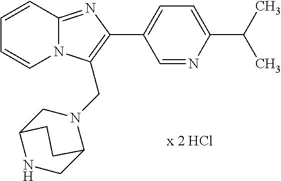 [SMILES: CC(C)c1ccc(-c2nc3ccccn3c2CN2CC3CCC2CN3)cn1]

To 820 mg (1.78 mmol) of tert-butyl 5-{[2-(6-isopropylpyridin-3-yl)imidazo[1,2-a]pyridin-3-yl]methyl}-2,5-diazabicyclo[2.2.2]octane-2-carboxylate (racemate) were added, while stirring, 4.44 ml of a 4 M solution of hydrogen chloride in dioxane and a further 10 ml of dioxane. The mixture was stirred at room temperature overnight. The solids obtained were then filtered off with suction, washed repeatedly with diethyl ether and dried under high vacuum at 40° C. 883 mg of a solid material were obtained, which was used in subsequent reactions without further purification.

LC-MS (Method 1): Rₜ=0.40 min; m/z=362 (M+H)⁺.

### Example 14A

### 7-{[2-(4-Chlorophenyl)imidazo[1,2-a]pyridin-3-yl]methyl}-3-oxa-7,9-diazabicyclo[3.3.1]nonane dihydrochloride

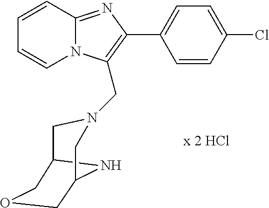 [SMILES: Clc1ccc(-c2nc3ccccn3c2CN2CC3COCC(C2)N3)cc1]

To 1.87 g (3.99 mmol) of tert-butyl 7-{[2-(4-chlorophenyl)imidazo[1,2-a]pyridin-3-yl]methyl}-3-oxa-7,9-diazabicyclo[3.3.1]nonane-9-carboxylate were added, while stirring, 10 ml of a 4 M solution of hydrogen chloride in dioxane. The mixture was stirred at room temperature overnight. The solids obtained were then filtered off with suction, washed repeatedly with diethyl ether and dried under high vacuum at 40° C. 1.99 g of a solid material were obtained, which was used in subsequent reactions without further purification.

LC-MS (Method 4): Rₜ=1.30 min; m/z=369/371 (M+H)⁺.

Analogously to Examples 11A-14A, the following compounds were prepared from the reactant specified in each case:

| Example | Name/Structure/Starting material | Analytical data |
|---|---|---|
| 15A | 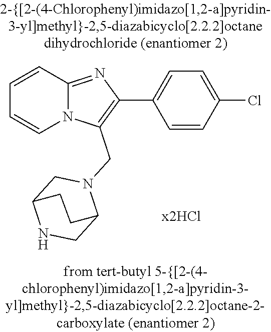 [SMILES: Clc1ccc(-c2nc3ccccn3c2CN2CC3CCC2CN3)cc1 ; "x2HCl"] | LC-MS (Method 5): Rₜ = 0.40 min; m/z = 353/355 (M + H)⁺. |
| 16A | 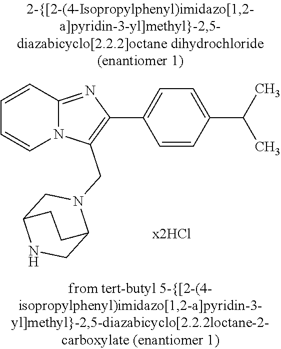 [SMILES: CC(C)c1ccc(-c2nc3ccccn3c2CN2CC3CCC2CN3)cc1 ; "x2HCl"] | LC-MS (Method 1): Rₜ = 0.48 min; m/z = 361 (M + H)⁺. |
| 17A | 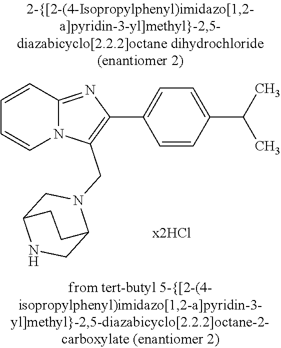 [SMILES: CC(C)c1ccc(-c2nc3ccccn3c2CN2CC3CCC2CN3)cc1 ; "x2HCl"] | LC-MS (Method 1): Rₜ = 0.49 min; m/z = 361 (M + H)⁺. |
| 18A | 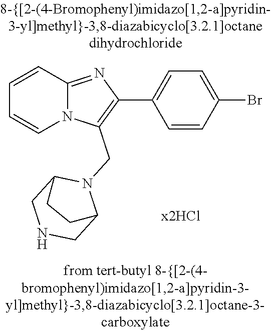 [SMILES: Brc1ccc(-c2nc3ccccn3c2CN2C3CCC2CNC3)cc1 ; "x2HCl"] | LC-MS (Method 1): Rₜ = 0.48 min; m/z = 397/399 (M + H)⁺. |
| 19A | 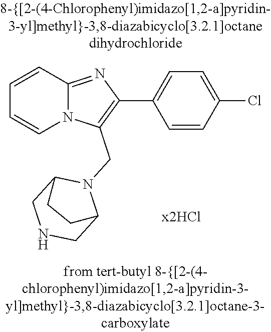 [SMILES: Clc1ccc(-c2nc3ccccn3c2CN2C3CCC2CNC3)cc1 ; "x2HCl"] | LC-MS (Method 4): Rₜ = 1.24 min; m/z = 353/355 (M + H)⁺. |
| 20A | 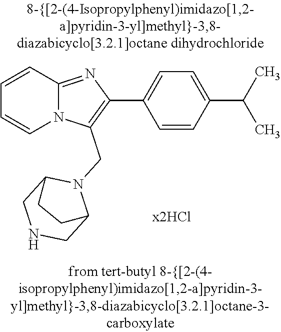 [SMILES: CC(C)c1ccc(-c2nc3ccccn3c2CN2C3CCC2CNC3)cc1 ; "x2HCl"] | LC-MS (Method 4): Rₜ = 1.41 min; m/z = 361 (M + H)⁺. |
| 21A | 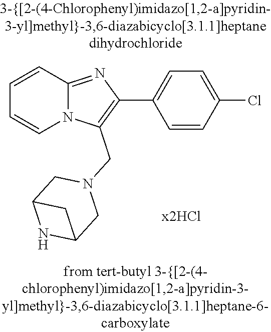 [SMILES: Clc1ccc(-c2nc3ccccn3c2CN2CC3CC(C2)N3)cc1 ; "x2HCl"] | LC-MS (Method 4): Rₜ = 1.22 min; m/z = 339/341 (M + H)⁺. |
| 22A | 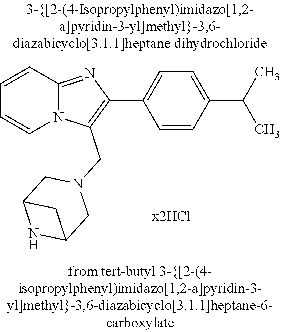 [SMILES: CC(C)c1ccc(-c2nc3ccccn3c2CN2CC3CC(C2)N3)cc1 ; "x2HCl"] | LC-MS (Method 4): Rₜ = 1.33 min; m/z = 347 (M + H)⁺. |
| 23A | 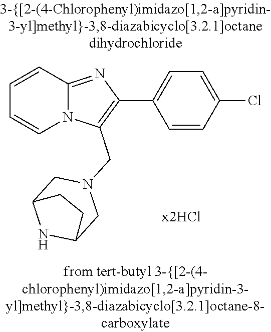 [SMILES: Clc1ccc(-c2nc3ccccn3c2CN2CC3CCC(C2)N3)cc1 ; "x2HCl"] | LC-MS (Method 4): Rₜ = 1.23 min; m/z = 353/355 (M + H)⁺. |
| 24A | 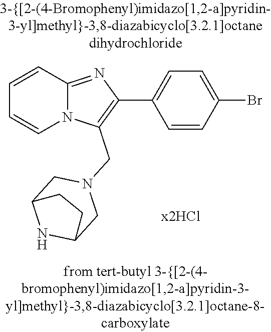 [SMILES: Brc1ccc(-c2nc3ccccn3c2CN2CC3CCC(C2)N3)cc1 ; "x2HCl"] | LC-MS (Method 1): Rₜ = 0.46 min; m/z = 397/399 (M + H)⁺. |
| 25A | 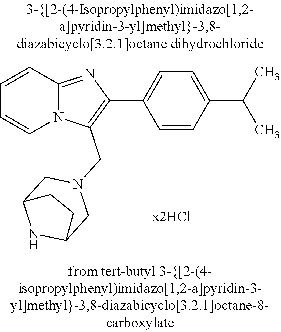 [SMILES: CC(C)c1ccc(-c2nc3ccccn3c2CN2CC3CCC(C2)N3)cc1 ; "x2HCl"] | LC-MS (Method 4): Rₜ = 1.38 min; m/z = 361 (M + H)⁺. |
| 26A | 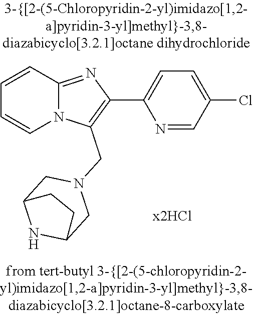 [SMILES: Clc1ccc(-c2nc3ccccn3c2CN2CC3CCC(C2)N3)nc1 ; "x2HCl"] | LC-MS (Method 2): Rₜ = 0.74 min; m/z = 354 (M + H)⁺. |
| 27A | 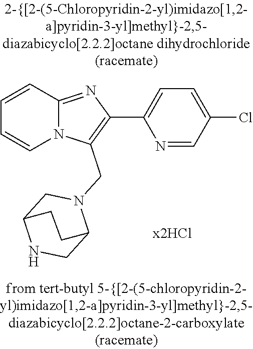 [SMILES: Clc1ccc(-c2nc3ccccn3c2CN2CC3CCC2CN3)nc1 ; "x2HCl"] | LC-MS (Method 4): Rₜ = 1.27 min; m/z = 354/356 (M + H)⁺. |
| 28A | 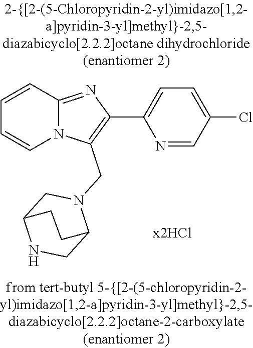 [SMILES: Clc1ccc(-c2nc3ccccn3c2CN2CC3CCC2CN3)nc1 ; "x2HCl"] | LC-MS (Method 2): Rₜ = 0.70 min; m/z = 354 (M + H)⁺. |
| 29A | 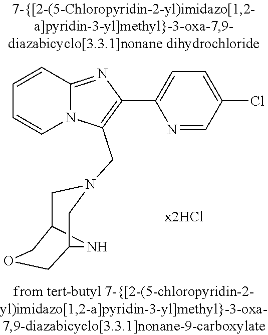 [SMILES: Clc1ccc(-c2nc3ccccn3c2CN2CC3COCC(C2)N3)nc1 ; "x2HCl"] | LC-MS (Method 2): Rₜ = 0.71 min; MS (ESIpos): m/z = 370 [M + H]⁺. |
| 30A | 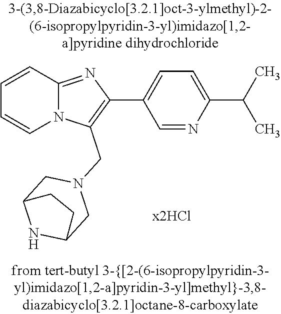 [SMILES: CC(C)c1ccc(-c2nc3ccccn3c2CN2CC3CCC(C2)N3)cn1 ; "x2HCl"] | LC-MS (Method 2): Rₜ = 0.64 min; MS (ESIpos): m/z = 362 [M + H]⁺. |
| 31A | 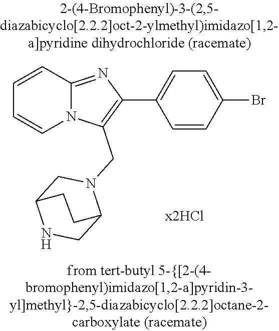 [SMILES: Brc1ccc(-c2nc3ccccn3c2CN2CC3CCC2CN3)cc1 ; "x2HCl"] | LC-MS (Method 5): Rₜ = 0.61 min; MS (ESIpos): m/z = 397 [M + H]⁺. |

### Example 32A

### 2-{[2-(6-Isopropylpyridin-3-yl)imidazo[1,2-a]pyridin-3-yl]methyl}-2,5-diazabicyclo[2.2.2]octane dihydrochloride (Enantiomer 1)

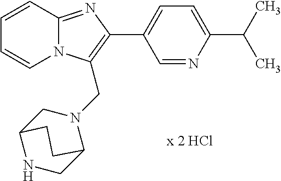 [SMILES: CC(C)c1ccc(-c2nc3ccccn3c2CN2CC3CCC2CN3)cn1]

To 1090 mg (2.36 mmol) of tert-butyl 5-{[2-(6-isopropylpyridin-3-yl)imidazo[1,2-a]pyridin-3-yl]methyl}-2,5-diazabicyclo[2.2.2]octane-2-carboxylate (enantiomer 1) were added, while stirring, 8.86 ml of a 4 M solution of hydrogen chloride in dioxane and a further 10 ml of dioxane. The mixture was stirred at room temperature overnight. The solids obtained were then filtered off with suction, washed repeatedly with diethyl ether and dried under high vacuum at 40° C. 1195 mg of a solid material were obtained, which was used in subsequent reactions without further purification.

LC-MS (Method 2): Rₜ=0.61 min; m/z=362 (M+H)⁺.

### Example 33A

### 2-{[2-(6-Isopropylpyridin-3-yl)imidazo[1,2-a]pyridin-3-yl]methyl}-2,5-diazabicyclo[2.2.2]octane dihydrochloride (Enantiomer 2)

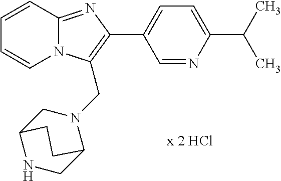 [SMILES: CC(C)c1ccc(-c2nc3ccccn3c2CN2CC3CCC2CN3)cn1]

To 1010 mg (2.19 mmol) of tert-butyl 5-{[2-(6-isopropylpyridin-3-yl)imidazo[1,2-a]pyridin-3-yl]methyl}-2,5-diazabicyclo[2.2.2]octane-2-carboxylate (enantiomer 2) were added, while stirring, 8.86 ml of a 4 M solution of hydrogen chloride in dioxane. The mixture was stirred at room temperature overnight. The solids obtained were then filtered off with suction, washed repeatedly with diethyl ether and dried under high vacuum at 40° C. 1050 mg of a solid material were obtained, which was used in subsequent reactions without further purification.

LC-MS (Method 2): Rₜ=0.63 min; m/z=362 (M+H)⁺.

### INVENTIVE EXAMPLES

### Example 1

### tert-Butyl 5-{[2-(4-chlorophenyl)imidazo[1,2-a]pyridin-3-yl]methyl}-2,5-diazabicyclo[2.2.2]octane-2-carboxylate (Racemate)

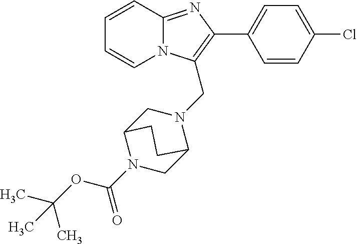 [SMILES: CC(C)(C)OC(=O)N1CC2CCC1CN2Cc1c(-c2ccc(Cl)cc2)nc2ccccn12]

Under argon and at room temperature, 5 g (19.48 mmol) of 2-(4-chlorophenyl)imidazo[1,2-a]pyridine-3-carbaldehyde were dissolved in 100 ml of THF, and 8.27 g (38.96 mmol) of tert-butyl 2,5-diazabicyclo[2.2.2]octane-2-carboxylate (racemate) and 2.23 ml (38.96 mmol) of acetic acid were added. Subsequently, 6.19 g (29.22 mmol) of sodium triacetoxyborohydride were added in portions, and the reaction solution was stirred at room temperature overnight. Then water was gradually and cautiously added dropwise (caution: evolution of gas) and then ethyl acetate was added. The resulting organic phase was removed and the aqueous phase was extracted twice with ethyl acetate. The combined organic phases were dried over magnesium sulphate, filtered and concentrated to dryness under reduced pressure on a rotary evaporator. The resulting residue was applied to silica gel and purified by column chromatography on silica gel (eluent: cyclohexane/ethyl acetate 1:1). 6.58 g (13.70 mmol, 70% of theory) of the target compound were obtained.

¹H-NMR (400 MHz, DMSO-d₆, δ/ppm): 1.36 (2 s, 9H), 1.43-1.54 (m, 1H), 1.57-1.73 (m, 2H), 1.79-1.89 (m, 1H), 2.61-2.78 (m, 3H), 3.13 (br. t, 1H), 3.50 (br. t, 1H), 3.81 (br. d, 1H), 4.16-4.27 (m, 2H), 6.97 (t, 1H), 7.31 (t, 1H), 7.52 (d, 2H), 7.59 (d, 1H), 7.82-7.90 (m, 2H), 8.57 (d, 1H).

LC-MS (Method 2): Rₜ=1.50 min; m/z=453/455 (M+H)⁺.

### Example 2

### tert-Butyl 5-{[2-(5-chloropyridin-2-yl)imidazo[1,2-a]pyridin-3-yl]methyl}-2,5-diazabicyclo[2.2.2]octane-2-carboxylate (Racemate)

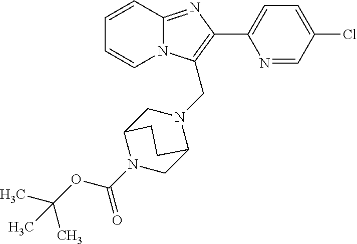 [SMILES: CC(C)(C)OC(=O)N1CC2CCC1CN2Cc1c(-c2ccc(Cl)cn2)nc2ccccn12]

Under argon and at room temperature, 1.1 g (4.27 mmol) of 2-(5-chloropyridin-2-yl)imidazo[1,2-a]pyridine-3-carbaldehyde were dissolved in 35 ml of THF, and 1.36 g (6.40 mmol) of tert-butyl 2,5-diazabicyclo[2.2.2]octane-2-carboxylate (racemate) and 0.49 ml (8.54 mmol) of acetic acid were added. Subsequently, 1.36 g (6.40 mmol) of sodium triacetoxyborohydride were added in portions, and the reaction solution was stirred at room temperature overnight. Then water was gradually and cautiously added dropwise (caution: evolution of gas) and then ethyl acetate was added. The resulting organic phase was removed and the aqueous phase was extracted twice with ethyl acetate. The combined organic phases were washed with saturated sodium chloride solution, dried over magnesium sulphate, filtered and concentrated to dryness under reduced pressure on a rotary evaporator. The residue obtained was purified by column chromatography on silica gel (Biotage SNAP cartridge KP-NH column; eluent: cyclohexane/ethyl acetate 1:1). 1.57 g (3.46 mmol, 81% of theory) of the target compound were obtained.

¹H-NMR (400 MHz, DMSO-d₆, δ/ppm): 1.39 (2 s, 9H), 1.44-1.58 (m, 1H), 1.70 (br. t, 2H), 1.85-2.01 (m, 1H), 2.70 (br. s, 0.5H), 2.78 (br. s, 0.5H), 2.82-2.96 (m, 2H), 3.14 (br. d, 1H), 3.63 (br. dd, 1H), 3.81 (br. s, 0.5H), 3.87 (br. s, 0.5H), 4.55-4.71 (m, 2H), 6.99 (t, 1H), 7.35 (t, 1H), 7.62 (d, 1H), 8.01 (br. d, 1H), 8.21 (d, 1H), 8.48 (d, 1H), 8.63 (dd, 1H).

LC-MS (Method 2): Rₜ=1.27 min; m/z=454/456 (M+H)⁺.

### Example 3

### tert-Butyl 5-{[2-(6-isopropylpyridin-3-yl)imidazo[1,2-a]pyridin-3-yl]methyl}-2,5-diazabicyclo[2.2.2]octane-2-carboxylate (Racemate)

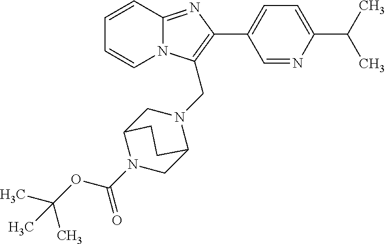 [SMILES: CC(C)c1ccc(-c2nc3ccccn3c2CN2CC3CCC2CN3C(=O)OC(C)(C)C)cn1]

Under argon and at room temperature, 670 mg (2.53 mmol) of 2-(6-isopropylpyridin-3-yl)imidazo[1,2-a]pyridine-3-carbaldehyde were dissolved in 15 ml of THF, and 643 mg (3.03 mmol) of tert-butyl 2,5-diazabicyclo[2.2.2]octane-2-carboxylate (racemate) and 0.29 ml (5.05 mmol) of acetic acid were added. Subsequently, 803 mg (3.79 mmol) of sodium triacetoxyborohydride were added in portions, and the reaction solution was stirred at room temperature overnight. Then water was gradually and cautiously added dropwise (caution: evolution of gas) and then ethyl acetate was added. The resulting organic phase was removed and the aqueous phase was extracted twice with ethyl acetate. The combined organic phases were dried over magnesium sulphate, filtered and concentrated to dryness under reduced pressure on a rotary evaporator. The residue was taken up in dichloromethane and filtered. The resulting filtrate was again concentrated to dryness. The residue thus obtained was purified by column chromatography on silica gel (Biotage SNAP cartridge KP-NH column; eluent: cyclohexane/ethyl acetate 1:1). 720 mg (1.56 mmol, 62% of theory) of the target compound were obtained.

¹H-NMR (400 MHz, DMSO-d₆, δ/ppm): 1.28 (d, 6H), 1.36 (2 s, 9H), 1.43-1.55 (m, 1H), 1.57-1.75 (m, 2H), 1.78-1.91 (m, 1H), 2.65-2.82 (m, 3H), 3.01-3.19 (m, 2H), 3.53 (dd, 1H), 3.81 (br. d, 1H), 4.17-4.28 (m, 2H), 6.98 (t, 1H), 7.31 (t, 1H), 7.38 (d, 1H), 7.61 (d, 1H), 8.13 (dt, 1H), 8.58 (d, 1H), 8.92 (dd, 1H).

LC-MS (Method 2): Rₜ=1.41 min; m/z=462 (M+H)⁺.

### Example 4

### tert-Butyl 7-{[2-(4-chlorophenyl)imidazo[1,2-a]pyridin-3-yl]methyl}-3-oxa-7,9-diazabicyclo[3.3.1]nonane-9-carboxylate

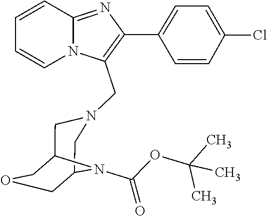 [SMILES: CC(C)(C)OC(=O)N1C2COCC1CN(Cc1c(-c3ccc(Cl)cc3)nc3ccccn13)C2]

Under argon and at room temperature, 1.406 g (5.48 mmol) of 2-(4-chlorophenyl)imidazo[1,2-a]pyridine-3-carbaldehyde were dissolved in 25 ml of THF, and 1.5 g (6.57 mmol) of tert-butyl 3-oxa-7,9-diazabicyclo[3.3.1]nonane-9-carboxylate and 0.63 ml (10.95 mmol) of acetic acid were added. Subsequently, 1.74 g (8.21 mmol) of sodium triacetoxyborohydride were added in portions, and the reaction solution was stirred at room temperature overnight. Then water was gradually and cautiously added dropwise (caution: evolution of gas) and then ethyl acetate was added. The resulting organic phase was removed and the aqueous phase was extracted twice with ethyl acetate. The combined organic phases were dried over magnesium sulphate, filtered and concentrated to dryness under reduced pressure on a rotary evaporator. The residue obtained was purified by column chromatography on silica gel (Biotage SNAP cartridge KP-NH column; eluent: cyclohexane/ethyl acetate 1:1). 1.87 g (3.99 mmol, 73% of theory) of the target compound were obtained.

¹H-NMR (400 MHz, DMSO-d₆, δ/ppm): 1.35-1.46 (m, 9H), 2.43 (br. d, 2H), 2.85 (br. d, 2H), 3.57 (br. d, 2H), 3.71 (d, 2H), 3.81-3.92 (m, 4H), 6.93 (td, 1H), 7.30 (ddd, 1H), 7.51 (d, 2H), 7.60 (d, 1H), 7.97 (d, 2H), 8.81 (d, 1H).

LC-MS (Method 2): Rₜ=1.52 min; m/z=469/471 (M+H)⁺.

Analogously to Examples 1-4, the following compounds were prepared from the reactants specified in each case:

| Example | Name/Structure/Starting materials | Analytical data |
|---|---|---|
| 5 | 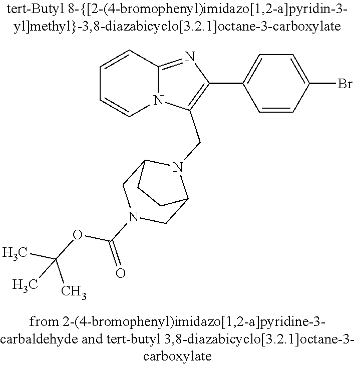 [SMILES: CC(C)(C)OC(=O)N1CC2CCC(C1)N2Cc1c(-c2ccc(Br)cc2)nc2ccccn12] | ¹H-NMR (400 MHz, DMSO-d₆, δ/ppm): 1.36 (s, 9H), 1.42-1.50 (m, 2H), 1.78-1.91 (m, 2H), 2.63-2.75 (m, 1H), 2.76-2.88 (m, 1H), 3.00-3.14 (m, 2H), 3.44-3.61 (m, 2H), 3.98 (s, 2H), 6.98 (td, 1H), 7.32 (ddd, 1H), 7.60 (d, 1H), 7.67 (d, 2H), 7.81 (d, 2H), 8.64 (d, 1H). LC-MS (Method 2): Rₜ = 1.64 min; m/z = 497/499 (M + H)⁺. |
| 6 | 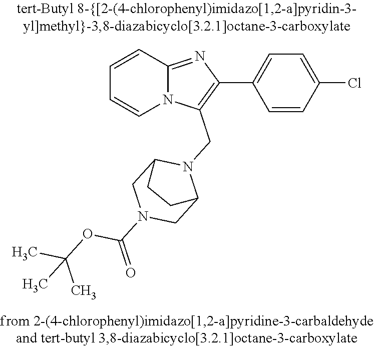 [SMILES: CC(C)(C)OC(=O)N1CC2CCC(C1)N2Cc1c(-c2ccc(Cl)cc2)nc2ccccn12] | ¹H-NMR (400 MHz, DMSO-d₆, δ/ppm): 1.35 (s, 9H), 1.46 (q, 2H), 1.79-1.89 (m, 2H), 2.63-2.76 (m, 1H), 2.77-2.88 (m, 1H), 3.00-3.14 (m, 2H), 3.42-3.61 (m, 2H), 3.98 (s, 2H), 6.98 (td, 1H), 7.32 (ddd, 1H), 7.53 (d, 2H), 7.60 (d, 1H), 7.87 (d, 2H), 8.64 (d, 1H). LC-MS (Method 1): Rₜ = 0.84 min; m/z = 453/455 (M + H)⁺. |
| 7 | 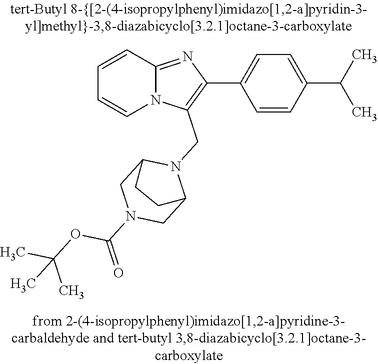 [SMILES: CC(C)c1ccc(-c2nc3ccccn3c2CN2C3CCC2CN(C(=O)OC(C)(C)C)C3)cc1] | ¹H-NMR (400 MHz, DMSO-d₆, δ/ppm): 1.24 (d, 6H), 1.35 (s, 9H), 1.46 (q, 2H), 1.78-1.89 (m, 2H), 2.65-2.75 (m, 1H), 2.77-2.87 (m, 1H), 2.87-3.01 (m, 1H), 3.04-3.17 (m, 2H), 3.43-3.62 (m, 2H), 3.98 (s, 2H), 6.96 (t, 1H), 7.29 (t, 1H), 7.34 (d, 2H), 7.58 (d, 1H), 7.74 (d, 2H), 8.64 (d, 1H). LC-MS (Method 2): Rₜ = 1.63 min; m/z = 461 (M + H)⁺. |
| 8 | 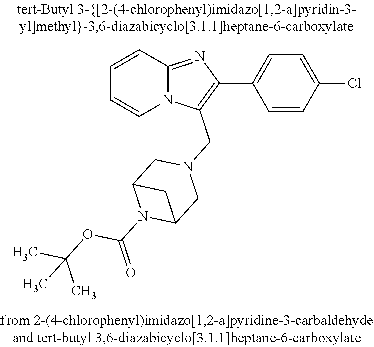 [SMILES: CC(C)(C)OC(=O)N1C2CC1CN(Cc1c(-c3ccc(Cl)cc3)nc3ccccn13)C2] | ¹H-NMR (400 MHz, DMSO-d₆, δ/ppm): 1.27 (s, 9H), 1.62 (d, 1H), 2.20 (q, 1H), 2.57-3.04 (m, 4H), 3.83-3.98 (m, 2H), 4.09-4.26 (m, 2H), 6.98 (t, 1H), 7.31 (t, 1H), 7.51 (d, 2H), 7.61 (d, 1H), 7.82 (d, 2H), 8.47 (d, 1H). LC-MS (Method 1): Rₜ = 0.87 min; m/z = 439/441 (M + H)⁺. |
| 9 | 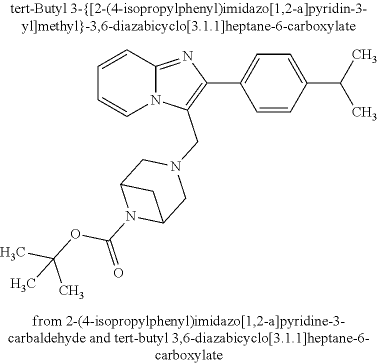 [SMILES: CC(C)c1ccc(-c2nc3ccccn3c2CN2CC3CC(C2)N3C(=O)OC(C)(C)C)cc1] | ¹H-NMR (400 MHz, DMSO-d₆, δ/ppm): 1.25 (d, 6H), 1.28 (s, 9H), 1.65 (d, 1H), 2.20 (q, 1H), 2.56-3.02 (m, 5H), 3.81-4.03 (m, 2H) 4.05-4.24 (m, 2H), 6.94 (t, 1H), 7.24-7.35 (m, 3H), 7.59 (d, 1H), 7.71 (d, 2H), 8.44 (d, 1H). LC-MS (Method 1): Rₜ = 0.88 min; m/z = 447 (M + H)⁺. |
| 10 | 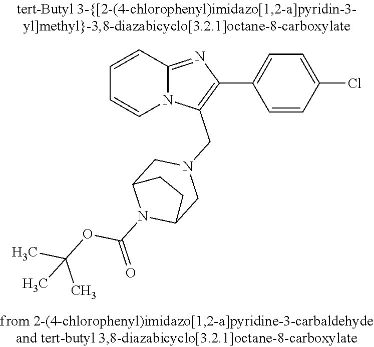 [SMILES: CC(C)(C)OC(=O)N1C2CCC1CN(Cc1c(-c3ccc(Cl)cc3)nc3ccccn13)C2] | ¹H-NMR (400 MHz, DMSO-d₆, δ/ppm): 1.39 (s, 9H), 1.64 (br. s, 4H), 2.27 (br. d, 2H), 2.48-2.58 (m, 2H, partly concealed by DMSO signal), 3.97 (br. s, 2H), 4.03 (br. s, 2H), 6.99 (t, 1H), 7.31 (t, 1H), 7.53 (d, 2H), 7.60 (d, 1H), 7.92 (d, 2H), 8.58 (d, 1H). LC-MS (Method 2): Rₜ = 1.81 min; m/z = 453/455 (M + H)⁺. |
| 11 | 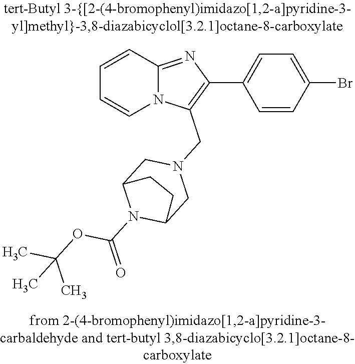 [SMILES: CC(C)(C)OC(=O)N1C2CCC1CN(Cc1c(-c3ccc(Br)cc3)nc3ccccn13)C2] | ¹H-NMR (400 MHz, DMSO-d₆, δ/ppm): 1.39 (s, 9H), 1.64 (br. s, 4H), 2.27 (br. d, 2H), 2.46-2.58 (m, 2H, concealed by DMSO signal), 3.97 (s, 2H), 4.03 (br. s, 2H), 6.99 (td, 1H), 7.31 (ddd, 1H), 7.60 (d, 1H), 7.67 (d, 2H), 7.85 (d, 2H), 8.58 (d, 1H). LC-MS (Method 2): Rₜ = 1.86 min; m/z = 497/499 (M + H)⁺. |
| 12 | 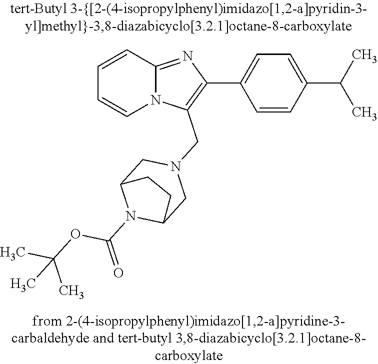 [SMILES: CC(C)c1ccc(-c2nc3ccccn3c2CN2CC3CCC(C2)N3C(=O)OC(C)(C)C)cc1] | ¹H-NMR (400 MHz, DMSO-d₆, δ/ppm): 1.25 (d, 6H), 1.39 (s, 9H), 1.65 (br. s, 4H), 2.26 (br. d, 2H), 2.46-2.59 (m, 2H, partly concealed by DMSO signal), 2.88-3.01 (m, 1H), 3.97 (s, 2H), 4.03 (br. s, 2H), 6.96 (t, 1H), 7.28 (t, 1H), 7.34 (d, 2H), 7.58 (d, 1H), 7.79 (d, 2H), 8.55 (d, 1H). LC-MS (Method 2): Rₜ = 1.74 min; m/z = 461 (M + H)⁺. |
| 13 | 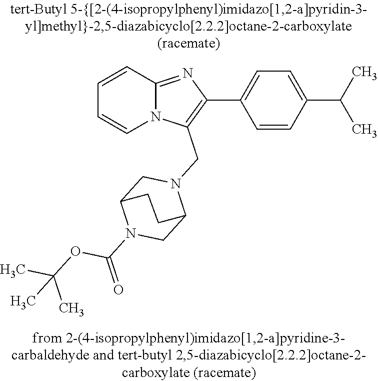 [SMILES: CC(C)c1ccc(-c2nc3ccccn3c2CN2CC3CCC2CN3C(=O)OC(C)(C)C)cc1] | ¹H-NMR (400 MHz, DMSO-d₆, δ/ppm): 1.25 (d, 6H), 1.36 (2 s, 9H), 1.43-1.56 (m, 1H), 1.58-1.76 (m, 2H), 1.77-1.93 (m, 1H), 2.64-2.82 (m, 3H), 2.87-3.01 (m, 1H), 3.13 (br. t, 1H), 3.52 (br. d, 1H), 3.82 (br. d, 1H), 4.21 (s, 2H), 6.95 (t, 1H), 7.28 (t, 1H), 7.34 (d, 2H), 7.58 (d, 1H), 7.70-7.79 (m, 2H), 8.55 (d, 1H). LC-MS (Method 2): Rₜ = 1.60 min; m/z = 461 (M + H)⁺. |
| 14 | 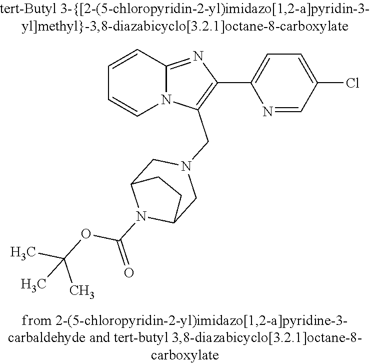 [SMILES: CC(C)(C)OC(=O)N1C2CCC1CN(Cc1c(-c3ccc(Cl)cn3)nc3ccccn13)C2] | ¹H-NMR (400 MHz, DMSO-d₆, δ/ppm): 1.38 (s, 9H), 1.62 (br. s, 4H), 2.25 (br. d, 2H), 2.45-2.59 (m, 2H, partly concealed by DMSO signal), 3.98 (br. s, 2H), 4.45 (s, 2H), 7.01 (td, 1H), 7.34 (t, 1H), 7.62 (d, 1H), 8.00 (dd, 1H), 8.20 (d, 1H), 8.54 (d, 1H), 8.65 (d, 1H). LC-MS (Method 2): Rₜ = 1.50 min; m/z = 454/456 (M + H)⁺. |

### Example 15 and Example 16

### tert-Butyl 5-{[2-(4-chlorophenyl)imidazo[1,2-a]pyridin-3-yl]methyl}-2,5-diazabicyclo[2.2.2]octane-2-carboxylate (Enantiomer 1 and 2)

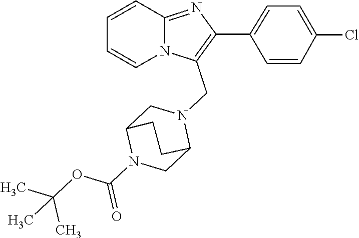 [SMILES: CC(C)(C)OC(=O)N1CC2CCC1CN2Cc1c(-c2ccc(Cl)cc2)nc2ccccn12]

5.86 g (12.94 mmol) of racemic tert-butyl 5-{[2-(4-chlorophenyl)imidazo[1,2-a]pyridin-3-yl]methyl}-2,5-diazabicyclo[2.2.2]octane-2-carboxylate (Example 1) were separated into the enantiomers by preparative HPLC on a chiral phase [column: Daicel Chiralpak IC, 5 μm, 250 mm×20 mm; eluent: isohexane/ethanol 80:20; flow rate: 15 ml/min; UV detection: 220 nm; temperature: 30° C.]:

### Example 15 (Enantiomer 1)

Yield: 2640 mg

Rₜ=9.85 min; chemical purity >99%; >99% ee

[Column: Daicel Chiralpak IC, 5 μm, 250 mm×4.6 mm; eluent: isohexane/ethanol 80:20; flow rate: 1 ml/min; temperature: 30° C.; UV detection: 220 nm].

LC-MS (Method 2): Rₜ=1.52 min; m/z=453/455 (M+H)⁺.

¹H-NMR (400 MHz, DMSO-d₆, δ/ppm): 1.36 (2 s, 9H), 1.43-1.54 (m, 1H), 1.57-1.72 (m, 2H), 1.78-1.91 (m, 1H), 2.61-2.79 (m, 3H), 3.13 (br. t, 1H), 3.50 (br. t, 1H), 3.80 (br. d, 1H), 4.16-4.27 (m, 2H), 6.97 (t, 1H), 7.31 (t, 1H), 7.52 (d, 2H), 7.59 (d, 1H), 7.82-7.90 (m, 2H), 8.57 (d, 1H).

### Example 16 (Enantiomer 2)

Yield: 2430 mg

Rₜ=10.62 min; chemical purity >99%; >99% ee

[Column: Daicel Chiralpak IC, 5 μm, 250 mm×4.6 mm; eluent: isohexane/ethanol 80:20; flow rate: 1 ml/min; temperature: 30° C.; UV detection: 220 nm].

LC-MS (Method 1): Rₜ=0.81 min; m/z=453/455 (M+H)⁺.

¹H-NMR (400 MHz, DMSO-d₆, δ/ppm): 1.36 (2 s, 9H), 1.42-1.55 (m, 1H), 1.59-1.72 (m, 2H), 1.78-1.90 (m, 1H), 2.61-2.79 (m, 3H), 3.13 (br. t, 1H), 3.50 (br. t, 1H), 3.81 (br. d, 1H), 4.16-4.26 (m, 2H), 6.97 (t, 1H), 7.31 (t, 1H), 7.52 (d, 2H), 7.59 (d, 1H), 7.82-7.91 (m, 2H), 8.57 (d, 1H).

### Example 17 and Example 18

### tert-Butyl 5-{[2-(4-isopropylphenyl)imidazo[1,2-a]pyridin-3-yl]methyl}-2,5-diazabicyclo[2.2.2]octane-2-carboxylate (Enantiomer 1 and 2)

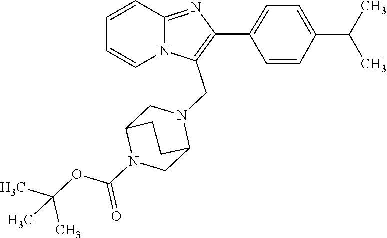 [SMILES: CC(C)c1ccc(-c2nc3ccccn3c2CN2CC3CCC2CN3C(=O)OC(C)(C)C)cc1]

3.91 g (14.79 mmol) of racemic tert-butyl 5-{[2-(4-isopropylphenyl)imidazo[1,2-a]pyridin-3-yl]methyl}-2,5-diazabicyclo[2.2.2]octane-2-carboxylate (Example 13) were separated into the enantiomers by preparative supercritical liquid chromatography (SFC) on a chiral phase [column: Daicel Chiralpak ID-H, 5 μm, 250 mm×20 mm; eluent: carbon dioxide/ethanol 67:33 (v/v); flow rate: 175 ml/min; pressure: 135 bar; UV detection: 210 nm; temperature: 38° C.]:

### Example 17 (Enantiomer 1)

Yield: 1889 mg

Rₜ=3.39 min; chemical purity >99%; >99% ee

[Column: Daicel Chiralpak AD-H, 3 μm, 50 mm×4.6 mm; eluent: carbon dioxide/methanol 5:95→50:50 (v/v); flow rate: 3 ml/min; pressure: 130 bar; temperature: 40° C.; UV detection: 220 nm].

LC-MS (Method 1): Rₜ=0.88 min; m/z=461 (M+H)⁺.

¹H-NMR (400 MHz, DMSO-d₆, δ/ppm): 1.25 (d, 6H), 1.36 (2 s, 9H), 1.43-1.56 (m, 1H), 1.58-1.76 (m, 2H), 1.77-1.93 (m, 1H), 2.64-2.82 (m, 3H), 2.87-3.01 (m, 1H), 3.13 (br. t, 1H), 3.52 (br. d, 1H), 3.82 (br. d, 1H), 4.21 (s, 2H), 6.95 (t, 1H), 7.28 (t, 1H), 7.34 (d, 2H), 7.58 (d, 1H), 7.70-7.79 (m, 2H), 8.55 (d, 1H).

### Example 18 (Enantiomer 2)

Yield: 1860 mg

Rₜ=3.72 min; chemical purity >99%; >99% ee

[Column: Daicel Chiralpak AD-H, 3 μm, 50 mm×4.6 mm; eluent: carbon dioxide/methanol 5:95→50:50 (v/v); flow rate: 3 ml/min; pressure: 130 bar; temperature: 40° C.; UV detection: 220 nm].

LC-MS (Method 1): Rₜ=0.87 min; m/z=461 (M+H)⁺.

¹H-NMR (400 MHz, DMSO-d₆, δ/ppm): 1.25 (d, 6H), 1.36 (2 s, 9H), 1.44-1.56 (m, 1H), 1.58-1.75 (m, 2H), 1.79-1.92 (m, 1H), 2.64-2.83 (m, 3H), 2.87-3.00 (m, 1H), 3.13 (br. t, 1H), 3.52 (br. d, 1H), 3.82 (br. d, 1H), 4.21 (s, 2H), 6.95 (t, 1H), 7.28 (t, 1H), 7.34 (d, 2H), 7.58 (d, 1H), 7.71-7.78 (m, 2H), 8.55 (d, 1H).

### Example 19 and Example 20

### tert-Butyl 5-{[2-(5-chloropyridin-2-yl)imidazo[1,2-a]pyridin-3-yl]methyl}-2,5-diazabicyclo[2.2.2]octane-2-carboxylate (Enantiomer 1 and 2)

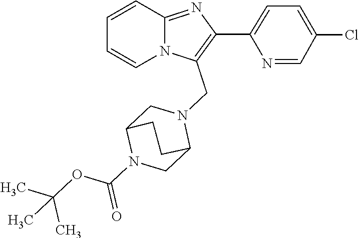 [SMILES: CC(C)(C)OC(=O)N1CC2CCC1CN2Cc1c(-c2ccc(Cl)cn2)nc2ccccn12]

950 mg (2.09 mmol) of racemic tert-butyl 5-{[2-(5-chloropyridin-2-yl)imidazo[1,2-a]pyridin-3-yl]methyl}-2,5-diazabicyclo[2.2.2]octane-2-carboxylate (Example 2) were separated into the enantiomers by preparative HPLC on a chiral phase [column: YMC Cellulose SC, 5 μm, 250 mm×20 mm; eluent: isohexane/isopropanol 50:50+0.2% diethylamine; flow rate: 15 ml/min; UV detection: 220 nm; temperature: 40° C.]:

### Example 19 (Enantiomer 1)

Yield: 450 mg

Rₜ=6.48 min; chemical purity >99%; >99% ee

[Column: YMC Cellulose SC, 5 μm, 250 mm×4.6 mm; eluent: n-heptane/isopropanol 70:30+0.2% diethylamine; flow rate: 1 ml/min; temperature: 40° C.; UV detection: 235 nm].

¹H-NMR (400 MHz, DMSO-d₆, δ/ppm): 1.39 (2 s, 9H), 1.44-1.58 (m, 1H), 1.70 (br. t, 2H), 1.86-2.00 (m, 1H), 2.70 (br. s, 0.5H), 2.78 (br. s, 0.5H), 2.82-2.95 (m, 2H), 3.14 (br. d, 1H), 3.63 (br. dd, 1H), 3.81 (br. s, 0.5H), 3.87 (br. s, 0.5H), 4.55-4.72 (m, 2H), 6.99 (t, 1H), 7.35 (t, 1H), 7.62 (d, 1H), 8.01 (dt, 1H), 8.22 (d, 1H), 8.48 (d, 1H), 8.63 (dd, 1H).

LC-MS (Method 1): Rₜ=0.71 min; m/z=454/456 (M+H)⁺.

### Example 20 (Enantiomer 2)

Yield: 448 mg

Rₜ=7.70 min; chemical purity >99%; >99% ee

[Column: YMC Cellulose SC, 5 μm, 250 mm×4.6 mm; eluent: n-heptane/isopropanol 70:30+0.2% diethylamine; flow rate: 1 ml/min; temperature: 40° C.; UV detection: 235 nm].

¹H-NMR (400 MHz, DMSO-d₆, δ/ppm): 1.39 (2 s, 9H), 1.44-1.58 (m, 1H), 1.70 (br. t, 2H), 1.85-2.01 (m, 1H), 2.70 (br. s, 0.5H), 2.78 (br. s, 0.5H), 2.82-2.96 (m, 2H), 3.14 (br. d, 1H), 3.63 (br. dd, 1H), 3.81 (br. s, 0.5H), 3.87 (br. s, 0.5H), 4.55-4.71 (m, 2H), 6.99 (t, 1H), 7.35 (t, 1H), 7.62 (d, 1H), 8.01 (dd, 1H), 8.21 (d, 1H), 8.48 (d, 1H), 8.63 (dd, 1H).

LC-MS (Method 1): Rₜ=0.71 min; m/z=454/456 (M+H)⁺.

### Example 21

### (−)-[(1S,4S)-5-{[2-(4-Chlorophenyl)imidazo[1,2-a]pyridin-3-yl]methyl}-2,5-diazabicyclo[2.2.2]oct-2-yl](6-methoxypyridin-2-yl)methanone (Enantiomer 1)

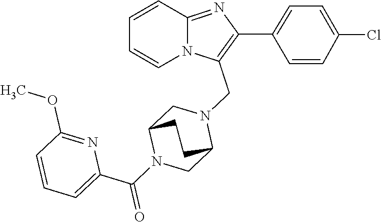 [SMILES: COc1cccc(C(=O)N2C[C@@H]3CC[C@H]2CN3Cc2c(-c3ccc(Cl)cc3)nc3ccccn23)n1]

59 mg (0.39 mmol) of 6-methoxypyridine-2-carboxylic acid were dissolved in 2 ml of DMF, 201 mg (0.53 mmol) of 2-(7-aza-1H-benzotriazol-1-yl)-1,1,3,3-tetramethyluronium hexafluorophosphate (HATU) were added and the mixture was stirred at room temperature for 30 min. 150 mg (0.35 mmol) of 2-{[2-(4-chlorophenyl)imidazo[1,2-a]pyridin-3-yl]methyl}-2,5-diazabicyclo[2.2.2]octane dihydrochloride (enantiomer 1) and 307 μl (1.76 mmol) of N,N-diisopropylethylamine were then added, and the mixture was stirred at room temperature overnight. Thereafter, the reaction mixture was separated directly into its components via preparative HPLC (Method 6). 100 mg (0.2 mmol, 58% of theory) of the title compound were obtained.

LC-MS (Method 1): Rₜ=0.73 min; m/z=488/490 (M+H)⁺.

[α]_{D}²⁰=−40.83° (c=0.320, methanol).

¹H-NMR (400 MHz, DMSO-d₆): δ [ppm]=1.47-1.99 (m, 4H), 2.64 (br. s, 0.25H), 2.71 (dd, 0.75H), 2.82-2.92 (m, 2H), 3.38 (dd, 0.75H), 3.47 (dd, 0.25H), 3.70-3.78 (m, 3H), 3.80 (s, 0.75H), 3.92 (br. d, 0.25H), 3.98 (br. s, 0.75H), 4.21-4.33 (m, 2H), 4.38 (br. s, 0.25H), 6.84-7.02 (m, 2H), 7.17 (d, 0.75H), 7.25-7.36 (m, 1.25H), 7.44-7.55 (m, 2H), 7.60 (d, 1H), 7.75-7.91 (m, 3H), 8.54-8.64 (m, 1H).

The absolute configuration of the compound was determined by means of VCD spectroscopy [cf. Kuppens, T., Bultinck, P., Langenaeker, W., “Determination of absolute configuration via vibrational circular dichroism”, Drug Discovery Today: Technologies 1 (3), 269-275 (2004); Stephens, P. J., “Vibrational circular dichroism spectroscopy: A new tool for the stereochemical characterization of chiral molecules”, Computational Medicinal Chemistry for Drug Discovery, 699-725 (2004)].

### Example 22

### (−)-(3-Chloro-6-methoxypyridin-2-yl)[(1S,4S)-5-{[2-(4-chlorophenyl)imidazo[1,2-a]pyridin-3-yl]methyl}-2,5-diazabicyclo[2.2.2]oct-2-yl]methanone (Enantiomer 1)

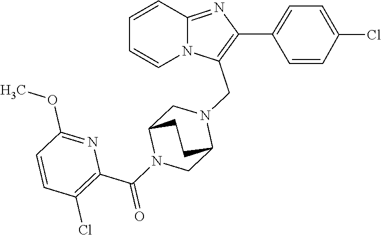 [SMILES: COc1ccc(Cl)c(C(=O)N2C[C@@H]3CC[C@H]2CN3Cc2c(-c3ccc(Cl)cc3)nc3ccccn23)n1]

363 mg (1.94 mmol) of 3-chloro-6-methoxypyridine-2-carboxylic acid were dissolved in 10 ml of DMF, 1005 mg (2.64 mmol) of 2-(7-aza-1H-benzotriazol-1-yl)-1,1,3,3-tetramethyluronium hexafluorophosphate (HATU) were added and the mixture was stirred at room temperature for 30 min. 750 mg (1.76 mmol) of 2-{[2-(4-chlorophenyl)imidazo[1,2-a]pyridin-3-yl]methyl}-2,5-diazabicyclo[2.2.2]octane dihydrochloride (enantiomer 1) and 1.53 ml (8.8 mmol) of N,N-diisopropylethylamine were then added, and the mixture was stirred at room temperature overnight. Thereafter, the reaction solution was added gradually to ice-water, and the precipitated solids were filtered off with suction, washed repeatedly with water and finally dried at 40° C. under high vacuum. The aqueous phase was extracted repeatedly with dichloromethane. The combined organic phases were dried over magnesium sulphate, filtered and concentrated to dryness. The residue was combined with the solids obtained beforehand and purified by column chromatography on silica gel (Biotage SNAP cartridge KP-NH column; eluent: cyclohexane/ethyl acetate 1:1). This gave 685 mg (1.24 mmol, 70% of theory) of the title compound. A portion of this (100 mg) was repurified once again by preparative HPLC (Method 6) and the specific optical rotation (see below) of this sample was determined.

LC-MS (Method 2): Rₜ=1.38 min; m/z=522/523/524 (M+H)⁺.

[α]_{D}²⁰=−62.64° (c=0.455, methanol).

¹H-NMR (400 MHz, DMSO-d₆): δ [ppm]=1.51-1.99 (m, 4H), 2.61 (br. d, 0.7H), 2.65-2.77 (m, 1H), 2.81 (br. s, 0.6H), 2.93-3.00 (m, 1H), 3.20 (br. s, 0.7H), 3.37 (br. d, 0.3H), 3.42 (br. d, 0.7H), 3.71-3.85 (m, 3.7H), 4.16-4.42 (m, 2.3H), 6.85-7.02 (m, 2H), 7.31 (br. t, 1H), 7.46-7.64 (m, 3H), 7.77-7.99 (m, 3H), 8.53-8.65 (m, 1H).

### Example 23

### (−)-[(1S,4S)-5-{[2-(4-Chlorophenyl)imidazo[1,2-a]pyridin-3-yl]methyl}-2,5-diazabicyclo[2.2.2]oct-2-yl](3-fluoro-6-methoxypyridin-2-yl)methanone (Enantiomer 1)

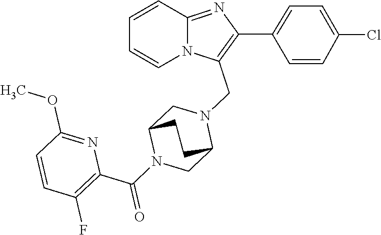 [SMILES: COc1ccc(F)c(C(=O)N2C[C@@H]3CC[C@H]2CN3Cc2c(-c3ccc(Cl)cc3)nc3ccccn23)n1]

332 mg (1.94 mmol) of 3-fluoro-6-methoxypyridine-2-carboxylic acid were dissolved in 10 ml of DMF, 1005 mg (2.64 mmol) of 2-(7-aza-1H-benzotriazol-1-yl)-1,1,3,3-tetramethyluronium hexafluorophosphate (HATU) were added and the mixture was stirred at room temperature for 30 min. 750 mg (1.76 mmol) of 2-{[2-(4-chlorophenyl)imidazo[1,2-a]pyridin-3-yl]methyl}-2,5-diazabicyclo[2.2.2]octane dihydrochloride (enantiomer 1) and 1.53 ml (8.8 mmol) of N,N-diisopropylethylamine were then added, and the mixture was stirred at room temperature overnight. Thereafter, the reaction solution was added gradually to ice-water, and the precipitated solids were filtered off with suction, washed repeatedly with water and finally dried at 40° C. under high vacuum. The aqueous phase was extracted repeatedly with dichloromethane. The combined organic phases were dried over magnesium sulphate, filtered and concentrated to dryness. The residue was combined with the solids obtained beforehand and purified by column chromatography on silica gel (Biotage SNAP cartridge KP-NH column; eluent: cyclohexane/ethyl acetate 1:1). This gave 581 mg (1.15 mmol, 65% of theory) of the title compound. A portion of this (100 mg) was repurified once again by preparative HPLC (Method 6) and the specific optical rotation (see below) of this sample was determined.

LC-MS (Method 1): Rₜ=0.74 min; m/z=505/506 (M+H)⁺.

[α]_{D}²⁰=−47.17° (c=0.460, methanol).

¹H-NMR (400 MHz, DMSO-d₆): δ [ppm]=1.51-2.10 (m, 4H), 2.63-2.72 (m, 2H), 2.77-2.86 (m, 1H), 2.87-2.92 (m, 1H), 3.42 (br. d, 1H), 3.48 (br. s, 1H), 3.73 (s, 3H), 4.22-4.41 (m, 2H), 6.89-7.04 (m, 2H), 7.26-7.35 (m, 1H), 7.47-7.56 (m, 2H), 7.60 (d, 1H), 7.74 (t, 1H), 7.82-7.91 (m, 2H), 8.54-8.63 (m, 1H).

### Example 24

### (5-{[2-(5-Chloropyridin-2-yl)imidazo[1,2-a]pyridin-3-yl]methyl}-2,5-diazabicyclo[2.2.2]oct-2-yl)(3-fluoro-6-methoxypyridin-2-yl)methanone (Enantiomer 1)

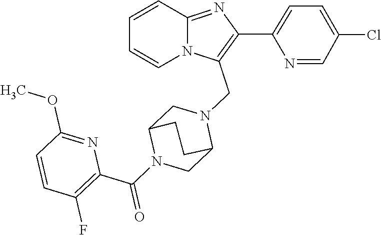 [SMILES: COc1ccc(F)c(C(=O)N2CC3CCC2CN3Cc2c(-c3ccc(Cl)cn3)nc3ccccn23)n1]

41 mg (0.24 mmol) of 3-fluoro-6-methoxypyridine-2-carboxylic acid were dissolved in 1.5 ml of DMF, 123 mg (0.32 mmol) of 2-(7-aza-1H-benzotriazol-1-yl)-1,1,3,3-tetramethyluronium hexafluorophosphate (HATU) were added and the mixture was stirred at room temperature for 30 min. 100 mg (0.22 mmol) of 2-{[2-(5-chloropyridin-2-yl)imidazo[1,2-a]pyridin-3-yl]methyl}-2,5-diazabicyclo[2.2.2]octane dihydrochloride (enantiomer 1) and 188 μl (1.08 mmol) of N,N-diisopropylethylamine were then added, and the mixture was stirred at room temperature overnight. Thereafter, the reaction mixture was separated directly into its components via preparative HPLC (Method 6). 79 mg (0.16 mmol, 72% of theory) of the title compound were obtained.

LC-MS (Method 2): Rₜ=1.13 min; m/z=507/509 (M+H)⁺.

[α]_{D}²⁰=−77.15° (c=0.270, methanol).

¹H-NMR (400 MHz, DMSO-d₆): δ [ppm]=1.52-2.09 (m, 4H), 2.76 (br. s, 0.3H), 2.83-3.08 (m, 2.7H), 3.15 (br. d, 0.3H), 3.42 (br. d, 0.7H), 3.50 (br. s, 0.7H), 3.73-3.89 (m, 4H), 4.42 (br. s, 0.3H), 4.58-4.75 (m, 2H), 6.93 (dd, 0.7H), 6.96-7.05 (m, 1.3H), 7.30-7.39 (m, 1H), 7.58-7.66 (m, 1H), 7.76 (t, 0.7H), 7.84 (t, 0.3H), 7.96-8.04 (m, 1H), 8.17-8.26 (m, 1H), 8.44-8.53 (m, 1.3H), 8.66 (d, 0.7H).

### Example 25

### (3-Chloro-6-methoxypyridin-2-yl)(5-{[2-(5-chloropyridin-2-yl)imidazo[1,2-a]pyridin-3-yl]methyl}-2,5-diazabicyclo[2.2.2]oct-2-yl)methanone (Enantiomer 1)

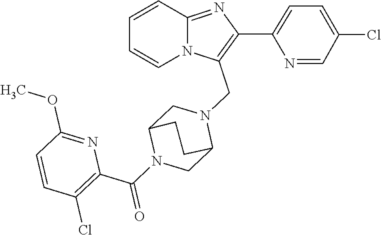 [SMILES: COc1ccc(Cl)c(C(=O)N2CC3CCC2CN3Cc2c(-c3ccc(Cl)cn3)nc3ccccn23)n1]

45 mg (0.24 mmol) of 3-chloro-6-methoxypyridine-2-carboxylic acid were dissolved in 1.5 ml of DMF, 123 mg (0.32 mmol) of 2-(7-aza-1H-benzotriazol-1-yl)-1,1,3,3-tetramethyluronium hexafluorophosphate (HATU) were added and the mixture was stirred at room temperature for 30 min. 100 mg (0.22 mmol) of 2-{[2-(5-chloropyridin-2-yl)imidazo[1,2-a]pyridin-3-yl]methyl}-2,5-diazabicyclo[2.2.2]octane dihydrochloride (enantiomer 1) and 188 μl (1.08 mmol) of N,N-diisopropylethylamine were then added, and the mixture was stirred at room temperature overnight. Thereafter, the reaction mixture was separated directly into its components via preparative HPLC (Method 6). 84 mg (0.16 mmol, 74% of theory) of the title compound were obtained.

LC-MS (Method 2): Rₜ=1.21 min; m/z=523/524/525 (M+H)⁺.

¹H-NMR (400 MHz, DMSO-d₆): δ [ppm]=1.51-2.08 (m, 4H), 2.74 (br. s, 0.3H), 2.83 (br. d, 0.7H), 2.90-3.08 (m, 2.3H), 3.24 (br. s, 0.7H), 3.44 (br. d, 0.7H) 3.66 (br. d, 0.3H), 3.76 (s, 2.3H), 3.82-3.90 (m, 1.4H), 4.43 (br. s, 0.3H), 4.62-4.74 (m, 2H), 6.90 (d, 0.7H), 6.94-7.06 (m, 1.3H), 7.30-7.39 (m, 1H), 7.58-7.67 (m, 1H), 7.85 (d, 0.7H), 7.95 (d, 0.3H), 7.97-8.05 (m, 1H), 8.16-8.27 (m, 1H), 8.44 (d, 0.3H), 8.46-8.53 (m, 1H), 8.65 (d, 0.7H).

### Example 26

### (−)-(5-{[2-(5-Chloropyridin-2-yl)imidazo[1,2-a]pyridin-3-yl]methyl}-2,5-diazabicyclo[2.2.2]oct-2-yl)(6-methoxypyridin-2-yl)methanone (Enantiomer 1)

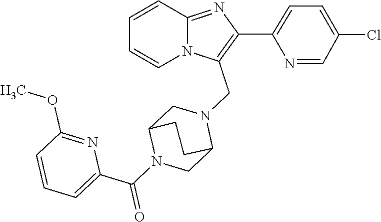 [SMILES: COc1cccc(C(=O)N2CC3CCC2CN3Cc2c(-c3ccc(Cl)cn3)nc3ccccn23)n1]

Batch 1: 40 mg (0.26 mmol) of 6-methoxypyridine-2-carboxylic acid were dissolved in 1.7 ml of DMF, 137 mg (0.36 mmol) of 2-(7-aza-1H-benzotriazol-1-yl)-1,1,3,3-tetramethyluronium hexafluorophosphate (HATU) were added and the mixture was stirred at room temperature for 30 min. 111 mg (0.24 mmol) of 2-{[2-(5-chloropyridin-2-yl)imidazo[1,2-a]pyridin-3-yl]methyl}-2,5-diazabicyclo[2.2.2]octane dihydrochloride (enantiomer 1) and 210 μl (1.20 mmol) of N,N-diisopropylethylamine were then added, and the mixture was stirred at room temperature overnight. Thereafter, the reaction mixture was separated directly into its components via preparative HPLC (Method 6). As a result of damage to the column during the purification, only 16 mg (0.03 mmol, 14% of theory) of the title compound were obtained.

LC-MS (Method 2): Rₜ=1.12 min; m/z=489/491 (M+H)⁺.

[α]_{D}²⁰=−74.46° (c=0.295, methanol).

Batch 2: 36 mg (0.24 mmol) of 6-methoxypyridine-2-carboxylic acid were dissolved in 1.5 ml of DMF, 123 mg (0.32 mmol) of 2-(7-aza-1H-benzotriazol-1-yl)-1,1,3,3-tetramethyluronium hexafluorophosphate (HATU) were added and the mixture was stirred at room temperature for 30 min. 100 mg (0.22 mmol) of 2-{[2-(5-chloropyridin-2-yl)imidazo[1,2-a]pyridin-3-yl]methyl}-2,5-diazabicyclo[2.2.2]octane dihydrochloride (enantiomer 1) and 188 μl (1.08 mmol) of N,N-diisopropylethylamine were then added, and the mixture was stirred at room temperature overnight. Thereafter, the reaction mixture was separated directly into its components via preparative HPLC (Method 6). 87 mg (0.18 mmol, 82% of theory) of the title compound were obtained.

LC-MS (Method 2): Rₜ=1.14 min; m/z=489/491 (M+H)⁺.

¹H-NMR (400 MHz, DMSO-d₆): δ [ppm]=1.50-2.07 (m, 4H), 2.68-2.73 (m, 0.3H), 2.85-2.94 (m, 1.4H), 2.99-3.09 (m, 1.3H), 3.37 (dd, 0.7H), 3.49 (dd, 0.3H), 3.77 (s, 2.3H), 3.81-3.89 (m, 1.4H), 4.01 (br. s, 0.7H), 4.08 (br. d, 0.3H), 4.42 (br. s, 0.3H), 4.57-4.76 (m, 2H), 6.89 (d, 0.7H), 6.94 (d, 0.3H), 6.96-7.04 (m, 1H), 7.21 (d, 0.7H), 7.30-7.39 (m, 1.3H), 7.58-7.65 (m, 1H), 7.77-7.89 (m, 1H), 7.97-8.04 (m, 1H), 8.17-8.25 (m, 1H), 8.43 (d, 0.3H), 8.47-8.53 (m, 1H), 8.63 (d, 0.7H).

### Example 27

### (5-{[2-(6-Isopropylpyridin-3-yl)imidazo[1,2-a]pyridin-3-yl]methyl}-2,5-diazabicyclo[2.2.2]oct-2-yl)(6-methoxypyridin-2-yl)methanone (Racemate)

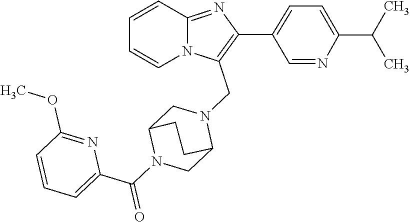 [SMILES: COc1cccc(C(=O)N2CC3CCC2CN3Cc2c(-c3ccc(C(C)C)nc3)nc3ccccn23)n1]

79 mg (0.51 mmol) of 6-methoxypyridine-2-carboxylic acid were dissolved in 2.5 ml of DMF, 266 mg (0.70 mmol) of 2-(7-aza-1H-benzotriazol-1-yl)-1,1,3,3-tetramethyluronium hexafluorophosphate (HATU) were added and the mixture was stirred at room temperature for 30 min. 220 mg (0.47 mmol) of 2-{[2-(6-isopropylpyridin-3-yl)imidazo[1,2-a]pyridin-3-yl]methyl}-2,5-diazabicyclo[2.2.2]octane dihydrochloride (racemate) and 410 μl (2.34 mmol) of N,N-diisopropylethylamine were then added, and the mixture was stirred at room temperature overnight. Thereafter, the reaction mixture was separated directly into its components via preparative HPLC (Method 6). 150 mg (0.30 mmol, 65% of theory) of the title compound were obtained.

LC-MS (Method 2): Rₜ=1.18 min; m/z=497 (M+H)⁺.

### Example 28

### (3-Fluoro-6-methoxypyridin-2-yl)(5-{[2-(6-isopropylpyridin-3-yl)imidazo[1,2-a]pyridin-3-yl]methyl}-2,5-diazabicyclo[2.2.2]oct-2-yl)methanone (Racemate)

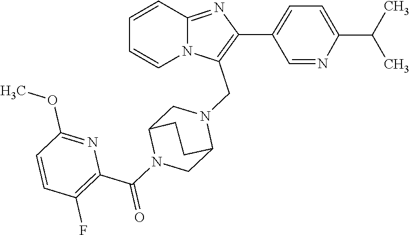 [SMILES: COc1ccc(F)c(C(=O)N2CC3CCC2CN3Cc2c(-c3ccc(C(C)C)nc3)nc3ccccn23)n1]

88 mg (0.51 mmol) of 3-fluoro-6-methoxypyridine-2-carboxylic acid were dissolved in 2.5 ml of DMF, 266 mg (0.70 mmol) of 2-(7-aza-1H-benzotriazol-1-yl)-1,1,3,3-tetramethyluronium hexafluorophosphate (HATU) were added and the mixture was stirred at room temperature for 30 min. 220 mg (0.47 mmol) of 2-{[2-(6-isopropylpyridin-3-yl)imidazo[1,2-a]pyridin-3-yl]methyl}-2,5-diazabicyclo[2.2.2]octane dihydrochloride (racemate) and 410 μl (2.34 mmol) of N,N-diisopropylethylamine were then added, and the mixture was stirred at room temperature overnight. Thereafter, the reaction mixture was separated directly into its components via preparative HPLC (Method 6). 147 mg (0.29 mmol, 61% of theory) of the title compound were obtained.

LC-MS (Method 2): Rₜ=1.19 min; m/z=515 (M+H)⁺.

### Example 29

### (7-{[2-(4-Chlorophenyl)imidazo[1,2-a]pyridin-3-yl]methyl}-3-oxa-7,9-diazabicyclo[3.3.1]non-9-yl)(6-methoxypyridin-2-yl)methanone

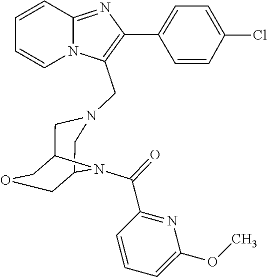 [SMILES: COc1cccc(C(=O)N2C3COCC2CN(Cc2c(-c4ccc(Cl)cc4)nc4ccccn24)C3)n1]

35 mg (0.23 mmol) of 6-methoxypyridine-2-carboxylic acid were dissolved in 1.5 ml of DMF, 119 mg (0.31 mmol) of 2-(7-aza-1H-benzotriazol-1-yl)-1,1,3,3-tetramethyluronium hexafluorophosphate (HATU) were added and the mixture was stirred at room temperature for 30 min. 100 mg (0.21 mmol) of 7-{[2-(4-chlorophenyl)imidazo[1,2-a]pyridin-3-yl]methyl}-3-oxa-7,9-diazabicyclo[3.3.1]nonane dihydrochloride and 150 μl (0.84 mmol) of N,N-diisopropylethylamine were then added, and the mixture was stirred at room temperature overnight. Thereafter, the reaction mixture was separated directly into its components via preparative HPLC (Method 6). 71 mg (0.14 mmol, 67% of theory) of the title compound were obtained.

LC-MS (Method 1): Rₜ=0.72 min; m/z=504/506 (M+H)⁺.

¹H-NMR (400 MHz, DMSO-d₆): δ [ppm]=2.56-2.69 (m, 2H), 2.90 (br. d, 1H), 3.04 (br. d, 1H), 3.66-3.78 (m, 3H), 3.80 (s, 3H), 3.89 (d, 1H), 3.94 (s, 2H), 4.21 (br. s, 1H), 4.46 (br. s, 1H), 6.89-6.97 (m, 2H), 7.26-7.33 (m, 2H), 7.51 (d, 2H), 7.60 (d, 1H), 7.83 (dd, 1H), 7.98 (d, 2H), 8.83 (d, 1H).

### Example 30

### (7-{[2-(4-Chlorophenyl)imidazo[1,2-a]pyridin-3-yl]methyl}-3-oxa-7,9-diazabicyclo[3.3.1]non-9-yl)(3-fluoro-6-methoxypyridin-2-yl)methanone

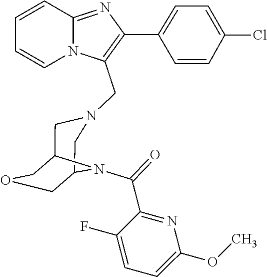 [SMILES: COc1ccc(F)c(C(=O)N2C3COCC2CN(Cc2c(-c4ccc(Cl)cc4)nc4ccccn24)C3)n1]

39 mg (0.23 mmol) of 3-fluoro-6-methoxypyridine-2-carboxylic acid were dissolved in 1.5 ml of DMF, 119 mg (0.31 mmol) of 2-(7-aza-1H-benzotriazol-1-yl)-1,1,3,3-tetramethyluronium hexafluorophosphate (HATU) were added and the mixture was stirred at room temperature for 30 min. 100 mg (0.21 mmol) of 7-{[2-(4-chlorophenyl)imidazo[1,2-a]pyridin-3-yl]methyl}-3-oxa-7,9-diazabicyclo[3.3.1]nonane dihydrochloride and 146 μl (0.84 mmol) of N,N-diisopropylethylamine were then added, and the mixture was stirred at room temperature overnight. Thereafter, the reaction mixture was separated into its components directly via preparative HPLC [instrument: Waters Prep LC/MS System; column: XBridge C18 5 μm, 100 mm×30 mm; eluent A: water, eluent B: acetonitrile; gradient profile: 0-2 min 10% B, 2-2.2 min to 30% B, 2.2-7 min to 70% B, 7-7.5 min to 92% B, 7.5-9 min 92% B; flow rate: 65 ml/min; also a constant 5 ml/min of 2% ammonia in water, room temperature; UV detection: 200-400 nm]. This gave 77 mg (0.15 mmol, 71% of theory) of the title compound.

LC-MS (Method 2): Rₜ=1.33 min; m/z=522/524 (M+H)⁺.

¹H-NMR (400 MHz, DMSO-d₆): δ [ppm]=2.47-2.62 (m, 2H, partly concealed by DMSO signal), 2.88 (br. d, 1H), 3.04 (br. d, 1H), 3.59-3.76 (m, 4H), 3.80 (s, 3H), 3.88 (d, 1H), 3.94 (s, 2H), 4.47 (br. s, 1H), 6.90-7.01 (m, 2H), 7.30 (ddd, 1H), 7.52 (d, 2H), 7.60 (d, 1H), 7.80 (t, 1H), 7.97 (d, 2H), 8.82 (d, 1H).

### Example 31

### (7-{[2-(4-Chlorophenyl)imidazo[1,2-a]pyridin-3-yl]methyl}-3-oxa-7,9-diazabicyclo[3.3.1]non-9-yl)[6-(cyclobutyloxy)pyridin-2-yl]methanone

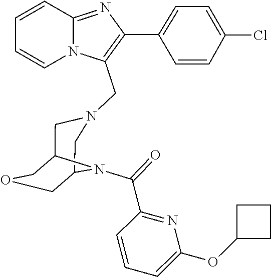 [SMILES: O=C(c1cccc(OC2CCC2)n1)N1C2COCC1CN(Cc1c(-c3ccc(Cl)cc3)nc3ccccn13)C2]

44 mg (0.23 mmol) of 6-(cyclobutyloxy)pyridine-2-carboxylic acid were dissolved in 1.5 ml of DMF, 119 mg (0.31 mmol) of 2-(7-aza-1H-benzotriazol-1-yl)-1,1,3,3-tetramethyluronium hexafluorophosphate (HATU) were added and the mixture was stirred at room temperature for 30 min. 100 mg (0.21 mmol) of 7-{[2-(4-chlorophenyl)imidazo[1,2-a]pyridin-3-yl]methyl}-3-oxa-7,9-diazabicyclo[3.3.1]nonane dihydrochloride and 146 μl (0.84 mmol) of N,N-diisopropylethylamine were then added, and the mixture was stirred at room temperature overnight. Thereafter, the reaction mixture was separated into its components directly via preparative HPLC [instrument: Waters Prep LC/MS System; column: XBridge C18 5 μm, 100 mm×30 mm; eluent A: water, eluent B: acetonitrile; gradient profile: 0-2 min 10% B, 2-2.2 min to 30% B, 2.2-7 min to 70% B, 7-7.5 min to 92% B, 7.5-9 min 92% B; flow rate: 65 ml/min; also a constant 5 ml/min of 2% ammonia in water, room temperature; UV detection: 200-400 nm]. This gave 79 mg (0.14 mmol, 69% of theory) of the title compound.

LC-MS (Method 2): Rₜ=1.60 min; m/z=544/546 (M+H)⁺.

¹H-NMR (400 MHz, DMSO-d₆): δ [ppm]=1.45-1.61 (m, 1H), 1.68-1.80 (m, 1H), 1.93-2.10 (m, 2H), 2.23-2.38 (m, 2H), 2.57-2.65 (m, 2H), 2.87 (br. d, 1H), 3.07 (br. d, 1H), 3.63-3.77 (m, 3H), 3.87-4.01 (m, 3H), 4.14 (br. s, 1H), 4.46 (br. s, 1H), 4.98-5.09 (m, 1H), 6.87 (dd, 1H), 6.93 (td, 1H), 7.26 (dd, 1H), 7.31 (td, 1H), 7.51 (d, 2H), 7.60 (d, 1H), 7.82 (dd, 1H), 7.99 (d, 2H), 8.84 (d, 1H).

### Example 32

### (3-Chloro-6-methoxypyridin-2-yl)(7-{[2-(4-chlorophenyl)imidazo[1,2-a]pyridin-3-yl]methyl}-3-oxa-7,9-diazabicyclo[3.3.1]non-9-yl)methanone

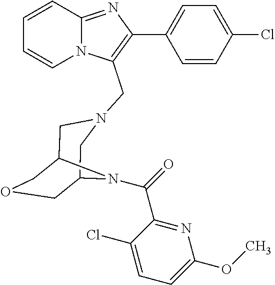 [SMILES: COc1ccc(Cl)c(C(=O)N2C3COCC2CN(Cc2c(-c4ccc(Cl)cc4)nc4ccccn24)C3)n1]

43 mg (0.23 mmol) of 3-chloro-6-methoxypyridine-2-carboxylic acid were dissolved in 1.4 ml of DMF, 119 mg (0.31 mmol) of 2-(7-aza-1H-benzotriazol-1-yl)-1,1,3,3-tetramethyluronium hexafluorophosphate (HATU) were added and the mixture was stirred at room temperature for 30 min. 100 mg (0.21 mmol) of 7-{[2-(4-chlorophenyl)imidazo[1,2-a]pyridin-3-yl]methyl}-3-oxa-7,9-diazabicyclo[3.3.1]nonane dihydrochloride and 182 μl (1.05 mmol) of N,N-diisopropylethylamine were then added, and the mixture was stirred at room temperature overnight. Thereafter, the reaction mixture was separated directly into its components via preparative HPLC (Method 6). 86 mg (0.16 mmol, 76% of theory) of the title compound were obtained.

LC-MS (Method 2): Rₜ=1.39 min; m/z=538/539/540 (M+H)⁺.

¹H-NMR (400 MHz, DMSO-d₆): δ [ppm]=2.46-2.60 (m, 2H, partly concealed by DMSO signal), 2.86 (br. d, 1H), 3.04 (br. d, 1H), 3.34 (br. s, 1H), 3.59-3.76 (m, 3H), 3.82 (s, 3H), 3.88 (d, 1H), 3.94 (s, 2H), 4.46 (br. s, 1H), 6.89-6.98 (m, 2H), 7.30 (t, 1H), 7.52 (d, 2H), 7.60 (d, 1H), 7.90 (d, 1H), 7.97 (d, 2H), 8.81 (d, 1H).

### Example 33

### (3-{[2-(5-Chloropyridin-2-yl)imidazo[1,2-a]pyridin-3-yl]methyl}-3,8-diazabicyclo[3.2.1]oct-8-yl)(6-methoxypyridin-2-yl)methanone

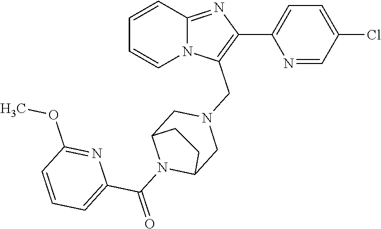 [SMILES: COc1cccc(C(=O)N2C3CCC2CN(Cc2c(-c4ccc(Cl)cn4)nc4ccccn24)C3)n1]

39 mg (0.26 mmol) of 6-methoxypyridine-2-carboxylic acid were dissolved in 1.5 ml of DMF, 134 mg (0.35 mmol) of 2-(7-aza-1H-benzotriazol-1-yl)-1,1,3,3-tetramethyluronium hexafluorophosphate (HATU) were added and the mixture was stirred at room temperature for 30 min. 100 mg (0.23 mmol) of 3-{[2-(5-chloropyridin-2-yl)imidazo[1,2-a]pyridin-3-yl]methyl}-3,8-diazabicyclo[3.2.1]octane dihydrochloride and 200 μl (1.17 mmol) of N,N-diisopropylethylamine were then added, and the mixture was stirred at room temperature overnight. Thereafter, the reaction mixture was separated directly into its components via preparative HPLC (Method 6). 93 mg (0.19 mmol, 81% of theory) of the title compound were obtained.

LC-MS (Method 2): Rₜ=1.31 min; m/z=489/491 (M+H)⁺.

¹H-NMR (400 MHz, DMSO-d₆): δ [ppm]=1.62-1.80 (m, 4H), 2.40 (br. d, 1H), 2.46-2.69 (m, 2H, partly concealed by DMSO signal), 2.76 (br. d, 1H), 3.72 (s, 3H), 4.44-4.64 (m, 4H), 6.91 (d, 1H), 7.02 (td, 1H), 7.30-7.39 (m, 2H), 7.62 (d, 1H), 7.80 (dd, 1H), 8.00 (dd, 1H), 8.19 (d, 1H), 8.57 (d, 1H), 8.66 (d, 1H).

Analogously to Example 21 and 33, the following compounds were prepared from the reactants specified in each case:

| Example | Name/Structure/Starting materials | Analytical data |
|---|---|---|
| 34 | 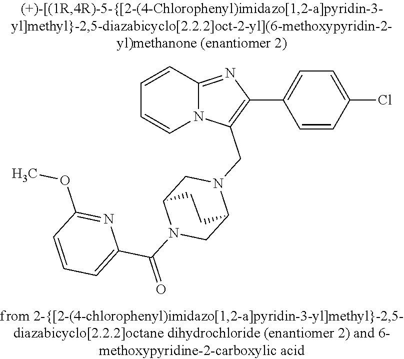 [SMILES: COc1cccc(C(=O)N2C[C@H]3CC[C@@H]2CN3Cc2c(-c3ccc(Cl)cc3)nc3ccccn23)n1] | ¹H-NMR (400 MHz, DMSO-d₆): δ [ppm] = 1.48-2.00 (m, 4H), 2.64 (br. s, 0.25H), 2.71 (dd, 0.75H), 2.81-2.93 (m, 2H), 3.38 (dd, 0.75H), 3.47 (dd, 0.25H), 3.70-3.78 (m, 3H), 3.80 (s, 0.75H), 3.92 (br. d, 0.25H), 3.98 (br. s, 0.75H), 4.21-4.32 (m, 2H), 4.38 (br. s, 0.25H), 6.85-7.02 (m, 2H), 7.17 (d, 0.75H), 7.25-7.35 (m, 1.25H), 7.46-7.56 (m, 2H), 7.60 (d, 1H), 7.75-7.92 (m, 3H), 8.55-8.63 (m, 1H). [α]_{D}²⁰ = +46.13° (c = 0.250, methanol). LC-MS (Method 1): Rₜ = 0.73 min; m/z = 488/490 (M + H)⁺. The absolute configuration was determined by means of VCD spectroscopy (cf. Example 21). |
| 35 | 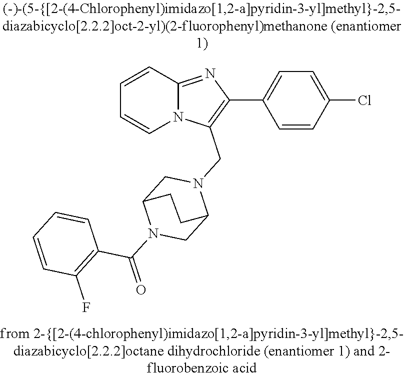 [SMILES: O=C(c1ccccc1F)N1CC2CCC1CN2Cc1c(-c2ccc(Cl)cc2)nc2ccccn12] | ¹H-NMR (400 MHz, DMSO-d₆): δ [ppm] = 1.48-1.97 (m, 4H), 2.63-2.71 (m, 1H), 2.74-2.88 (m, 1.25H), 2.90 (br. s, 0.75H), 3.01 (d, 0.25H), 3.27-3.36 (m, 0.75H, partly concealed by H₂O signal), 3.42 (br. d, 1H), 3.76 (br. d, 0.75H), 4.17-4.32 (m, 2H), 4.39 (br. s, 0.25H), 6.93-7.02 (m, 1H), 7.20-7.39 (m, 4H), 7.42-7.63 (m, 4H), 7.84 (d, 0.5H), 7.88 (d, 1.5H), 8.54-8.63 (m, 1H). [α]_{D}²⁰ = −29.87° (c = 0.250, methanol). LC-MS (Method 1): Rₜ = 0.75 min; m/z = 475/477 (M + H)⁺. |
| 36 | 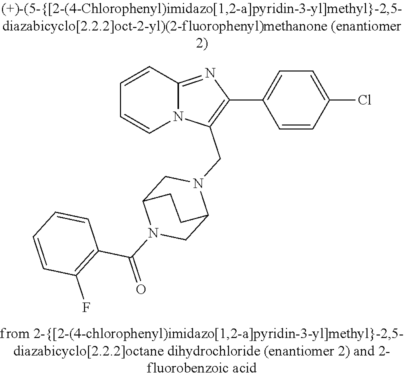 [SMILES: O=C(c1ccccc1F)N1CC2CCC1CN2Cc1c(-c2ccc(Cl)cc2)nc2ccccn12] | ¹H-NMR (400 MHz, DMSO-d₆): δ [ppm] = 1.48-1.97 (m, 4H), 2.62-2.71 (m, 1H), 2.74-2.88 (m, 1.25H), 2.90 (br. s, 0.75H), 3.01 (d, 0.25H), 3.27-3.36 (m, 0.75H, partly concealed by H₂O signal), 3.42 (br. d, 1H), 3.76 (br. d, 0.75H), 4.17-4.32 (m, 2H), 4.39 (br. s, 0.25H), 6.93-7.02 (m, 1H), 7.20-7.39 (m, 4H), 7.41-7.63 (m, 4H), 7.83 (d, 0.5H), 7.88 (d, 1.5H), 8.54-8.63 (m, 1H). [α]_{D}²⁰ = +19.64° (c = 0.275, methanol). LC-MS (Method 1): Rₜ = 0.74 min; m/z = 475/477 (M + H)⁺. |
| 37 | 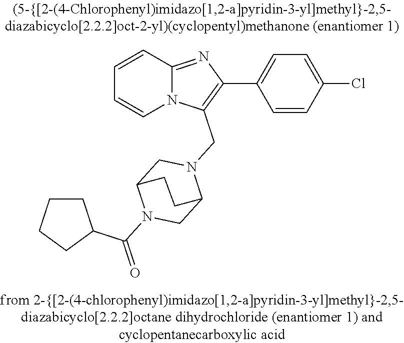 [SMILES: O=C(C1CCCC1)N1CC2CCC1CN2Cc1c(-c2ccc(Cl)cc2)nc2ccccn12] | ¹H-NMR (400 MHz, DMSO-d₆): δ [ppm] = 1.39-1.79 (m, 11H), 1.81-1.95 (m, 1H), 2.63-2.86 (m, 4H), 3.18 (dd, 0.5H), 3.36 (br. d, 0.5H), 3.54 (br. d, 0.5H), 3.73 (br. d, 0.5H), 3.93 (br. s, 0.5H), 4.15-4.29 (m, 2.5H), 6.97 (t, 1H), 7.25-7.36 (m, 1H), 7.53 (d, 2H), 7.59 (d, 1H), 7.86 (d, 2H), 8.54-8.62 (m, 1H). LC-MS (Method 1): Rₜ = 0.74 min; m/z = 449/451 (M + H)⁺. |
| 38 | 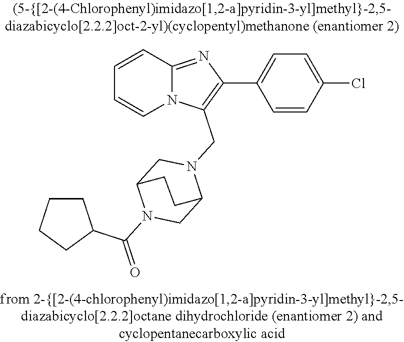 [SMILES: O=C(C1CCCC1)N1CC2CCC1CN2Cc1c(-c2ccc(Cl)cc2)nc2ccccn12] | ¹H-NMR (400 MHz, DMSO-d₆): δ [ppm] = 1.38-1.78 (m, 11H), 1.80-1.95 (m, 1H), 2.64-2.86 (m, 4H), 3.18 (dd, 0.5H), 3.36 (br. d, 0.5H), 3.54 (br. d, 0.5H), 3.73 (br. d, 0.5H), 3.93 (br. s, 0.5H), 4.16-4.30 (m, 2.5H), 6.97 (t, 1H), 7.25-7.35 (m, 1H), 7.53 (d, 2H), 7.59 (d, 1H), 7.86 (d, 2H), 8.53-8.62 (m, 1H). LC-MS (Method 1): Rₜ = 0.75 min; m/z = 449/451 (M + H)⁺. |
| 39 | 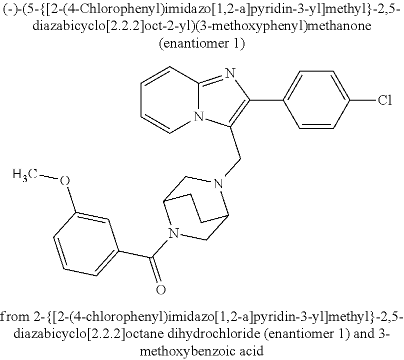 [SMILES: COc1cccc(C(=O)N2CC3CCC2CN3Cc2c(-c3ccc(Cl)cc3)nc3ccccn23)c1] | ¹H-NMR (400 MHz, DMSO-d₆): δ [ppm] = 1.46-1.67 (m, 1.75H), 1.69-1.98 (m, 2.25H), 2.68 (br. d, 1H), 2.81 (br. d, 1H), 2.88 (br. s, 0.75H), 2.92 (br. d, 0.25H), 3.17 (br. d, 0.25H), 3.37 (br. d, 0.75H), 3.53 (br. s, 0.75H), 3.62 (br. d, 0.25H), 3.67-3.82 (m, 3.75H), 4.19-4.32 (m, 2.25H), 6.80-6.87 (m, 1.5H), 6.91-7.06 (m, 2.5H), 7.26-7.40 (m, 2H), 7.46-7.64 (m, 3H), 7.83-7.92 (m, 2H), 8.56-8.63 (m, 1H). [α]_{D}²⁰ = −36.15° (c = 0.260, methanol). LC-MS (Method 1): Rₜ = 0.75 min; m/z = 487/489 (M + H)⁺. |
| 40 | 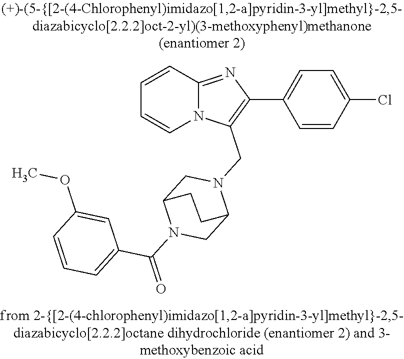 [SMILES: COc1cccc(C(=O)N2CC3CCC2CN3Cc2c(-c3ccc(Cl)cc3)nc3ccccn23)c1] | ¹H-NMR (400 MHz, DMSO-d₆): δ [ppm] = 1.46-1.67 (m, 1.75H), 1.70-1.97 (m, 2.25H), 2.68 (br. d, 1H), 2.81 (br. d, 1H), 2.88 (br. s, 0.75H), 2.92 (br. d, 0.25H), 3.17 (br. d, 0.25H), 3.37 (br. d, 0.75H), 3.53 (br. s, 0.75H), 3.62 (br. d, 0.25H), 3.68-3.82 (m, 3.75H), 4.20-4.32 (m, 2.25H), 6.80-6.87 (m, 1.5H), 6.92-7.06 (m, 2.5H), 7.26-7.39 (m, 2H), 7.47-7.63 (m, 3H), 7.84-7.92 (m, 2H), 8.56-8.64 (m, 1H). [α]_{D}²⁰ = +43.73° (c = 0.250, methanol). LC-MS (Method 1): Rₜ = 0.75 min; m/z = 487/489 (M + H)⁺. |
| 41 | 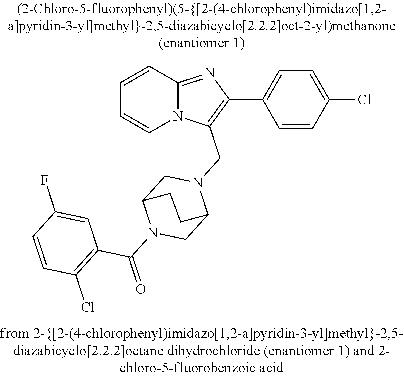 [SMILES: O=C(c1cc(F)ccc1Cl)N1CC2CCC1CN2Cc1c(-c2ccc(Cl)cc2)nc2ccccn12] | ¹H-NMR (400 MHz, DMSO-d₆): δ [ppm] = 1.46-1.99 (m, 4H), 2.56-3.01 (m, 3.6H), 3.14 (br. s, 0.4H), 3.23 (br. s, 0.4H), 3.41 (br. t, 0.6H), 3.77 (br. t, 4.17-4.33 (m, 2H), 4.39 (br. s, 0.4H), 6.92-7.03 (m, 1H), 7.14-7.38 (m, 3H), 7.46-7.64 (m, 4H), 7.77-7.93 (m, 2H), 8.53-8.64 (m, 1H). LC-MS (Method 1): Rₜ = 0.79 min; m/z = 509/511 (M + H)⁺. |
| 42 | 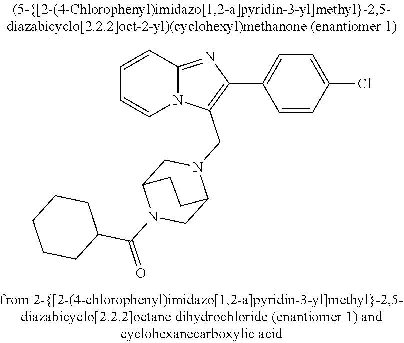 [SMILES: O=C(C1CCCCC1)N1CC2CCC1CN2Cc1c(-c2ccc(Cl)cc2)nc2ccccn12] | ¹H-NMR (400 MHz, DMSO-d₆): δ [ppm] = 1.04-1.38 (m, 5H), 1.42-1.78 (m, 8H), 1.79-1.94 (m, 1H), 2.23-2.45 (m, 1H), 2.61-2.84 (m, 3H), 3.16 (dd, 0.6H), 3.27-3.39 (m, 0.4H, partly concealed by H₂O signal), 3.51 (d, 0.6H), 3.72 (d, 0.4H), 3.89 (br. s, 0.6H), 4.15-4.29 (m, 2.4H), 6.97 (t, 1H), 7.31 (t, 1H), 7.52 (d, 2H), 7.59 (d, 1H), 7.82-7.90 (m, 2H), 8.57 (d, 1H). LC-MS (Method 1): Rₜ = 0.77 min; m/z = 463/465 (M + H)⁺. |
| 43 | 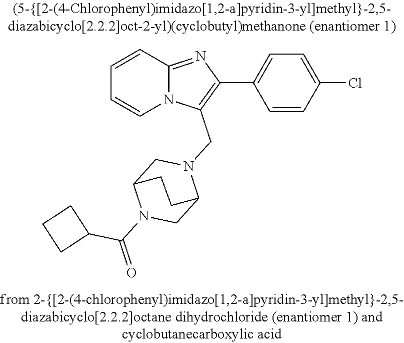 [SMILES: O=C(C1CCC1)N1CC2CCC1CN2Cc1c(-c2ccc(Cl)cc2)nc2ccccn12] | ¹H-NMR (400 MHz, DMSO-d₆): δ [ppm] = 1.41-1.55 (m, 1H), 1.56-2.18 (m, 9H), 2.64-2.83 (m, 3H), 3.10-3.24 (m, 2H), 3.48-3.60 (m, 1H), 3.63 (br. s, 0.6H), 4.15-4.28 (m, 2.4H), 6.97 (t, 1H), 7.31 (t, 1H), 7.53 (d, 2H), 7.59 (d, 1H), 7.81-7.90 (m, 2H), 8.57 (d, 1H). LC-MS (Method 1): Rₜ = 0.69 min; m/z = 435/437 (M + H)⁺. |
| 44 | 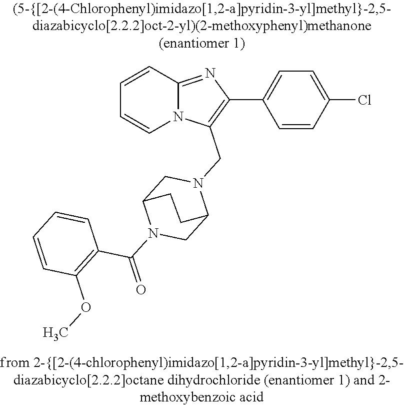 [SMILES: COc1ccccc1C(=O)N1CC2CCC1CN2Cc1c(-c2ccc(Cl)cc2)nc2ccccn12] | ¹H-NMR (400 MHz, DMSO-d₆): δ [ppm] = 1.45-1.67 (m, 2.2H), 1.69-1.95 (m, 1.8H), 2.46-2.84 (m, 3H, partly concealed by DMSO signal), 2.90 (br. s, 1H), 3.18 (br. s, 0.8H), 3.28-3.47 (m, 1H, partly concealed by H₂O signal), 3.58 (br. s, 1.2H), 3.74 (br. s, 1.8H), 4.17-4.30 (m, 2H), 4.33-4.39 (m, 0.2H), 6.86-7.14 (m, 4H), 7.26-7.41 (m, 2H), 7.47-7.63 (m, 3H), 7.79-7.94 (m, 2H). LC-MS (Method 2): Rₜ = 1.33 min; m/z = 487/489 (M + H)⁺. |
| 45 | 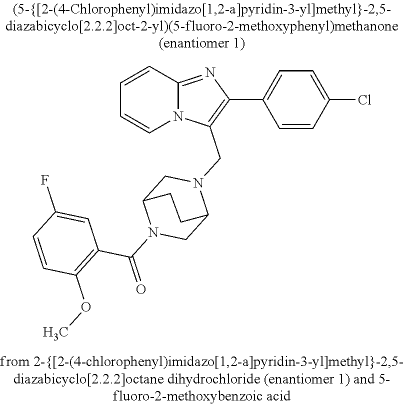 [SMILES: COc1ccc(F)cc1C(=O)N1CC2CCC1CN2Cc1c(-c2ccc(Cl)cc2)nc2ccccn12] | ¹H-NMR (400 MHz, DMSO-d₆): δ [ppm] = 1.47-1.97 (m, 4H), 2.69 (br. d, 1H), 2.77-2.92 (m, 2H), 3.03 (br. d, 0.25H), 3.34-3.48 (m, 1.75H), 3.68-3.80 (m, 3.75H), 4.17-4.30 (m, 2H), 4.38 (br. s, 0.25H), 6.80-6.91 (m, 1H), 6.93-7.05 (m, 2H), 7.13-7.25 (m, 1H), 7.27-7.35 (m, 1H), 7.46-7.57 (m, 2H), 7.57-7.63 (m, 1H), 7.81-7.92 (m, 2H), 8.54-8.63 (m, 1H). LC-MS (Method 2): Rₜ = 1.41 min; m/z = 505/507 (M + H)⁺. |
| 46 | 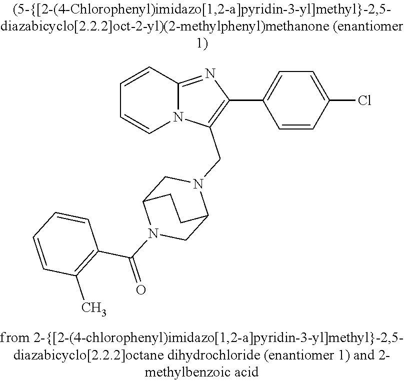 [SMILES: Cc1ccccc1C(=O)N1CC2CCC1CN2Cc1c(-c2ccc(Cl)cc2)nc2ccccn12] | ¹H-NMR (400 MHz, DMSO-d₆): δ [ppm] = 1.46-1.97 (m, 4H), 2.00-2.22 (m, 3H), 2.56-2.69 (m, 1.5H), 2.73-2.80 (m, 0.5H), 2.86 (br. t, 0.5H), 2.94 (br. s, 0.75H), 3.12-3.27 (m, 1H), 3.40 (d, 0.75H), 3.80 (br. d, 0.75H), 4.16-4.30 (m, 2H), 4.40 (br. s, 0.25H), 6.91-7.02 (m, 1.3H), 7.09-7.36 (m, 4.7H), 7.46-7.63 (m, 3H), 7.79-7.91 (m, 2H), 8.52-8.62 (m, 1H). LC-MS (Method 2): Rₜ = 1.40 min; m/z = 471/473 (M + H)⁺. |
| 47 | 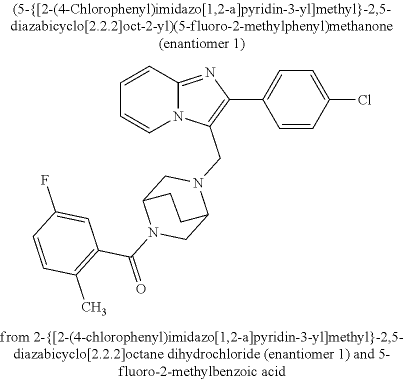 [SMILES: Cc1ccc(F)cc1C(=O)N1CC2CCC1CN2Cc1c(-c2ccc(Cl)cc2)nc2ccccn12] | ¹H-NMR (400 MHz, DMSO-d₆): δ [ppm] = 1.48-1.96 (m, 4H), 1.97-2.20 (m, 3H), 2.57-2.69 (m, 1.4H), 2.74-3.01 (m, 2H), 3.13-3.29 (m, 1H), 3.40 (dd, 0.7H), 3.78 (br. d, 0.6H), 4.16-4.31 (m, 2H), 4.39 (br. s, 0.3H), 6.90-7.17 (m, 3H), 7.21-7.35 (m, 2H), 7.47-7.56 (m, 2H), 7.56-7.63 (m, 1H), 7.81-7.92 (m, 2H), 8.53-8.63 (m, 1H). LC-MS (Method 2): Rₜ = 1.45 min; m/z = 489/491 (M + H)⁺. |
| 48 | 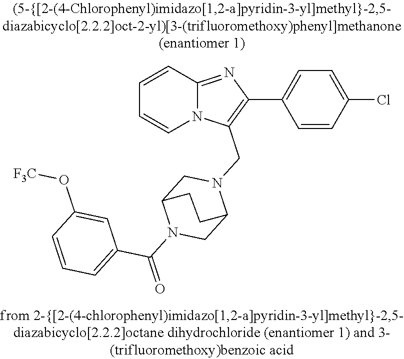 [SMILES: O=C(c1cccc(OC(F)(F)F)c1)N1CC2CCC1CN2Cc1c(-c2ccc(Cl)cc2)nc2ccccn12] | ¹H-NMR (400 MHz, DMSO-d₆): δ [ppm] = 1.46-1.99 (m, 4H), 2.63-2.72 (m, 1H), 2.84 (br. d, 1H), 2.87-2.97 (m, 1H), 3.18 (br. d, 0.3H), 3.39 (dd, 0.7H), 3.47 (br. s, 0.7H), 3.63 (br. d, 0.3H), 3.74 (br. d, 0.7H), 4.20-4.35 (m, 2.3H), 6.92-7.02 (m, 1H), 7.26-7.37 (m, 2.4H), 7.40-7.63 (m, 5.6H), 7.83-7.92 (m, 2H), 8.56-8.64 (m, 1H). LC-MS (Method 4): Rₜ = 2.09 min; m/z = 541/543 (M + H)⁺. |
| 49 | 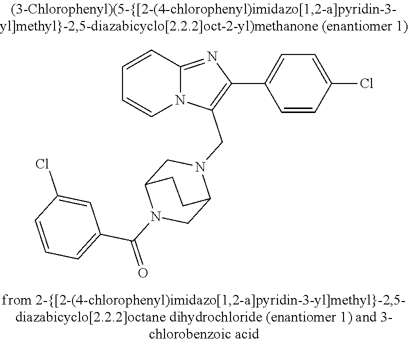 [SMILES: O=C(c1cccc(Cl)c1)N1CC2CCC1CN2Cc1c(-c2ccc(Cl)cc2)nc2ccccn12] | ¹H-NMR (400 MHz, DMSO-d₆): δ [ppm] = 1.48-1.98 (m, 4H), 2.62-2.70 (m, 1H), 2.83 (br. d, 1H), 2.87-2.95 (m, 1H), 3.17 (br. d, 0.3H), 3.38 (dd, 0.7H), 3.49 (br. s, 0.7H), 3.65 (br. d, 0.3H), 3.73 (br. d, 0.7H), 4.19-4.32 (m, 2.3H), 6.93-7.02 (m, 1H), 7.22-7.63 (m, 8H), 7.84-7.92 (m, 2H), 8.56-8.64 (m, 1H). LC-MS (Method 4): Rₜ = 2.00 min; m/z = 491/493 (M + H)⁺. |
| 50 | 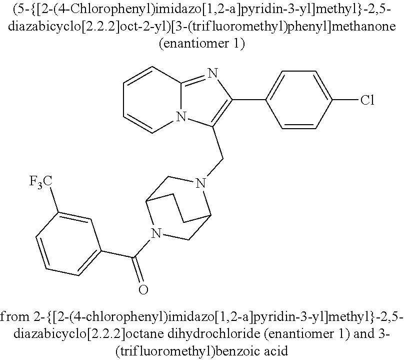 [SMILES: O=C(c1cccc(C(F)(F)F)c1)N1CC2CCC1CN2Cc1c(-c2ccc(Cl)cc2)nc2ccccn12] | ¹H-NMR (400 MHz, DMSO-d₆): δ [ppm] = 1.47-1.99 (m, 4H), 2.63-2.71 (m, 1H), 2.80-2.99 (m, 2H), 3.19 (br. d, 0.3H), 3.41 (d, 0.7H), 3.47 (br. s, 0.7H), 3.63 (br. d, 0.3H), 3.75 (br. d, 0.7H), 4.19-4.36 (m, 2.3H), 6.92-7.03 (m, 1H), 7.26-7.35 (m, 1H), 7.45-7.73 (m, 5H), 7.76-7.92 (m, 4H), 8.56-8.65 (m, 1H). LC-MS (Method 4): Rₜ = 2.05 min; m/z = 525/527 (M + H)⁺. |
| 51 | 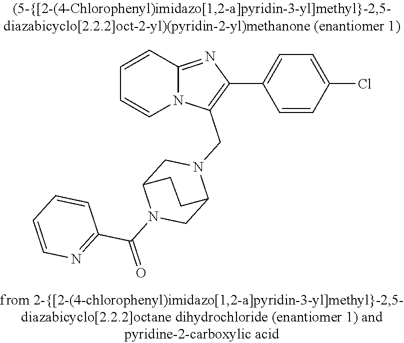 [SMILES: O=C(c1ccccn1)N1CC2CCC1CN2Cc1c(-c2ccc(Cl)cc2)nc2ccccn12] | ¹H-NMR (400 MHz, DMSO-d₆): δ [ppm] = 1.47-1.99 (m, 4H), 2.68 (dd, 1H), 2.81-2.94 (m, 2H), 3.34-3.45 (m, 1H), 3.71-3.81 (m, 1H), 3.87 (br. s, 0.75H), 4.20-4.33 (m, 2H), 4.39 (br. s, 0.25H), 6.92-7.02 (m, 1H), 7.26-7.35 (m, 1H), 7.41-7.66 (m, 5H), 7.81-7.97 (m, 3H), 8.50-8.64 (m, 2H). LC-MS (Method 1): Rₜ = 0.64 min; m/z = 458/460 (M + H)⁺. |
| 52 | 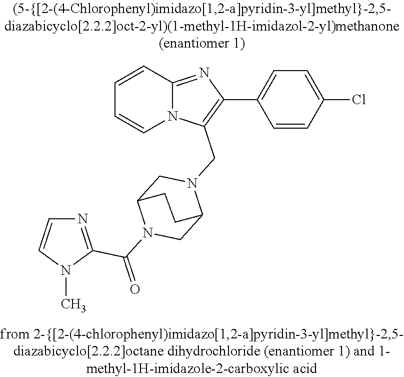 [SMILES: Cn1ccnc1C(=O)N1CC2CCC1CN2Cc1c(-c2ccc(Cl)cc2)nc2ccccn12] | ¹H-NMR (400 MHz, DMSO-d₆): δ [ppm] = 1.48-1.98 (m, 4H), 2.64-2.93 (m, 3H), 3.36 (dd, 1H), 3.66-3.81 (m, 3.75H), 4.09 (d, 0.25H), 4.19-4.31 (m, 2H), 4.38 (br. s, 0.25H), 4.74 (br. s, 0.75H), 6.87-7.02 (m, 2H), 7.23-7.35 (m, 2H), 7.46-7.56 (m, 2H), 7.60 (d, 1H), 7.82-7.92 (m, 2H), 8.60 (d, 1H). LC-MS (Method 1): Rₜ = 0.61 min; m/z = 461/463 (M + H)⁺. |
| 53 | 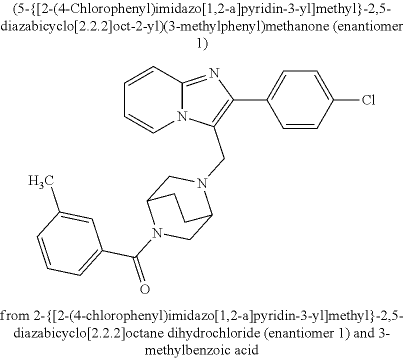 [SMILES: Cc1cccc(C(=O)N2CC3CCC2CN3Cc2c(-c3ccc(Cl)cc3)nc3ccccn23)c1] | ¹H-NMR (400 MHz, DMSO-d₆): δ [ppm] = 1.45-1.98 (m, 4H), 2.30 (s, 2H), 2.35 (s, 1H), 2.62-2.69 (m, 1H), 2.75-2.84 (m, 1H), 2.86-2.94 (m, 1H), 3.16 (br. d, 0.3H), 3.37 (br. d, 0.7H), 3.52 (br. d, 0.7H), 3.62 (br. d, 0.3H), 3.74 (br. d, 0.7H), 4.20-4.32 (m, 2.3H), 6.92-7.13 (m, 2.4H), 7.18-7.36 (m, 3.6H), 7.46-7.64 (m, 3H), 7.83-7.92 (m, 2H), 8.56-8.63 (m, 1H). LC-MS (Method 2): Rₜ = 1.41 min; m/z = 471/473 (M + H)⁺. |
| 54 | 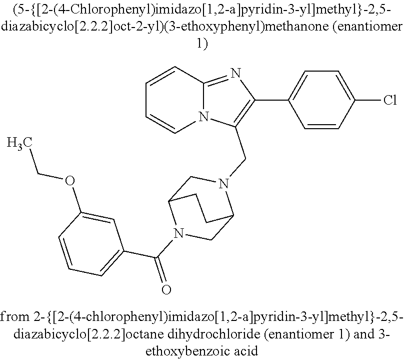 [SMILES: CCOc1cccc(C(=O)N2CC3CCC2CN3Cc2c(-c3ccc(Cl)cc3)nc3ccccn23)c1] | ¹H-NMR (400 MHz, DMSO-d₆): δ [ppm] = 1.22-1.38 (m, 3H), 1.44-1.96 (m, 4H), 2.62-2.97 (m, 3H), 3.16 (br. d, 0.3H), 3.36 (br. d, 0.7H), 3.52 (br. s, 0.7H), 3.63 (br. d, 0.3H), 3.73 (br. d, 0.7H), 3.95-4.10 (m, 2H), 4.17-4.32 (m, 2.3H), 6.77-6.85 (m, 1.4H), 6.91-7.03 (m, 2.6H), 7.23-7.38 (m, 2H), 7.46-7.63 (m, 3H), 7.82-7.92 (m, 2H), 8.55-8.64 (m, 1H). LC-MS (Method 2): Rₜ = 1.43 min; m/z = 501/503 (M + H)⁺. |
| 55 | 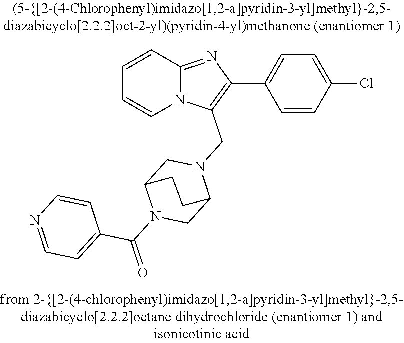 [SMILES: O=C(c1ccncc1)N1CC2CCC1CN2Cc1c(-c2ccc(Cl)cc2)nc2ccccn12] | ¹H-NMR (400 MHz, DMSO-d₆): δ [ppm] = 1.47-1.99 (m, 4H), 2.61-2.70 (m, 1H), 2.78-2.87 (m, 1H), 2.91 (br. s, 1H), 3.14 (br. d, 0.3H), 3.36-3.46 (m, 1.4H), 3.58 (br. d, 0.3H), 3.74 (br. d, 0.7H), 4.18-4.36 (m, 2.3H), 6.92-7.02 (m, 1H), 7.25 (m, 2.5H), 7.38-7.44 (m, 0.5H), 7.47-7.63 (m, 3H), 7.81-7.91 (m, 2H), 8.55-8.64 (m, 2.5H), 8.65-8.70 (m, 0.5H). LC-MS (Method 2): Rₜ = 1.06 min; m/z = 458/460 (M + H)⁺. |
| 56 | 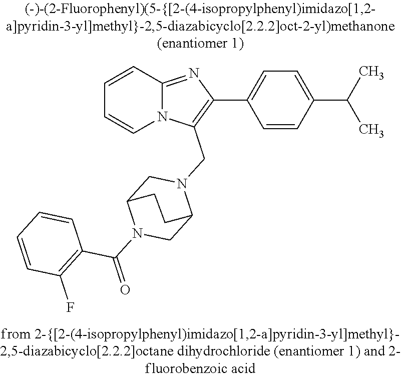 [SMILES: CC(C)c1ccc(-c2nc3ccccn3c2CN2CC3CCC2CN3C(=O)c2ccccc2F)cc1] | ¹H-NMR (400 MHz, DMSO-d₆): δ [ppm] = 1.20-1.30 (m, 6H), 1.49-1.99 (m, 4H), 2.65-2.73 (m, 1H), 2.76-2.88 (m, 1.25H), 2.88-3.06 (m, 2H), 3.27-3.36 (m, 0.75H, partly concealed by H₂O signal), 3.39-3.49 (m, 1H), 3.77 (br. d, 0.75H), 4.22 (s, 0.5H), 4.26 (s, 1.5H), 4.40 (br. s, 0.25H), 6.91-7.00 (m, 1H), 7.18-7.39 (m, 6H), 7.41-7.53 (m, 1H), 7.55-7.62 (m, 1H), 7.73 (d, 0.5H), 7.76 (d, 1.5H), 8.52-8.61 (m, 1H). [α]_{D}²⁰ = −27.07° (c = 0.250, methanol). LC-MS (Method 1): Rₜ = 0.81 min; m/z = 483 (M + H)⁺. |
| 57 | 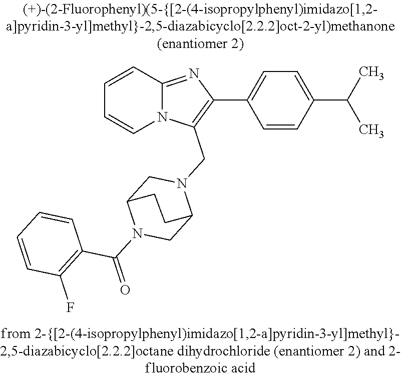 [SMILES: CC(C)c1ccc(-c2nc3ccccn3c2CN2CC3CCC2CN3C(=O)c2ccccc2F)cc1] | ¹H-NMR (400 MHz, DMSO-d₆): δ [ppm] = 1.20-1.30 (m, 6H), 1.48-1.97 (m, 4H), 2.63-2.74 (m, 1H), 2.75-2.88 (m, 1.25H), 2.89-3.05 (m, 2H), 3.25-3.36 (m, 0.75H, partly concealed by H₂O signal), 3.38-3.51 (m, 1H), 3.77 (br. d, 0.75H), 4.22 (s, 0.5H), 4.26 (s, 1.5H), 4.40 (br. s, 0.25H), 6.90-7.00 (m, 1H), 7.17-7.40 (m, 6H), 7.40-7.53 (m, 1H), 7.54-7.61 (m, 1H), 7.73 (d, 0.5H), 7.76 (d, 1.5H), 8.51-8.61 (m, 1H). [α]_{D}²⁰ = +26.29° (c = 0.265, methanol). LC-MS (Method 4): Rₜ = 2.02 min; m/z = 483 (M + H)⁺. |
| 58 | 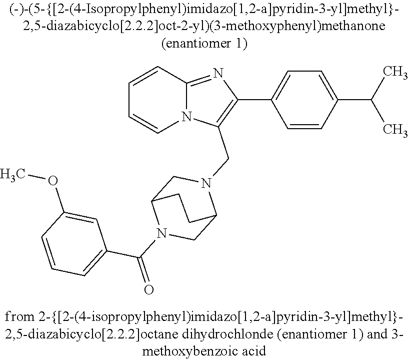 [SMILES: COc1cccc(C(=O)N2CC3CCC2CN3Cc2c(-c3ccc(C(C)C)cc3)nc3ccccn23)c1] | ¹H-NMR (400 MHz, DMSO-d₆): δ [ppm] = 1.20-1.30 (m, 6H), 1.47-1.69 (m, 1.75H), 1.71-1.98 (m, 2.25H), 2.66-2.74 (m, 1H), 2.82 (br. d, 1H), 2.88-3.01 (m, 2H), 3.17 (br. d, 0.25H), 3.37 (br. d, 0.75H), 3.54 (br. s, 0.75H), 3.64 (br. d, 0.25H), 3.70-3.82 (m, 3.75H), 4.22 (s, 0.5H), 4.26 (s, 1.5H), 4.31 (br. s, 0.25H), 6.80-6.87 (m, 1.5H), 6.90-7.06 (m, 2.5H), 7.24-7.39 (m, 4H), 7.54-7.62 (m, 1H), 7.73-7.80 (m, 2H), 8.53-8.62 (m, 1H). [α]_{D}²⁰ = −27.50° (c = 0.280, methanol). LC-MS (Method 1): Rₜ = 0.81 min; m/z = 495 (M + H)⁺. |
| 59 | 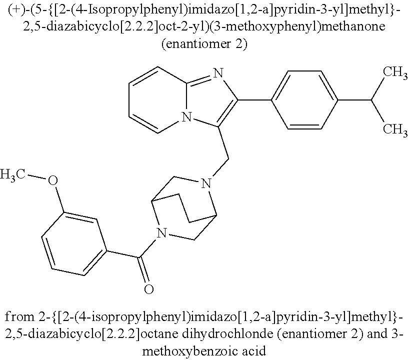 [SMILES: COc1cccc(C(=O)N2CC3CCC2CN3Cc2c(-c3ccc(C(C)C)cc3)nc3ccccn23)c1] | ¹H-NMR (400 MHz, DMSO-d₆): δ [ppm] = 1.19-1.30 (m, 6H), 1.47-1.69 (m, 1.75H), 1.71-1.98 (m, 2.25H), 2.66-2.74 (m, 1H), 2.82 (br. d, 1H), 2.87-3.02 (m, 2H), 3.17 (br. d, 0.25H), 3.37 (br. d, 0.75H), 3.54 (br. s, 0.75H), 3.64 (br. d, 0.25H), 3.69-3.82 (m, 3.75H), 4.22 (s, 0.5H), 4.26 (s, 1.5H), 4.31 (br. s, 0.25H), 6.80-6.87 (m, 1.5H), 6.89-7.05 (m, 2.5H), 7.23-7.39 (m, 4H), 7.53-7.61 (m, 1H), 7.71-7.82 (m, 2H), 8.53-8.62 (m, 1H). [α]_{D}²⁰ = +30.79° (c = 0.275, methanol). LC-MS (Method 4): Rₜ = 2.02 min; m/z = 495 (M + H)⁺. |
| 60 | 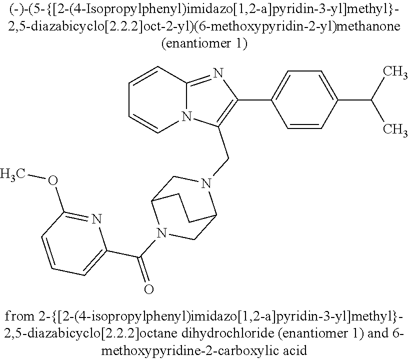 [SMILES: COc1cccc(C(=O)N2CC3CCC2CN3Cc2c(-c3ccc(C(C)C)cc3)nc3ccccn23)n1] | ¹H-NMR (400 MHz, DMSO-d₆): δ [ppm] = 1.24 (d, 6H), 1.49-2.00 (m, 4H), 2.67-2.78 (m, 1H), 2.84-3.01 (m, 3H), 3.39 (dd, 0.75H), 3.46 (d, 0.25H), 3.71-3.78 (m, 3H), 3.82 (s, 0.75H), 3.94-4.03 (m, 1H), 4.21-4.31 (m, 2H), 4.39 (br. s, 0.25H), 6.85-7.00 (m, 2H), 7.17 (d, 0.75H), 7.24-7.38 (m, 3.25H), 7.58 (d, 1H), 7.70-7.86 (m, 3H), 8.54-8.61 (m, 1H). [α]_{D}²⁰ = −32.98° (c = 0.285, methanol). LC-MS (Method 2): Rₜ = 1.34 min; m/z = 496 (M + H)⁺. |
| 61 | 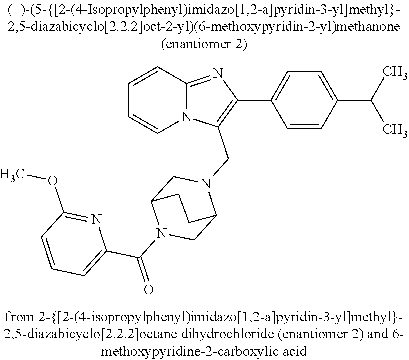 [SMILES: COc1cccc(C(=O)N2CC3CCC2CN3Cc2c(-c3ccc(C(C)C)cc3)nc3ccccn23)n1] | ¹H-NMR (400 MHz, DMSO-d₆): δ [ppm] = 1.24 (d, 6H), 1.49-2.01 (m, 4H), 2.68-2.78 (m, 1H), 2.84-3.01 (m, 3H), 3.39 (dd, 0.75H), 3.46 (d, 0.25H), 3.71-3.78 (m, 3H), 3.82 (s, 0.75H), 3.94-4.03 (m, 1H), 4.22-4.31 (m, 2H), 4.39 (br. s, 0.25H), 6.85-7.00 (m, 2H), 7.17 (d, 0.75H), 7.23-7.38 (m, 3.25H), 7.58 (d, 1H), 7.70-7.85 (m, 3H), 8.54-8.61 (m, 1H). [α]_{D}²⁰ = +36.23° (c = 0.265, methanol). LC-MS (Method 4): Rₜ = 2.02 min; m/z = 496 (M + H)⁺. |
| 62 | 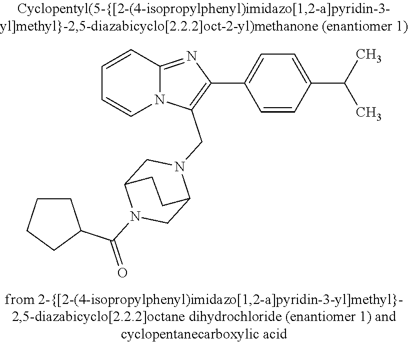 [SMILES: CC(C)c1ccc(-c2nc3ccccn3c2CN2CC3CCC2CN3C(=O)C2CCCC2)cc1] | ¹H-NMR (400 MHz, DMSO-d₆): δ [ppm] = 1.25 (d, 6H), 1.39-1.79 (m, 11H), 1.80-1.96 (m, 1H), 2.64-2.86 (m, 4H), 2.88-3.01 (m, 1H), 3.19 (dd, 0.5H), 3.27-3.38 (m, 0.5H, partly concealed by H₂O signal), 3.55 (br. d, 0.5H), 3.73 (br. d, 0.5H), 3.94 (br. s, 0.5H), 4.17-4.30 (m, 2.5H), 6.95 (t, 1H), 7.28 (t, 1H), 7.34 (d, 2H), 7.58 (dd, 1H), 7.71-7.79 (m, 2H), 8.56 (d, 1H). LC-MS (Method 2): Rₜ = 1.44 min; m/z = 457 (M + H)⁺. |
| 63 | 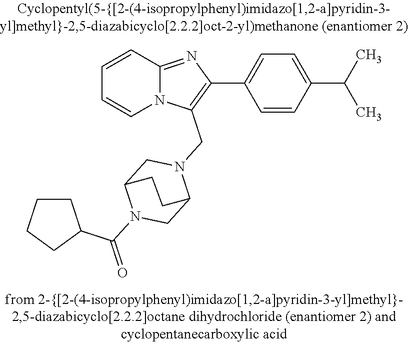 [SMILES: CC(C)c1ccc(-c2nc3ccccn3c2CN2CC3CCC2CN3C(=O)C2CCCC2)cc1] | ¹H-NMR (400 MHz, DMSO-d₆): δ [ppm] = 1.25 (d, 6H), 1.38-1.80 (m, 11H), 1.80-1.96 (m, 1H), 2.64-2.86 (m, 4H), 2.88-3.01 (m, 1H), 3.19 (dd, 0.5H), 3.27-3.38 (m, 0.5H, partly concealed by H₂O signal), 3.55 (br. d, 0.5H), 3.73 (br. d, 0.5H), 3.94 (br. s, 0.5H), 4.17-4.30 (m, 2.5H), 6.95 (t, 1H), 7.28 (t, 1H), 7.34 (d, 2H), 7.58 (dd, 1H), 7.70-7.79 (m, 2H), 8.56 (d, 1H). LC-MS (Method 4): Rₜ = 2.10 min; m/z = 457 (M + H)⁺. |
| 64 | 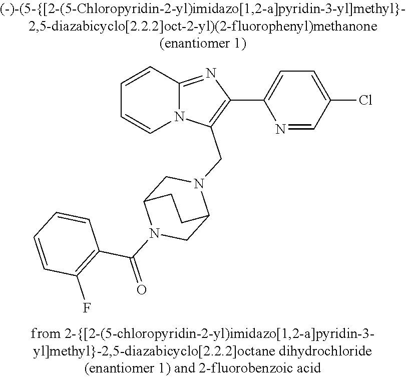 [SMILES: O=C(c1ccccc1F)N1CC2CCC1CN2Cc1c(-c2ccc(Cl)cn2)nc2ccccn12] | ¹H-NMR (400 MHz, DMS0-d₆): δ [ppm] = 1.49-1.93 (m, 3.3H), 1.94-2.07 (m, 0.7H), 2.65-2.73 (m, 0.3H), 2.87 (br. d, 0.7H), 2.91-3.09 (m, 2.3H), 3.36 (br. s, 0.7H), 3.43 (dd, 0.7H), 3.73 (br. d, 0.3H), 3.85 (br. d, 0.7H), 4.43 (br. s, 0.3H), 4.53-4.75 (m, 2H), 6.94-7.05 (m, 1H), 7.21-7.40 (m, 3.7H), 7.41-7.57 (m, 1.3H), 7.58-7.66 (m, 1H), 7.94-8.05 (m, 1H), 8.19 (d, 0.3H), 8.22 (d, 0.7H), 8.41 (d, 0.3H), 8.45-8.52 (m, 1H), 8.66 (d, 0.7H). [α]_{D}²⁰ = −55.55° (c = 0.270, methanol). LC-MS (Method 2): Rₜ = 1.13 min; m/z = 476/478 (M + H)⁺. |
| 65 | 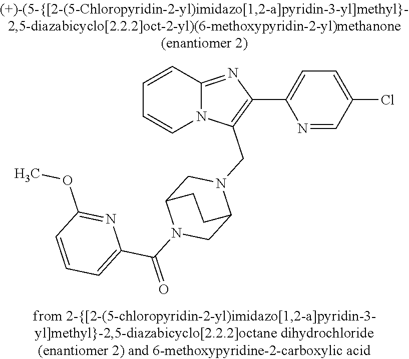 [SMILES: COc1cccc(C(=O)N2CC3CCC2CN3Cc2c(-c3ccc(Cl)cn3)nc3ccccn23)n1] | ¹H-NMR (400 MHz, DMS0-d₆): δ [ppm] = 1.50-2.07 (m, 4H), 2.68-2.72 (m, 0.3H), 2.85-2.95 (m, 14H), 3.00-3.09 (m, 1.3H), 3.37 (dd, 0.7H), 3.49 (dd, 0.3H), 3.77 (s, 2.3H), 3.81-3.89 (m, 1.4H), 4.01 (br. s, 0.7H), 4.08 (br. d, 0.3H), 4.42 (br. s, 0.3H), 4.57-4.76 (m, 2H), 6.89 (d, 0.7H), 6.94 (d, 0.3H), 6.96-7.04 (m, 1H), 7.21 (d, 0.7H), 7.31-7.39 (m, 1.3H), 7.57-7.65 (m, 1H), 7.77-7.89 (m, 1H), 7.97-8.04 (m, 1H), 8.17-8.25 (m, 1H), 8.43 (d, 0.3H), 8.47-8.53 (m, 1H), 8.63 (d, 0.7H). [α]_{D}²⁰ = +76.24° (c = 0.275, methanol). LC-MS (Method 2): Rₜ = 1.16 min; m/z = 489/491 (M + H)⁺. |
| 66 | 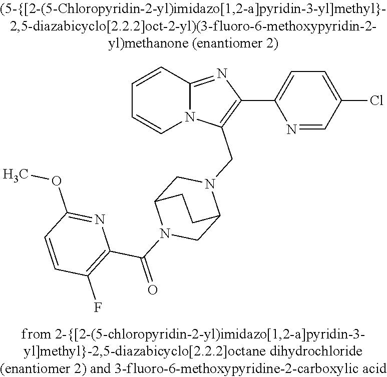 [SMILES: COc1ccc(F)c(C(=O)N2CC3CCC2CN3Cc2c(-c3ccc(Cl)cn3)nc3ccccn23)n1] | ¹H-NMR (400 MHz, DMSO-d₆): δ [ppm] = 1.53-2.08 (m, 4H), 2.76 (br. s, 0.3H), 2.87 (dd, 0.7H), 2.91-3.09 (m, 2H), 3.15 (br. d, 0.3H), 3.42 (dd, 0.7H), 3.50 (br. s, 0.7H), 3.72-3.89 (m, 4H), 4.42 (br. s, 0.3H), 4.58-4.76 (m, 2H), 6.93 (dd, 0.7H), 6.96-7.04 (m, 1.3H), 7.31-7.39 (m, 1H), 7.59-7.65 (m, 1H), 7.76 (t, 0.7H), 7.84 (t, 0.3H), 7.96-8.04 (m, 1H), 8.16-8.25 (m, 1H), 8.44-8.52 (m, 1.3H), 8.65 (d, 0.7H). LC-MS (Method 2): Rₜ = 1.13 min; m/z = 507/509 (M + H)⁺. |
| 67 | 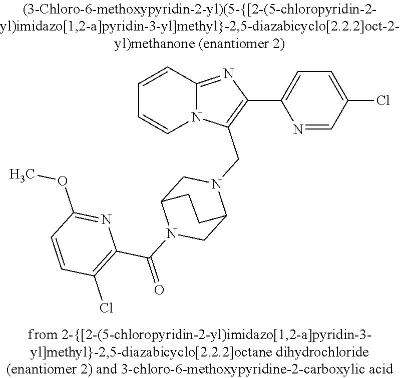 [SMILES: COc1ccc(Cl)c(C(=O)N2CC3CCC2CN3Cc2c(-c3ccc(Cl)cn3)nc3ccccn23)n1] | ¹H-NMR (400 MHz, DMSO-d₆): δ [ppm] = 1.51-2.08 (m, 4H), 2.74 (br. s, 0.3H), 2.83 (dd, 0.7H), 2.91-3.08 (m, 2.3H), 3.24 (br. s, 0.7H), 3.44 (br. d, 0.7H), 3.66 (br. d, 0.3H), 3.76 (s, 2.3H), 3.82-3.90 (m, 1.4H), 4.43 (br. s, 0.3H), 4.62-4.74 (m, 2H), 6.90 (d, 0.7H), 6.93-7.06 (m, 1.3H), 7.30-7.39 (m, 1H), 7.58-7.67 (m, 1H), 7.85 (d, 0.7H), 7.95 (d, 0.3H), 7.97-8.05 (m, 1H), 8.16-8.27 (m, 1H), 8.44 (d, 0.3H), 8.46-8.53 (m, 1H), 8.65 (d, 0.7H). LC-MS (Method 2): Rₜ = 1.21 min; m/z = 523/524/525 (M + H)⁺. |
| 68 | 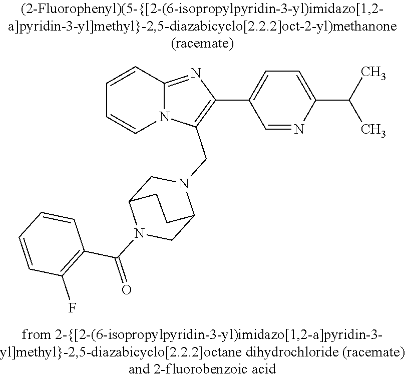 [SMILES: CC(C)c1ccc(-c2nc3ccccn3c2CN2CC3CCC2CN3C(=O)c2ccccc2F)cn1] | LC-MS (Method 2): Rₜ = 1.19 min; m/z = 484 (M + H)⁺. |
| 69 | 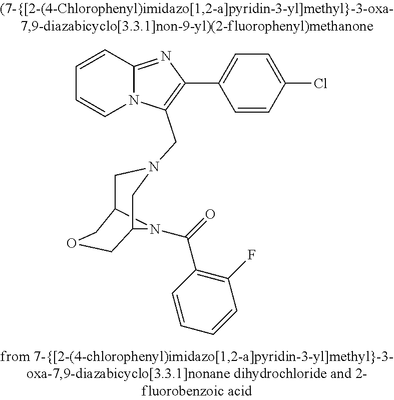 [SMILES: O=C(c1ccccc1F)N1C2COCC1CN(Cc1c(-c3ccc(Cl)cc3)nc3ccccn13)C2] | ¹H-NMR (400 MHz, DMSO-d₆, δ/ppm): 2.45 (br. d, 1H), 2.57 (br. d, 1H, partly concealed by DMSO signal), 2.86 (br. d, 1H), 3.00 (br. d, 1H), 3.39 (br. s, 1H), 3.59 (br. d, 1H), 3.70 (br. t, 2H), 3.86 (d, 1H), 3.93 (s, 2H), 4.49 (br. s, 1H), 6.93 (td, 1H), 7.25-7.35 (m, 3H), 7.41-7.55 (m, 4H), 7.59 (d, 1H), 7.96 (d, 2H), 8.82 (d, 1H). LC-MS (Method 1): Rₜ = 0.73 min; m/z = 491/493 (M + H)⁺. |
| 70 | 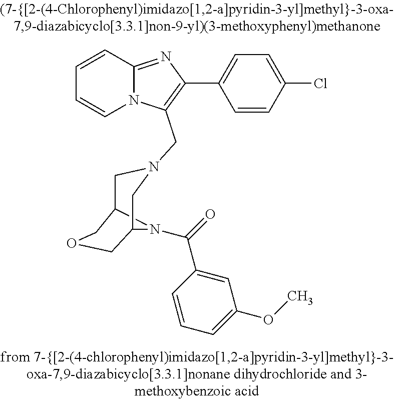 [SMILES: COc1cccc(C(=O)N2C3COCC2CN(Cc2c(-c4ccc(Cl)cc4)nc4ccccn24)C3)c1] | ¹H-NMR (400 MHz, DMSO-d₆, δ/ppm): 2.46-2.57 (m, 1H, concealed by DMSO signal), 2.61 (br. d, 1H), 2.83 (br. d, 1H), 2.96 (br. d, 1H), 3.56-3.75 (m, 4H), 3.77 (s, 3H), 3.84 (d, 1H), 3.94 (s, 2H), 4.41 (br. s, 1H), 6.88-7.06 (m, 4H), 7.30 (t, 1H), 7.36 (t, 1H), 7.52 (d, 2H), 7.59 (d, 1H), 7.98 (d, 2H), 8.83 (d, 1H). LC-MS (Method 1): Rₜ = 0.74 min; m/z = 503/505 (M + H)⁺. |
| 71 | 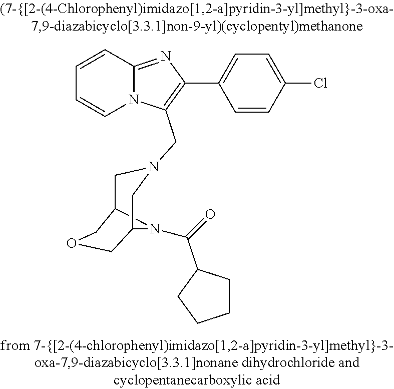 [SMILES: O=C(C1CCCC1)N1C2COCC1CN(Cc1c(-c3ccc(Cl)cc3)nc3ccccn13)C2] | ¹H-NMR (400 MHz, DMSO-d₆, δ/ppm): 1.43-1.81 (m, 8H), 2.40 (br. d, 1H), 2.45-2.57 (m, 1H, partly concealed by DMSO signal), 2.83-2.96 (m, 3H), 3.51 (br. d, 1H), 3.60 (br. d, 1H), 3.76 (dd, 2H), 3.85-3.95 (m, 2H), 4.05 (br. s, 1H), 4.34 (br. s, 1H), 6.93 (td, 1H), 7.30 (ddd, 1H), 7.51 (d, 2H), 7.60 (d, 1H), 7.98 (d, 2H), 8.82 (d, 1H). LC-MS (Method 1): Rₜ = 0.74 min; m/z = 465/467 (M + H)⁺. |
| 72 | 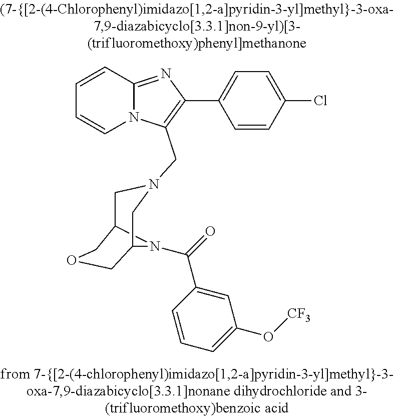 [SMILES: O=C(c1cccc(OC(F)(F)F)c1)N1C2COCC1CN(Cc1c(-c3ccc(Cl)cc3)nc3ccccn13)C2] | ¹H-NMR (400 MHz, DMSO-d₆, δ/ppm): 2.47-2.58 (m, 1H, partly concealed by DMSO signal), 2.64 (br. d, 1H), 2.83 (br. d, 1H), 2.96 (br. d, 1H), 3.53 (br. s, 1H), 3.63-3.77 (m, 3H), 3.84 (d, 1H), 3.95 (s, 2H), 4.42 (br. s, 1H), 6.93 (td, 1H), 7.30 (dd, 1H), 7.40-7.55 (m, 5H), 7.56-7.64 (m, 2H), 7.97 (d, 2H), 8.82 (d, 1H). LC-MS (Method 2): Rₜ = 1.60 min; m/z = 557/559 (M + H)⁺. |
| 73 | 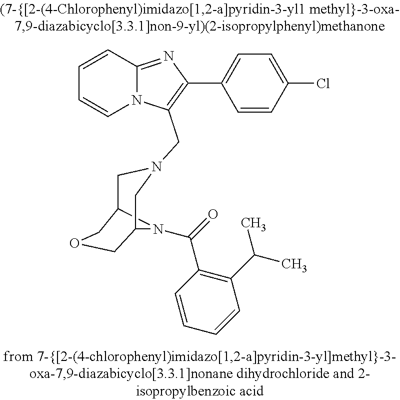 [SMILES: CC(C)c1ccccc1C(=O)N1C2COCC1CN(Cc1c(-c3ccc(Cl)cc3)nc3ccccn13)C2] | ¹H-NMR (400 MHz, DMSO-d₆, δ/ppm): 1.11-1.26 (m, 6H), 2.26 (br. d, 0.5H), 2.46-2.59 (m, 1H, partly concealed by DMSO signal), 2.62 (br. d, 0.5H), 2.77-2.89 (m, 1H), 2.90-3.05 (m, 2H), 3.25-3.35 (m, 1H, partly concealed by H₂O signal), 3.44 (br. d, 0.5H), 3.61-3.76 (m, 2.5H), 3.78-3.88 (m, 1H), 3.88-4.01 (m, 2H), 4.52 (br. s, 1H), 6.94 (td, 1H), 7.15-7.26 (m, 2H), 7.30 (ddd, 1H), 7.35-7.45 (m, 2H), 7.51 (t, 2H), 7.60 (d, 1H), 7.93 (d, 1H), 7.98 (d, 1H), 8.76 (d, 0.5H), 8.83 (d, 0.5H). LC-MS (Method 2): Rₜ = 1.60 min; m/z = 515/517 (M + H)⁺. |
| 74 | 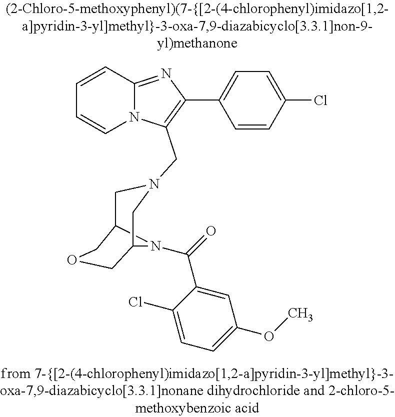 [SMILES: COc1ccc(Cl)c(C(=O)N2C3COCC2CN(Cc2c(-c4ccc(Cl)cc4)nc4ccccn24)C3)c1] | ¹H-NMR (400 MHz, DMSO-d₆, δ/ppm): 2.47-2.60 (m, 1.5H, partly concealed by DMSO signal), 2.65 (br. d, 0.5H), 2.77-2.92 (m, 1H), 2.92-3.04 (m, 1H), 3.26 (br. d, 1H), 3.62-3.98 (m, 9H), 4.46 (br. s, 1H), 6.93 (t, 1H), 7.01 (d, 2H), 7.30 (t, 1H), 7.42 (d, 1H), 7.52 (d, 2H), 7.59 (d, 1H), 7.97 (t, 2H), 8.82 (t, 1H). LC-MS (Method 2): Rₜ = 1.44 min; m/z = 537/538/539 (M + H)⁺. |
| 75 | 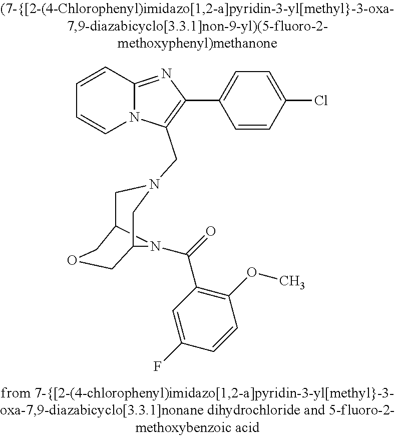 [SMILES: COc1ccc(F)cc1C(=O)N1C2COCC1CN(Cc1c(-c3ccc(Cl)cc3)nc3ccccn13)C2] | ¹H-NMR (400 MHz, DMSO-d₆, δ/ppm): 2.38-2.60 (m, 2H, partly concealed by DMSO signal), 2.75-2.89 (m, 1H), 2.89-3.04 (m, 1H), 3.26 (br. s, 1H), 3.47-3.97 (m, 9H), 4.44 (br. s, 1H), 6.94 (t, 1H), 7.05-7.18 (m, 2H), 7.23 (tt, 1H), 7.30 (t, 1H), 7.52 (d, 2H), 7.59 (d, 1H), 7.96 (d, 2H), 8.82 (d, 1H). LC-MS (Method 2): Rₜ = 1.35 min; m/z = 521/523 (M + H)⁺. |
| 76 | 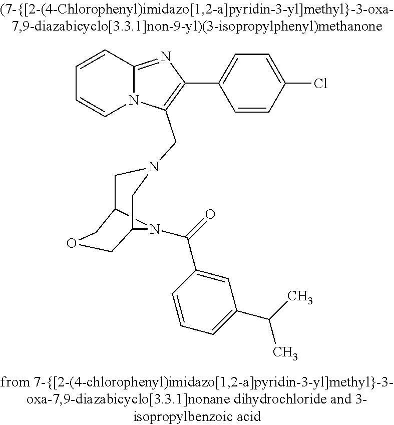 [SMILES: CC(C)c1cccc(C(=O)N2C3COCC2CN(Cc2c(-c4ccc(Cl)cc4)nc4ccccn24)C3)c1] | ¹H-NMR (400 MHz, DMSO-d₆, δ/ppm): 1.19 (d, 6H), 2.47-2.57 (m, 1H, concealed by DMSO signal), 2.63 (br. d, 1H), 2.80-3.00 (m, 3H), 3.55-3.75 (m, 4H), 3.84 (br. d, 1H), 3.94 (s, 2H), 4.42 (br. s, 1H), 6.93 (td, 1H), 7.20-7.39 (m, 5H), 7.52 (d, 2H), 7.59 (d, 1H), 7.98 (d, 2H), 8.83 (d, 1H). LC-MS (Method 2): Rₜ = 1.62 min; m/z = 515/517 (M + H)⁺. |
| 77 | 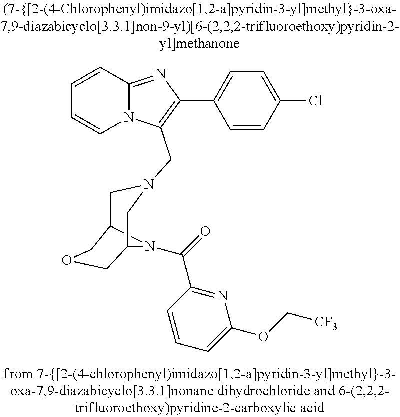 [SMILES: O=C(c1cccc(OCC(F)(F)F)n1)N1C2COCC1CN(Cc1c(-c3ccc(Cl)cc3)nc3ccccn13)C2] | ¹H-NMR (400 MHz, DMSO-d₆, δ/ppm): 2.58-2.69 (m, 2H), 2.87 (br. d, 1H), 3.07 (br. d, 1H), 3.66-3.76 (m, 3H), 3.87-4.00 (m, 3H), 4.16 (br. s, 1H), 4.47 (br. s, 1H), 4.92 (q, 2H), 6.92 (td, 1H), 7.11 (d, 1H), 7.30 (ddd, 1H), 7.42 (d, 1H), 7.51 (d, 2H), 7.60 (d, 1H), 7.91-8.01 (m, 3H), 8.84 (d, 1H). LC-MS (Method 2): Rₜ = 1.60 min; m/z = 572/574 (M + H)⁺. |
| 78 | 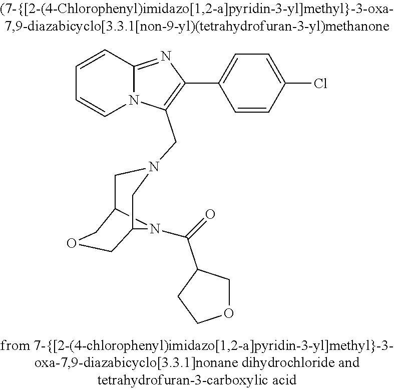 [SMILES: O=C(C1CCOC1)N1C2COCC1CN(Cc1c(-c3ccc(Cl)cc3)nc3ccccn13)C2] | ¹H-NMR (400 MHz, DMSO-d₆, δ/ppm): 1.73-2.16 (m, 4H), 2.42 (br. t, 1H), 2.46-2.59 (m, 1H, partly concealed by DMSO signal), 2.91 (br. d, 2H), 3.47-3.58 (m, 1H), 3.64 (br. d, 1H), 3.68-3.82 (m, 4H), 3.84-3.96 (m, 2H), 4.09 (br. s, 1H), 4.28 (br. s, 1H), 4.60 (td, 1H), 6.93 (td, 1H), 7.30 (ddd, 1H), 7.51 (dd, 2H), 7.60 (d, 1H), 7.93-8.02 (m, 2H), 8.82 (d, 1H). LC-MS (Method 2): Rₜ = 1.13 min; m/z = 467/469 (M + H)⁺. |
| 79 | 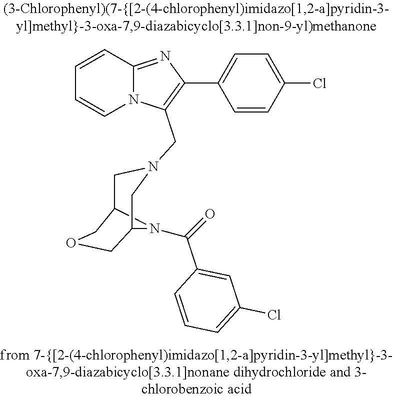 [SMILES: O=C(c1cccc(Cl)c1)N1C2COCC1CN(Cc1c(-c3ccc(Cl)cc3)nc3ccccn13)C2] | ¹H-NMR (400 MHz, DMSO-d₆, δ/ppm): 2.46-2.58 (m, 1H, partly concealed by DMSO signal), 2.63 (d, 1H), 2.83 (br. d, 1H), 2.96 (br. d, 1H), 3.56 (br. s, 1H), 3.62-3.76 (m, 3H), 3.84 (d, 1H), 3.95 (s, 2H), 4.41 (br. s, 1H), 6.93 (t, 1H), 7.30 (t, 1H), 7.39 (d, 1H), 7.45-7.56 (m, 5H), 7.60 (d, 1H), 7.97 (d, 2H), 8.83 (d, 1H). LC-MS (Method 2): Rₜ = 1.54 min; m/z = 507/508/509 (M + H)⁺. |
| 80 | 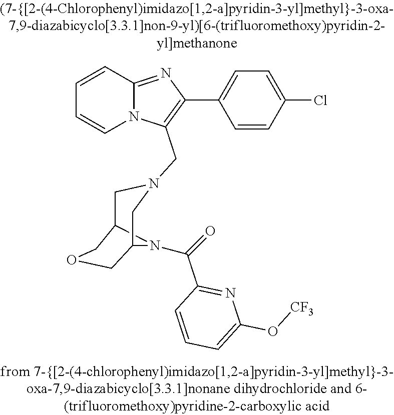 [SMILES: O=C(c1cccc(OC(F)(F)F)n1)N1C2COCC1CN(Cc1c(-c3ccc(Cl)cc3)nc3ccccn13)C2] | ¹H-NMR (400 MHz, DMSO-d₆, δ/ppm): 2.56-2.64 (m, 2H), 2.88 (br. d, 1H), 3.03 (br. d, 1H), 3.64-3.79 (m, 3H), 3.88 (d, 1H), 3.93 (s, 2H), 4.01 (br. s, 1H), 4.46 (br. s, 1H), 6.93 (t, 1H), 7.31 (t, 1H), 7.41 (d, 1H), 7.51 (d, 2H), 7.60 (d, 1H), 7.73 (d, 1H), 7.97 (d, 2H), 8.18 (t, 1H), 8.81 (d, 1H). LC-MS (Method 2): Rₜ = 1.47 min; m/z = 558/560 (M + H)⁺. |
| 81 | 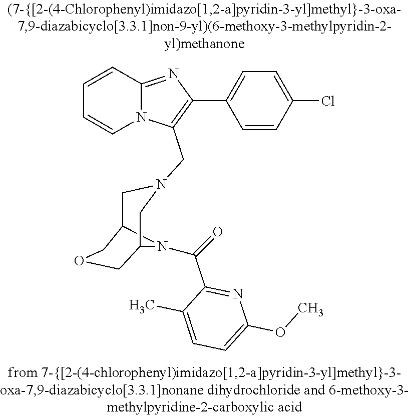 [SMILES: COc1ccc(C)c(C(=O)N2C3COCC2CN(Cc2c(-c4ccc(Cl)cc4)nc4ccccn24)C3)n1] | ¹H-NMR (400 MHz, DMSO-d₆, δ/ppm): 2.21 (s, 3H), 2.46-2.61 (m, 2H, partly concealed by DMSO signal), 2.86 (br. d, 1H), 3.03 (br. d, 1H), 3.38 (br. s, 1H), 3.57-3.74 (m, 3H), 3.77 (s, 3H), 3.87 (d, 1H), 3.93 (s, 2H), 4.48 (br. s, 1H), 6.79 (d, 1H), 6.93 (td, 1H), 7.30 (t, 1H), 7.52 (d, 2H), 7.60 (d, 1H), 7.64 (d, 1H), 7.97 (d, 2H), 8.81 (d, 1H). LC-MS ((Method 1): Rₜ = 0.75 min; m/z = 518/520 (M + H)⁺. |
| 82 | 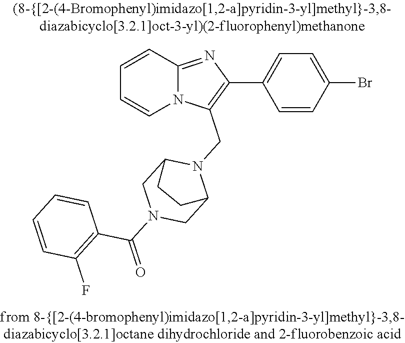 [SMILES: O=C(c1ccccc1F)N1CC2CCC(C1)N2Cc1c(-c2ccc(Br)cc2)nc2ccccn12] | ¹H-NMR (400 MHz, DMSO-d₆, δ/ppm): 1.33-1.60 (m, 2H), 1.78-1.97 (m, 2H), 2.79 (br. d, 1H), 2.99 (br. d, 1H), 3.05 (br. s, 1H), 3.12 (br. d, 1H), 3.21 (br. s, 1H), 3.93-4.05 (m, 2H), 4.16 (br. d, 1H), 6.99 (t, 1H), 7.25 (t, 2H), 7.32 (t, 2H), 7.46 (q, 1H), 7.60 (d, 1H), 7.66 (d, 2H), 7.80 (d, 2H), 8.67 (d, 1H). LC-MS (Method 1): Rₜ = 0.80 min; m/z = 519/521 (M + H)⁺. |
| 83 | 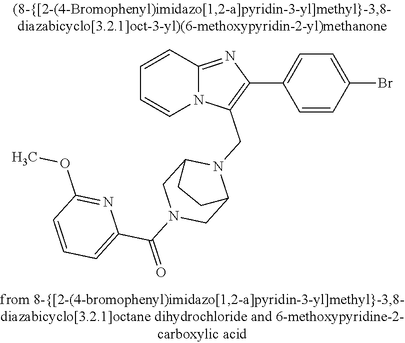 [SMILES: COc1cccc(C(=O)N2CC3CCC(C2)N3Cc2c(-c3ccc(Br)cc3)nc3ccccn23)n1] | ¹H-NMR (400 MHz, DMSO-d₆, δ/ppm): 1.49-1.60 (m, 1H), 1.62-1.71 (m, 1H), 1.79-1.98 (m, 2H), 2.81 (d, 1H), 3.05 (br. s, 1H), 3.11 (br. d, 1H), 3.23 (br. s, 1H), 3.39 (br. d, 1H), 3.80 (s, 3H), 3.93-4.07 (m, 2H), 4.13 (br. d, 1H), 6.85 (d, 1H), 6.99 (t, 1H), 7.09 (d, 1H), 7.32 (t, 1H), 7.60 (d, 1H), 7.66 (d, 2H), 7.77 (t, 1H), 7.81 (d, 2H), 8.68 (d, 1H). LC-MS (Method 2): Rₜ = 1.41 min; m/z = 532/534 (M + H)⁺. |
| 84 | 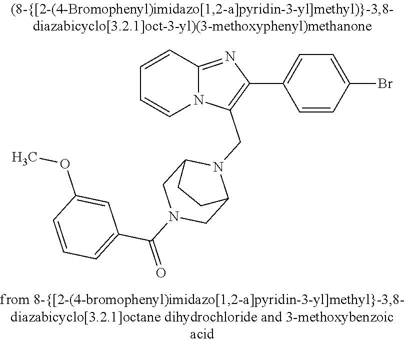 [SMILES: COc1cccc(C(=O)N2CC3CCC(C2)N3Cc2c(-c3ccc(Br)cc3)nc3ccccn23)c1] | ¹H-NMR (400 MHz, DMSO-d₆, δ/ppm): 1.33-1.47 (m, 1H), 1.49-1.63 (m, 1H), 1.77-1.95 (m, 2H), 2.77 (br. d, 1H), 3.05 (br. s, 1H), 3.16 (br. s, 3H), 3.75 (s, 3H), 3.98 (br. s, 2H), 4.16 (br. d, 1H), 6.80-6.89 (m, 2H), 6.92-7.02 (m, 2H), 7.26-7.36 (m, 2H), 7.60 (d, 1H), 7.66 (d, 2H), 7.80 (d, 2H), 8.66 (d, 1H). LC-MS (Method 2): Rₜ = 1.46 min; m/z = 531/533 (M + H)⁺. |
| 85 | 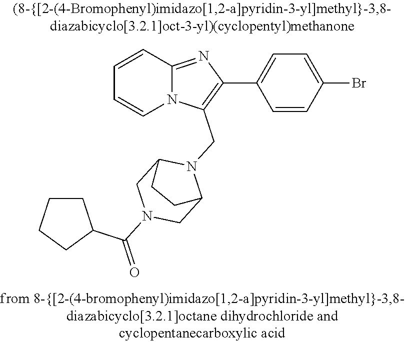 [SMILES: O=C(C1CCCC1)N1CC2CCC(C1)N2Cc1c(-c2ccc(Br)cc2)nc2ccccn12] | ¹H-NMR (400 MHz, DMSO-d₆, δ/ppm): 1.29-1.39 (m, 1H), 1.39-1.92 (m, 11H), 2.44-2.61 (m, 1H, partly concealed by DMSO signal), 2.78-2.90 (m, 1H), 3.01 (br. d, 1H), 3.08 (br. s, 1H), 3.13 (br. s, 1H), 3.58 (br. d, 1H), 3.95 (br. d, 1H), 4.01 (s, 2H), 6.99 (t, 1H), 7.32 (t, 1H), 7.60 (d, 1H), 7.67 (d, 2H), 7.82 (d, 2H), 8.66 (d, 1H). LC-MS (Method 2): Rₜ = 1.48 min; m/z = 493/495 (M + H)⁺. |
| 86 | 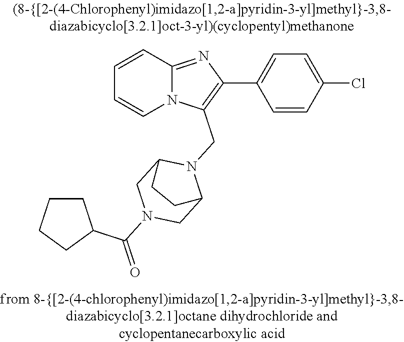 [SMILES: O=C(C1CCCC1)N1CC2CCC(C1)N2Cc1c(-c2ccc(Cl)cc2)nc2ccccn12] | ¹H-NMR (400 MHz, DMSO-d₆, δ/ppm): 1.31-1.38 (m, 1H), 1.41-1.68 (m, 8H), 1.69-1.78 (m, 1H), 1.78-1.89 (m, 2H), 2.57 (br. d, 1H), 2.80-2.88 (m, 1H), 3.01 (br. d, 1H), 3.08 (br. s, 1H), 3.13 (br. s, 1H), 3.58 (br. d, 1H), 3.95 (br. d, 1H), 4.01 (s, 2H), 6.99 (td, 1H), 7.32 (ddd, 1H), 7.53 (d, 2H), 7.60 (d, 1H), 7.87 (d, 2H), 8.66 (d, 1H). LC-MS (Method 1): Rₜ = 0.80 min; m/z = 449/451 (M + H)⁺. |
| 87 | 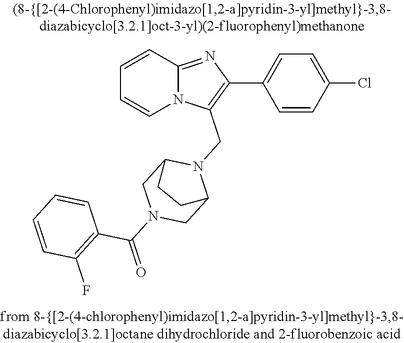 [SMILES: O=C(c1ccccc1F)N1CC2CCC(C1)N2Cc1c(-c2ccc(Cl)cc2)nc2ccccn12] | ¹H-NMR (400 MHz, DMSO-d₆, δ/ppm): 1.38-1.49 (m, 1H), 1.50-1.59 (m, 1H), 1.77-1.95 (m, 2H), 2.79 (br. d, 1H), 2.99 (br. d, 1H), 3.05 (br. s, 1H), 3.12 (br. d, 1H), 3.21 (br. d, 1H), 4.00 (q, 2H), 4.15 (br. d, 1H), 6.99 (td, 1H), 7.25 (br. t, 2H), 7.32 (ddd, 2H), 7.42 (m, 1H), 7.53 (d, 2H), 7.60 (d, 1H), 7.85 (d, 2H), 8.66 (d, 1H). LC-MS (Method 1): Rₜ = 0.78 min; m/z = 475/477 (M + H)⁺. |
| 88 | 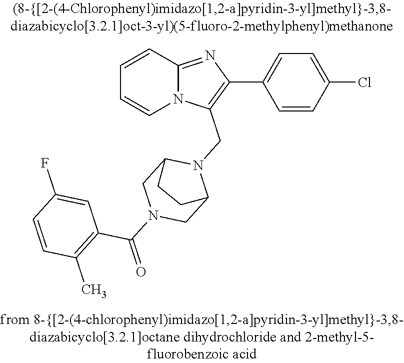 [SMILES: Cc1ccc(F)cc1C(=O)N1CC2CCC(C1)N2Cc1c(-c2ccc(Cl)cc2)nc2ccccn12] | ¹H-NMR (400 MHz, DMSO-d₆, δ/ppm): 1.27-1.41 (m, 0.5H), 1.41-1.66 (m, 1.5H), 1.77-1.97 (m, 2H), 2.04 (br. s, 1.5H), 2.21 (br. s, 1.5H), 2.79 (br. d, 1H), 2.87 (br. s, 1H), 3.02 (br. s, 1.5H), 3.09-3.19 (m, 0.5H), 3.22 (br. s, 1H), 3.92-4.07 (m, 2H), 4.14 (br. d, 1H), 6.93-7.06 (m, 2H), 7.10 (td, 1H), 7.20-7.37 (m, 2H), 7.52 (d, 2H), 7.60 (d, 1H), 7.86 (d, 2H), 8.65 (d, 1H). LC-MS (Method 1): Rₜ = 0.85 min; m/z = 489/491 (M + H)⁺. |
| 89 | 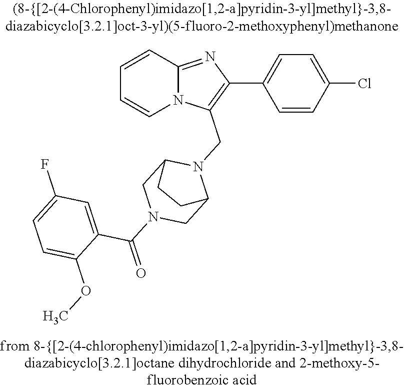 [SMILES: COc1ccc(F)cc1C(=O)N1CC2CCC(C1)N2Cc1c(-c2ccc(Cl)cc2)nc2ccccn12] | ¹H-NMR (400 MHz, DMSO-d₆, δ/ppm): 1.32-1.43 (m, 0.5H), 1.53 (br. d, 1H), 1.60-1.70 (m, 0.5H), 1.74-1.95 (m, 2H), 2.75 (br. d, 1H), 2.82-2.94 (m, 1H), 2.95-3.08 (m, 2H), 3.20 (br. s, 1H), 3.70 (br. s, 1.6H), 3.77 (br. s, 1.4H), 3.93-4.02 (m, 2H), 4.05 (br. d, 0.5H), 4.12 (br. d, 0.5H), 6.93-7.11 (m, 3H), 7.13-7.24 (m, 1H), 7.32 (br. t, 1H), 7.53 (d, 2H), 7.60 (d, 1H), 7.81-7.90 (m, 2H), 8.65 (d, 1H). LC-MS (Method 1): Rₜ = 0.81 min; m/z = 505/506 (M + H)⁺. |
| 90 | 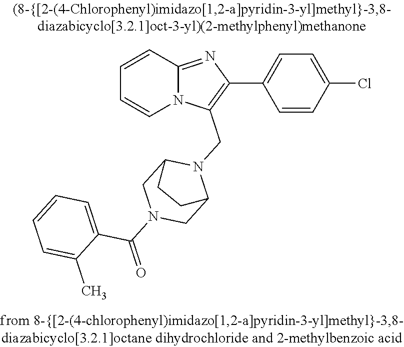 [SMILES: Cc1ccccc1C(=O)N1CC2CCC(C1)N2Cc1c(-c2ccc(Cl)cc2)nc2ccccn12] | ¹H-NMR (400 MHz, DMSO-d₆, δ/ppm): 1.28-1.42 (m, 0.5H), 1.42-1.65 (m, 1.5H), 1.76-1.97 (m, 2H), 2.08 (br. s, 1.5H), 2.24 (br. s, 1.5H), 2.78 (br. d, 1H), 2.88 (br. d, 1H), 2.96-3.15 (m, 2H), 3.22 (br. s, 1H), 3.92-4.07 (m, 2H), 4.17 (br. d, 1H), 6.99 (t, 1H), 7.02-7.29 (m, 4H), 7.32 (t, 1H), 7.52 (d, 2H), 7.60 (d, 1H), 7.85 (d, 2H), 8.65 (d, 1H). LC-MS (Method 1): Rₜ = 0.82 min; m/z = 471/473 (M + H)⁺. |
| 91 | 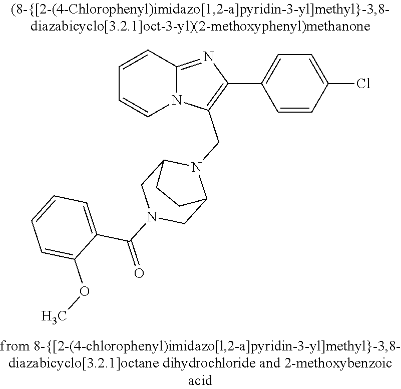 [SMILES: COc1ccccc1C(=O)N1CC2CCC(C1)N2Cc1c(-c2ccc(Cl)cc2)nc2ccccn12] | ¹H-NMR (400 MHz, DMSO-d₆, δ/ppm): 1.27-1.41 (m, 0.5H), 1.53 (br. t, 1H), 1.61-1.72 (m, 0.5H), 1.73-1.95 (m, 2H), 2.69-2.80 (m, 1H), 2.82-3.08 (m, 3H), 3.19 (br. s, 1H), 3.71 (s, 1.7H), 3.78 (s, 1.3H), 3.92-4.05 (m, 2H), 4.08 (br. d, 0.5H), 4.18 (br. d, 0.5H), 6.89-7.08 (m, 3.5H), 7.18 (br. d, 0.5H), 7.27-7.39 (m, 2H), 7.52 (d, 2H), 7.60 (d, 1H), 7.80-7.90 (m, 2H), 8.65 (d, 1H). LC-MS (Method 1): Rₜ = 0.78 min; m/z = 487/489 (M + H)⁺. |
| 92 | 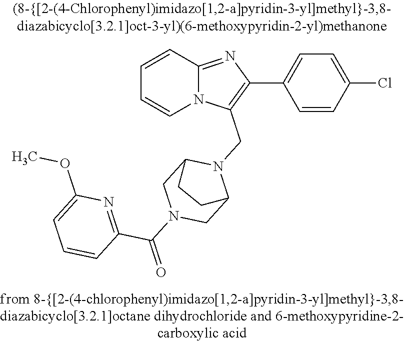 [SMILES: COc1cccc(C(=O)N2CC3CCC(C2)N3Cc2c(-c3ccc(Cl)cc3)nc3ccccn23)n1] | ¹H-NMR (400 MHz, DMSO-d₆, δ/ppm): 1.50-1.60 (m, 1H), 1.62-1.71 (m, 1H), 1.79-1.97 (m, 2H), 2.81 (br. d, 1H), 3.05 (br. s, 1H), 3.11 (br. d, 1H), 3.23 (br. s, 1H), 3.39 (br. d, 1H), 3.80 (s, 3H), 3.96-4.06 (m, 2H), 4.13 (br. d, 1H), 6.85 (d, 1H), 6.99 (t, 1H), 7.09 (d, 1H), 7.32 (t, 1H), 7.53 (d, 2H), 7.60 (d, 1H), 7.77 (t, 1H), 7.87 (d, 2H), 8.68 (d, 1H). LC-MS (Method 1): Rₜ = 0.75 min; m/z = 488/490 (M + H)⁺. |
| 93 | 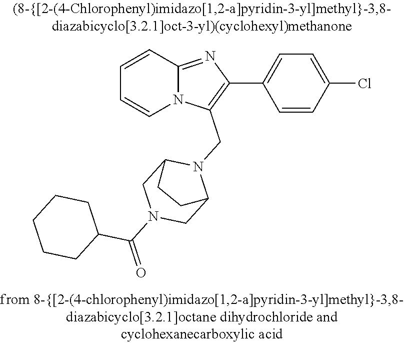 [SMILES: O=C(C1CCCCC1)N1CC2CCC(C1)N2Cc1c(-c2ccc(Cl)cc2)nc2ccccn12] | ¹H-NMR (400 MHz, DMSO-d₆, δ/ppm): 1.04-1.41 (m, 6H), 1.42-1.55 (m, 2H), 1.55-1.72 (m, 4H), 1.76-1.89 (m, 2H), 2.39-2.48 (m, 1H), 2.48-2.58 (br. d, 1H, partly concealed by DMSO signal), 3.03 (br. d, 1H), 3.10 (br. d, 2H), 3.55 (br. d, 1H), 3.94 (br. d, 1H), 4.01 (s, 2H), 6.99 (td, 1H), 7.32 (ddd, 1H), 7.53 (d, 2H), 7.61 (d, 1H), 7.87 (d, 2H), 8.66 (d, 1H). LC-MS (Method 1): Rₜ = 0.81 min; m/z = 463/465 (M + H)⁺. |
| 94 | 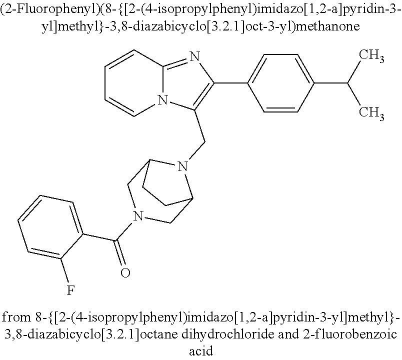 [SMILES: CC(C)c1ccc(-c2nc3ccccn3c2CN2C3CCC2CN(C(=O)c2ccccc2F)C3)cc1] | ¹H-NMR (400 MHz, DMSO-d₆, δ/ppm): 1.24 (d, 6H), 1.36-1.61 (m, 2H), 1.76-1.97 (m, 2H), 2.80 (br. d, 1H), 2.87-3.04 (m, 2H), 3.05-3.18 (m, 2H), 3.20-3.27 (m, 1H), 3.93-4.06 (m, 2H), 4.16 (br. d, 1H), 6.97 (td, 1H), 7.20-7.39 (m, 6H), 7.42-7.51 (m, 1H), 7.58 (d, 1H), 7.73 (d, 2H), 8.66 (d, 1H). LC-MS (Method 2): Rₜ = 1.46 min; m/z = 483 (M + H)⁺. |
| 95 | 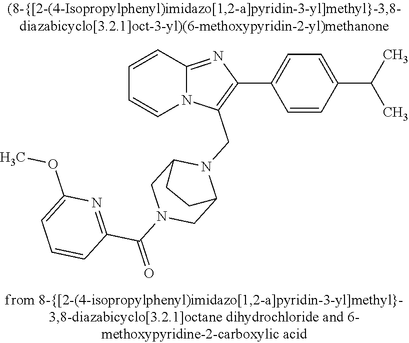 [SMILES: COc1cccc(C(=O)N2CC3CCC(C2)N3Cc2c(-c3ccc(C(C)C)cc3)nc3ccccn23)n1] | ¹H-NMR (400 MHz, DMSO-d₆, δ/ppm): 1.24 (d, 6H), 1.50-1.60 (m, 1H), 1.63-1.72 (m, 1H), 1.79-1.97 (m, 2H), 2.81 (br. d, 1H), 2.87-3.00 (m, 1H), 3.06-3.15 (m, 2H), 3.22-3.28 (m, 1H), 3.40 (br. d, 1H), 3.80 (s, 3H), 4.01 (s, 2H), 4.13 (br. d, 1H), 6.95 (dd, 1H), 6.97 (td, 1H), 7.09 (dd, 1H), 7.26-7.37 (m, 3H), 7.34 (d, 2H), 7.59 (d, 1H), 7.74 (d, 2H), 7.78 (d, 1H), 8.67 (d, 1H). LC-MS (Method 2): Rₜ = 1.45 min; m/z = 496 (M + H)⁺. |
| 96 | 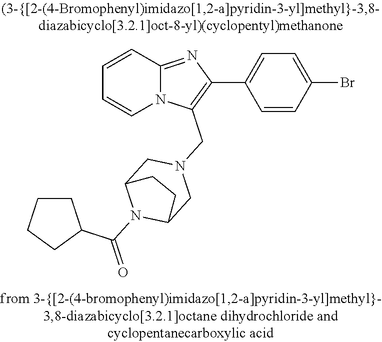 [SMILES: O=C(C1CCCC1)N1C2CCC1CN(Cc1c(-c3ccc(Br)cc3)nc3ccccn13)C2] | ¹H-NMR (400 MHz, DMSO-d₆, δ/ppm): 1.43-1.67 (m, 7H), 1.67-1.80 (m, 5H), 2.27 (br. dd, 2H), 2.55 (br. d, 1H, partly concealed by DMSO signal), 2.61 (br. d, 1H), 2.80-2.91 (m, 1H), 3.92-4.04 (m, 2H), 4.29 (br. s, 1H), 4.42 (br. d, 1H), 6.99 (td, 1H), 7.31 (ddd, 1H), 7.60 (d, 1H), 7.67 (d, 2H), 7.87 (d, 2H), 8.61 (d, 1H). LC-MS (Method 1): Rₜ = 0.89 min; m/z = 493/495 (M + H)⁺. |
| 97 | 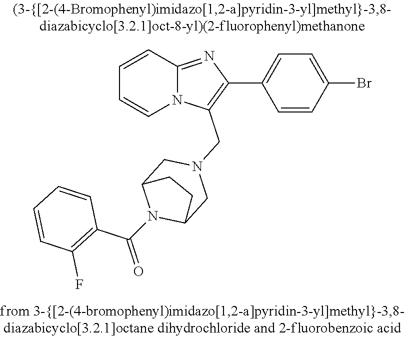 [SMILES: O=C(c1ccccc1F)N1C2CCC1CN(Cc1c(-c3ccc(Br)cc3)nc3ccccn13)C2] | ¹H-NMR (400 MHz, DMSO-d₆, δ/ppm): 1.63-1.80 (m, 4H), 2.26 (br. d, 1H), 2.43 (br. d, 1H), 2.55 (br. d, 1H, partly concealed by DMSO signal), 2.66 (br. d, 1H), 3.67 (br. s, 1H), 4.02 (s, 2H), 4.60 (br. d, 1H), 6.99 (td, 1H), 7.24-7.34 (m, 3H), 7.41-7.54 (m, 2H), 7.60 (d, 1H), 7.67 (d, 2H), 7.86 (d, 2H), 8.60 (d, 1H). LC-MS (Method 1): Rₜ = 0.86 min; m/z = 519/521 (M + H)⁺. |
| 98 | 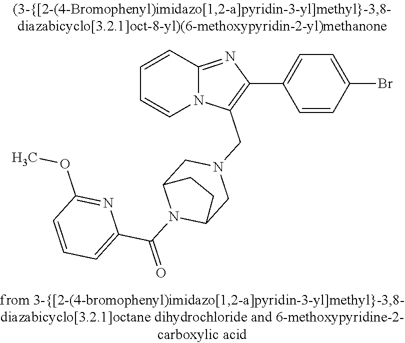 [SMILES: COc1cccc(C(=O)N2C3CCC2CN(Cc2c(-c4ccc(Br)cc4)nc4ccccn24)C3)n1] | ¹H-NMR (400 MHz, DMSO-d₆, δ/ppm): 1.65-1.84 (m, 4H), 2.46 (br. d, 1H), 2.60 (s, 2H), 2.73 (dd, 1H), 3.77 (s, 3H), 4.04 (s, 2H), 4.67 (br. d, 2H), 6.93 (d, 1H), 6.99 (td, 1H), 7.27-7.38 (m, 2H), 7.61 (d, 1H), 7.67 (d, 2H), 7.82 (t, 1H), 7.87 (d, 2H), 8.62 (d, 1H). LC-MS (Method 1): Rₜ = 0.86 min; m/z = 532/534 (M + H)⁺. |
| 99 | 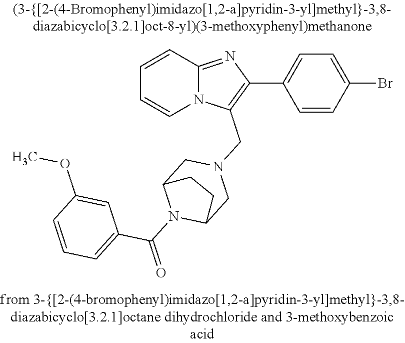 [SMILES: COc1cccc(C(=O)N2C3CCC2CN(Cc2c(-c4ccc(Br)cc4)nc4ccccn24)C3)c1] | ¹H-NMR (400 MHz, DMSO-d₆, δ/ppm): 1.64-1.79 (m, 4H), 2.36-2.69 (m, 4H, partly concealed by DMSO signal), 3.78 (s, 3H), 3.94 (br. s, 1H), 4.03 (s, 2H), 4.56 (br. s, 1H), 6.94-7.06 (m, 4H), 7.27-7.39 (m, 2H), 7.60 (d, 1H), 7.67 (d, 2H), 7.87 (d, 2H), 8.62 (d, 1H). LC-MS (Method 1): Rₜ = 0.86 min; m/z = 531/533 (M + H)⁺. |
| 100 | 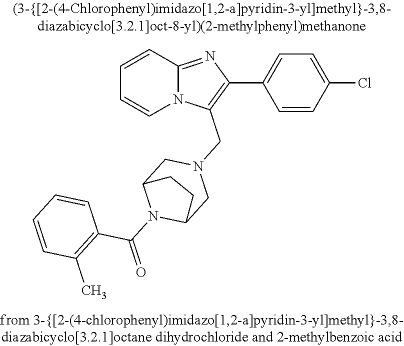 [SMILES: Cc1ccccc1C(=O)N1C2CCC1CN(Cc1c(-c3ccc(Cl)cc3)nc3ccccn13)C2] | ¹H-NMR (400 MHz, DMSO-d₆, δ/ppm): 1.62-1.80 (m, 4H), 2.20-2.29 (m, 4H), 2.24 (s, 3H), 2.43 (br. d, 1H), 2.53 (br. d, 1H), 2.66 (br. d, 1H), 3.52 (br. s, 1H), 4.02 (s, 2H), 4.60 (br. s, 1H), 6.99 (t, 1H), 7.19-7.33 (m, 5H), 7.54 (d, 2H), 7.60 (d, 1H), 7.92 (d, 2H), 8.60 (d, 1H). LC-MS (Method 1): Rₜ = 0.83 min; m/z = 471/473 (M + H)⁺. |
| 101 | 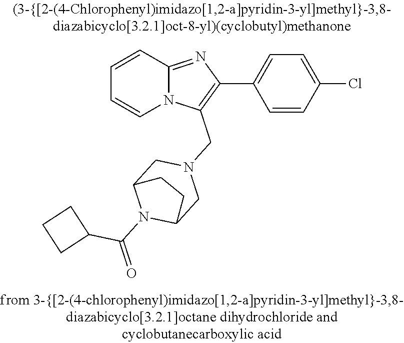 [SMILES: O=C(C1CCC1)N1C2CCC1CN(Cc1c(-c3ccc(Cl)cc3)nc3ccccn13)C2] | ¹H-NMR (400 MHz, DMSO-d₆, δ/ppm): 1.51-1.79 (m, 5H), 1.81-1.96 (m, 1H), 1.97-2.13 (m, 3H), 2.17-2.30 (m, 3H), 2.48-2.63 (m, 2H, partly concealed by DMSO signal), 3.21-3.34 (m, 1H, partly concealed by H₂O signal), 3.92-4.03 (m, 2H), 4.06 (br. s, 1H), 4.38 (br. d, 1H), 6.98 (td, 1H), 7.31 (ddd, 1H), 7.53 (d, 2H), 7.60 (d, 1H), 7.92 (d, 2H), 8.59 (d, 1H). LC-MS (Method 2): Rₜ = 1.46 min; m/z = 435/437 (M + H)⁺. |
| 102 | 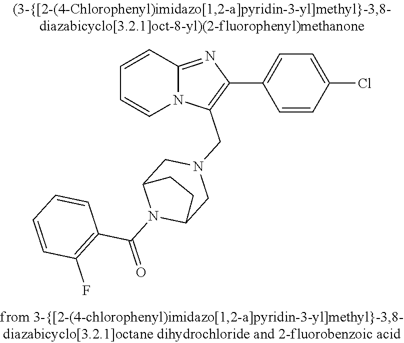 [SMILES: O=C(c1ccccc1F)N1C2CCC1CN(Cc1c(-c3ccc(Cl)cc3)nc3ccccn13)C2] | ¹H-NMR (400 MHz, DMSO-d₆, δ/ppm): 1.63-1.79 (m, 4H), 2.26 (br. d, 1H), 2.43 (br. d, 1H), 2.46-2.59 (m, 1H, partly concealed by DMSO signal), 2.66 (br. d, 1H), 3.66 (br. s, 1H), 4.03 (s, 2H), 4.60 (br. s, 1H), 6.99 (td, 1H), 7.23-7.35 (m, 3H), 7.40-7.51 (m, 2H), 7.54 (d, 2H), 7.60 (d, 1H), 7.92 (d, 2H), 8.60 (d, 1H). LC-MS (Method 2): Rₜ = 1.50 min; m/z = 475/477 (M + H)⁺. |
| 103 | 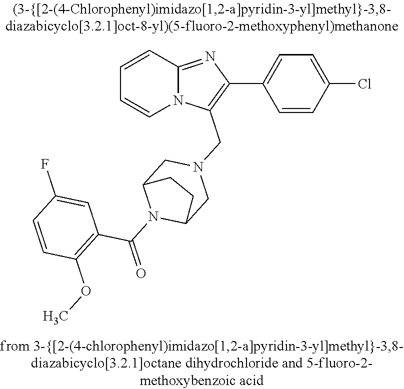 [SMILES: COc1ccc(F)cc1C(=O)N1C2CCC1CN(Cc1c(-c3ccc(Cl)cc3)nc3ccccn13)C2] | ¹H-NMR (400 MHz, DMSO-d₆, δ/ppm): 1.61-1.77 (m, 4H), 2.26 (br. d, 1H), 2.40 (br. d, 1H), 2.46-2.54 (m, 1H, partly concealed by DMSO signal), 2.63 (br. d, 1H), 3.54 (br. s, 1H), 3.75 (s, 3H), 4.01 (s, 2H), 4.55 (br. s, 1H), 6.99 (td, 1H), 7.05-7.16 (m, 2H), 7.22 (td, 1H), 7.31 (ddd, 1H), 7.54 (d, 2H), 7.60 (d, 1H), 7.92 (d, 2H), 8.59 (d, 1H). LC-MS (Method 1): Rₜ = 0.81 min; m/z = 505/507 (M + H)⁺. |
| 104 | 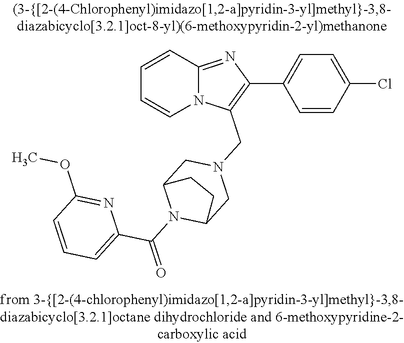 [SMILES: COc1cccc(C(=O)N2C3CCC2CN(Cc2c(-c4ccc(Cl)cc4)nc4ccccn24)C3)n1] | ¹H-NMR (400 MHz, DMSO-d₆, δ/ppm): 1.62-1.82 (m, 4H), 2.45 (br. d, 1H), 2.57-2.64 (m, 2H), 2.73 (dd, 1H), 3.78 (s, 3H), 4.04 (s, 2H), 4.67 (br. d, 2H), 6.93 (d, 1H), 6.99 (td, 1H), 7.32 (ddd, 1H), 7.35 (d, 1H), 7.54 (d, 2H), 7.60 (d, 1H), 7.82 (dd, 1H), 7.93 (d, 2H), 8.62 (d, 1H). LC-MS (Method 2): Rₜ = 1.52 min; m/z = 488/490 (M + H)⁺. |
| 105 | 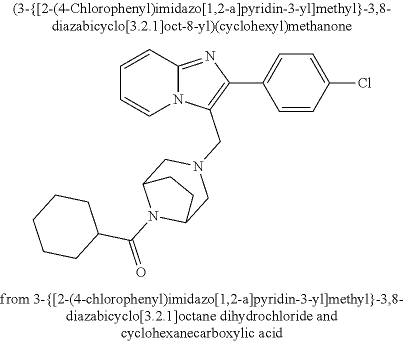 [SMILES: O=C(C1CCCCC1)N1C2CCC1CN(Cc1c(-c3ccc(Cl)cc3)nc3ccccn13)C2] | ¹H-NMR (400 MHz, DMSO-d₆, δ/ppm): 1.02-1.46 (m, 5H), 1.50-1.79 (m, 9H), 2.25 (br. dd, 2H), 2.38-2.48 (m, 1H), 2.48-2.65 (m, 2H, partly concealed by DMSO signal), 3.93-4.06 (m, 2H), 4.26 (br. s, 1H), 4.41 (br. d, 1H), 6.99 (td, 1H), 7.32 (ddd, 1H), 7.53 (d, 2H), 7.60 (d, 1H), 7.93 (d, 2H), 8.61 (d, 1H). LC-MS (Method 2): Rₜ = 1.65 min; m/z = 463/465 (M + H)⁺. |
| 106 | 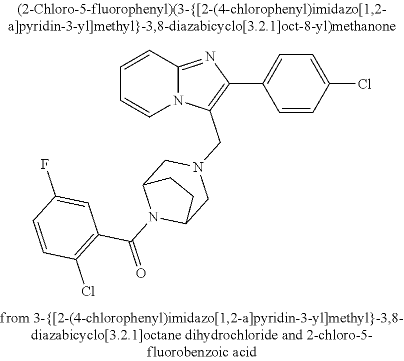 [SMILES: O=C(c1cc(F)ccc1Cl)N1C2CCC1CN(Cc1c(-c3ccc(Cl)cc3)nc3ccccn13)C2] | ¹H-NMR (400 MHz, DMSO-d₆, δ/ppm): 1.64-1.86 (m, 4H), 2.34 (br. d, 1H), 2.44 (br. d, 1H), 2.47-2.57 (m, 1H, concealed by DMSO signal), 2.64 (br. d, 1H), 3.55 (br. s, 1H), 4.03 (s, 2H), 4.58 (br. s, 1H), 7.00 (t, 1H), 7.27-7.35 (m, 2H), 7.36-7.49 (m, 1H), 7.51-7.63 (m, 4H), 7.91 (d, 2H), 8.59 (d, 1H). LC-MS (Method 2): Rₜ = 1.61 min; m/z = 509/510/511 (M + H)⁺. |
| 107 | 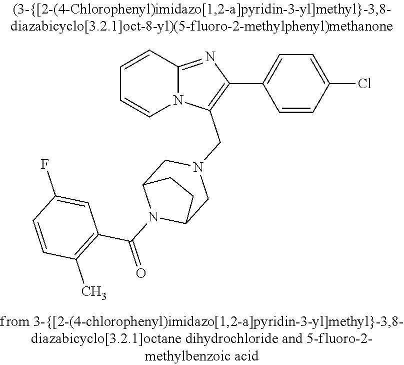 [SMILES: Cc1ccc(F)cc1C(=O)N1C2CCC1CN(Cc1c(-c3ccc(Cl)cc3)nc3ccccn13)C2] | ¹H-NMR (400 MHz, DMSO-d₆, δ/ppm): 1.64-1.81 (m, 4H), 2.20 (s, 3H), 2.29 (br. d, 1H), 2.44 (br. d, 1H), 2.54 (br. d, 1H), 2.63 (br. d, 1H), 3.54 (br. s, 1H), 4.03 (s, 2H), 4.58 (br. s, 1H), 7.00 (td, 1H), 7.09-7.18 (m, 2H), 7.27-7.34 (m, 2H), 7.54 (d, 2H), 7.60 (d, 1H), 7.93 (d, 2H), 8.60 (d, 1H). LC-MS (Method 1): Rₜ = 0.84 min; m/z = 489/491 (M + H)⁺. |
| 108 | 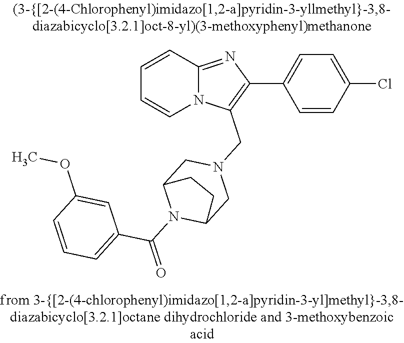 [SMILES: COc1cccc(C(=O)N2C3CCC2CN(Cc2c(-c4ccc(Cl)cc4)nc4ccccn24)C3)c1] | ¹H-NMR (400 MHz, DMSO-d₆, δ/ppm): 1.62-1.79 (m, 4H), 2.35-2.69 (m, 4H, partly concealed by DMSO signal), 3.78 (s, 3H), 3.94 (br. s, 1H), 4.04 (s, 2H), 4.56 (br. s, 1H), 6.94-7.07 (m, 4H), 7.26-7.38 (m, 2H), 7.54 (d, 2H), 7.60 (d, 1H), 7.94 (d, 2H), 8.62 (d, 1H). LC-MS (Method 2): Rₜ = 1.51 min; m/z = 487/489 (M + H)⁺. |
| 109 | 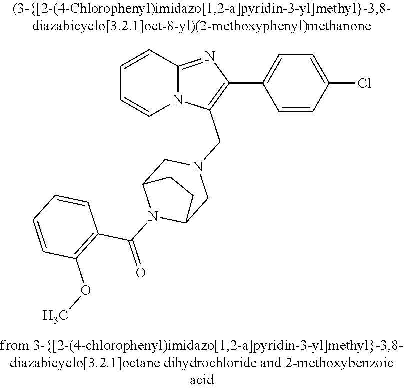 [SMILES: COc1ccccc1C(=O)N1C2CCC1CN(Cc1c(-c3ccc(Cl)cc3)nc3ccccn13)C2] | ¹H-NMR (400 MHz, DMSO-d₆, δ/ppm): 1.57-1.80 (m, 4H), 2.17-2.30 (m, 1H), 2.39 (br. d, 1H), 2.43-2.57 (m, 1H, partly concealed by DMSO signal), 2.65 (br. d, 1H), 3.52 (br. s, 1H), 3.76 (s, 3H), 4.01 (s, 2H), 4.56 (br. s, 1H), 6.93-7.02 (m, 2H), 7.06 (d, 1H), 7.17-7.26 (m, 1H), 7.27-7.41 (m, 2H), 7.54 (d, 2H), 7.60 (d, 1H), 7.91 (d, 2H), 8.59 (d, 1H). LC-MS (Method 2): Rₜ = 1.46 min; m/z = 487/489 (M + H)⁺. |
| 110 | 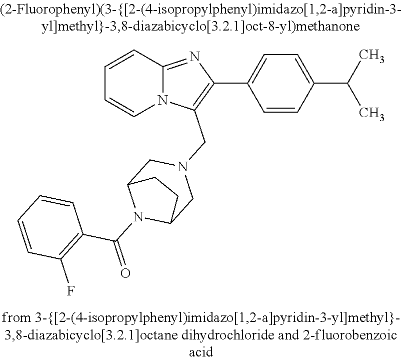 [SMILES: CC(C)c1ccc(-c2nc3ccccn3c2CN2CC3CCC(C2)N3C(=O)c2ccccc2F)cc1] | ¹H-NMR (400 MHz, DMSO-d₆, δ/ppm): 1.25 (d, 6H), 1.66-1.80 (m, 4H), 2.27 (br. d, 1H), 2.42 (br. d, 1H), 2.58 (br. d, 1H), (br. d, 1H), 2.88-3.01 (m, 1H), 3.68 (br. s, 1H), 4.02 (s, 2H), 4.60 (br. s, 1H), 6.97 (td, 1H), 7.24-7.32 (m, 3H), 7.35 (d, 2H), 7.42-7.54 (m, 2H), 7.58 (d, 1H), 7.80 (d, 2H), 8.58 (d, 1H). LC-MS (Method 2): Rₜ = 1.46 min; m/z = 483 (M + H)⁺. |
| 111 | 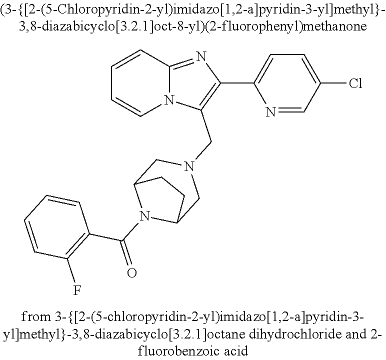 [SMILES: O=C(c1ccccc1F)N1C2CCC1CN(Cc1c(-c3ccc(Cl)cn3)nc3ccccn13)C2] | ¹H-NMR (400 MHz, DMSO-d₆, δ/ppm): 1.61-1.77 (m, 4H), 2.23 (br. d, 1H), 2.39 (br. d, 1H), 2.57 (br. d, 1H), 2.71 (dd, 1H), 3.63 (br. s, 1H), 4.50 (s, 2H), 4.57 (br. s, 1H), 7.01 (td, 1H), 7.24-7.31 (m, 2H), 7.34 (ddd, 1H), 7.42 (td, 1H), 7.45-7.52 (m, 1H), 7.60 (d, 1H), 7.99 (dd, 1H), 8.20 (d, 1H), 8.53 (d, 1H), 8.68 (d, 1H). LC-MS (Method 2): Rₜ = 1.47 min; m/z = 476/478 (M + H)⁺. |
| 112 | 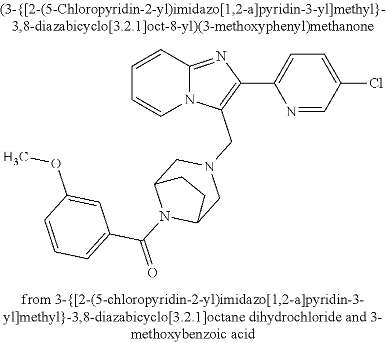 [SMILES: COc1cccc(C(=O)N2C3CCC2CN(Cc2c(-c4ccc(Cl)cn4)nc4ccccn24)C3)c1] | ¹H-NMR (400 MHz, DMSO-d₆, δ/ppm): 1.60-1.76 (m, 4H), 2.28-2.45 (m, 2H), 2.55-2.76 (m, 2H), 3.77 (s, 3H), 3.91 (br. s, 1H), 4.51 (br. s, 3H), 6.95 dd, 1H), 6.97-7.05 (m, 3H), 7.34 (t, 2H), 7.62 (d, 1H), 7.99 (dd, 1H), 8.20 (d, 1H), 8.55 (d, 1H), 8.68 (d, 1H). LC-MS (Method 2): Rₜ = 1.45 min; m/z = 488/490 (M + H)⁺. |
| 113 | 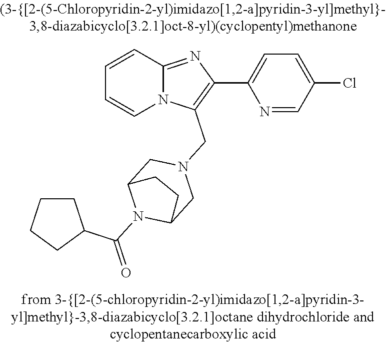 [SMILES: O=C(C1CCCC1)N1C2CCC1CN(Cc1c(-c3ccc(Cl)cn3)nc3ccccn13)C2] | ¹H-NMR (400 MHz, DMSO-d₆, δ/ppm): 1.43-1.78 (m, 12H), 2.16-2.28 (m, 2H), 2.55-2.67 (m, 2H), 2.83 (quin, 1H), 4.25 (br. s, 1H), 4.38 (br. d, 1H), 4.42-4.52 (m, 2H), 7.02 (td, 1H), 7.35 (ddd, 1H), 7.60 (d, 1H), 7.99 (dd, 1H), 8.20 (d, 1H), 8.56 (d, 1H), 8.66 (d, 1H). LC-MS (Method 2): Rₜ = 1.39 min; m/z = 450/452 (M + H)⁺. |
| 114 | 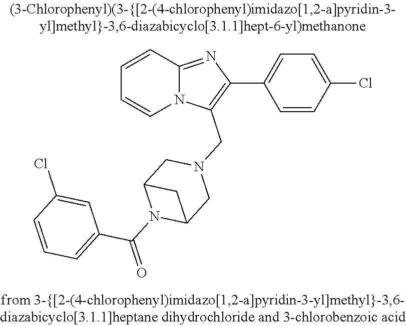 [SMILES: O=C(c1cccc(Cl)c1)N1C2CC1CN(Cc1c(-c3ccc(Cl)cc3)nc3ccccn13)C2] | ¹H-NMR (400 MHz, DMSO-d₆, δ/ppm): 1.70 (d, 1H), 2.47-2.60 (m, 1H, partly concealed by DMSO signal), 2.73 (br. d, 1H), 2.79 (br. d, 1H), 3.25 (br. d, 1H), 3.28-3.34 (m, 1H, partly concealed by H₂O signal), 4.20 (s, 2H), 4.32 (br. s, 1H), 4.47 (br. s, 1H), 6.97 (t, 1H), 7.31 (t, 1H), 7.41 (d, 1H), 7.44-7.55 (m, 5H), 7.59 (d, 1H), 7.85 (d, 2H), 8.44 (d, 1H). LC-MS (Method 1): Rₜ = 0.82 min; m/z = 477/479 (M + H)⁺. |
| 115 | 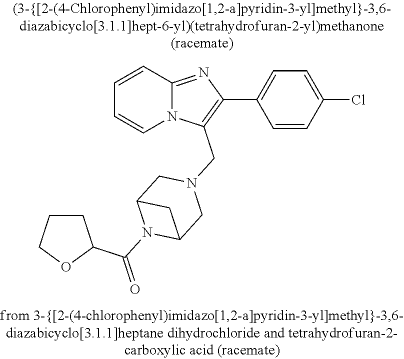 [SMILES: O=C(C1CCCO1)N1C2CC1CN(Cc1c(-c3ccc(Cl)cc3)nc3ccccn13)C2] | ¹H-NMR (400 MHz, DMSO-d₆, δ/ppm): 1.65-2.05 (m, 5H), 2.29-2.42 (m, 1H), 2.70-2.93 (m, 3.5H), 3.01 (br. d, 0.5H), 3.61-3.75 (m, 2H), 4.13-4.22 (m, 3.5H), 4.26 (dd, 0.5H), 4.41-4.50 (m, 1H), 6.96 (td, 1H), 7.31 (t, 1H), 7.49-7.56 (m, 2H), 7.60 (d, 1H), 7.82-7.92 (m, 2H), 8.51 (d, 1H). LC-MS (Method 1): Rₜ = 0.64 min; m/z = 437/439 (M + H)⁺. |
| 116 | 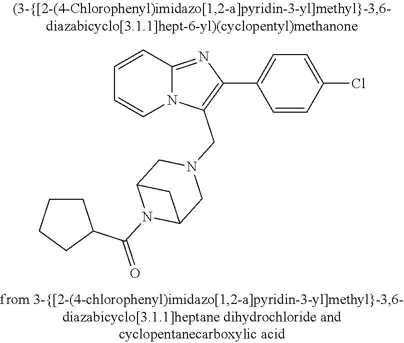 [SMILES: O=C(C1CCCC1)N1C2CC1CN(Cc1c(-c3ccc(Cl)cc3)nc3ccccn13)C2] | ¹H-NMR (400 MHz, DMSO-d₆, δ/ppm): 1.34-1.62 (m, 7H), 1.65-1.77 (m, 2H), 2.27-2.36 (m, 1H), 2.41-2.52 (m, 1H, partly concealed by DMSO signal), 2.70-2.82 (m, 3H), 2.97 (br. d, 1H), 4.08-4.24 (m, 3H), 4.29-4.37 (m, 1H), 6.96 (td, 1H), 7.31 (t, 1H), 7.52 (d, 2H), 7.60 (d, 1H), 7.86 (d, 2H), 8.48 (d, 1H). LC-MS (Method 1): Rₜ = 0.76 min; m/z = 435/437 (M + H)⁺. |
| 117 | 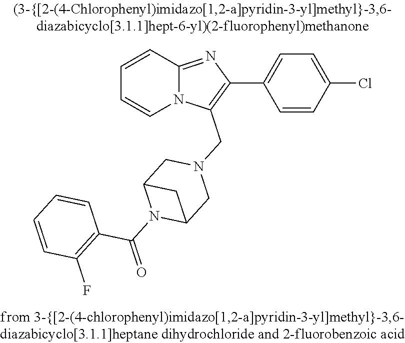 [SMILES: O=C(c1ccccc1F)N1C2CC1CN(Cc1c(-c3ccc(Cl)cc3)nc3ccccn13)C2] | ¹H-NMR (400 MHz, DMSO-d₆, δ/ppm): 1.82 (d, 1H), 2.39-2.48 (m, 2H), 2.70 (dd, 1H), 2.89 (dd, 1H), 3.14 (d, 1H), 4.07 (br. s, 1H), 4.15-4.27 (m, 2H), 4.37 (br. s, 1H), 6.98 (t, 1H), 7.15-7.27 (m, 2H), 7.27-7.39 (m, 2H), 7.43-7.56 (m, 3H), 7.60 (d, 1H), 7.85 (d, 2H), 8.49 (d, 1H). LC-MS (Method 1): Rₜ = 0.75 min; m/z = 461/463 (M + H)⁺. |
| 118 | 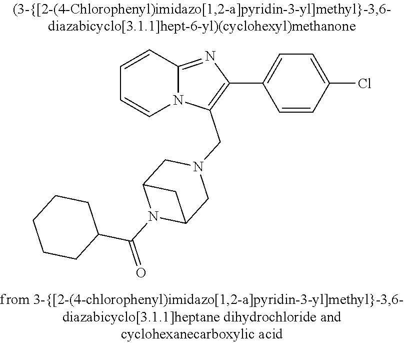 [SMILES: O=C(C1CCCCC1)N1C2CC1CN(Cc1c(-c3ccc(Cl)cc3)nc3ccccn13)C2] | ¹H-NMR (400 MHz, DMSO-d₆, δ/ppm): 1.01-1.40 (m, 6H), 1.52-1.67 (m, 4H), 1.71 (d, 1H), 1.95-2.06 (m, 1H), 2.28 (q, 1H), 2.65 (br. d, 1H), 2.74-2.83 (m, 2H), 2.91 (br. d, 1H), 4.09 (br. s, 1H), 4.12-4.25 (m, 2H), 4.33 (br. s, 1H), 6.96 (td, 1H), 7.31 (t, 1H), 7.52 (d, 2H), 7.60 (d, 1H), 7.85 (d, 2H), 8.48 (d, 1H). LC-MS (Method 1): Rₜ = 0.80 min; m/z = 449/451 (M + H)⁺. |
| 119 | 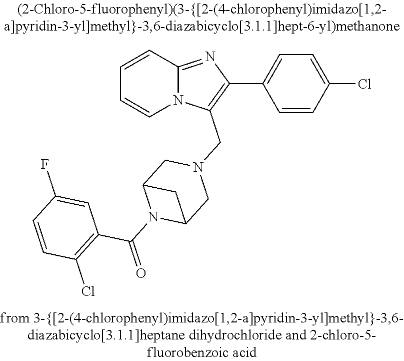 [SMILES: O=C(c1cc(F)ccc1Cl)N1C2CC1CN(Cc1c(-c3ccc(Cl)cc3)nc3ccccn13)C2] | ¹H-NMR (400 MHz, DMSO-d₆, δ/ppm): 1.92 (d, 1H), 2.40-2.52 (m, 2H, partly concealed by DMSO signal), 2.74 (dd, 1H), 2.95 (dd, 1H), 3.09 (d, 1H), 3.99 (br. s, 1H), 4.25 (s, 2H), 4.41 (br. s, 1H), 6.97 (t, 1H), 7.26-7.36 (m, 3H), 7.49-7.58 (m, 3H), 7.60 (d, 1H), 7.88 (d, 2H), 8.52 (d, 1H). LC-MS (Method 1): Rₜ = 0.79 min; m/z = 495/497 (M + H)⁺. |
| 120 | 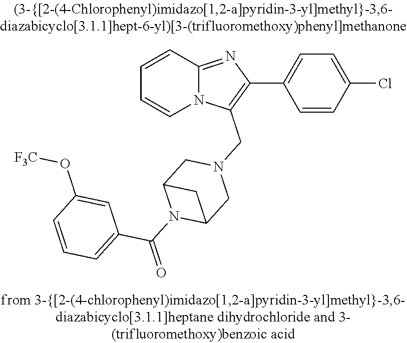 [SMILES: O=C(c1cccc(OC(F)(F)F)c1)N1C2CC1CN(Cc1c(-c3ccc(Cl)cc3)nc3ccccn13)C2] | ¹H-NMR (400 MHz, DMSO-d₆, δ/ppm): 1.73 (d, 1H), 2.47-2.60 (m, 2H, partly concealed by DMSO signal), 2.74 (br. d, 1H), 2.82 (br. d, 1H), 3.26 (br. d, 1H), 4.14-4.25 (m, 2H), 4.35 (br. s, 1H), 4.47 (br. s, 1H), 6.95 (t, 1H), 7.30 (t, 1H), 7.41-7.61 (m, 7H), 7.85 (d, 2H), 8.46 (d, 1H). LC-MS (Method 1): Rₜ = 0.90 min; m/z = 527/529 (M + H)⁺. |
| 121 | 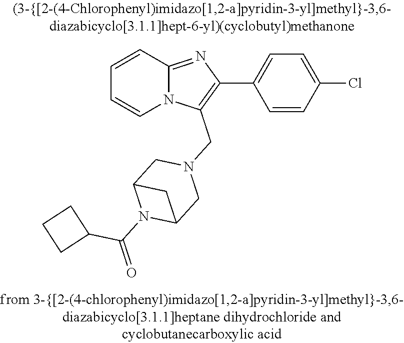 [SMILES: O=C(C1CCC1)N1C2CC1CN(Cc1c(-c3ccc(Cl)cc3)nc3ccccn13)C2] | ¹H-NMR (400 MHz, DMSO-d₆, δ/ppm): 1.62-1.89 (m, 4H), 1.91-2.13 (m, 3H), 2.31 (q, 1H), 2.66 (dd, 1H), 2.70-2.81 (m, 2H), 2.87-3.02 (m, 2H), 4.08-4.24 (m, 4H), 6.95 (t, 1H), 7.30 (t, 1H), 7.52 (d, 2H), 7.60 (d, 1H), 7.86 (d, 2H), 8.46 (d, 1H). LC-MS (Method 1): Rₜ = 0.71 min; m/z = 421/423 (M + H)⁺. |
| 122 | 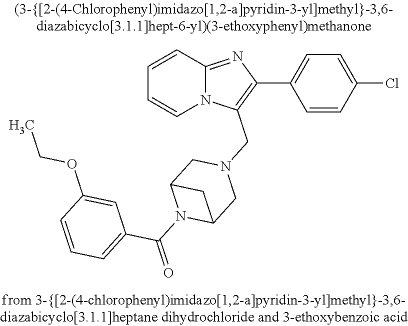 [SMILES: CCOc1cccc(C(=O)N2C3CC2CN(Cc2c(-c4ccc(Cl)cc4)nc4ccccn24)C3)c1] | ¹H-NMR (400 MHz, DMSO-d₆, δ/ppm): 1.30 (t, 3H), 1.72 (d, 1H), 2.47-2.61 (m, 2H, partly concealed by DMSO signal), 2.73 (br. d, 1H), 2.81 (br. d, 1H), 3.27 (br. d, 1H), 3.83-3.99 (dq, 2H), 4.11-4.23 (m, 2H), 4.31 (br. s, 1H), 4.43 (br. s, 1H), 6.91-7.02 (m, 3H), 7.07 (d, 1H), 7.23-7.33 (m, 2H), 7.50 (d, 2H), 7.58 (d, 1H), 7.87 (d, 2H), 8.49 (d, 1H). LC-MS (Method 2): Rₜ = 1.56 min; m/z = 487/489 (M + H)⁺. |
| 123 | 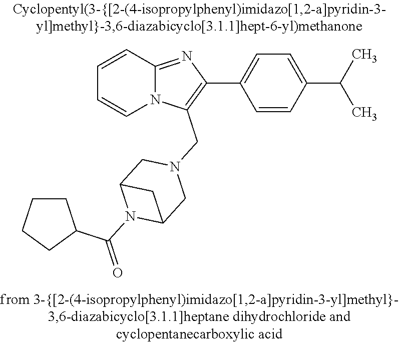 [SMILES: CC(C)c1ccc(-c2nc3ccccn3c2CN2CC3CC(C2)N3C(=O)C2CCCC2)cc1] | ¹H-NMR (400 MHz, DMSO-d₆, δ/ppm): 1.25 (d, 6H), 1.35-1.63 (m, 7H), 1.66-1.79 (m, 2H), 2.31 (q, 1H), 2.42-2.57 (m, 1H, partly concealed by DMSO signal), 2.70-2.83 (m, 3H), 2.87-3.01 (m, 2H), 4.07-4.24 (m, 3H), 4.34 (br. s, 1H), 6.93 (t, 1H), 7.28 (t, 1H), 7.33 (d, 2H), 7.58 (d, 1H), 7.75 (d, 2H), 8.44 (d, 1H). LC-MS (Method 2): Rₜ = 1.48 min; m/z = 443 (M + H)⁺. |

### Example 124 and Example 125

### (5-{[2-(6-Isopropylpyridin-3-yl)imidazo[1,2-a]pyridin-3-yl]methyl}-2,5-diazabicyclo[2.2.2]oct-2-yl)(6-methoxypyridin-2-yl)methanone (Enantiomer 1 and 2)

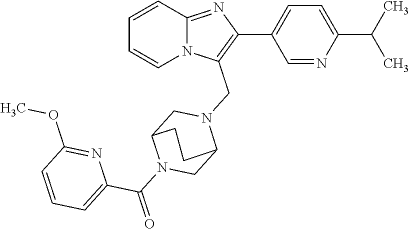 [SMILES: COc1cccc(C(=O)N2CC3CCC2CN3Cc2c(-c3ccc(C(C)C)nc3)nc3ccccn23)n1]

139 mg (0.28 mmol) of racemic (5-{[2-(6-isopropylpyridin-3-yl)imidazo[1,2-a]pyridin-3-yl]methyl}-2,5-diazabicyclo[2.2.2]oct-2-yl)(6-methoxypyridin-2-yl)methanone (Example 27) were separated into the enantiomers by preparative HPLC on a chiral phase [column: YMC Cellulose SC, 5 pin, 250 mm×20 mm; eluent: n-heptane/isopropanol 25:75+0.2% diethylamine; flow rate: 15 m/min; UV detection: 220 nm; temperature: 55° C.]:

### Example 124 (Enantiomer 1)

Yield: 65 mg

Rₜ=14.77 min; chemical purity >99%; >99% ee

[Column: Daicel Chiralpak IC, 5 μm, 250 mm×4.6 mm; eluent: isohexane/isopropanol 25:75+0.2% diethylamine; flow rate: 1 ml/min; temperature: 55° C.; UV detection: 235 nm].

¹H-NMR (400 MHz, DMSO-d₆, δ/ppm): 1.28 (d, 6H), 1.47-2.00 (m, 4H), 2.74 (br. d, 1H), 2.82-2.95 (m, 2H), 3.07 (dt, 1H), 3.39 (br. d, 0.7H), 3.50 (br. d, 0.3H), 3.71-3.81 (m, 3H), 3.84 (s, 0.7H), 3.99 (br. s, 1H), 4.22-4.34 (m, 2H), 4.39 (br. s, 0.3H), 6.84-7.03 (m, 2H), 7.17 (d, 0.7H), 7.27-7.41 (m, 2.3H), 7.62 (d, 1H), 7.73-7.85 (m, 1H), 8.09-8.18 (m, 1H), 8.55-8.65 (m, 1H), 8.88-8.98 (m, 1H).

LC-MS (Method 2): Rₜ=1.19 min; m/z=497 (M+H)⁺.

### Example 125 (Enantiomer 2)

Yield: 66 mg

Rₜ=19.54 min; chemical purity >99%; >99% ee

[Column: Daicel Chiralpak IC, 5 μm, 250 mm×4.6 mm; eluent: isohexane/isopropanol 25:75+0.2% diethylamine; flow rate: 1 ml/min; temperature: 55° C.; UV detection: 235 nm].

¹H-NMR (400 MHz, DMSO-d₆, δ/ppm): 1.28 (d, 6H), 1.48-2.00 (m, 4H), 2.74 (br. d, 1H), 2.82-2.96 (m, 2H), 3.08 (dt, 1H), 3.39 (br. d, 0.7H), 3.50 (br. d, 0.3H), 3.71-3.81 (m, 3H), 3.84 (s, 0.7H), 4.00 (br. s, 1H), 4.22-4.34 (m, 2H), 4.39 (br. s, 0.3H), 6.85-7.03 (m, 2H), 7.17 (d, 0.7H), 7.27-7.42 (m, 2.3H), 7.62 (d, 1H), 7.74-7.85 (m, 1H), 8.08-8.18 (m, 1H), 8.56-8.65 (m, 1H), 8.88-8.98 (m, 1H).

LC-MS (Method 2): Rₜ=1.19 min; m/z=497 (M+H)⁺.

### Example 126 and Example 127

### (3-Fluoro-6-methoxypyridin-2-yl)(5-{[2-(6-isopropylpyridin-3-yl)imidazo[1,2-a]pyridin-3-yl]methyl}-2,5-diazabicyclo[2.2.2]oct-2-yl)methanone (Enantiomer 1 and 2)

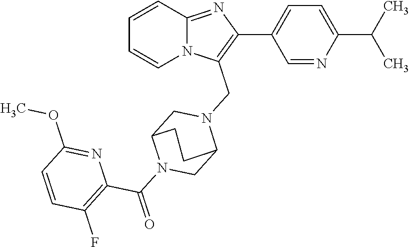 [SMILES: COc1ccc(F)c(C(=O)N2CC3CCC2CN3Cc2c(-c3ccc(C(C)C)nc3)nc3ccccn23)n1]

134 mg (0.26 mmol) of racemic (3-fluoro-6-methoxypyridin-2-yl)(5-{[2-(6-isopropylpyridin-3-yl)imidazo[1,2-a]pyridin-3-yl]methyl}-2,5-diazabicyclo[2.2.2]oct-2-yl)methanone (Example 28) were separated into the enantiomers by preparative HPLC on a chiral phase [column: YMC Cellulose SC, 5 μm, 250 mm×20 mm; eluent: n-heptane/isopropanol 25:75+0.2% diethylamine; flow rate: 15 ml/min; UV detection: 220 nm; temperature: 55° C.]:

### Example 126 (Enantiomer 1)

Yield: 60 mg

Rₜ=15.10 min; chemical purity >99%; >99% ee

[Column: Daicel Chiralpak IC, 5 μm, 250 mm×4.6 mm; eluent: isohexane/isopropanol 25:75+0.2% diethylamine; flow rate: 1 ml/min; temperature: 55° C.; UV detection: 235 nm].

¹H-NMR (400 MHz, DMSO-d₆, δ/ppm): 1.22-1.31 (m, 6H), 1.50-1.99 (m, 4H), 2.65-2.77 (m, 1H), 2.77-2.87 (m, 1.3H), 2.94 (br. s, 0.7H), 3.01-3.18 (m, 1.3H), 3.39-3.51 (m, 1.4H), 3.60 (br. d, 0.3H), 3.68-3.85 (m, 3.7H), 4.20-4.35 (m, 2H), 4.40 (br. s, 0.3H), 6.87-7.03 (m, 2H), 7.27-7.42 (m, 2H), 7.62 (d, 1H), 7.70-7.83 (m, 1H), 8.07-8.18 (m, 1H), 8.55-8.64 (m, 1H), 8.86-8.98 (m, 1H).

LC-MS (Method 2): Rₜ=1.22 min; m/z=515 (M+H)⁺.

### Example 127 (Enantiomer 2)

Yield: 57 mg

Rₜ=20.80 min; chemical purity >99%; >99% ee

[Column: Daicel Chiralpak IC, 5 μm, 250 mm×4.6 mm; eluent: isohexane/isopropanol 25:75+0.2% diethylamine; flow rate: 1 ml/min; temperature: 55° C.; UV detection: 235 nm].

¹H-NMR (400 MHz, DMSO-d₆, δ/ppm): 1.21-1.33 (m, 6H), 1.50-1.99 (m, 4H), 2.65-2.77 (m, 1H), 2.77-2.88 (m, 1.3H), 2.94 (br. s, 0.7H), 3.01-3.18 (m, 1.3H), 3.39-3.51 (m, 1.4H), 3.60 (br. d, 0.3H), 3.69-3.86 (m, 3.7H), 4.19-4.34 (m, 2H), 4.39 (br. s, 0.3H), 6.88-7.04 (m, 2H), 7.27-7.41 (m, 2H), 7.62 (d, 1H), 7.70-7.83 (m, 1H), 8.07-8.18 (m, 1H), 8.56-8.64 (m, 1H), 8.87-8.98 (m, 1H).

LC-MS (Method 2): Rₜ=1.22 min; m/z=515 (M+H)⁺.

The enantiomerically pure compound from Example 126 (enantiomer 1) was also obtainable by an alternative method as follows:

80 mg (0.47 mmol) of 3-fluoro-6-methoxypyridine-2-carboxylic acid were dissolved in 2 ml of DMF, 242 mg (0.64 mmol) of 2-(7-aza-1H-benzotriazol-1-yl)-1,1,3,3-tetramethyluronium hexafluorophosphate (HATU) were added and the mixture was stirred at room temperature for 30 min. 200 mg (0.43 mmol) of 2-{[2-(6-isopropylpyridin-3-yl)imidazo[1,2-a]pyridin-3-yl]methyl}-2,5-diazabicyclo[2.2.2]octane dihydrochloride (enantiomer 1; Example 32A) and 370 μl (2.12 mmol) of N,N-diisopropylethylamine were then added, and the mixture was stirred at room temperature overnight. Thereafter, the reaction mixture was separated directly into its components via preparative HPLC (Method 6). 126 mg (0.24 mmol, 57% of theory) of the title compound were obtained.

[α]_{D}²⁰=−92.16° (c=0.285, methanol).

LC-MS (Method 2): Rₜ=1.27 min; m/z=515 (M+H)⁺.

### Example 128 and Example 129

### (2-Fluorophenyl)(5-{[2-(6-isopropylpyridin-3-yl)imidazo[1,2-a]pyridin-3-yl]methyl}-2,5-diazabicyclo[2.2.2]oct-2-yl)methanone (Enantiomer 1 and 2)

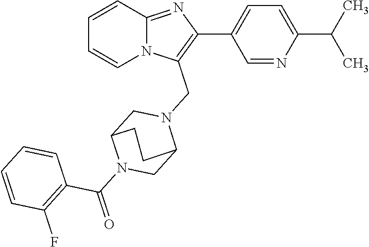 [SMILES: CC(C)c1ccc(-c2nc3ccccn3c2CN2CC3CCC2CN3C(=O)c2ccccc2F)cn1]

121 mg (0.25 mmol) of racemic (2-fluorophenyl)(5-{[2-(6-isopropylpyridin-3-yl)imidazo[1,2-a]pyridin-3-yl]methyl}-2,5-diazabicyclo[2.2.2]oct-2-yl)methanone (Example 68) were separated into the enantiomers by preparative HPLC on a chiral phase [column: YMC Cellulose SC, 5 μm, 250 mm×20 mm; eluent: n-heptane/isopropanol 25:75+0.2% diethylamine; flow rate: 15 ml/min; UV detection: 220 nm; temperature: 55° C.]:

### Example 128 (Enantiomer 1)

Yield: 57 mg

Rₜ=14.26 min; chemical purity >99%; >99% ee

[Column: Daicel Chiralpak IC, 5 μm, 250 mm×4.6 mm; eluent: isohexane/isopropanol 25:75+0.2% diethylamine; flow rate: 1 ml/min; temperature: 55° C.; UV detection: 235 nm].

¹H-NMR (400 MHz, DMSO-d₆, δ/ppm): 1.24-1.32 (m, 6H), 1.48-1.98 (m, 4H), 2.65-2.89 (m, 2.3H), 2.94 (br. s, 0.7H), 3.00-3.16 (m, 1.3H), 3.28-3.36 (m, 0.7H, partly concealed by water signal), 3.39-3.50 (m, 1H), 3.78 (br. d, 0.7H), 4.20-4.33 (m, 2H), 4.39 (br. s, 0.3H), 6.94-7.03 (m, 1H), 7.19-7.53 (m, 6H), 7.58-7.65 (m, 1H), 8.08-8.18 (m, 1H), 8.55-8.64 (m, 1H), 8.90 (d, 0.3H), 8.94 (d, 0.7H).

LC-MS (Method 1): Rₜ=0.68 min; m/z=484 (M+H)⁺.

### Example 129 (Enantiomer 2)

Yield: 60 mg

Rₜ=23.23 min; chemical purity >99%; >99% ee

[Column: Daicel Chiralpak IC, 5 μm, 250 mm×4.6 mm; eluent: isohexane/isopropanol 25:75+0.2% diethylamine; flow rate: 1 ml/min; temperature: 55° C.; UV detection: 235 nm].

¹H-NMR (400 MHz, DMSO-d₆, δ/ppm): 1.22-1.32 (m, 6H), 1.52-1.97 (m, 4H), 2.65-2.89 (m, 2.3H), 2.94 (br. s, 0.7H), 3.01-3.14 (m, 1.3H), 3.27-3.36 (m, 0.7H, partly concealed by water signal), 3.39-3.49 (m, 1H), 3.78 (br. d, 0.7H), 4.21-4.33 (m, 2H), 4.39 (br. s, 0.3H), 6.93-7.04 (m, 1H), 7.18-7.53 (m, 6H), 7.58-7.65 (m, 1H), 8.08-8.18 (m, 1H), 8.55-8.64 (m, 1H), 8.90 (d, 0.3H), 8.94 (d, 0.7H).

LC-MS (Method 1): Rₜ=0.67 min; m/z=484 (M+H)⁺.

Analogously to Examples 1-4, the following compounds were also prepared from the reactants specified in each case:

| Example | Name/Structure/Starting materials | Analytical data |
|---|---|---|
| 130 | 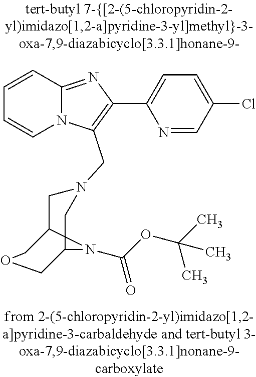 [SMILES: CN(Cc1c(-c2ccc(Cl)cn2)nc2ccccn12)CC1COCCN1C(=O)OC(C)(C)C] | ¹H-NMR (400 MHz, DMSO- d₆): δ [ppm] = 1.39 (s, 9H), 2.38 (br. s, 2H), 2.85 (br. d, 2H), 3.49-3.59 (m, 2H), 3.60- 3.70 (m, 2H), 3.83 (br. d, 2H), 4.42 (s, 2H), 6.93 (td, 1H), 7.33 (ddd, 1H), 7.61 (d, 1H), 8.00 (dd, 1H), 8.21 (d, 1H), 8.67 (dd, 1H), 8.83 (d, 1H). LC-MS (Method 2): Rₜ = 1.22 min; MS (ESIpos): m/z = 470 [M + H]⁺. |
| 131 | 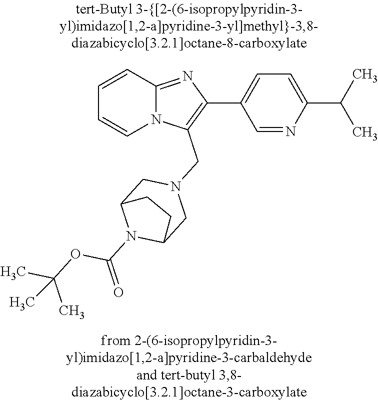 [SMILES: CC(C)c1ccc(-c2nc3ccccn3c2CN2CC3CCC(C2)N3C(=O)OC(C)(C)C)cn1] | LC-MS (Method 2): Rₜ = 1.68 min; MS (ESIpos): m/z = 462 [M + H]⁺. |
| 132 | 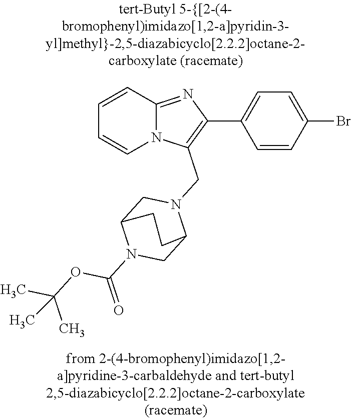 [SMILES: CC(C)(C)OC(=O)N1CC2CCC1CN2Cc1c(-c2ccc(Br)cc2)nc2ccccn12] | ¹H-NMR (400 MHz, DMSO- d₆): δ [ppm] = 1.13-1.25 (m, 1H), 1.33-1.40 (m, 9H), 1.50 (br. d, 1H), 1.66 (br. s, 2H), 1.85 (br. s, 1H), 2.52-2.55 (m, 4H), 2.62-2.77 (m, 3H), 3.13 (br. t, 1H), 3.46-3.54 (m, 1H), 4.17-4.25 (m, 2H), 6.97 (t, 1H), 7.31 (t, 1H), 7.58-7.69 (m, 3H), 7.74-7.83 (m, 2H), 8.57 (d, 1H). LC-MS (Method 5): Rₜ = 1.16 min; MS (ESIpos): m/z = 497 [M + H]⁺. |

### Example 133 and Example 134

### tert-Butyl 5-{[2-(6-isopropylpyridin-3-yl)imidazo[1,2-a]pyridin-3-yl]methyl}-2,5-diazabicyclo[2.2.2]octane-2-carboxylate (Enantiomer 1 and 2)

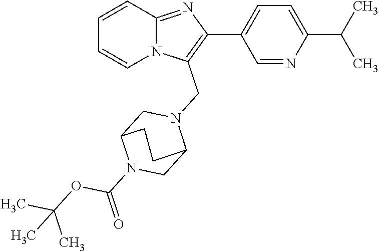 [SMILES: CC(C)c1ccc(-c2nc3ccccn3c2CN2CC3CCC2CN3C(=O)OC(C)(C)C)cn1]

4700 mg (10.38 mmol) of racemic tert-butyl 5-{[2-(6-isopropylpyridin-3-yl)imidazo[1,2-a]pyridin-3-yl]methyl}-2,5-diazabicyclo[2.2.2]octane-2-carboxylate (Example 3) were separated into the enantiomers by preparative HPLC on a chiral phase [column: Daicel Chiralpak IG, 5 μm, 250 mm×20 mm; eluent: isohexane/isopropanol 50:50+0.2% diethylamine; flow rate: 15 ml/min; UV detection: 220 nm; temperature: 50° C.]:

### Example 133 (Enantiomer 1)

Yield: 2310 mg

Rₜ=8.97 min; chemical purity >99%; >99% ee

[Column: Daicel Chiralpak IF, 5 μm, 250 mm×4.6 mm; eluent: isohexane/isopropanol 60:40+0.2% diethylamine; flow rate: 1 ml/min; temperature: 40° C.; UV detection: 235 nm].

¹H-NMR (400 MHz, DMSO-d₆, δ/ppm): 1.28 (d, 6H), 1.36 (2s, 9H), 1.43-1.56 (m, 1H), 1.57-1.74 (m, 2H), 1.79-1.92 (m, 1H), 2.65-2.82 (m, 3H), 3.01-3.19 (m, 2H), 3.53 (dd, 1H), 3.81 (br. d, 1H), 4.17-4.28 (m, 2H), 6.98 (t, 1H), 7.31 (t, 1H), 7.38 (d, 1H), 7.61 (d, 1H), 8.09-8.16 (m, 1H), 8.58 (d, 1H), 8.92 (dd, 1H).

LC-MS (Method 2): Rₜ=1.44 min; m/z=462 (M+H)⁺.

[α]_{D}²⁰=+8.89° (c=0.270, methanol).

### Example 134 (Enantiomer 2)

Yield: 2110 mg

Rₜ=7.28 min; chemical purity >99%; >99% ee

[Column: Daicel Chiralpak IF, 5 μm, 250 mm×4.6 mm; eluent: isohexane/isopropanol 60:40+0.2% diethylamine; flow rate: 1 ml/min; temperature: 40° C.; UV detection: 235 nm].

¹H-NMR (400 MHz, DMSO-d₆, δ/ppm): 1.28 (d, 6H), 1.36 (2s, 9H), 1.43-1.56 (m, 1H), 1.57-1.74 (m, 2H), 1.79-1.92 (m, 1H), 2.65-2.82 (m, 3H), 3.01-3.19 (m, 2H), 3.53 (dd, 1H), 3.81 (br. d, 1H), 4.17-4.28 (m, 2H), 6.98 (t, 1H), 7.31 (t, 1H), 7.38 (d, 1H), 7.61 (d, 1H), 8.09-8.16 (m, 1H), 8.58 (d, 1H), 8.92 (dd, 1H).

LC-MS (Method 2): Rₜ=1.44 min; m/z=462 (M+H)⁺.

[α]_{D}²⁰=−10.53° (c=0.285, methanol).

Analogously to Example 21 and 33, the following compounds also were prepared from the reactants specified in each case:

| Example | Name/Structure/Starting materials | Analytical data |
|---|---|---|
| 135 | 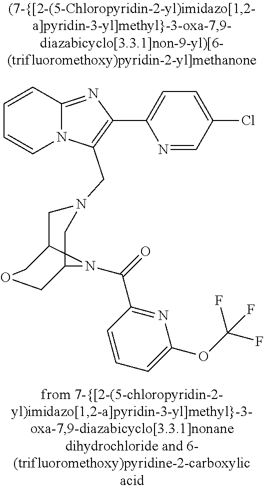 [SMILES: CN(Cc1c(-c2ccc(Cl)cn2)nc2ccccn12)CC1COCCN1C(=O)c1cccc(OC(F)(F)F)n1] | ¹H-NMR (400 MHz, DMSO- d₆): δ [ppm] = 2.87 (br. d, 1H), 3.07 (br. d, 1H), 3.59-3.73 (m, 3H), 3.84 (d, 1H), 3.95 (br. s, 1H), 4.39-4.53 (m, 3H), 6.94 (t, 1H), 7.33 (t, 1H), 7.39 (d, 1H), 7.61 (d, 1H), 7.71 (d, 1H), 7.96- 8.03 (m, 1H), 8.11-8.24 (m, 2H), 8.66 (d, 1H), 8.82 (d, 1H). LC-MS (Method 2): Rₜ = 1.36 min; MS (ESIpos): m/z = 559 [M + H]⁺. |
| 136 | 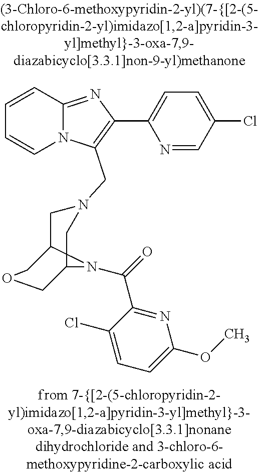 [SMILES: COc1ccc(Cl)c(C(=O)N2CCOCC2CN(C)Cc2c(-c3ccc(Cl)cn3)nc3ccccn23)n1] | ¹H-NMR (400 MHz, DMSO- d₆): δ [ppm] = 3.57 (br. t, 1H), 3.67 (br. t, 1H), 3.74-3.85 (m, 2H), 4.10 (br. d, 1H), 4.25 (br. d, 1H), 4.82 (br. s, 1H), 4.93- 5.10 (m, 2H), 7.02 (d, 1H), 7.20 (t, 1H), 7.48 (t, 1H), 7.78 (d, 1H), 7.96 (d, 1H), 8.19-8.31 (m, 2H), 8.72 (d, 1H), 8.90 (d, 1H). LC-MS (Method 2): Rₜ = 1.21 min; MS (ESIpos): m/z = 539 [M + H]⁺. |
| 137 | 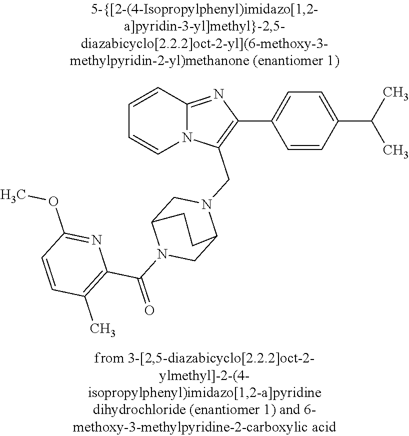 [SMILES: COc1ccc(C)c(C(=O)N2CC3CCC2CN3Cc2c(-c3ccc(C(C)C)cc3)nc3ccccn23)n1] | LC-MS (Method 2): Rₜ = 1.49 min; MS (ESIpos): m/z = 510 [M + H]⁺. |
| 138 | 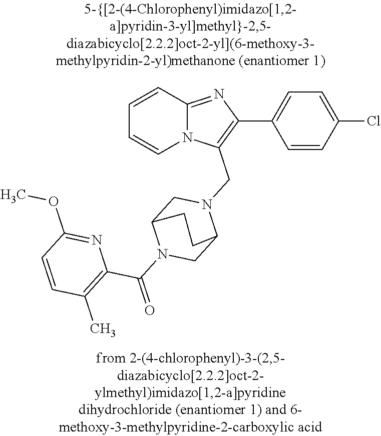 [SMILES: COc1ccc(C)c(C(=O)N2CC3CCC2CN3Cc2c(-c3ccc(Cl)cc3)nc3ccccn23)n1] | ¹H-NMR (400 MHz, DMSO- d₆): δ [ppm] = 1.50-1.97 (m, 4H), 2.02-2.11 (m, 3H), 2.59- 2.86 (m, 2.3H), 2.89-3.00 (m, 1H), 3.23 (br. s, 0.7H), 3.33- 3.45 (m, 1H), 3.66-3.82 (m, 3.75H), 4.16-4.31 (m, 2H), 4.39 (m, 0.25H), 6.71-6.80 (m, 1H), 6.92-7.02 (m, 1H), 7.26-7.35 (m, 1H), 7.45-7.65 (m, 4H), 7.79-7.91 (m, 2H), 8.53-8.62 (m, 1H). LC-MS (Method 2): Rₜ = 1.39 min; MS (ESIpos): m/z = 502/504 [M + H]⁺. |
| 139 | 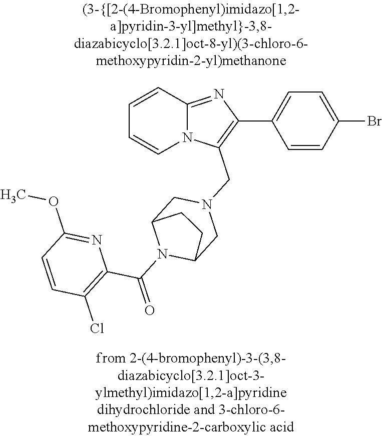 [SMILES: COc1ccc(Cl)c(C(=O)N2C3CCC2CN(Cc2c(-c4ccc(Br)cc4)nc4ccccn24)C3)n1] | ¹H-NMR (400 MHz, DMSO- d₆): δ [ppm] = 1.62-1.82 (m, 4H), 2.37-2.55 (m, 3H, partly concealed by DMSO signal), 2.73 (m, 1H), 3.63 (br. s, 1H), 3.79 (s, 3H), 4.04 (s, 2H), 4.60 (br. s, 1H), 6.92 (d, 1H), 6.99 (t, 1H), 7.31 (dd, 1H), 7.60 (d, 1H), 7.67 (d, 2H), 7.86 (t, 3H), 8.59 (d, 1H). LC-MS (Method 2): Rₜ = 1.58 min; MS (ESIpos): m/z = 566/568/569 [M + H]⁺. |
| 140 | 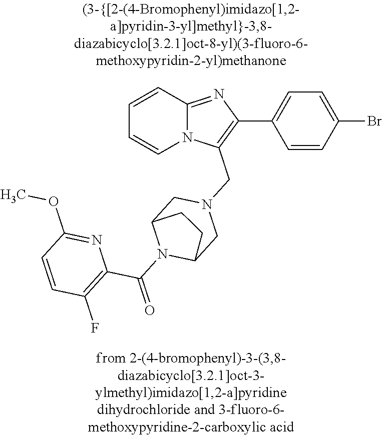 [SMILES: COc1ccc(F)c(C(=O)N2C3CCC2CN(Cc2c(-c4ccc(Br)cc4)nc4ccccn24)C3)n1] | ¹H-NMR (400 MHz, DMSO- d₆): δ [ppm] = 1.25 (br. d, 1H), 1.63-1.82 (m, 4H), 2.41-2.54 (m, 3H, partly concealed by DMSO signal), 2.70-2.79 (m, 1H), 3.76 (s, 3H), 3.92 (br. s, 1H), 3.99-4.10 (m, 2H), 4.61 (br. s, 1H), 6.93-7.01 (m, 2H), 7.31 (t, 1H), 7.60 (d, 1H), 7.65- 7.70 (m, 2H), 7.77 (t, 1H), 7.83-7.88 (m, 2H), 8.61 (d, 1H). LC-MS (Method 2): Rₜ = 1.50 min; MS (ESIpos): m/z = 550 [M + H]⁺. |
| 141 | 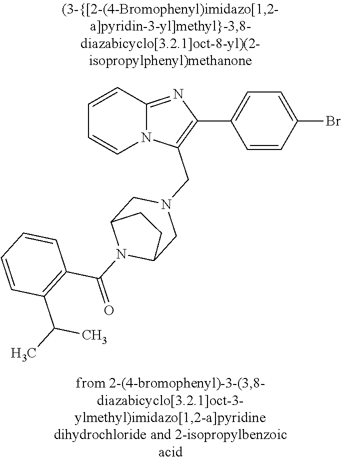 [SMILES: CC(C)c1ccccc1C(=O)N1C2CCC1CN(Cc1c(-c3ccc(Br)cc3)nc3ccccn13)C2] | ¹H-NMR (400 MHz, DMSO- d₆): δ [ppm] = 1.16 (br. d, 6H), 1.51-1.83 (m, 4H), 2.25-2.57 (m, 3H, partly concealed by DMSO signal), 2.67 (br. s, 1H), 2.78-3.15 (m, 1H), 3.50 (br. s, 1H), 4.02 (br. s, 2H), 4.62 (br. s, 1H), 6.99 (t, 1H), 7.22 (br. s, 1H), 7.25-7.42 (m, 4H), 7.57- 7.72 (m, 3H), 7.84 (br. s, 2H), 8.59 (br. d, 1H). LC-MS (Method 1): Rₜ = 0.92 min; MS (ESIpos): m/z = 545 [M + H]⁺. |
| 142 | 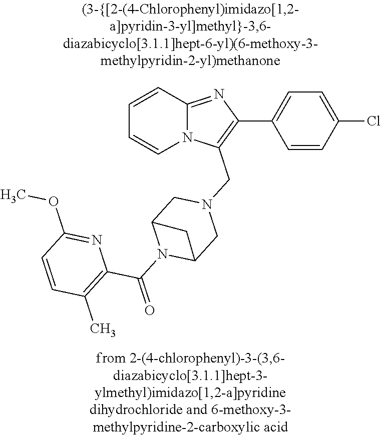 [SMILES: COc1ccc(C)c(C(=O)N2C3CC2CN(Cc2c(-c4ccc(Cl)cc4)nc4ccccn24)C3)n1] | ¹H-NMR (400 MHz, DMSO- d₆): δ [ppm] = 1.89 (d, 1H), 2.26 (s, 3H), 2.32-2.47 (m, 1H), 2.71-2.79 (m, 1H), 2.81-2.88 (m, 1H), 2.96-3.04 (m, 2H), 3.63 (s, 3H), 4.17-4.26 (m, 2H), 4.34-4.41 (m, 2H), 6.80 (d, 1H), 6.93 (t, 1H), 7.29 (t, 1H), 7.50 (d, 2H), 7.59 (d, 2H), 7.84 (d, 2H), 8.52 (d, 1H). LC-MS (Method 1): Rₜ = 0.77 min; MS (ESIpos): m/z = 488/490 [M + H]⁺. |
| 143 | 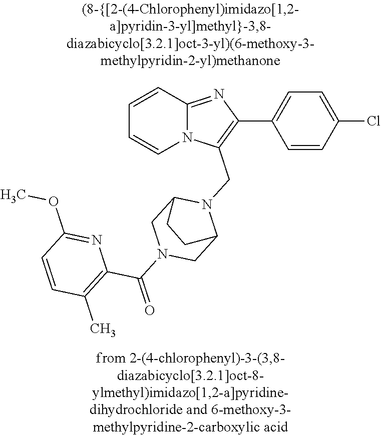 [SMILES: COc1ccc(C)c(C(=O)N2CC3CCC(C2)N3Cc2c(-c3ccc(Cl)cc3)nc3ccccn23)n1] | ¹H-NMR (400 MHz, DMSO- d₆): δ [ppm] = 1.49-1.70 (m, 2H), 1.79-1.97 (m, 2H), 2.09 (s, 3H), 2.78-2.92 (m, 2H), 2.98- 3.07 (m, 2H), 3.24 (br. d, 1H), 3.76 (s, 3H), 3.94-4.05 (m, 2H), 4.14 (br. d, 1H), 6.74 (d, 1H), 6.99 (td, 1H), 7.32 (ddd, 1H), 7.53 (d, 2H), 7.56-7.63 (m, 2H), 7.86 (d, 2H), 8.66 (d, 1H). LC-MS (Method 1): Rₜ = 0.78 min; MS (ESIpos): m/z = 502/504 [M + H]⁺. |
| 144 | 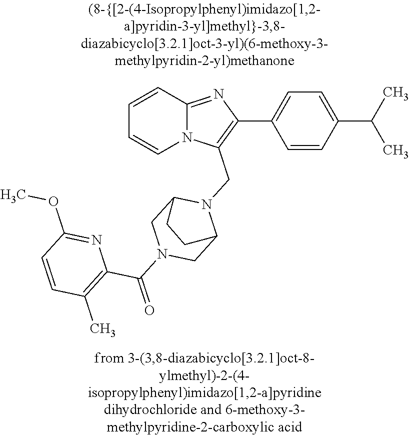 [SMILES: COc1ccc(C)c(C(=O)N2CC3CCC(C2)N3Cc2c(-c3ccc(C(C)C)cc3)nc3ccccn23)n1] | ¹H-NMR (400 MHz, DMSO- d₆): δ [ppm] = 1.24 (d, 6H), 1.49-1.70 (m, 2H), 1.80-1.97 (m, 2H), 2.09 (s, 3H), 2.81 (br. d, 1H), 2.87-2.97 (m, 2H), 3.01-3.08 (m, 2H), 3.27 (m, 1H), 3.76 (s, 3H), 3.94-4.05 (m, 2H), 4.14 (br. d, 1H), 6.74 (d, 1H), 6.97 (td, 1H), 7.25-7.37 (m, 3H), 7.58 (d, 2H), 7.73 (d, 2H), 8.66 (d, 1H). LC-MS (Method 1): Rₜ = 0.81 min; MS (ESIpos): m/z = 510 [M + H]⁺. |
| 145 | 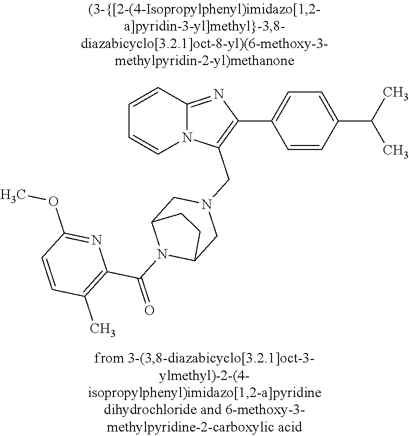 [SMILES: COc1ccc(C)c(C(=O)N2C3CCC2CN(Cc2c(-c4ccc(C(C)C)cc4)nc4ccccn24)C3)n1] | ¹H-NMR (400 MHz, DMSO- d₆): δ [ppm] = 1.25 (d, 6H), 1.61-1.84 (m, 4H), 2.17 (s, 3H), 2.43 (br. d, 2H), 2.47-2.56 (m, 1H, concealed by DMSO signal), 2.75 (dd, 1H), 2.88- 3.01 (m, 1H), 3.70 (s, 1H), 3.97-4.09 (m, 2H), 4.62 (br. s, 1H), 6.77 (d, 1H), 6.97 (td, 1H), 7.34 (d, 2H), 7.60 (t, 2H), 7.80 (d, 2H), 8.57 (d, 1H). LC-MS (Method 1): Rₜ = 0.82 min; MS (ESIpos): m/z = 510 [M + H]⁺. |
| 146 | 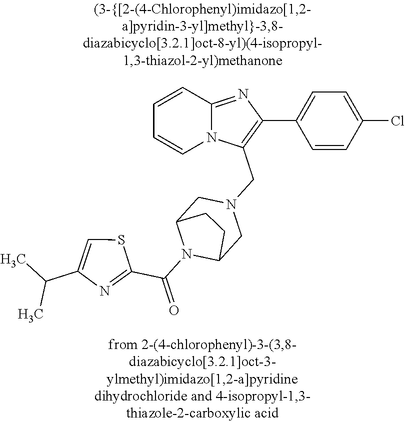 [SMILES: CC(C)c1csc(C(=O)N2C3CCC2CN(Cc2c(-c4ccc(Cl)cc4)nc4ccccn24)C3)n1] | ¹H-NMR (400 MHz, DMSO- d₆): δ [ppm] = 1.24 (d, 6H), 1.67-1.78 (m, 2H), 1.79-1.90 (m, 2H), 2.44 (br. d, 1H), 2.48- 2.56 (m, 1H, concealed by DMSO signal), 2.64 (br. d, 1H), 2.74 (br. d, 1H), 2.98-3.12 (m, 1H), 4.03 (s, 2H), 4.60 (br. s, 1H), 5.59 (br. s, 1H), 6.99 (t, 1H), 7.32 (t, 1H), 7.52 (d, 2H), 7.57 (s, 1H), 7.61 (d, 1H), 7.94 (d, 2H), 8.63 (d, 1H). LC-MS (Method 2): Rₜ = 1.93 min; MS (ESIpos): m/z = 506/508 [M + H]⁺. |
| 147 | 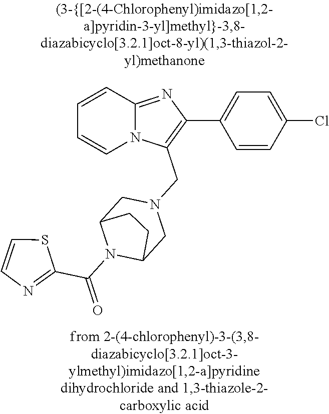 [SMILES: O=C(c1nccs1)N1C2CCC1CN(Cc1c(-c3ccc(Cl)cc3)nc3ccccn13)C2] | ¹H-NMR (400 MHz, DMSO- d₆): δ [ppm] = 1.67-1.92 (m, 4H), 2.42-2.57 (m, 1H, partly concealed by DMSO signal), 2.70 (br. t, 2H), 4.03 (s, 2H), 4.62 (br. s, 1H), 5.62 (br. s, 1H), 6.99 (t, 1H), 7.32 (t, 1H), 7.53 (d, 2H), 7.61 (d, 1H), 7.95 (d, 2H), 8.02 (q, 2H), 8.63 (d, 1H). LC-MS (Method 2): Rₜ = 1.49 min; MS (ESIpos): m/z = 464/466 [M + H]⁺. |
| 148 | 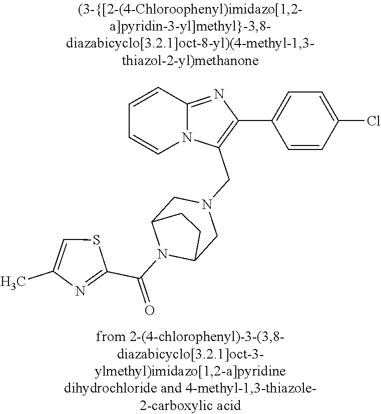 [SMILES: Cc1csc(C(=O)N2C3CCC2CN(Cc2c(-c4ccc(Cl)cc4)nc4ccccn24)C3)n1] | ¹H-NMR (400 MHz, DMSO- d₆): δ [ppm] = 1.67-1.77 (m, 2H), 1.79-1.92 (m, 2H), 2.41 (s, 3H), 2.42-2.57 (m, 2H, partly concealed by DMSO signal), 2.64 (br. d, 1H), 2.72 (br. d, 1H), 4.03 (s, 2H), 4.59 (br. s, 1H), 5.64 (br. s, 1H), 6.99 (t, 1H), 7.32 (t, 1H), 7.53 (d, 2H), 7.57 (s, 1H), 7.61 (d, 1H), 7.95 (d, 2H), 8.63 (d, 1H). LC-MS (Method 2): Rₜ = 1.69 min; MS (ESIpos): m/z = 478/480 [M + H]⁺. |
| 149 | 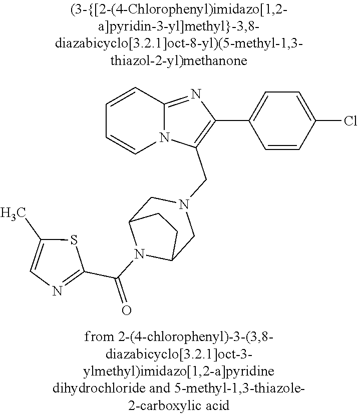 [SMILES: Cc1cnc(C(=O)N2C3CCC2CN(Cc2c(-c4ccc(Cl)cc4)nc4ccccn24)C3)s1] | ¹H-NMR (400 MHz, DMSO- d₆): δ [ppm] = 1.64-1.90 (m, 4H), 2.40-2.57 (m, 5H, partly concealed by DMSO signal), 2.68 (br. t, 2H), 4.02 (s, 2H), 4.59 (br. s, 1H), 5.59 (br. s, 1H), 6.99 (td, 1H), 7.32 (t, 1H), 7.53 (d, 2H), 7.61 (d, 1H), 7.69 (d, 1H), 7.95 (d, 2H), 8.62 (d, 1H). LC-MS (Method 2): Rₜ = 1.68 min; MS (ESIpos): m/z = 478/480 [M + H]⁺. |
| 150 | 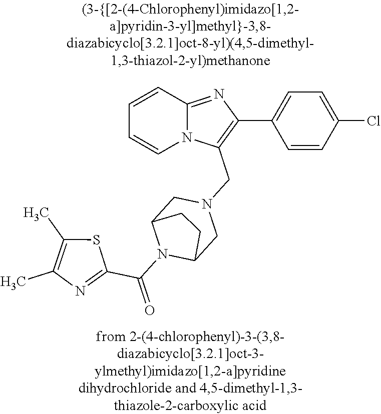 [SMILES: Cc1nc(C(=O)N2C3CCC2CN(Cc2c(-c4ccc(Cl)cc4)nc4ccccn24)C3)sc1C] | ¹H-NMR (400 MHz, DMSO- d₆): δ [ppm] = 1.65-1.89 (m, 4H), 2.29 (s, 3H), 2.38 (s, 3H), 2.43 (br. d, 2H), 2.60-2.75 (m, 2H), 4.02 (s, 2H), 4.57 (br. s, 1H), 5.62 (br. s, 1H), 6.99 (t, 1H), 7.32 (t, 1H), 7.53 (d, 2H), 7.61 (d, 1H), 7.95 (d, 2H), 8.63 (d, 1H). LC-MS (Method 2): Rₜ = 1.75 min; MS (ESIpos): m/z = 492/494 [M + H]⁺. |
| 151 | 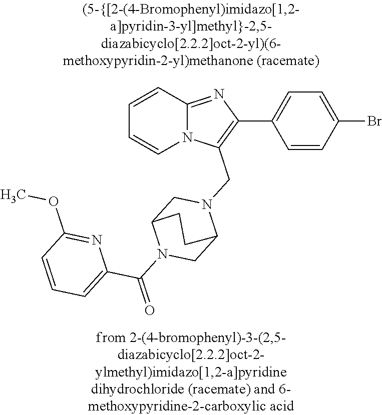 [SMILES: COc1cccc(C(=O)N2CC3CCC2CN3Cc2c(-c3ccc(Br)cc3)nc3ccccn23)n1] | ¹H-NMR (400 MHz, DMSO- d₆): δ [ppm] = 1.46-1.99 (m, 4H), 2.62-2.75 (m, 1H), 2.81- 2.93 (m, 2H), 3.35-3.50 (m, 1H), 3.70-3.82 (m, 3.75H), 3.89-4.01 (m, 1H), 4.20-4.41 (m, 2.25H), 6.83-7.02 (m, 2H), 7.17 (d, 0.75H), 7.25-7.35 (m, 1.25H), 7.55-7.70 (m, 3H), 7.73-7.86 (m, 3H), 8.54-8.64 (m, 1H). LC-MS (Method 2): Rₜ = 1.39 min; MS (ESIpos): m/z = 532/534 [M + H]⁺. |
| 152 | 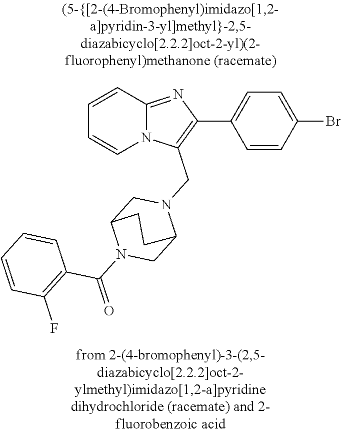 [SMILES: O=C(c1ccccc1F)N1CC2CCC1CN2Cc1c(-c2ccc(Br)cc2)nc2ccccn12] | ¹H-NMR (400 MHz, DMSO- d₆): δ [ppm] = 1.48-1.97 (m, 4H), 2.61-3.04 (m, 4H), 3.42 (br. d, 1H), 3.76 (br. d, 0.75H), 4.16-4.29 (m, 2H), 4.39 (br. s, 0.25H), 6.92-7.02 (m, 1H), 7.20-7.39 (m, 4H), 7.41-7.54 (m, 1H), 7.56-7.72 (m, 3H), 7.74-7.86 (m, 2H), 8.54-8.63 (m, 1H). LC-MS (Method 5): Rₜ = 1.06 min; MS (ESIpos): m/z = 519 [M + H]⁺. |
| 153 | 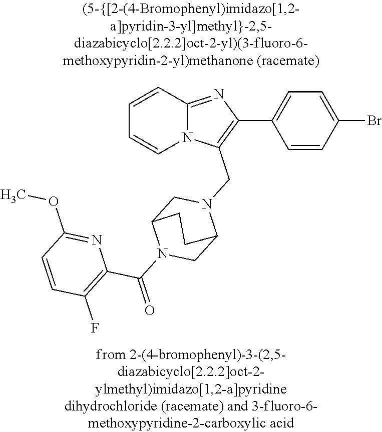 [SMILES: COc1ccc(F)c(C(=O)N2CC3CCC2CN3Cc2c(-c3ccc(Br)cc3)nc3ccccn23)n1] | ¹H-NMR (400 MHz, DMSO- d₆): δ [ppm] = 1.51-2.00 (m, 4H), 2.63-2.73 (m, 1H), 2.77- 2.85 (m, 1H), 2.91 (br. s, 0.75H), 3.14 (br. d, 0.25H), 3.38-3.59 (m, 2H), 3.69-3.83 (m, 3.75H), 4.17-4.32 (m, 2H), 4.39 (br. s, 0.25H), 6.87-7.03 (m, 2H), 7.31 (dd, 1H), 7.56- 7.69 (m, 3H), 7.71-7.86 (m, 3H), 8.54-8.64 (m, 1H). LC-MS (Method 5): Rₜ = 1.07 min; MS (ESIpos): m/z = 550/552 [M + H]⁺. |

### Example 154

### 3-{[2-(4-Chlorophenyl)imidazo[1,2-a]pyridin-3-yl]methyl}-N-isopropyl-3,8-diazabicyclo[3.2.1]octane-8-carboxamide

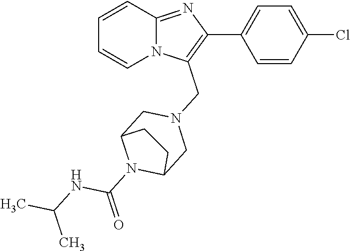 [SMILES: CC(C)NC(=O)N1C2CCC1CN(Cc1c(-c3ccc(Cl)cc3)nc3ccccn13)C2]

8.5 mg (0.10 mmol) of isopropyl isocyanate were initially charged in a well of a 96-well multititre plate and cooled to 0° C. Separately, 42.6 mg (0.10 mmol) of 3-{[2-(4-chlorophenyl)imidazo[1,2-a]pyridin-3-yl]methyl}-3,8-diazabicyclo[3.2.1]octane dihydrochloride were dissolved in 0.8 ml of 1,2-dichloroethane, 0.052 ml (0.3 mmol) of N,N-diisopropylethylamine was added, and the mixture was cooled to 8° C. The two solutions were combined in the multititre plate and first agitated at 0° C. for 1 h.

Subsequently, the mixture was allowed to warm up to RT and subjected to further agitation at RT overnight. Thereafter, the solvent was removed completely by means of a centrifugal dryer. The residue was dissolved in 0.6 ml of DMF and filtered, and the filtrate was separated into its components by preparative LC-MS by one of the following methods:

MS instrument: Waters; HPLC instrument: Waters; Waters X-Bridge C18 column, 19 mm×50 mm, 5 μm, eluent A: water+0.375% ammonia, eluent B: acetonitrile+0.375% ammonia, with eluent gradient; flow rate: 40 ml/min; UV detection: DAD, 210-400 nm

or

MS instrument: Waters; HPLC instrument: Waters; Phenomenex Luna 5μ C18 (2) 100A column, AXIA Tech., 50 mm×21.2 mm, eluent A: water+0.0375% formic acid, eluent B: acetonitrile+0.0375% formic acid, with eluent gradient; flow rate: 40 m/min; UV detection: DAD; 210-400 nm.

In this way, 2.8 mg (6% of theory, 100% purity) of the title compound were obtained.

LC-MS (Method 7, ESIpos): Rₜ=0.85 min; m/z=438 (M+H)⁺.

By way of parallel synthesis analogously to Example 154, the following compounds were prepared proceeding from 3-{[2-(4-chlorophenyl)imidazo[1,2-a]pyridin-3-yl]methyl}-3,8-diazabicyclo[3.2.1]octane dihydrochloride (in Examples 155-167 and 170-187) or 7-{[2-(4-chlorophenyl)imidazo[1,2-a]pyridin-3-yl]methyl}-3-oxa-7,9-diazabicyclo[3.3.1]nonane dihydrochloride (in Examples 168, 169 and 188-198) and the appropriate isocyanate, carbamoyl chloride or chloroformate:

| | Name/structure | |
|---|---|---|
| Example | (yield; purity) | LC-MS (Method 7) |
| 155 | 3-{[2-(4-Chlorophenyl)imidazo[1,2- | Rₜ = 0.91 min; m/z = 490 |
| | a]pyridin-3-yl]methyl}-N-(2-fluorophenyl)- | (M + H)⁺ |
| | 3,8-diazabicyclo[3.2.1]octane-8- | |
| | carboxamide | |
| | 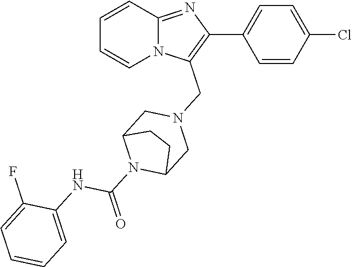 [SMILES: O=C(Nc1ccccc1F)N1C2CCC1CN(Cc1c(-c3ccc(Cl)cc3)nc3ccccn13)C2] | |
| | (38% of theory; purity 97%) | |
| 156 | 3-{[2-(4-Chlorophenyl)imidazo[1,2- | Rₜ = 0.93 min; m/z = 540 |
| | a]pyridin-3-yl]methyl}-N-(2,6- | (M + H)⁺ |
| | dichlorophenyl)-3,8- | |
| | diazabicyclo[3.2.1]octane-8-carboxamide | |
| | 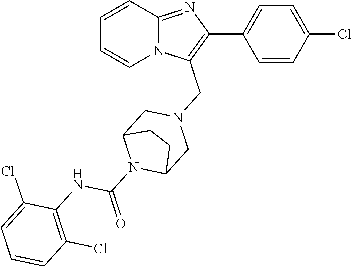 [SMILES: O=C(Nc1c(Cl)cccc1Cl)N1C2CCC1CN(Cc1c(-c3ccc(Cl)cc3)nc3ccccn13)C2] | |
| | (8% of theory; purity 96%) | |
| 157 | 3-{[2-(4-Chlorophenyl)imidazo[1,2- | Rₜ = 0.92 min; m/z = 500 |
| | a]pyridin-3-yl]methyl}-N-(2,6- | (M + H)⁺ |
| | dimethylphenyl)-3,8- | |
| | diazabicyclo[3.2.1]octane-8-carboxamide | |
| | 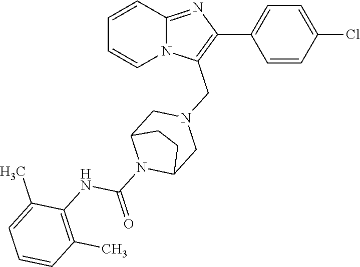 [SMILES: Cc1cccc(C)c1NC(=O)N1C2CCC1CN(Cc1c(-c3ccc(Cl)cc3)nc3ccccn13)C2] | |
| | (14% of theory; purity 100%) | |
| 158 | 3-{[2-(4-Chlorophenyl)imidazo[1,2- | Rₜ = 0.93 min; m/z = 466 |
| | a]pyridin-3-yl]methyl}-N-pentyl-3,8- | (M + H)⁺ |
| | diazabicyclo[3.2.1]octane-8-carboxamide | |
| | 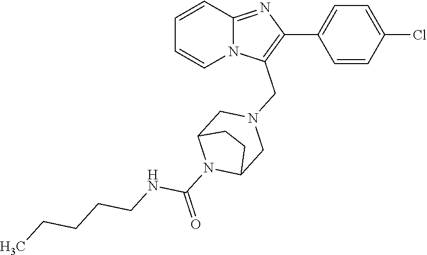 [SMILES: CCCCCNC(=O)N1C2CCC1CN(Cc1c(-c3ccc(Cl)cc3)nc3ccccn13)C2] | |
| | (15% of theory; purity 100%) | |
| 159 | 3-{[2-(4-Chlorophenyl)imidazo[1,2- | Rₜ = 0.92 min; m/z = 486 |
| | a]pyridin-3-yl]methyl}-N-(2- | (M + H)⁺ |
| | methylphenyl)-3,8- | |
| | diazabicyclo[3.2.1]octane-8-carboxamide | |
| | 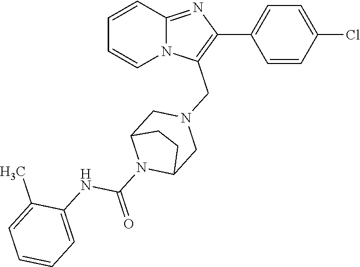 [SMILES: Cc1ccccc1NC(=O)N1C2CCC1CN(Cc1c(-c3ccc(Cl)cc3)nc3ccccn13)C2] | |
| | (8% of theory; purity 96%) | |
| 160 | 3-{[2-(4-Chlorophenyl)imidazo[1,2- | Rₜ = 1.06 min; m/z = 574 |
| | a]pyridin-3-yl]methyl}-N-[2-chloro-5- | (M + H)⁺ |
| | (trifluoromethyl)phenyl]-3,8- | |
| | diazabicyclo[3.2.1]octane-8-carboxamide | |
| | 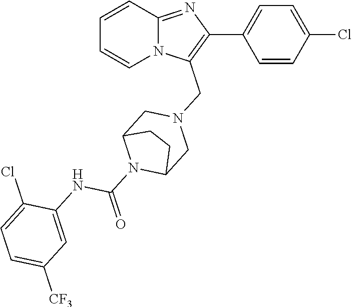 [SMILES: O=C(Nc1cc(C(F)(F)F)ccc1Cl)N1C2CCC1CN(Cc1c(-c3ccc(Cl)cc3)nc3ccccn13)C2] | |
| | (45% of theory; purity 92%) | |
| 161 | N-(4-Chlorophenyl)-3-{[2-(4- | Rₜ = 0.98 min; m/z = 506 |
| | chlorophenyl)imidazo[1,2-a]pyridin-3- | (M + H)⁺ |
| | yl]methyl}-3,8-diazabicyclo[3.2.1]octane-8- | |
| | carboxamide | |
| | 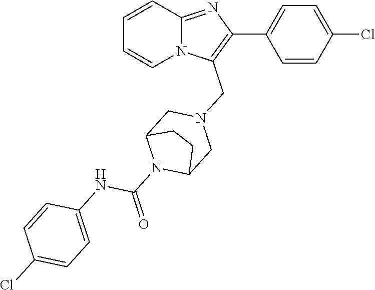 [SMILES: O=C(Nc1ccc(Cl)cc1)N1C2CCC1CN(Cc1c(-c3ccc(Cl)cc3)nc3ccccn13)C2] | |
| | (50% of theory; purity 100%) | |
| 162 | 3-{[2-(4-Chlorophenyl)imidazo[1,2- | Rₜ = 0.96 min; m/z = 514 |
| | a]pyridin-3-yl]methyl}-N-(2-ethyl-6- | (M + H)⁺ |
| | methylphenyl)-3,8- | |
| | diazabicyclo[3.2.1]octane-8-carboxamide | |
| | 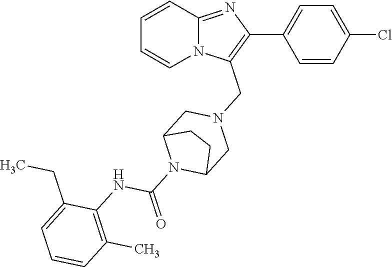 [SMILES: CCc1cccc(C)c1NC(=O)N1C2CCC1CN(Cc1c(-c3ccc(Cl)cc3)nc3ccccn13)C2] | |
| | (15% of theory; purity 97%) | |
| 163 | 3-{[2-(4-Chlorophenyl)imidazo[1,2- | Rₜ = 0.96 min; m/z = 500 |
| | a]pyridin-3-yl]methyl}-N-(2,5- | (M + H)⁺ |
| | dimethylphenyl)-3,8- | |
| | diazabicyclo[3.2.1]octane-8-carboxamide | |
| | 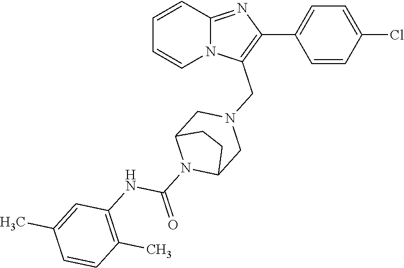 [SMILES: Cc1ccc(C)c(NC(=O)N2C3CCC2CN(Cc2c(-c4ccc(Cl)cc4)nc4ccccn24)C3)c1] | |
| | (19% of theory; purity 100%) | |
| 164 | 3-{[2-(4-Chlorophenyl)imidazo[1,2- | Rₜ = 0.93 min; m/z = 478 |
| | a]pyridin-3-yl]methyl}-N-cyclohexyl-3,8- | (M + H)⁺ |
| | diazabicyclo[3.2.1]octane-8-carboxamide | |
| | 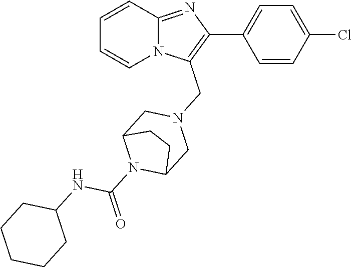 [SMILES: O=C(NC1CCCCC1)N1C2CCC1CN(Cc1c(-c3ccc(Cl)cc3)nc3ccccn13)C2] | |
| | (35% of theory; purity 100%) | |
| 165 | N-(2-Chloro-6-methylphenyl)-3-{[2-(4- | Rₜ = 0.93 min; m/z = 520 |
| | chlorophenyl)imidazo[1,2-a]pyridin-3- | (M + H)⁺ |
| | yl]methyl}-3,8-diazabicyclo[3.2.1]octane-8- | |
| | carboxamide | |
| | 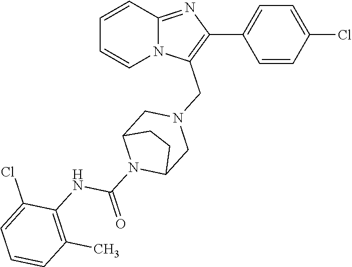 [SMILES: Cc1cccc(Cl)c1NC(=O)N1C2CCC1CN(Cc1c(-c3ccc(Cl)cc3)nc3ccccn13)C2] | |
| | (37% of theory; purity 97%) | |
| 166 | 3-{[2-(4-Chlorophenyl)imidazo[1,2- | Rₜ = 0.90 min; m/z = 508 |
| | a]pyridin-3-yl]methyl}-N-(2,6- | (M + H)⁺ |
| | difluorophenyl)-3,8- | |
| | diazabicyclo[3.2.1]octane-8-carboxamide | |
| | 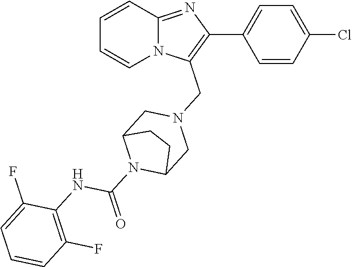 [SMILES: O=C(Nc1c(F)cccc1F)N1C2CCC1CN(Cc1c(-c3ccc(Cl)cc3)nc3ccccn13)C2] | |
| | (23% of theory; purity 100%) | |
| 167 | 3-{[2-(4-Chlorophenyl)imidazo[1,2- | Rₜ = 0.96 min; m/z = 500 |
| | a]pyridin-3-yl]methyl}-N-(2,4- | (M + H)⁺ |
| | dimethylphenyl)-3,8- | |
| | diazabicyclo[3.2.1]octane-8-carboxamide | |
| | 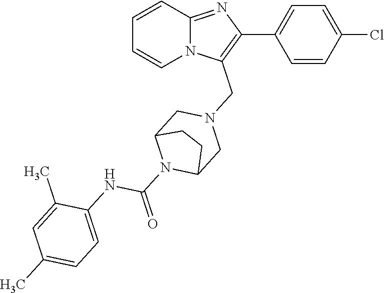 [SMILES: Cc1ccc(NC(=O)N2C3CCC2CN(Cc2c(-c4ccc(Cl)cc4)nc4ccccn24)C3)c(C)c1] | |
| | (5% of theory; purity 100%) | |
| 168 | 7-{[2-(4-Chlorophenyl)imidazo[1,2- | Rₜ = 0.77 min; m/z = 454 |
| | a]pyridin-3-yl]methyl}-N-isopropyl-3-oxa- | (M + H)⁺ |
| | 7,9-diazabicyclo[3.3.1]nonane-9- | |
| | carboxamide | |
| | 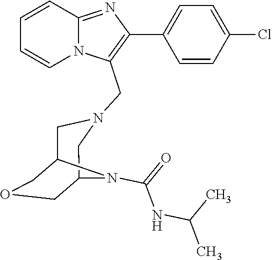 [SMILES: CC(C)NC(=O)N1C2COCC1CN(Cc1c(-c3ccc(Cl)cc3)nc3ccccn13)C2] | |
| | (16% of theory; purity 94%) | |
| 169 | N-(2-Chloro-6-methylphenyl)-7-{[2-(4- | Rₜ = 0.82 min; m/z = 536 |
| | chlorophenyl)imidazo[1,2-a]pyridin-3- | (M + H)⁺ |
| | yl]methyl}-3-oxa-7,9- | |
| | diazabicyclo[3.3.1]nonane-9-carboxamide | |
| | 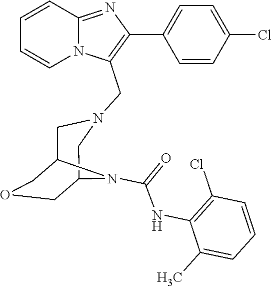 [SMILES: Cc1cccc(Cl)c1NC(=O)N1C2COCC1CN(Cc1c(-c3ccc(Cl)cc3)nc3ccccn13)C2] | |
| | (2% of theory; purity 93%) | |
| 170 | 3-{[2-(4-Chlorophenyl)imidazo[1,2- | Rₜ = 0.79 min; m/z = 436 |
| | a]pyridin-3-yl]methyl}-N-cyclopropyl-3,8- | (M + H)⁺ |
| | diazabicyclo[3.2.1]octane-8-carboxamide | |
| | 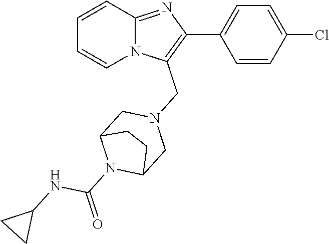 [SMILES: O=C(NC1CC1)N1C2CCC1CN(Cc1c(-c3ccc(Cl)cc3)nc3ccccn13)C2] | |
| | (4% of theory; purity 100%) | |
| 171 | N-(2-Chlorophenyl)-3-{[2-(4- | Rₜ = 0.93 min; m/z = 506 |
| | chlorophenyl)imidazo[1,2-a]pyridin-3- | (M + H)⁺ |
| | yl]methyl}-3,8-diazabicyclo[3.2.1]octane-8- | |
| | carboxamide | |
| | 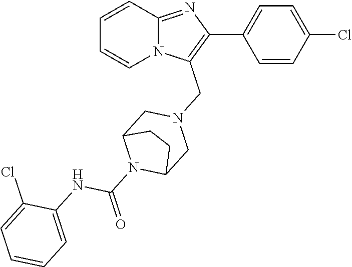 [SMILES: O=C(Nc1ccccc1Cl)N1C2CCC1CN(Cc1c(-c3ccc(Cl)cc3)nc3ccccn13)C2] | |
| | (6% of theory; purity 97%) | |
| 172 | 3-{[2-(4-Chlorophenyl)imidazo[1,2- | Rₜ = 0.95 min; m/z = 486 |
| | a]pyridin-3-yl]methyl}-N-methyl-N-phenyl- | (M + H)⁺ |
| | 3,8-diazabicyclo[3.2.1]octane-8- | |
| | carboxamide | |
| | 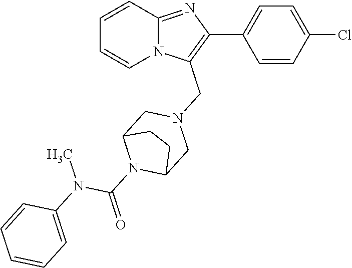 [SMILES: CN(C(=O)N1C2CCC1CN(Cc1c(-c3ccc(Cl)cc3)nc3ccccn13)C2)c1ccccc1] | |
| | (38% of theory; purity 100%) | |
| 173 | (3-{[2-(4-Chlorophenyl)imidazo[1,2- | Rₜ = 0.81 min; m/z = 466 |
| | a]pyridin-3-yl]methyl}-3,8- | (M + H)⁺ |
| | diazabicyclo[3.2.1]oct-8-yl)(morpholin-4- | |
| | yl)methanone | |
| | 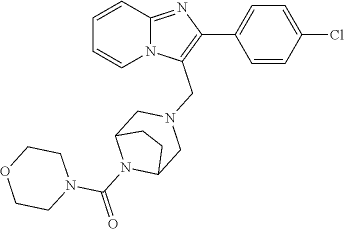 [SMILES: O=C(N1CCOCC1)N1C2CCC1CN(Cc1c(-c3ccc(Cl)cc3)nc3ccccn13)C2] | |
| | (6% of theory; purity 98%) | |
| 174 | 3-{[2-(4-Chlorophenyl)imidazo[1,2- | Rₜ = 0.98 min; m/z = 480 |
| | a]pyridin-3-yl]methyl}-N,N-diisopropyl- | (M + H)⁺ |
| | 3,8-diazabicyclo[3.2.1]octane-8- | |
| | carboxamide | |
| | 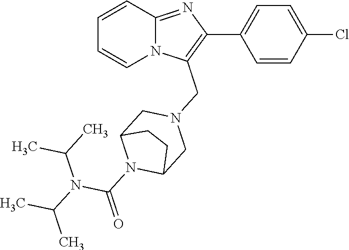 [SMILES: CC(C)N(C(=O)N1C2CCC1CN(Cc1c(-c3ccc(Cl)cc3)nc3ccccn13)C2)C(C)C] | |
| | (20% of theory; purity 100%) | |
| 175 | 3-{[2-(4-Chlorophenyl)imidazo[1,2- | Rₜ = 1.04 min; m/z = 506 |
| | a]pyridin-3-yl]methyl}-N-cyclohexyl-N- | (M + H)⁺ |
| | ethyl-3,8-diazabicyclo[3.2.1]octane-8- | |
| | carboxamide | |
| | 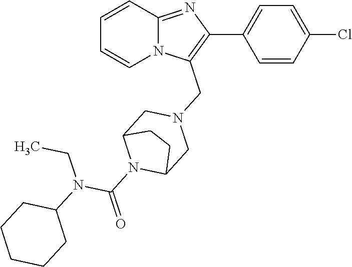 [SMILES: CCN(C(=O)N1C2CCC1CN(Cc1c(-c3ccc(Cl)cc3)nc3ccccn13)C2)C1CCCCC1] | |
| | (44% of theory; purity 100%) | |
| 176 | (3-{[2-(4-Chlorophenyl)imidazo[1,2- | Rₜ = 0.87 min; m/z = 450 |
| | a]pyridin-3-yl]methyl}-3,8- | (M + H)⁺ |
| | diazabicyclo[3.2.1]oct-8-yl)(pyrrolidin-1- | |
| | yl)methanone | |
| | 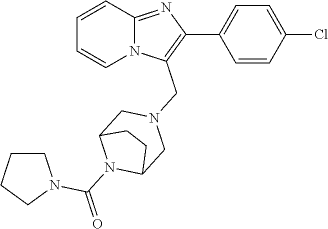 [SMILES: O=C(N1CCCC1)N1C2CCC1CN(Cc1c(-c3ccc(Cl)cc3)nc3ccccn13)C2] | |
| | (6% of theory; purity 93%) | |
| 177 | 3-{[2-(4-Chlorophenyl)imidazo[1,2- | Rₜ = 0.98 min; m/z = 500 |
| | a]pyridin-3-yl]methyl}-N-ethyl-N-phenyl- | (M + H)⁺ |
| | 3,8-diazabicyclo[3.2.1]octane-8- | |
| | carboxamide | |
| | 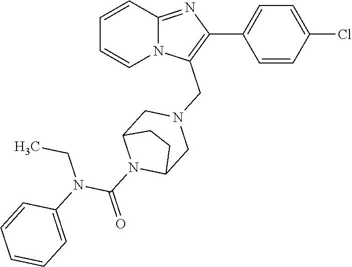 [SMILES: CCN(C(=O)N1C2CCC1CN(Cc1c(-c3ccc(Cl)cc3)nc3ccccn13)C2)c1ccccc1] | |
| | (40% of theory; purity 100%) | |
| 178 | 3-{[2-(4-Chlorophenyl)imidazo[1,2- | Rₜ = 0.90 min; m/z = 452 |
| | a]pyridin-3-yl]methyl}-N-isopropyl-N- | (M + H)⁺ |
| | methyl-3,8-diazabicyclo[3.2.1]octane-8- | |
| | carboxamide | |
| | 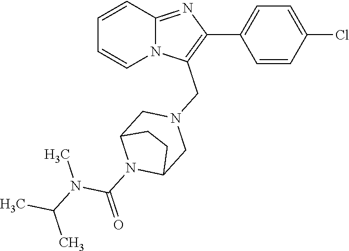 [SMILES: CC(C)N(C)C(=O)N1C2CCC1CN(Cc1c(-c3ccc(Cl)cc3)nc3ccccn13)C2] | |
| | (2% of theory; purity 100%) | |
| 179 | (3-{[2-(4-Chlorophenyl)imidazo[1,2- | Rₜ = 0.92 min; m/z = 464 |
| | a]pyridin-3-yl]methyl}-3,8- | (M + H)⁺ |
| | diazabicyclo[3.2.1]oct-8-yl)(piperidin-1- | |
| | yl)methanone | |
| | 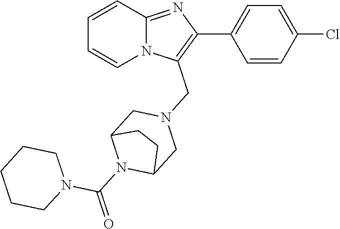 [SMILES: O=C(N1CCCCC1)N1C2CCC1CN(Cc1c(-c3ccc(Cl)cc3)nc3ccccn13)C2] | |
| | (19% of theory; purity 97%) | |
| 180 | 3-{[2-(4-Chlorophenyl)imidazo[1,2- | Rₜ = 0.83 min; m/z = 424 |
| | a]pyridin-3-yl]methyl}-N, N-dimethyl-3,8- | (M + H)⁺ |
| | diazabicyclo[3.2.1]octane-8-carboxamide | |
| | 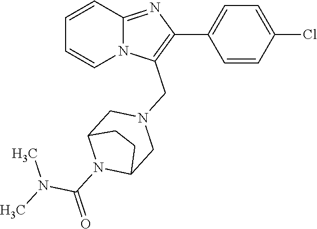 [SMILES: CN(C)C(=O)N1C2CCC1CN(Cc1c(-c3ccc(Cl)cc3)nc3ccccn13)C2] | |
| | (11% of theory; purity 100%) | |
| 181 | 3-{[2-(4-Chlorophenyl)imidazo[1,2- | Rₜ = 1.02 min; m/z = 514 |
| | a]pyridin-3-yl]methyl}-N-ethyl-N-(4- | (M + H)⁺ |
| | methylphenyl)-3,8- | |
| | diazabicyclo[3.2.1]octane-8-carboxamide | |
| | 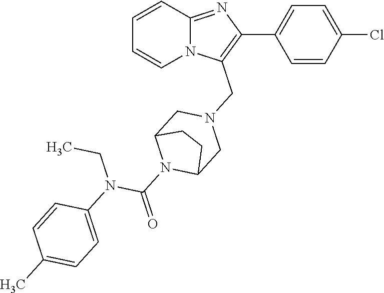 [SMILES: CCN(C(=O)N1C2CCC1CN(Cc1c(-c3ccc(Cl)cc3)nc3ccccn13)C2)c1ccc(C)cc1] | |
| | (38% of theory; purity 95%) | |
| 182 | N-(4-Chlorophenyl)-3-{[2-(4- | Rₜ = 1.05 min; m/z = 548 |
| | chlorophenyl)imidazo[1,2-a]pyridin-3- | (M + H)⁺ |
| | yl]methyl}-N-isopropyl-3,8- | |
| | diazabicyclo[3.2.1]octane-8-carboxamide | |
| | 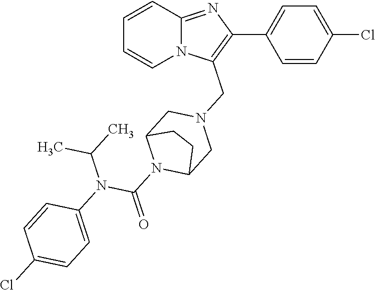 [SMILES: CC(C)N(C(=O)N1C2CCC1CN(Cc1c(-c3ccc(Cl)cc3)nc3ccccn13)C2)c1ccc(Cl)cc1] | |
| | (19% of theory; purity 99%) | |
| 183 | (3-{[2-(4-Chlorophenyl)imidazo[1,2- | Rₜ = 0.89 min; m/z = 482 |
| | a]pyridin-3-yl]methyl}-3,8- | (M + H)⁺ |
| | diazabicyclo[3.2.1]oct-8-yl)(thiomorpholin- | |
| | 4-yl)methanone | |
| | 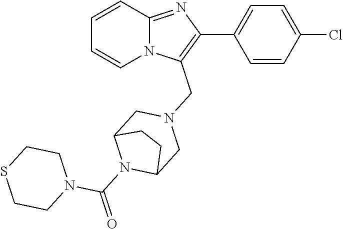 [SMILES: O=C(N1CCSCC1)N1C2CCC1CN(Cc1c(-c3ccc(Cl)cc3)nc3ccccn13)C2] | |
| | (22% of theory; purity 100%) | |
| 184 | Methyl 3-{[2-(4-chlorophenyl)imidazo[1,2- | Rₜ = 0.85 min; m/z = 411 |
| | a]pyridin-3-yl]methyl}-3,8- | (M + H)⁺ |
| | diazabicyclo[3.2.1]octane-8-carboxylate | |
| | 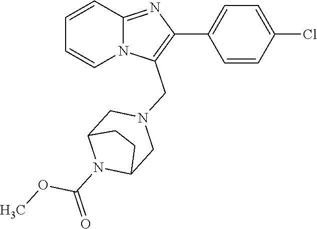 [SMILES: COC(=O)N1C2CCC1CN(Cc1c(-c3ccc(Cl)cc3)nc3ccccn13)C2] | |
| | (7% of theory; purity 100%) | |
| 185 | Ethyl 3-{[2-(4-chlorophenyl)imidazo[1,2- | Rₜ = 0.89 min; m/z = 425 |
| | a]pyridin-3-yl]methyl}-3,8- | (M + H)⁺ |
| | diazabicyclo[3.2.1]octane-8-carboxylate | |
| | 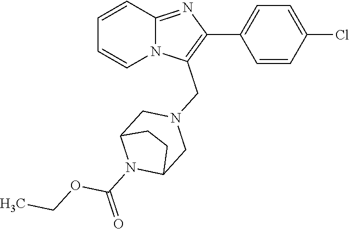 [SMILES: CCOC(=O)N1C2CCC1CN(Cc1c(-c3ccc(Cl)cc3)nc3ccccn13)C2] | |
| | (15% of theory; purity 100%) | |
| 186 | Cyclopentyl 3-{[2-(4- | Rₜ = 0.98 min; m/z = 465 |
| | chlorophenyl)imidazo[1,2-a]pyridin-3- | (M + H)⁺ |
| | yl]methyl}-3,8-diazabicyclo[3.2.1]octane-8- | |
| | carboxylate | |
| | 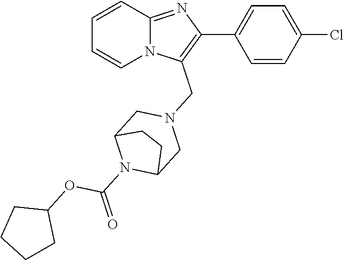 [SMILES: O=C(OC1CCCC1)N1C2CCC1CN(Cc1c(-c3ccc(Cl)cc3)nc3ccccn13)C2] | |
| | (12% of theory; purity 99%) | |
| 187 | Cyclohexyl 3-{[2-(4- | Rₜ = 1.02 min; m/z = 479 |
| | chlorophenyl)imidazo[1,2-a]pyridin-3- | (M + H)⁺ |
| | yl]methyl}-3,8-diazabicyclo[3.2.1]octane-8- | |
| | carboxylate | |
| | 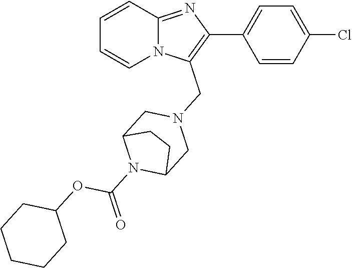 [SMILES: O=C(OC1CCCCC1)N1C2CCC1CN(Cc1c(-c3ccc(Cl)cc3)nc3ccccn13)C2] | |
| | (8% of theory; purity 100%) | |
| 188 | 7-{[2-(4-Chlorophenyl)imidazo[1,2- | Rₜ = 0.78 min; m/z = 468 |
| | a]pyridin-3-yl]methyl}-N,N-diethyl-3-oxa- | (M + H)⁺ |
| | 7,9-diazabicyclo[3.3.1]nonane-9- | |
| | carboxamide | |
| | 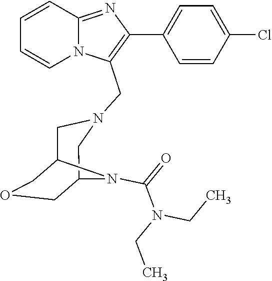 [SMILES: CCN(CC)C(=O)N1C2COCC1CN(Cc1c(-c3ccc(Cl)cc3)nc3ccccn13)C2] | |
| | (35% of theory; purity 100%) | |
| 189 | (7-{[2-(4-Chlorophenyl)imidazo[1,2- | Rₜ = 0.72 min; m/z = 482 |
| | a]pyridin-3-yl]methyl}-3-oxa-7,9- | (M + H)⁺ |
| | diazabicyclo[3.3.1]non-9-yl)(morpholin-4- | |
| | yl)methanone | |
| | 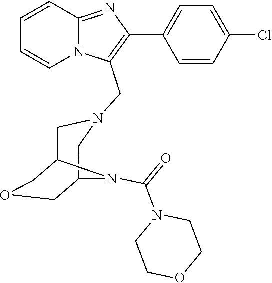 [SMILES: O=C(N1CCOCC1)N1C2COCC1CN(Cc1c(-c3ccc(Cl)cc3)nc3ccccn13)C2] | |
| | (27% of theory; purity 97%) | |
| 190 | 7-{[2-(4-Chlorophenyl)imidazo[1,2- | Rₜ = 0.84 min; m/z = 496 |
| | a]pyridin-3-yl]methyl}-N,N-diisopropyl-3- | (M + H)⁺ |
| | oxa-7,9-diazabicyclo[3.3.1]nonane-9- | |
| | carboxamide | |
| | 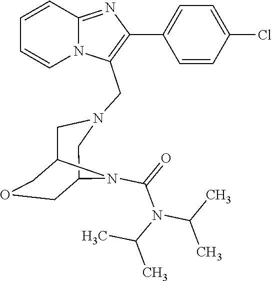 [SMILES: CC(C)N(C(=O)N1C2COCC1CN(Cc1c(-c3ccc(Cl)cc3)nc3ccccn13)C2)C(C)C] | |
| | (27% of theory; purity 100%) | |
| 191 | 7-{[2-(4-Chlorophenyl)imidazo[1,2- | Rₜ = 0.90 min; m/z = 522 |
| | a]pyridin-3-yl]methyl}-N-cyclohexyl-N- | (M + H)⁺ |
| | ethyl-3-oxa-7,9-diazabicyclo[3.3.1]nonane- | |
| | 9-carboxamide | |
| | 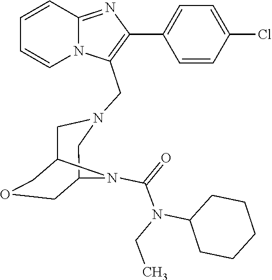 [SMILES: CCN(C(=O)N1C2COCC1CN(Cc1c(-c3ccc(Cl)cc3)nc3ccccn13)C2)C1CCCCC1] | |
| | (43% of theory; purity 100%) | |
| 192 | (7-{[2-(4-Chlorophenyl)imidazo[1,2- | Rₜ = 0.76 min; m/z = 466 |
| | a]pyridin-3-yl]methyl}-3-oxa-7,9- | (M + H)⁺ |
| | diazabicyclo[3.3.1]non-9-yl)(pyrrolidin-1- | |
| | yl)methanone | |
| | 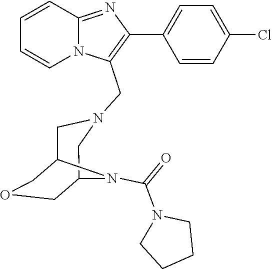 [SMILES: O=C(N1CCCC1)N1C2COCC1CN(Cc1c(-c3ccc(Cl)cc3)nc3ccccn13)C2] | |
| | (27% of theory; purity 96%) | |
| 193 | 7-{[2-(4-Chlorophenyl)imidazo[1,2- | Rₜ = 0.86 min; m/z = 516 |
| | a]pyridin-3-yl]methyl}-N-ethyl-N-phenyl-3- | (M + H)⁺ |
| | oxa-7,9-diazabicyclo[3.3.1]nonane-9- | |
| | carboxamide | |
| | 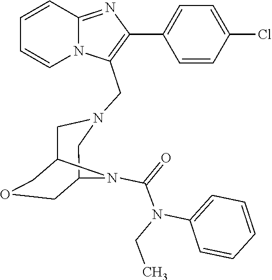 [SMILES: CCN(C(=O)N1C2COCC1CN(Cc1c(-c3ccc(Cl)cc3)nc3ccccn13)C2)c1ccccc1] | |
| | (30% of theory; purity 99%) | |
| 194 | 7-{[2-(4-Chlorophenyl)imidazo[1,2- | Rₜ = 0.78 min; m/z = 468 |
| | a]pyridin-3-yl]methyl}-N-isopropyl-N- | (M + H)⁺ |
| | methyl-3-oxa-7,9- | |
| | diazabicyclo[3.3.1]nonane-9-carboxamide | |
| | 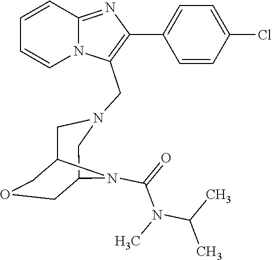 [SMILES: CC(C)N(C)C(=O)N1C2COCC1CN(Cc1c(-c3ccc(Cl)cc3)nc3ccccn13)C2] | |
| | (44% of theory; purity 100%) | |
| 195 | Ethyl 7-{[2-(4-chlorophenyl)imidazo[1,2- | Rₜ = 0.79 min; m/z = 441 |
| | a]pyridin-3-yl]methyl}-3-oxa-7,9- | (M + H)⁺ |
| | diazabicyclo[3.3.1]nonane-9-carboxylate | |
| | 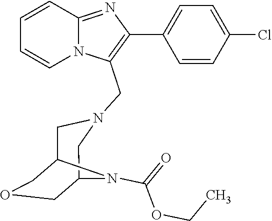 [SMILES: CCOC(=O)N1C2COCC1CN(Cc1c(-c3ccc(Cl)cc3)nc3ccccn13)C2] | |
| | (9% of theory; purity 100%) | |
| 196 | Cyclopentyl 7-{[2-(4- | Rₜ = 0.87 min; m/z = 481 |
| | chlorophenyl)imidazo[1,2-a]pyridin-3- | (M + H)⁺ |
| | yl]methyl}-3-oxa-7,9- | |
| | diazabicyclo[3.3.1]nonane-9-carboxylate | |
| | 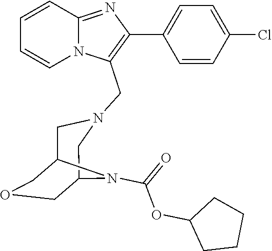 [SMILES: O=C(OC1CCCC1)N1C2COCC1CN(Cc1c(-c3ccc(Cl)cc3)nc3ccccn13)C2] | |
| | (6% of theory; purity 100%) | |
| 197 | Propyl 7-{[2-(4-chlorophenyl)imidazo[1,2- | Rₜ = 0.83 min; m/z = 455 |
| | a]pyridin-3-yl]methyl}-3-oxa-7,9- | (M + H)⁺ |
| | diazabicyclo[3.3.1]nonane-9-carboxylate | |
| | 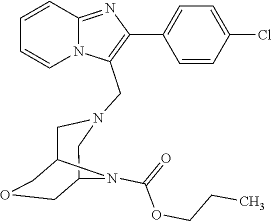 [SMILES: CCCOC(=O)N1C2COCC1CN(Cc1c(-c3ccc(Cl)cc3)nc3ccccn13)C2] | |
| | (7% of theory; purity 100%) | |
| 198 | (7-{[2-(4-Chlorophenyl)imidazo[1,2- | Rₜ = 0.79 min; m/z = 480 |
| | a]pyridin-3-yl]methyl}-3-oxa-7,9- | (M + H)⁺ |
| | diazabicyclo[3.3.1]non-9-yl)(piperidin-1- | |
| | yl)methanone | |
| | 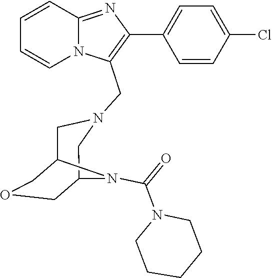 [SMILES: O=C(N1CCCCC1)N1C2COCC1CN(Cc1c(-c3ccc(Cl)cc3)nc3ccccn13)C2] | |
| | (7% of theory; purity 98%) | |

Analogously to Examples 21 and 33, the following compound was prepared from the reactants specified:

| Example | Name/structure/reactants | Analytical data |
|---|---|---|
| 199 | (5-{[2-(4-Bromophenyl)imidazo[1,2- | ¹H NMR (400 MHz, DMSO- |
| | a]pyridin-3-yl]methyl}-2,5- | d₆): δ [ppm] = 1.51-1.98 (m, |
| | diazabicyclo[2.2.2]oct-2-yl)(3-chlor-6- | 4H), 2.60 (dd, 0.75H), 2.69- |
| | methoxypyridin-2-yl)methanone (racemate) | 2.77 (m, 1H), 2.80 (br. s, 0.5H), |
| | 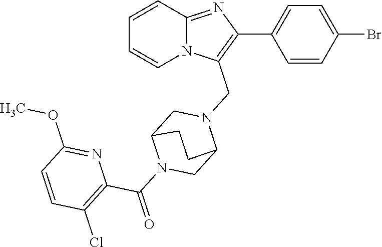 [SMILES: COc1ccc(Cl)c(C(=O)N2CC3CCC2CN3Cc2c(-c3ccc(Br)cc3)nc3ccccn23)n1] | 2.92-3.00 (m, 1H), 3.20 (br. s, 0.5H), 3.35-3.46 (m, 1.25H), 3.71-3.85 (m, 3.75H), 4.18-4.35 (m, 2H), 4.38 (br. s, 0.25H), 6.85-7.02 (m, 2H), 7.27-7.35 (m, 1H), 7.56-7.70 (m, 3H), 7.72-7.94 (m, 3H), 8.53-8.62 (m, 1H). LC-MS (Method 2): Rₜ = 1.54 min; MS (ESIpos): m/z = 566/567/568/569 [M + H]⁺. |
| | from 2-(4-bromophenyl)-3-(2,5- | |
| | diazabicyclo[2.2.2]oct-2- | |
| | ylmethyl)imidazo[1,2-a]pyridine | |
| | dihydrochloride (racemate) and 3-chloro- | |
| | 6-methoxypyridine-2-carboxylic acid | |

### Example 200

### (5-{[2-(5-Chloropyridin-2-yl)imidazo[1,2-a]pyridin-3-yl]methyl}-2,5-diazabicyclo[2.2.2]oct-2-yl)[6-(difluoromethoxy)pyridin-2-yl]methanone (Enantiomer 1)

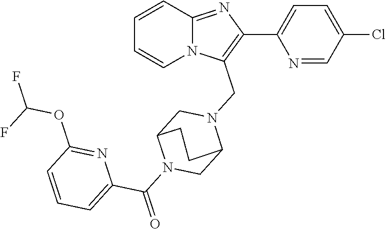 [SMILES: O=C(c1cccc(OC(F)F)n1)N1CC2CCC1CN2Cc1c(-c2ccc(Cl)cn2)nc2ccccn12]

45 mg (0.24 mmol) of 6-(difluoromethoxy)pyridine-2-carboxylic acid were dissolved in 1.5 ml of DMF, 123 mg (0.32 mmol) of 2-(7-aza-1H-benzotriazol-1-yl)-1,1,3,3-tetramethyluronium hexafluorophosphate (HATU) were added and the mixture was stirred at room temperature for 30 min. Subsequently, 100 mg of 2-{[2-(5-chloropyridin-2-yl)imidazo[1,2-a]pyridin-3-yl]methyl}-2,5-diazabicyclo[2.2.2]octane dihydrochloride (enantiomer 1) and 190 μl (1.08 mmol) of N,N-diisopropylethylamine were added and the mixture was stirred at room temperature overnight. Thereafter, the reaction mixture was separated into its components directly via preparative HPLC (Method 6). 79 mg (0.15 mmol, 70% of theory) of the title compound were obtained.

LC-MS (Method 2): Rₜ=1.29 min; m/z=525/527 [M+H]⁺.

¹H NMR (400 MHz, DMSO-d₆): δ [ppm]=1.51-2.08 (m, 4H), 2.73 (br. s, 0.25H), 2.85-2.94 (m, 1.5H), 2.98-3.10 (m, 1.25H), 3.38 (dd, 0.75H), 3.49 (d, 0.25H), 3.78-4.05 (m, 1.75H), 4.41 (br. s, 0.25H), 4.56-4.79 (m, 2H), 6.94-7.05 (m, 1H), 7.15-7.25 (m, 1H), 7.30-7.39 (m, 1.25H), 7.45-7.87 (m, 2.75H), 7.95-8.12 (m, 2H), 8.17-8.26 (m, 1H), 8.45 (d, 0.25H), 8.47-8.53 (m, 1H), 8.65 (d, 0.75H).

### B. ASSESSMENT OF PHARMACOLOGICAL EFFICACY

The pharmacological activity of the compounds of the invention can be demonstrated by in vitro and in vivo studies as known to the person skilled in the art. The application examples which follow describe the biological action of the compounds of the invention, without restricting the invention to these examples.

B-1. In Vitro Electrophysiological Analysis of the Human TASK-1 and TASK-3 Channels Via Two-Electrode Voltage Clamp Technique in Xenopus laevis Oocytes

Xenopus laevis oocytes were selected as described elsewhere by way of illustration [Decher et al., FEBS Lett. 492, 84-89 (2001)]. Subsequently, the oocytes were injected with 0.5-5 ng of a cRNA solution coding for TASK-1 or TASK-3. For the electrophysiological analysis of the channel proteins expressed in the oocytes, the two-electrode voltage clamp technique [Stühmer, Methods Enzymol. 207, 319-339 (1992)] was used. The measurements were conducted as described [Decher et al., FEBS Lett. 492, 84-89 (2001)] at room temperature (21-22° C.) using a Turbo TEC 10CD amplifier (NPI), recorded at 2 kHz and filtered with 0.4 kHz. Substance administration was performed using a gravitation-driven perfusion system. Here, the oocyte is located in a measuring chamber and exposed to the solution stream of 10 ml/min. The level in the measuring chamber is monitored and regulated by sucking off the solution using a peristaltic pump. Table 1 below shows the half-maximum inhibition, determined in this test, of human TASK-1 and TASK-3 channels (IC₅₀) by representative working examples of the invention:

TABLE 1

| Example | TASK-1 | TASK-3 |
|---|---|---|
| No. | IC₅₀ [nM] | IC₅₀ [nM] |
| 21 | 27.3 ± 5.4 | 16.3 ± 0.9 |
| 24 | | 31.2 ± 5.8 |
| 26 | 54.0 ± 16.1 | 12.0 ± 3.3 |
| 34 | 18.6 ± 4.3 | 10.3 ± 0.9 |
| 86 | 17.1 ± 3.5 | 58.4 ± 12.1 |
| 92 | 13.5 ± 1.2 | 5.9 ± 1.8 |
| 102 | 8.7 ± 3.6 | 4.3 ± 3.5 |
| 104 | 60.7 ± 12.0 | 4.2 ± 1.0 |
| 115 | 22.8 ± 3.7 | 186.2 ± 5.4 |
| 126 | 127.4 ± 8.5 | 714.2 ± 0.9 |

From the data in Table 1 it is evident that both TASK-1 and TASK-3 are blocked. The results in Table 1 thus confirm the mechanism of action of the compounds according to the invention as dual TASK-1/3 inhibitors.

B-2. Inhibition of Recombinant TASK-1 and TASK-3 In Vitro

The investigations on the inhibition of the recombinant TASK-1 and TASK-3 channels were conducted using stably transfected CHO cells. The compounds of the invention were tested here with administration of 40 mM of potassium chloride in the presence of a voltage-sensitive dye using the method described in detail in the following references [Whiteaker et al., Validation of FLIPR membrane potential dye for high-throughput screening of potassium channel modulators, J. Biomol. Screen. 6 (5), 305-312 (2001); Molecular Devices FLIPR Application Note: Measuring membrane potential using the FLIPR® membrane potential assay kit on Fluorometric Imaging Plate Reader (FLIPR®) systems, http://www.moleculardevices.com/reagents-supplies/assay-kits/ion-channels/flipr-membrane-potential-assay-kits]. The activity of the test substances was determined as their ability to inhibit a depolarization induced in the recombinant cells by 40 mM potassium chloride. The concentration which can block half of this depolarization is referred to as IC₅₀.

Table 2 below lists the IC₅₀ values from this assay determined for individual working examples of the invention (some as mean values from multiple independent individual determinations):

TABLE 2

| Example | TASK-1 | TASK-3 |
|---|---|---|
| No. | IC₅₀ [nM] | IC₅₀ [nM] |
| 4 | 560 | 43 |
| 5 | 200 | 150 |
| 6 | 394 | 410 |
| 10 | 1090 | 230 |
| 11 | 1400 | 940 |
| 12 | 3000 | 8300 |
| 14 | 5100 | 860 |
| 15 | 397 | 82 |
| 16 | 1300 | 600 |
| 21 | 240 | 60 |
| 22 | 170 | 13 |
| 23 | 790 | 170 |
| 24 | 140 | 4.5 |
| 25 | 120 | 5.6 |
| 26 | 690 | 42 |
| 29 | 460 | 50 |
| 30 | 770 | 21 |
| 31 | 310 | 26 |
| 32 | 270 | 9.6 |
| 33 | 140 | 26 |
| 34 | 855 | 84 |
| 35 | 350 | 200 |
| 36 | 670 | 250 |
| 37 | 350 | 63 |
| 38 | 790 | 380 |
| 39 | 640 | 370 |
| 40 | 533 | 104 |
| 41 | 76 | 3.9 |
| 42 | 880 | 17 |
| 43 | 320 | 8.9 |
| 44 | 130 | 3.3 |
| 45 | 340 | 13 |
| 46 | 73 | 5 |
| 47 | 70 | 15 |
| 48 | 390 | 17 |
| 49 | 740 | 12 |
| 51 | 290 | 34 |
| 52 | 170 | 10 |
| 53 | 140 | 34 |
| 54 | 470 | 76 |
| 55 | 2000 | 320 |
| 57 | 1400 | 240 |
| 60 | | 610 |
| 61 | 680 | 87 |
| 62 | 2800 | 760 |
| 63 | 490 | 140 |
| 64 | 280 | 17 |
| 65 | 2000 | 460 |
| 66 | 1100 | 110 |
| 67 | 990 | 220 |
| 69 | 870 | 101 |
| 70 | 1900 | 160 |
| 71 | 130 | 24 |
| 72 | 2000 | 120 |
| 73 | 160 | 9.6 |
| 74 | 330 | 23 |
| 75 | 380 | 30 |
| 76 | 240 | 14 |
| 77 | 810 | 44 |
| 78 | 1600 | 65 |
| 79 | 1000 | 190 |
| 80 | 310 | 15 |
| 81 | 520 | 25 |
| 82 | 230 | 420 |
| 83 | 134 | 14 |
| 84 | 1100 | 400 |
| 85 | 520 | 53 |
| 86 | 331 | 126 |
| 87 | 583 | 160 |
| 88 | 766 | 210 |
| 89 | 827 | 220 |
| 90 | 958 | 79 |
| 91 | 1350 | 89 |
| 92 | 895 | 29 |
| 93 | 2300 | 510 |
| 94 | 530 | 130 |
| 95 | 1700 | 350 |
| 96 | 240 | 4.5 |
| 97 | 390 | 7.8 |
| 98 | 450 | 14 |
| 99 | 470 | 16 |
| 100 | 250 | 17 |
| 101 | 370 | 7.2 |
| 102 | 277 | 56 |
| 103 | 540 | 20 |
| 104 | 255 | 32 |
| 105 | 830 | 20 |
| 106 | 870 | 110 |
| 107 | 1000 | 33 |
| 108 | 1100 | 95 |
| 109 | 693 | 92 |
| 110 | 780 | 300 |
| 111 | 110 | 25 |
| 112 | 200 | 29 |
| 113 | 89 | 21 |
| 114 | 480 | 340 |
| 115 | 4240 | 2400 |
| 116 | 535 | 340 |
| 117 | 635 | 520 |
| 118 | 860 | 1000 |
| 119 | 1100 | 1500 |
| 120 | 1100 | 180 |
| 121 | 1700 | 2800 |
| 122 | 1100 | 120 |
| 123 | 2000 | 1600 |
| 126 | 17900 | 713 |
| 127 | 9450 | 670 |
| 135 | 4100 | 160 |
| 136 | 9300 | 44 |
| 137 | 190 | 14 |
| 138 | 75 | 6.8 |
| 139 | 290 | 18 |
| 140 | 160 | 21 |
| 141 | 250 | 59 |
| 142 | 240 | 98 |
| 143 | 130 | 14 |
| 144 | 180 | 27 |
| 145 | 280 | 27 |
| 146 | 920 | 340 |
| 147 | 12 | 15 |
| 148 | 23 | 2.7 |
| 149 | 3000 | 300 |
| 150 | 2700 | 210 |
| 151 | 72 | 1.8 |
| 152 | 2900 | 75 |
| 153 | 1000 | 34 |
| 154 | 130 | 29 |
| 155 | 610 | 210 |
| 156 | 1200 | 15 |
| 157 | 24 | 7.4 |
| 158 | 760 | 110 |
| 159 | 2900 | 220 |
| 160 | 7600 | 1000 |
| 161 | 7700 | 1000 |
| 162 | 53 | 13 |
| 163 | 1400 | 160 |
| 164 | 3100 | 1000 |
| 165 | 25 | |
| 166 | 990 | 44 |
| 167 | 3000 | 350 |
| 168 | 1400 | 130 |
| 169 | 3600 | 110 |
| 170 | 1200 | 64 |
| 171 | 1800 | 26 |
| 172 | 5200 | 80 |
| 173 | 1700 | 32 |
| 174 | 91 | 13 |
| 175 | 43 | 200 |
| 176 | 49 | 10 |
| 177 | 4000 | 130 |
| 178 | 290 | 17 |
| 179 | 270 | 2.9 |
| 180 | 220 | 23 |
| 181 | 4100 | 1000 |
| 182 | 6100 | 1000 |
| 183 | 450 | 26 |
| 184 | 2900 | 72 |
| 185 | 120 | |
| 186 | 730 | 44 |
| 187 | 3800 | 200 |
| 188 | 60 | 5.9 |
| 189 | 8300 | 520 |
| 190 | 170 | 19 |
| 191 | 9500 | 190 |
| 192 | 870 | 73 |
| 193 | 7100 | 100 |
| 194 | 3000 | 290 |
| 195 | 230 | 8.1 |
| 196 | 440 | 52 |
| 197 | 150 | 13 |
| 198 | 810 | 60 |
| 199 | 33 | 1.8 |
| 200 | 877 | 75 |

From the data in Table 2 it is evident that both TASK-1 and TASK-3 are blocked. The results in Table 2 thus confirm the mechanism of action of the compounds according to the invention as dual TASK-1/3 inhibitors.

B-3. Animal Model of Obstructive Sleep Apnoea in the Pig

Using negative pressure, it is possible to induce collapse and thus obstruction of the upper respiratory tract in anaesthetized, spontaneously breathing pigs [Wirth et al., Sleep 36, 699-708 (2013)].

German Landrace pigs are used for the model. The pigs are anaesthetized and tracheotomized. One cannula each is inserted into the rostral and the caudal part of the trachea. Using a T connector, the rostral cannula is connected on the one hand to a device generating negative pressures and on the other hand to the caudal cannula. Using a T connector, the caudal cannula is connected to the rostral cannula and to a tube which allows spontaneous breathing circumventing the upper respiratory tract. By appropriate closing and opening of the tubes it is thus possible for the pig to change from normal nasal breathing to breathing via the caudal cannula during the time when the upper respiratory tract is isolated and connected to the device for generating negative pressures. The muscle activity of the Musculus genioglossus is recorded by electromyogram (EMG).

At certain points in time, the collapsibility of the upper respiratory tract is tested by having the pig breathe via the caudal cannula and applying negative pressures of −50, −100 and −150 cm water head (cm H₂O) to the upper respiratory tract. This causes the upper respiratory tract to collapse, which manifests itself in an interruption of the airflow and a pressure drop in the tube system. This test is conducted prior to the administration of the test substance and at certain intervals after the administration of the test substance. An appropriately effective test substance can prevent this collapse of the respiratory tract in the inspiratory phase.

After changeover from nasal breathing to breathing via the caudal cannula, it is not possible to measure any EMG activity of the Musculus genioglossus in the anaesthetized pig. As a further test, the negative pressure at which EMG activity restarts is then determined. This threshold value is, if a test substance is effective, shifted to more positive values. The test is likewise conducted prior to the administration of the test substance and at certain intervals after the administration of the test substance. Administration of the test substance can be intranasal, intravenous, subcutaneous, intraperitoneal or intragastral.

B-4. In Vitro Electrophysiological Analysis of the Washout Rate of Compounds after Binding to the Human TASK-1 Channel Via Two-Electrode Voltage Clamp Technique in Xenopus laevis Oocytes

Xenopus laevis oocytes were obtained from animals that had been anaesthetized with tricaine. Ovaries were treated with collagenase (1 mg/ml, Worthington, type II), stored in OR2 solution (82.5 mM NaCl, 2 mM KCl, 1 mM MgCl₂, 5 mM HEPES; pH 7.4) for 120 min and then kept in the ND96 standard solution (96 mM NaCl, 2 mM KCl, 1.8 mM CaCl₂), 1 mM MgCl₂, 5 mM HEPES; pH 7.5) with additional sodium pyruvate (275 mg/1), theophylline (90 mg/1) and gentamicin (50 mg/1) at 18° C. hTASK-1 and hTASK-3 were subcloned into the pSGEM vector, and cRNA was produced after linearization with NHEI and in vitro transcription with T7 polymerase. Oocytes were injected individually with 5-ng of cRNA solution that encodes hTASK-1. Standard two-electrode voltage clamp recordings [Stühmer, Methods Enzymol. 207, 319-339 (1992)] were conducted at room temperature (21-22° C.) with a Turbo-TEC-10CD amplifier (NPI) as described above [Decher et al., FEBS Lett. 492, 84-89 (2001)]. The measurement interval was 2 kHz, and the data were filtered at 0.4 kHz. Substances were applied in a gravity-driven manner via the bath solution, using ND96. In summary, Xenopus laevis oocytes were selected as described above, injected with TASK-1 cRNA and subjected to an electrophysiological analysis via the two-electrode voltage clamp technique.

The TASK-1 channels were inhibited beforehand by a value of about 40% by administration of one of the compounds of the invention. The concentrations shown in Table 3 below were established here, which had been ascertained beforehand by determining the IC₅₀ values in question. Subsequently, the restoration of the TASK-1-related membrane current was recorded in the voltage clamp over at least one hour. This restoration is caused by the washout of the compound in question from the TASK-1 channel.

At least 6 oocytes were examined for every compound. The voltage clamp measurements took a total of at least 1.5 hours (administration of the inhibitor plus at least one subsequent hour of washout measurement). Oocytes that showed leaks during the measurement were discarded; the results shown in Table 3 only included those oocytes that were stable over the entire measurement.

TABLE 3

| Example | Concentration | Proportion of | Washout rate |
|---|---|---|---|
| No. | [nM] | measurements used | [% h⁻¹] |
| 21 | 30 | 6/6 | 40 |
| 26 | 60 | 6/7 | 17 |
| 29 | 45 | 5/6 | 17 |
| 33 | 80 | 5/6 | 55 |

### C. WORKING EXAMPLES OF PHARMACEUTICAL COMPOSITIONS

The compounds of the invention can be converted to pharmaceutical preparations as follows:

Tablet:

Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

Production:

The mixture of compound of the invention, lactose and starch is granulated with a 5% solution (w/w) of the PVP in water. The granules are dried and then mixed with the magnesium stearate for 5 minutes. This mixture is compressed using a conventional tableting press (see above for format of the tablet). The guide value used for the pressing is a pressing force of 15 kN.

Suspension for Oral Administration:

Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

Production:

The Rhodigel is suspended in ethanol; the compound of the invention is added to the suspension. The water is added while stirring. The mixture is stirred for about 6 h until the swelling of the Rhodigel is complete.

Solution for Oral Administration:

Composition:

500 mg of the compound of the invention, 2.5 g of polysorbate and 97 g of polyethylene glycol 400. 20 g of oral solution correspond to a single dose of 100 mg of the compound of the invention.

Production:

The compound of the invention is suspended in the mixture of polyethylene glycol and polysorbate with stirring. The stirring operation is continued until dissolution of the compound of the invention is complete.

i.v. Solution:

The compound of the invention is dissolved in a concentration below the saturation solubility in a physiologically acceptable solvent (e.g. isotonic saline solution, glucose solution 5% and/or PEG 400 solution 30%). The solution is subjected to sterile filtration and dispensed into sterile and pyrogen-free injection vessels.

Solution for Nasal Administration:

The compound of the invention is dissolved in a concentration below the saturation solubility in a physiologically acceptable solvent (e.g. purified water, phosphate buffer, citrate buffer). The solution may contain further additives for isotonization, for preservation, for adjusting the pH, for improvement in the solubility and/or for stabilization.

1: A compound of formula (I) 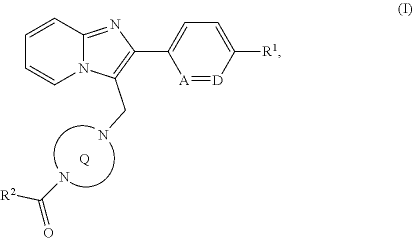
wherein
the ring Q is a diazaheterobicyclic system of the formula 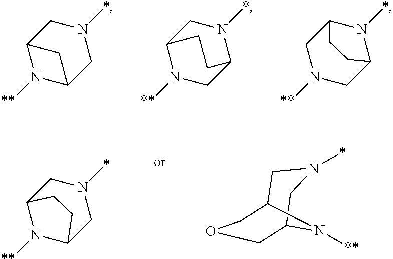
wherein * denotes the bond to the adjoining methylene group and ** the bond to the carbonyl group;
A and D are each CH or one of these ring members is CH and the other is N,
R¹ is halogen, cyano, (C₁-C₄)-alkyl, cyclopropyl, or cyclobutyl,
wherein said (C₁-C₄)-alkyl may be up to trisubstituted by fluorine and said cyclopropyl or said cyclobutyl may be up to disubstituted by fluorine;
and
R² is (C₄-C₆)-cycloalkyl, wherein a ring CH₂ group may be replaced by —O—;
or
R² is a phenyl group of the formula (a), a pyridyl group of the formula (b) or (c), or an azole group of the formula (d); 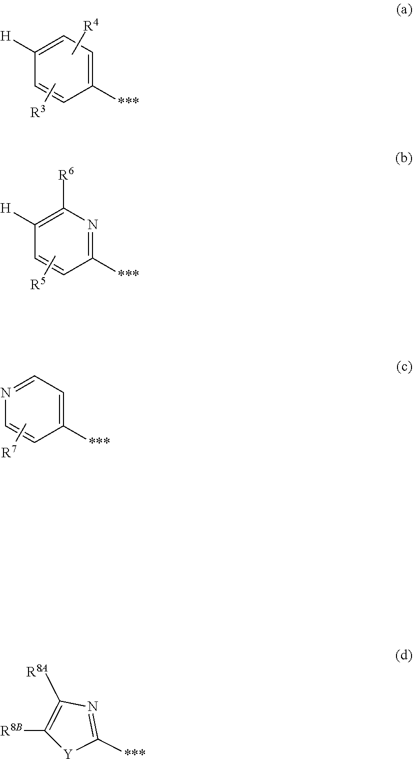
wherein *** marks the bond to the adjacent carbonyl group, and wherein
R³ is hydrogen, fluorine, chlorine, bromine, or methyl;
R⁴ is hydrogen, fluorine, chlorine, bromine, cyano, (C₁-C₃)-alkyl, or (C₁-C₃)-alkoxy,
wherein said (C₁-C₃)-alkyl or said (C₁-C₃)-alkoxy may be up to trisubstituted by fluorine;
R⁵ is hydrogen, fluorine, chlorine, bromine, or methyl;
R⁶ is hydrogen, (C₁-C₃)-alkoxy, cyclobutyloxy, oxetan-3-yloxy, tetrahydrofuran-3-yloxy, or tetrahydro-2H-pyran-4-yloxy,
wherein said (C₁-C₃)-alkoxy may be up to trisubstituted by fluorine;
R⁷ is hydrogen, fluorine, chlorine, bromine, (C₁-C₃)-alkyl or (C₁-C₃)-alkoxy;
R^{8A} and R^{8B} are the same or different and are independently hydrogen or (C₁-C₃)-alkyl;
Y is N(R⁹), O or S, wherein
R⁹ is hydrogen or (C₁-C₃)-alkyl;
or
R² is an —OR¹⁰ or —NR¹¹R¹² group, wherein
R¹⁰ is (C₁-C₄)-alkyl or (C₄-C₆)-cycloalkyl;
R¹¹ is hydrogen or (C₁-C₃)-alkyl;
R¹² is (C₁-C₆)-alkyl, (C₃-C₆)-cycloalkyl, phenyl, or benzyl,
wherein said (C₁-C₆)-alkyl may be up to trisubstituted by fluorine,
and
wherein said phenyl r the phenyl group in said benzyl may be up to disubstituted, identically or differently, by a radical selected from the group consisting of fluorine, chlorine, methyl, ethyl, and trifluoromethyl;
or
R¹¹ and R¹² are taken together with the nitrogen atom to which they are bonded to form a pyrrolidine, piperidine, morpholine, or thiomorpholine ring,
or a salt, a solvate, or solvate of a salt thereof.

2: A compound of formula (I) according to claim 1, wherein
the ring Q is a diazaheterobicyclic system of the formula 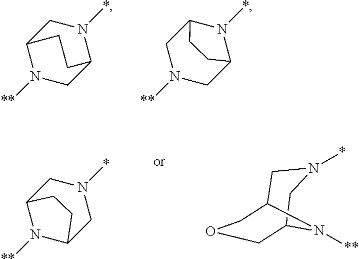
wherein * denotes the bond to the adjoining methylene group and ** the bond to the carbonyl group;
A and D are each CH or one of these ring members is CH and the other is N;
R¹ is fluorine, chlorine, bromine, methyl, isopropyl, tert-butyl, cyclopropyl, or cyclobutyl;
R² is cyclobutyl, cyclopentyl, or cyclohexyl;
or
R² is a phenyl group of the formula (a), a pyridyl group of the formula (b), or an azole group of the formula (d); 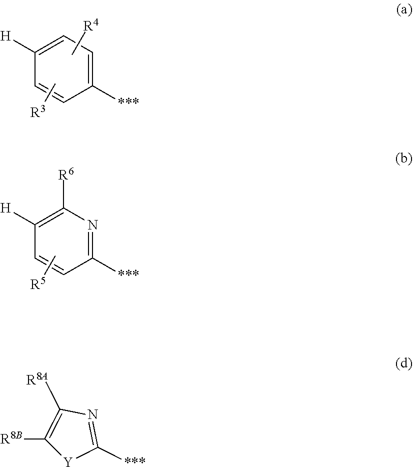
wherein *** marks the bond to the adjacent carbonyl group, and wherein
R³ is hydrogen, fluorine, or chlorine;
R⁴ is fluorine, chlorine, cyano, (C₁-C₃)-alkyl, (C₁-C₃)-alkoxy, or trifluoromethoxy;
R⁵ is hydrogen, fluorine, chlorine, bromine, or methyl;
R⁶ is (C₁-C₃)-alkoxy which may be up to trisubstituted by fluorine, or cyclobutyloxy;
R^{8A} and R^{8B} are independently hydrogen or methyl;
Y is N(CH₃), O or S;
or
R² is a —NR¹¹R¹² group, wherein
R¹¹ is hydrogen or (C₁-C₃)-alkyl;
R¹² is (C₁-C₆)-alkyl or phenyl,
wherein said phenyl may be up to disubstituted, identically or differently, by a radical selected from the group consisting of fluorine, chlorine, methyl, ethyl, and trifluoromethyl;
or
R¹¹ and R¹² are taken together with the nitrogen atom to which they are bonded to form a pyrrolidine, piperidine, or thiomorpholine ring,
or a salt, a solvate, or a solvate of a salt thereof.

3: A compound of formula (I) according to claim 1, wherein
the ring Q is a diazaheterobicyclic system of the formula 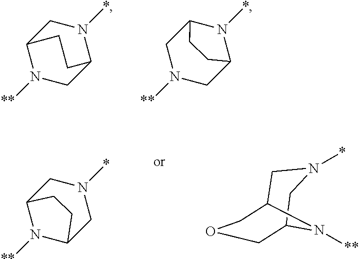
wherein * denotes the bond to the adjoining methylene group and ** the bond to the carbonyl group;
A is CH or N;
D is CH;
R¹ is fluorine, chlorine, bromine, methyl, isopropyl, tert-butyl, cyclopropyl, or cyclobutyl;
R² is cyclobutyl, cyclopentyl, or cyclohexyl;
or
R² is a phenyl group of the formula (a), a pyridyl group of the formula (b), or an azole group of the formula (d); 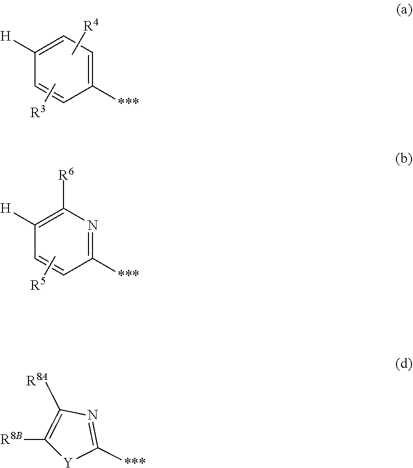
wherein *** marks the bond to the adjacent carbonyl group, and wherein
R³ is hydrogen, fluorine, or chlorine;
R⁴ is fluorine, chlorine, cyano, (C₁-C₃)-alkyl, (C₁-C₃)-alkoxy, or trifluoromethoxy;
R⁵ is hydrogen, fluorine, chlorine, bromine, or methyl;
R⁶ is (C₁-C₃)-alkoxy which may be up to trisubstituted by fluorine, or cyclobutyloxy;
R^{8A} and R^{8B} are independently hydrogen or methyl;
Y is N(CH₃), O or S;
or
R² is a —NR¹¹R¹² group, wherein
R¹¹ is hydrogen or (C₁-C₃)-alkyl;
R¹² is (C₁-C₆)-alkyl or phenyl,
wherein said phenyl may be up to disubstituted, identically or differently, by a radical selected from the group consisting of fluorine, chlorine, methyl, ethyl and trifluoromethyl;
or
R¹¹ and R¹² are taken together with the nitrogen atom to which they are bonded to form a pyrrolidine, piperidine, or thiomorpholine ring,
or a salt, a solvate, or a solvate of a salt thereof.

4: A compound of formula (I) according to claim 1, wherein
the ring Q is a diazaheterobicyclic system of the formula 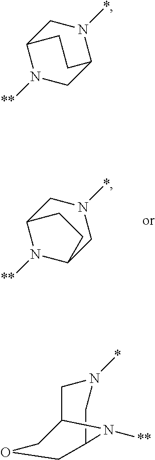
wherein * denotes the bond to the adjoining methylene group and ** the bond to the carbonyl group;
A and D are each CH or one of these ring members is CH and the other is N;
R¹ is chlorine, bromine, isopropyl, or cyclopropyl;
R² is cyclobutyl, cyclopentyl, or cyclohexyl;
or
R² is a phenyl group of the formula (a), a pyridyl group of the formula (b), or an azole group of the formula (d); 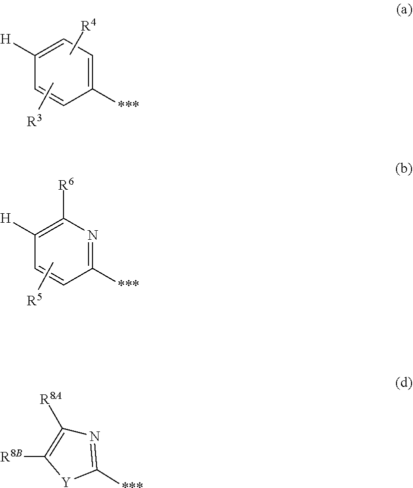
wherein *** marks the bond to the adjacent carbonyl group, and wherein
R³ is hydrogen, fluorine, or chlorine;
R⁴ is fluorine, chlorine, methyl, isopropyl, methoxy, or ethoxy;
R⁵ is hydrogen, fluorine, chlorine, bromine, or methyl;
R⁶ is methoxy, difluoromethoxy, trifluoromethoxy, isopropoxy, or cyclobutyloxy;
R^{8A} and R^{8B} are each hydrogen;
and
Y is N(CH₃),
or a salt, a solvate, or a solvate of a salt thereof.

5: A compound of formula (I) according to claim 1, wherein
the ring Q is a diazaheterobicyclic system of the formula 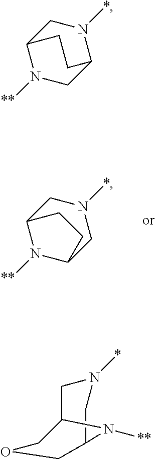
wherein * denotes the bond to the adjoining methylene group and ** the bond to the carbonyl group;
A is CH or N;
D is CH;
R¹ is chlorine, bromine, isopropyl, or cyclopropyl;
R² is cyclobutyl, cyclopentyl, or cyclohexyl;
or
R² is a phenyl group of the formula (a), a pyridyl group of the formula (b), or an azole group of the formula (d); 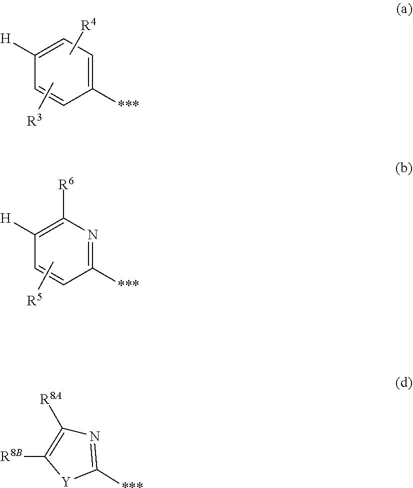
wherein *** marks the bond to the adjacent carbonyl group, and wherein
R³ is hydrogen, fluorine, or chlorine;
R⁴ is fluorine, chlorine, methyl, isopropyl, methoxy, or ethoxy;
R⁵ is hydrogen, fluorine, chlorine, bromine, or methyl;
R⁶ is methoxy, difluoromethoxy, trifluoromethoxy, isopropoxy, or cyclobutyloxy;
R^{8A} and R^{8B} are each hydrogen;
and
Y is N(CH₃),
or a salt, a solvate, or a solvate of a salt thereof.

6: A process for preparing a compound of formula (I) according to claim 1, comprising:
reacting a compound of formula (II) 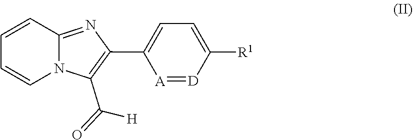
wherein A, D and R¹ are as defined in claim 1,
in the presence of a suitable reducing agent with either
[A] a compound of formula (III) 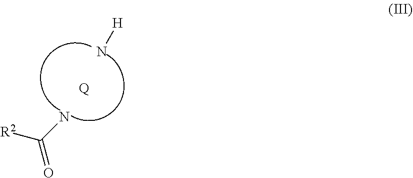
wherein R² and the ring Q are as defined in claim 1,
to give a compound of formula (I)
or
[B] w a protected diazaheterobicyclic system of formula (IV) 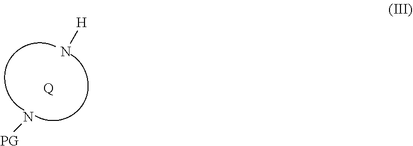
wherein the ring Q is as defined in claim 1, and
PG is a suitable amino protecting group, f,
at first to give a compound of formula (V) 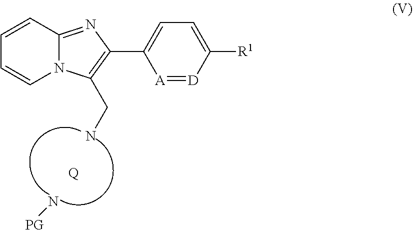
wherein A, D, PG, R¹ and the ring Q are as defined above,
then removing the protecting group PG to give a compound of formula (VI) 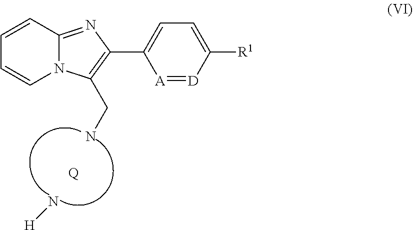
wherein A, D, R¹ and the ring Q are as defined above;
then depending on the specific definition of the R² radical, reacting the compound of formula (VI) with either:
[B-1] a carboxylic acid of formula (VII) 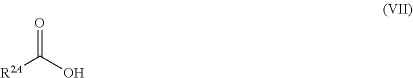
wherein
R^{2A} is (C₄-C₆)-cycloalkyl, wherein a ring CH₂ group may be replaced by —O—, or
R^{2A} is a phenyl group of the formula (a), a pyridyl group of the formula (b) or (c), or an azole group of the formula (d) as defined in claim 1,
with activation of the carboxylic acid function in (VII), or
the corresponding acid chloride of the formula (VIII) 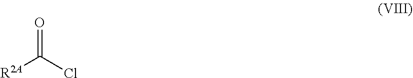
wherein R^{2A} is as defined above,
to give a compound of formula (I-A) 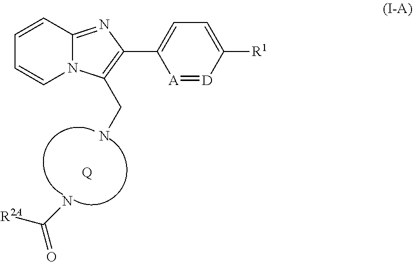
wherein A, D, R¹, R^{2A} and the ring Q are as defined above,
or
[B-2] a chloroformate or carbamoyl chloride of formula (IX) 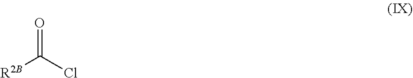
wherein
R^{2B} is an —OR¹⁰ or —NR^{11A}R¹² group, wherein
R¹⁰ and R¹² are as defined in claim 1,
and
R^{11A} is as defined in claim 1 but is not hydrogen,
to give a compound of formula (I-B) 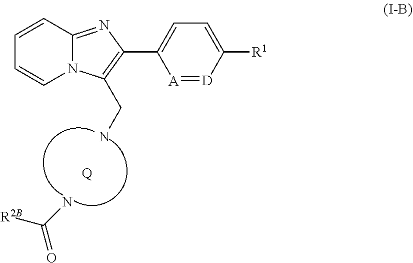
wherein A, D, R¹, R^{2B} and the ring Q are as defined above:
or
[B-3] an isocyanate of formula (X)
R¹²—N═C═O (X)
wherein R¹² is as defined in claim 1,
to give a compound of formula (I-C) 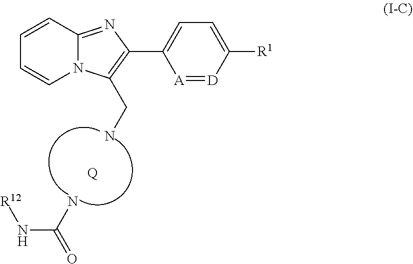
wherein A, D, R¹, R¹² and the ring Q are as defined above,
and
optionally separating the compound of formulae (I), (I-A), (I-B) or (I-C) thus obtained into the enantiomers or diastereomers thereof,
and/or
optionally converting the compound of formulae (I), (I-A), (I-B) or (I-C) with appropriate (i) solvents and/or (ii) acids to a solvate, a salt, or a solvate of a salt thereof.

7: A method of discovering a compound having TASK-1- and/or TASK-3-blocking properties, comprising subjecting at least one compound to at least one assay selected from the group consisting of:
determining the inhibitory concentration (IC₅₀) in relation to the K⁺ conductivity of a TASK-1 or TASK-3 channel;
determining the washout rate;
and
determining the maximum possible bioavailability after administration (“Fₘₐₓ well-stirred”);
and optionally at least one further assay selected from the group consisting of:
determining the brain/plasma concentration ratio C_{br}/Cₚ;
determining c Log D [pH 7.5] and/or c Log P and/or tPSA;
determining the selectivity for TASK-1 and/or TASK-3 with respect to other K⁺ channels;
determining passive apparent permeability (cPAPP, passive);
and
determining blood clearance (CL_{blood}).

8: A method for preparing a pharmaceutical formulation comprising a compound having TASK-1- and/or TASK-3-blocking properties and suitability for nasal administration, comprising:
producing and/or providing a library of compounds,
testing at least one compound from this library in an assay according to claim 7,
isolating at least one compound after this step,
and optionally
converting the at least one compound to a pharmaceutical formulation suitable for nasal administration.

9: The method according to claim 7, wherein the compound has to fulfil at least one of the conditions fixed in the following group:
a) the inhibitory concentration (IC₅₀) in relation to the K⁺ conductivity of the TASK-1 or TASK-3 channel is ≤200 nM, measured by the two-electrode voltage clamp technique (TEVC) in Xenopus laevis oocytes that have been injected with TASK-1 cRNA or TASK-3 cRNA;
b) the washout rate is ≤50% h¹, measured by the two-electrode voltage clamp technique (TEVC) in Xenopus laevis oocytes that have been injected with TASK-1 cRNA or TASK-3 cRNA;
c) the maximum possible bioavailability (“Fₘₐₓ well-stirred”) is ≤40%, measured by means of the hepatocyte in vitro clearance test described herein;
d) the brain/plasma concentration ratio C_{br}/Cₚ is ≤1, measured after nasal and/or intravenous administration of the compound to rats and subsequent LC-MS/MS analysis of processed plasma and brain tissue samples;
e) c Log D [pH 7.5] is between ≥2.5 and ≤5;
f) c Log P is between ≥1 and ≤5;
g) tPSA is between ≥25 and ≤100 Å²;
h) the inhibitory concentration (IC₅₀) relating to the K⁺ conductivity of the TASK-1 or TASK-3 channel is at least 10³ times less than that relating to the cardiac hERG K⁺ channel, measured by the two-electrode voltage clamp technique (TEVC) in Xenopus laevis oocytes;
i) cPAPP, passive is ≥100, measured in Caco-2 cells based on the determination of apparent permeability (PAPP);
j) blood clearance (CL_{blood}) is ≥60% of the species-specific liver perfusion; or
k) oral bioavailability is ≤40%, expressed as the quotient of AUC_{standard} (peroral administration)/AUC_{standard} (intravenous administration).

10: The method according to claim 7, wherein the compound is
suitable for the prevention or treatment of obstructive sleep apnoea (OSA) or of one or more symptoms associated therewith,
suitable for nasal administration
and/or
brings about inhibition of the collapsibility of the upper respiratory tract in a pig model for OSA, where the duration of inhibition of the collapsibility of the upper respiratory tract in the OSA pig model, preferably after intranasal administration of between 0.3 and 300 μg of the compound, is more than 240 min, measured at a reduced pressure of 100 cm water column.

11: A compound having TASK-1- and/or TASK-3-blocking properties, obtainable by the method according to claim 7, wherein the compound has at least one functional feature selected from the group consisting of:
a) the inhibitory concentration (IC₅₀) relating to the K⁺ conductivity of the TASK-1 or TASK-3 channel is ≤200 nM;
b) the washout rate is ≤50% h⁻¹; and
c) the maximum possible bioavailability (“Fₘₐₓ well-stirred”) is ≤40%;
and optionally has at least one of the further features according to claim 7.

12: A compound according to claim 11, wherein the compound is an (imidazo[1,2-a]pyridin-3-yl)methyl-substituted diazaheterobicyclic compound and/or a compound with the proviso that the compounds disclosed in EP patent application 15199270.8 and in EP patent application 15199268.2 are not included.

13: A compound according to claim 11, wherein the washout rate of the compound is preferably ≤40% h⁻¹.

14: A compound that competes with a compound according to claim 11 for interaction with TASK-1 and/or TASK-3, wherein the term “interaction” relates to at least one feature from the group consisting of:
reduction in the K⁺ conductivity of the TASK-1 or TASK-3 channel, and
binding to one or more epitopes and/or domains of TASK-1 and/or TASK-3.

15: A method for treatment or prevention of respiratory disorders, sleep-related respiratory disorders, obstructive sleep apnoea, central sleep apnoea, snoring, cardiac arrhythmias, neurodegenerative disorders, neuroinflammatory disorders or neuroimmunological disorders, comprising administering an effective amount of a compound according to claim 1 to a patient in need thereof.

16: A method for treatment or prevention respiratory disorders, sleep-related respiratory disorders, obstructive sleep apnoea, central sleep apnoea, snoring, cardiac arrhythmias, neurodegenerative disorders, neuroinflammatory disorders or neuroimmunological disorders, comprising administering an effective amount of a compound according to claim 11 to a patient in need thereof.

17: (canceled)

18: A pharmaceutical composition comprising a compound according to claim 1 in combination with one or more inert, nontoxic, pharmaceutically suitable excipients.

19: A pharmaceutical combination comprising a compound according to claim 1 in combination with one or more further active ingredients selected from the group consisting of respiratory stimulants, psychostimulating compounds, serotonin reuptake inhibitors, noradrenergic, serotonergic and tricyclic antidepressants, sGC stimulators, mineralocorticoid receptor antagonists, antiinflammatory drugs, immunomodulators, immunosuppressants and cytotoxic drugs.

20: A method for treatment or prevention of respiratory disorders, sleep-related respiratory disorders, obstructive sleep apnoea, central sleep apnoea, snoring, cardiac arrhythmias, neurodegenerative disorders, neuroinflammatory disorders or neuroimmunological disorders, comprising administering an effective amount of a pharmaceutical composition according to claim 18 to a patient in need thereof.

21: Me A method for treatment or prevention of respiratory disorders, sleep-related respiratory disorders, obstructive sleep apnoea, central sleep apnoea, snoring, cardiac arrhythmias, neurodegenerative disorders, neuroinflammatory disorders or neuroimmunological disorders in humans and animals, comprising administering an effective amount of at least one compound according to claim 1.

22: A method for treatment or prevention of respiratory disorders, sleep-related respiratory disorders, obstructive sleep apnoea, central sleep apnoea, snoring, cardiac arrhythmias, neurodegenerative disorders, neuroinflammatory disorders or neuroimmunological disorders, comprising administering an effective amount of a pharmaceutical combination according to claim 19 to a patient in need thereof.
